(19) United States
(12) Patent Application Publication
(10) Pub. No.: US 2003/0104530 A1
(43) Pub. Date: Jun. 5, 2003

(54) Human sodium-hydrogen exchanger like protein 1

(76) Inventor: Yizhong Gu, Cupertino, CA (US)

Correspondence Address: Stephen G. Ryan, Amersham Biosciences, 800 Centennial Avenue, Piscataway, NJ, 08855, US

(21) Appl. No.: 10/060,998

(22) Filed: Jan. 30, 2002

Related U.S. Application Data
(63) Continuation-in-part of application No. 09/864,761, filed on May 23, 2001.
(60) Provisional application No. 60/343,331, filed on Dec. 21, 2001.

(30) Foreign Application Priority Data
Jan. 30, 2001 (WO) PCT/US01/00666

(51) Int. Cl. C07K 14/435; C07H 21/04; C12P 21/02; C12N 5/06

(52) U.S. Cl. 435/69.1; 435/325; 435/320.1; 530/350; 536/23.2

(57) ABSTRACT

The invention provides isolated nucleic acids that encode human sodium-hydrogen exchanger like protein 1 (NHELP1), and fragments thereof, vectors for propagating and expressing NHELP1 nucleic acids, host cells comprising the nucleic acids and vectors of the present invention, proteins, protein fragments, and protein fusions of the novel NHELP1 isoforms, and antibodies thereto. The invention further provides transgenic cells and non-human organisms comprising human NHELP1 nucleic acids, and transgenic cells and non-human organisms with targeted disruption of the endogenous orthologue of the human NHELP1 gene. The invention further provides pharmaceutical formulations of the nucleic acids, proteins, and antibodies of the present invention, and diagnostic, investigational, and therapeutic methods based on the NHELP1 nucleic acids, proteins, and antibodies of the present invention.

### CROSS REFERENCE TO RELATED APPLICATIONS

This application claims priority under 35 U.S.C. § 365(c) to international patent application no. PCT/US01/00666, filed Jan. 30, 2001; claims priority under 35 U.S.C. § 120 to commonly owned and copending U.S. application Ser. No. 09/864,761, filed May 23, 2001; claims priority to U.S. provisional application serial No. 60/343,331, filed Dec. 21, 2001; the disclosures of which are incorporated herein by reference in their entireties.

### REFERENCE TO SEQUENCE LISTING SUBMITTED ON COMPACT DISC

The present application includes a Sequence Listing filed on a single CD-R disc, provided in duplicate, containing a single file named pto_PB01108.txt, having 442 kilobytes, last modified on Jan. 23, 2002 and recorded Jan. 25, 2002. The Sequence Listing contained in said file on said disc is incorporated herein by reference in its entirety.

### FIELD OF THE INVENTION

The present invention relates to novel human sodium-hydrogen exchanger like protein 1 (NHELP1). More specifically, the invention provides isolated nucleic acid molecules encoding NHELP1, fragments thereof, vectors and host cells comprising isolated nucleic acid molecules encoding NHELP1, NHELP1 polypeptides, antibodies, transgenic cells and non-human organisms, and diagnostic, therapeutic, and investigational methods of using the same.

### BACKGROUND OF THE INVENTION

The luminal ionic composition of many, if not all, intracellular compartments differs from the surrounding cytoplasm and is an important determinant of their function. The establishment of this differential composition is achieved through the concerted actions of distinct integral membrane ion carriers, including pumps, channels, and transporters. For example, alkalinization of the mitochondrial matrix, driven by the respiratory chain, contributes to the inner membrane H⁺ gradient used to drive ATP synthesis (Saraste, Science 283:1488-1493 (1999)) and, indirectly, to extrude matrix Ca²⁺ through the functional coupling of Na⁺/H⁺ and Na⁺/Ca²⁺ antiport pathways (Garlid et al., Methods Enzymol. 260:331-348 (1995); Brierley et al., J. Bioenerg. Biomembr. 26:519-526 (1994); Babcock et al., J. Cell Biol. 136:833-844 (1997)).

Organelles of the secretory and endocytic pathways are distinguished by their luminal acidity, which is generated by the activity of a vacuolar-type H⁺-ATPase (V-ATPase). Mellman et al., Annu. Rev. Biochem. 55:663-700 (1986); Forgac, J. Biol. Chem. 274:12951-12954 (1999). At present, little is known about the mechanisms controlling the steady-state [H⁺] within the lumen of different endomembrane compartments. Although distinct isoforms of some of the V-ATPase subunits have been reported in different tissues (Puopolo et al., J. Biol. Chem. 267:3696-3706 (1992); Nishi and Forgac, J. Biol. Chem. 275:6824-6830 (2000)) or specialized cell types (Gluck, J. Exp. Biol. 172:29-37 (1992)), there is no clear evidence that the V-ATPase functions differently in particular organelles within a single mammalian cell. Rather, the luminal [H⁺] is thought to be regulated by a complex interplay between the V-ATPase and unidentified leak pathways for protons, based on the rapid dissipation of the transmembrane proton chemical gradient (DpH) observed after inhibiting the V-ATPase with macrolide antibiotics. Demaurex et al., J. Biol. Chem. 273:2044-2051 (1998); Farinas and verkman, J. Biol. Chem. 274:7603-7606 (1999); Kim et al., J. Cell Biol. 134:1387-1399 (1996).

A few components of this leak pathway have been identified recently. For example, one such component in the Golgi complex was recently identified as a Zn²⁺-inhibitable H⁺ conductance. Schapiro and Grinstein, J. Biol. Chem. 275:21025-21032 (2000). Several Na+/H+ exchangers (NHEs) have also been identified which are involved in the controlling of luminal cation composition of various cellular organelles. Orlowski and Grinstein, J. Biol. Chem. 272:22373-22376 (1997); Yun et al., Am. J. Physiol. 269:G1-G11 (1995); Numata and Orlowski, J. Biol. Chem. 276:17387-17394 (2001). These findings highlights the H⁺ leak as a key determinant of organellar pH and emphasizes the need to identify additional molecular components of this pathway.

To date, seven NHE isoforms have been identified. These isoforms exhibit considerable differences in their primary structures, tissue distribution, membrane localization, biochemical and pharmacological properties, and responsiveness to various stimuli. Numata et al., J. Biol. Chem. 273:6951-6959 (1998); Numata and Orlowski, J. Biol. Chem. 276:17387-17394 (2001). Mammalian NHEs participate in a wide array of other essential cellular processes, including control of intracellular pH, maintenance of cellular volume, and reabsorption of Na⁺ across renal, intestinal, and other epithelia. Activation of NHE activity also facilitates growth factor-induced proliferation of certain cell types (Grinstein et al., Biochim. Biophys. Acta 988:73-97 (1989)) and is associated with events leading to apoptosis. Rajotte et al., J. Biol. Chem. 267:9980-9987 (1992); Li and Eastman, J. Biol. Chem. 270:3203-3211 (1995); Zhu and Loh, Biochim. Biophys. Acta 1269:122-128 (1995).

Imbalance of the luminal ionic composition could lead to severe consequences. Studies have shown that V-ATPases are involved in the development of drug resistance to breast cancer treatments. Martinez-Zaguilan et al., Biochem. Pharm. 57:1037-1046 (1999). The overexpression of some V-ATPases may also play a crucial role in tumor progression as it is characteristic of invasive pancreatic tumors. Ohta et al, Br. J. Cancer 73:1511-1517 (1996). Another series of recent studies have implicated the endocytotic role of V-ATPase domains in the development of acquired immunodeficiency syndrome (AIDS). Lu et al., Immunity 8:647-656 (1998); Mandic et al., Mol. Biol. Cell 12:463-473 (2001).

Given the roles of NHE in maintaining cation ion homeostasis and normal cellular function, there is a need to identify and to characterize additional human proteins with structural and functional similarities to NHE.

### SUMMARY OF THE INVENTION

The present invention solves these and other needs in the art by providing isolated nucleic acids that encode NHELP1, and fragments thereof.

In other aspects, the invention provides vectors for propagating and expressing the nucleic acids of the present invention, host cells comprising the nucleic acids and vectors of the present invention, proteins, protein fragments, and protein fusions of the NHELP1, and antibodies thereto.

The invention further provides pharmaceutical formulations of the nucleic acids, proteins, and antibodies of the present invention.

In other aspects, the invention provides transgenic cells and non-human organisms comprising NHELP1 nucleic acids, and transgenic cells and non-human organisms with targeted disruption of the endogenous orthologue of the NHELP1.

The invention additionally provides diagnostic, investigational, and therapeutic methods based on the NHELP1 nucleic acids, proteins, and antibodies of the present invention.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1(a) schematizes the protein domain structure of NHELP1 and FIG. 1(b) shows the alignment of the Na_H_Exchanger dommain with that of other proteins;

FIG. 2 is a map showing the genomic structure of NHELP1 encoded at chromosome 3q23; and

FIG. 3 presents the nucleotide and predicted amino acid sequences of NHELP1.

### DETAILED DESCRIPTION OF THE INVENTION

Mining the sequence of the human genome for novel human genes, the present inventors have identified a human sodium-hydrogen exchanger like protein (NHELP1), which functions in maintaining cation ion homeostasis of a cellular organelle.

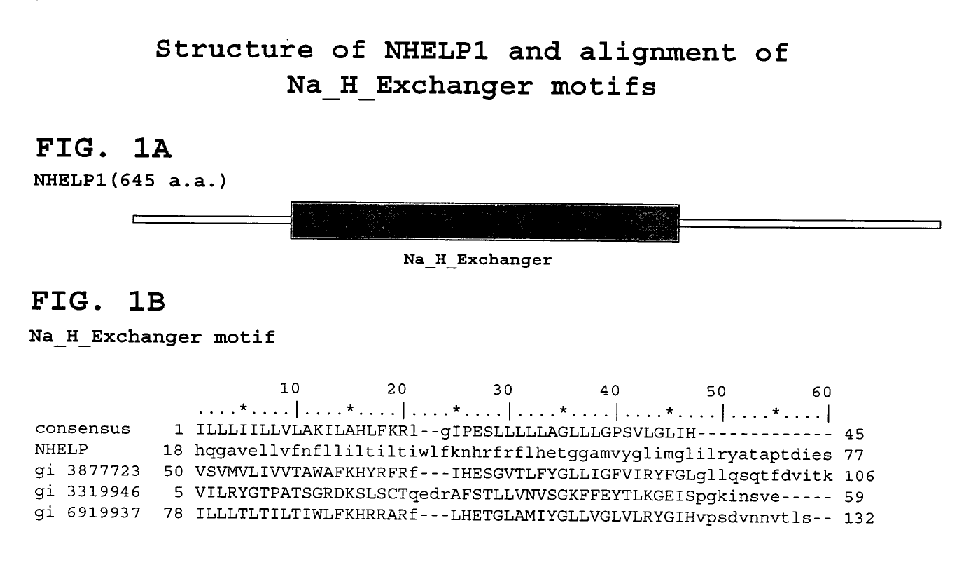

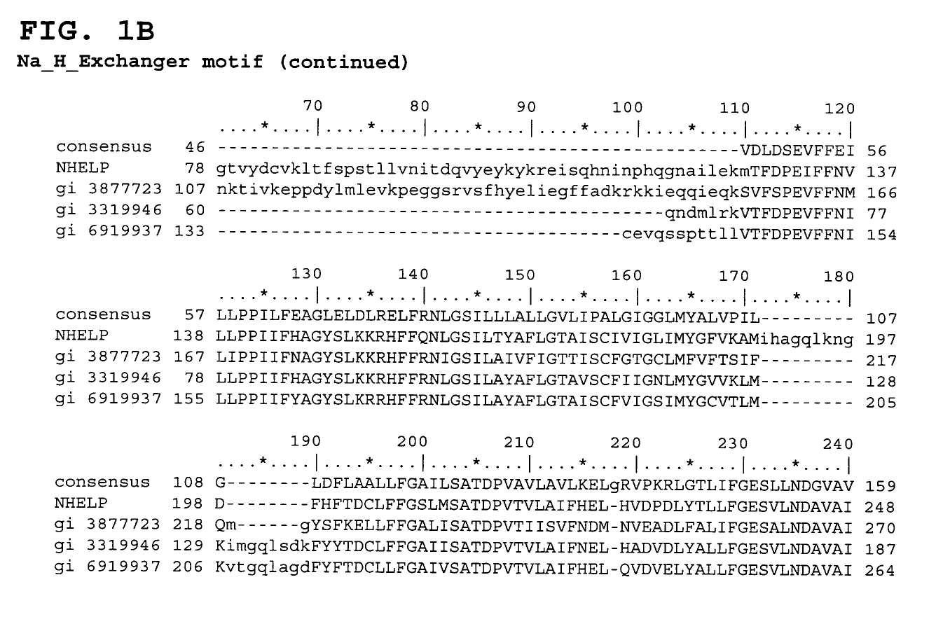

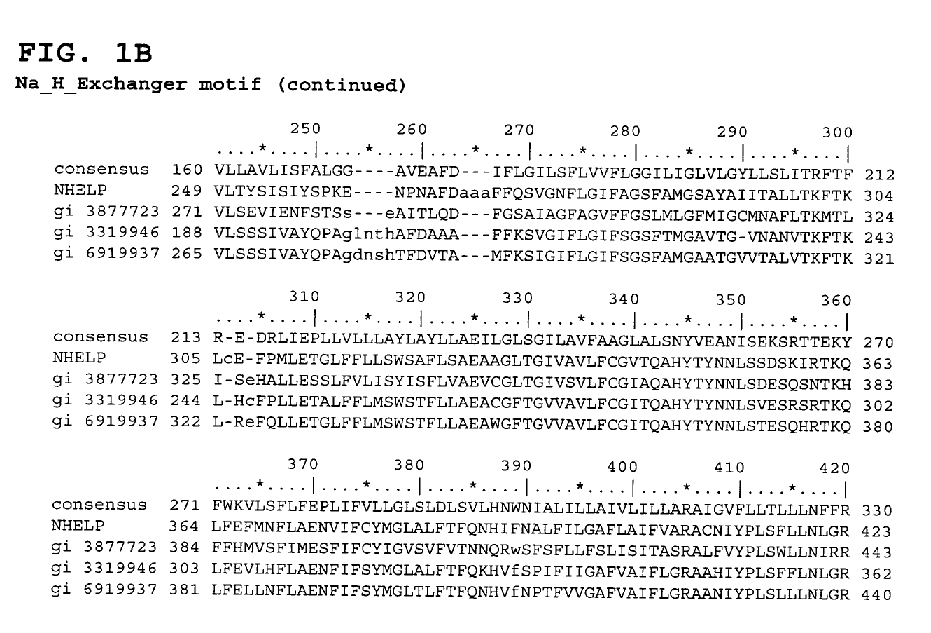

As schematized in FIG. 1, the newly isolated gene product shares certain protein domains and an overall structural organization with other human sodium/hydrogen exchangers. The shared structural features strongly imply that NHELP1 plays a role similar to that of other human sodium/hydrogen exchangers in maintaining cation ion homeostasis.

Like the other human sodium/hydrogen exchanger isoforms, NHELP1 contains a Na_H_Exchanger domain. In NHELP1, the Na_H_Exchanger motif ocurrs at amino acids 128-454 (http://www.ncbi.nlm.nih.gov/Structure/cdd/wrpsb.cgi/). Other signatures of the newly isolated NHELP1 proteins were identified by searching the PROSITE database (http://www.expasy.ch/tools/scnpsit1.html). These include three N-glycosylation sites (96-99, 352-355, 516-519), four protein kinase C phosphorylation sites (9-11, 149-151, 258-260 and 522-524), one tyrosine kinase phosphorylation site (6-14), a leucine zipper (311-332), a amidation site, seven Casein kinase II phosphorylation sites, and eleven N-myristoylation sites.

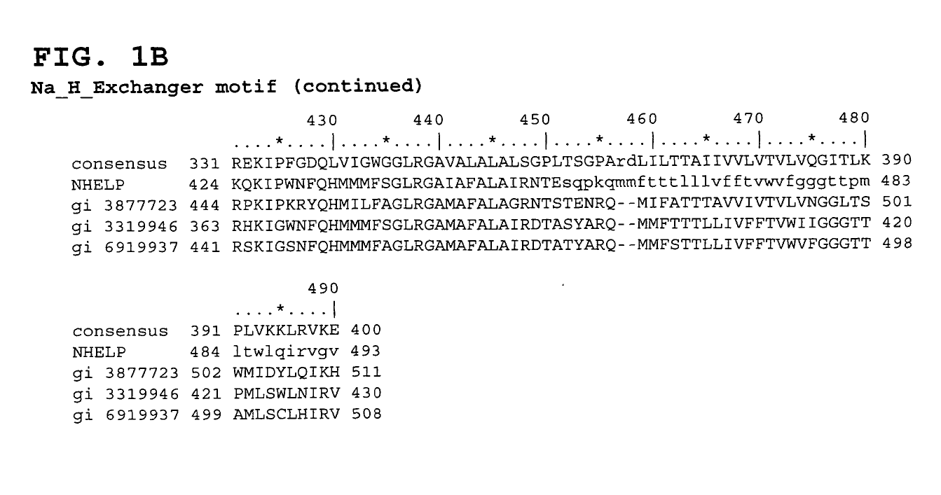

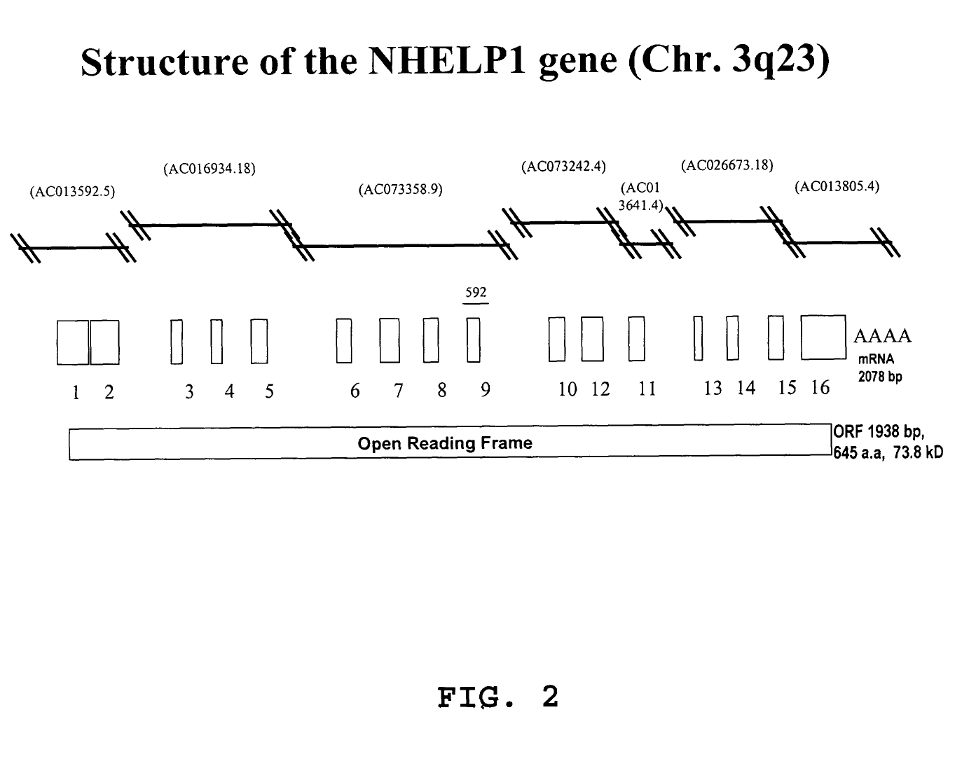

FIG. 2 shows the genomic organization of NHELP1.

At the top is shown the seven bacterial artificial chromosomes (BACs), with GenBank accession numbers, that span the NHELP1 locus. The genome-derived single-exon probe first used to demonstrate expression from this locus is shown below the BACs and labeled “592”. The 592 bp probe includes sequence drawn from exon 9 and surrounding introns.

As shown in FIG. 2, NHELP1, encoding a protein of 645 amino acids, comprises exons 1-16. The predicted molecular weights, prior to any post translational modification, is 72.6 kD. The cDNA clone appears full length, with the open reading frame starting with a methionine and terminating with a stop codon.

As further discussed in the examples herein, expression of NHELP1 was assessed using hybridization to genome-derived single exon microarrays. Microarray analysis of exon nine showed expression in all tissues tested, including brain, adult liver, fetal liver, adrenal, bone marrow, prostate, testis as well as a cell line, hela.

As more fully described below, the present invention provides isolated nucleic acids that encode NHELP1 and fragments thereof. The invention further provides vectors for propagation and expression of the nucleic acids of the present invention, host cells comprising the nucleic acids and vectors of the present invention, proteins, protein fragments, and protein fusions of the present invention, and antibodies specific for all or any one of the isoforms. The invention provides pharmaceutical formulations of the nucleic acids, proteins, and antibodies of the present invention. The invention further provides transgenic cells and non-human organisms comprising human NHELP1 nucleic acids, and transgenic cells and non-human organisms with targeted disruption of the endogenous orthologue of the human NHELP1. The invention additionally provides diagnostic, investigational, and therapeutic methods based on the NHELP1 nucleic acids, proteins, and antibodies of the present invention.

### DEFINITIONS

Unless defined otherwise, all technical and scientific terms used herein have the meaning commonly understood by one of ordinary skill in the art to which this invention belongs.

As used herein, “nucleic acid” (synonymously, “polynucleotide”) includes polynucleotides having natural nucleotides in native 51-31 phosphodiester linkage—e.g., DNA or RNA—as well as polynucleotides that have nonnatural nucleotide analogues, nonnative internucleoside bonds, or both, so long as the nonnatural polynucleotide is capable of sequence-discriminating basepairing under experimentally desired conditions. Unless otherwise specified, the term “nucleic acid” includes any topological conformation; the term thus explicitly comprehends single-stranded, double-stranded, partially duplexed, triplexed, hairpinned, circular, and padlocked conformations.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment.

For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature.

As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

As used herein, an isolated nucleic acid “encodes” a reference polypeptide when at least a portion of the nucleic acid, or its complement, can be directly translated to provide the amino acid sequence of the reference polypeptide, or when the isolated nucleic acid can be used, alone or as part of an expression vector, to express the reference polypeptide in vitro, in a prokaryotic host cell, or in a eukaryotic host cell.

As used herein, the term “exon” refers to a nucleic acid sequence found in genomic DNA that is bioinformatically predicted and/or experimentally confirmed to contribute contiguous sequence to a mature mRNA transcript.

As used herein, the phrase “open reading frame” and the equivalent acronym “ORF” refer to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence intends all nucleic acid sequences that can be directly translated, using the standard genetic code, to provide an amino acid sequence identical to that translated from the reference nucleic acid sequence.

As used herein, the term “microarray” and the equivalent phrase “nucleic acid microarray” refer to a substrate-bound collection of plural nucleic acids, hybridization to each of the plurality of bound nucleic acids being separately detectable. The substrate can be solid or porous, planar or non-planar, unitary or distributed.

As so defined, the term “microarray” and phrase “nucleic acid microarray” include all the devices so called in Schena (ed.), DNA Microarrays: A Practical Approach (Practical Approach Series), Oxford University Press (1999) (ISBN: 0199637768); Nature Genet. 21(1) (suppl):1-60 (1999); and Schena (ed.), Microarray Biochip: Tools and Technology, Eaton Publishing Company/BioTechniques Books Division (2000) (ISBN: 1881299376), the disclosures of which are incorporated herein by reference in their entireties.

As so defined, the term “microarray” and phrase “nucleic acid microarray” also include substrate-bound collections of plural nucleic acids in which the plurality of nucleic acids are distributably disposed on a plurality of beads, rather than on a unitary planar substrate, as is described, inter alia, in Brenner et al., Proc. Natl. Acad. Sci. USA 97(4):166501670 (2000), the disclosure of which is incorporated herein by reference in its entirety; in such case, the term “microarray” and phrase “nucleic acid microarray” refer to the plurality of beads in aggregate.

As used herein with respect to solution phase hybridization, the term “probe”, or equivalently, “nucleic acid probe” or “hybridization probe”, refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to nucleic acid intended to be bound to probe by sequence complementarity.

As used herein, the expression “probe comprising SEQ ID NO: X”, and variants thereof, intends a nucleic acid probe, at least a portion of which probe has either (i) the sequence directly as given in the referenced SEQ ID NO: X, or (ii) a sequence complementary to the sequence as given in the referenced SEQ ID NO: X, the choice as between sequence directly as given and complement thereof dictated by the requirement that the probe be complementary to the desired target.

As used herein, the phrases “expression of a probe” and “expression of an isolated nucleic acid” and their linguistic equivalents intend that the probe or, (respectively, the isolated nucleic acid), or a probe (or, respectively, isolated nucleic acid) complementary in sequence thereto,can hybridize detectably under high stringency conditions to a sample of nucleic acids that derive from mRNA transcripts from a given source. For example, and by way of illustration only, expression of a probe in “liver” means that the probe can hybridize detectably under high stringency conditions to a sample of nucleic acids that derive from mRNA obtained from liver.

As used herein, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and that consist of one or more exons that are found adjacent to the reference exon in the genome.

For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for at least 8 hours, followed by one or more washes in 0.2×SSC, 0.1% SDS at 65° C. “Moderate stringency conditions” are defined for solution phase hybridization as aqueous hybridization (i.e., free of formamide) in 6×SSC, 1% SDS at 65° C. for at least 8 hours, followed by one or more washes in 2×SSC, 0.1% SDS at room temperature.

For microarray-based hybridization, standard “high stringency conditions” are defined as hybridization in 50% formamide, 5×SSC, 0.2 μg/μl poly(dA), 0.2 μg/μl human cot1 DNA, and 0.5% SDS, in a humid oven at 42° C. overnight, followed by successive washes of the microarray in 1×SSC, 0.2% SDS at 55° C. for 5 minutes, and then 0.1×SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds. The terms “protein”, “polypeptide”, and “peptide” explicitly permits of post-translational and post-synthetic modifications, such as glycosylation.

The term “oligopeptide” herein denotes a protein, polypeptide, or peptide having 25 or fewer monomeric subunits.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment.

For example, a protein can be said to be “isolated” when it includes amino acid analogues or derivatives not found in nature, or includes linkages other than standard peptide bonds.

When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

A “purified protein” (equally, a purified polypeptide, peptide, or oligopeptide) is an isolated protein, as above described, present at a concentration of at least 95%, as measured on a weight basis with respect to total protein in a composition. A “substantially purified protein” (equally, a substantially purified polypeptide, peptide, or oligopeptide) is an isolated protein, as above described, present at a concentration of at least 70%, as measured on a weight basis with respect to total protein in a composition.

As used herein, the phrase “protein isoforms” refers to a plurality of proteins having nonidentical primary amino acid sequence but that share amino acid sequence encoded by at least one common exon.

As used herein, the phrase “alternative splicing” and its linguistic equivalents includes all types of RNA processing that lead to expression of plural protein isoforms from a single gene; accordingly, the phrase “splice variant(s)” and its linguistic equivalents embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region (“5′ UT”) and alternatives in the 3′ untranslated region (“3′ UT”). Such 3′ alternatives include, for example, differences in the site of RNA transcript cleavage and site of poly(A) addition. See, e.g., Gautheret et al., Genome Res. 8:524-530 (1998).

As used herein, “orthologues” are separate occurrences of the same gene in multiple species. The separate occurrences have similar, albeit nonidentical, amino acid sequences, the degree of sequence similarity depending, in part, upon the evolutionary distance of the species from a common ancestor having the same gene.

As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the term “homologues” is generic to “orthologues” and “paralogues”.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

As used herein, antibodies can be produced by any known technique, including harvest from cell culture of native B lymphocytes, harvest from culture of hybridomas, recombinant expression systems, and phage display.

As used herein, “antigen” refers to a ligand that can be bound by an antibody; an antigen need not itself be immunogenic. The portions of the antigen that make contact with the antibody are denominated “epitopes”.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, and biotin to any of avidin, streptavidin, neutrAvidin and captAvidin.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “portion”, as used with respect to nucleic acids, proteins, and antibodies, is synonymous with “fragment”.

### NUCLEIC ACID MOLECULES

In a first aspect, the invention provides isolated nucleic acids that encode NHELP1, variants having at least 65% sequence identity thereto, degenerate variants thereof, variants that encode NHELP1 proteins having conservative or moderately conservative substitutions, cross-hybridizing nucleic acids, and fragments thereof.

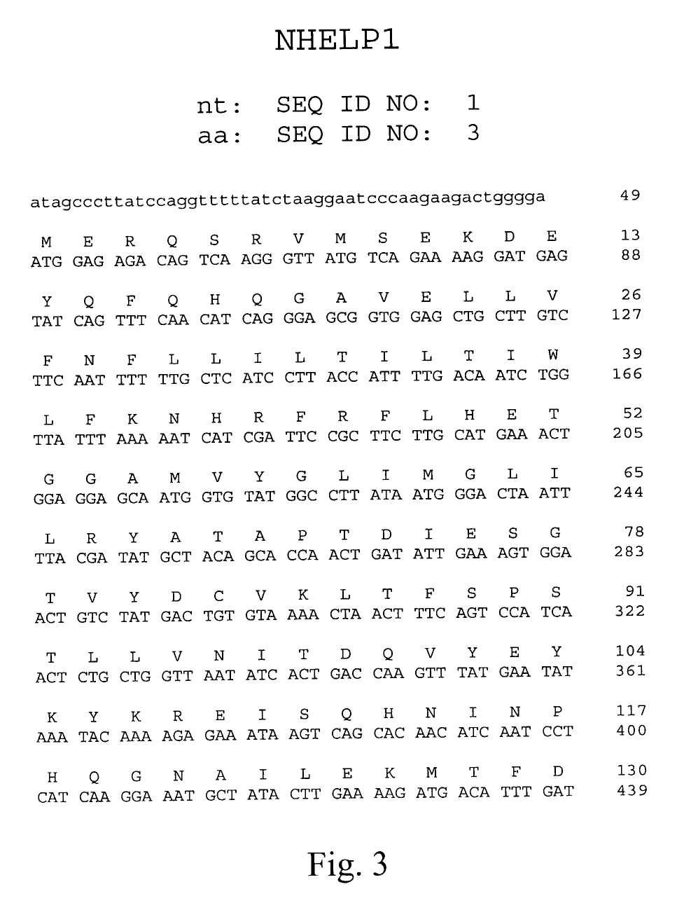

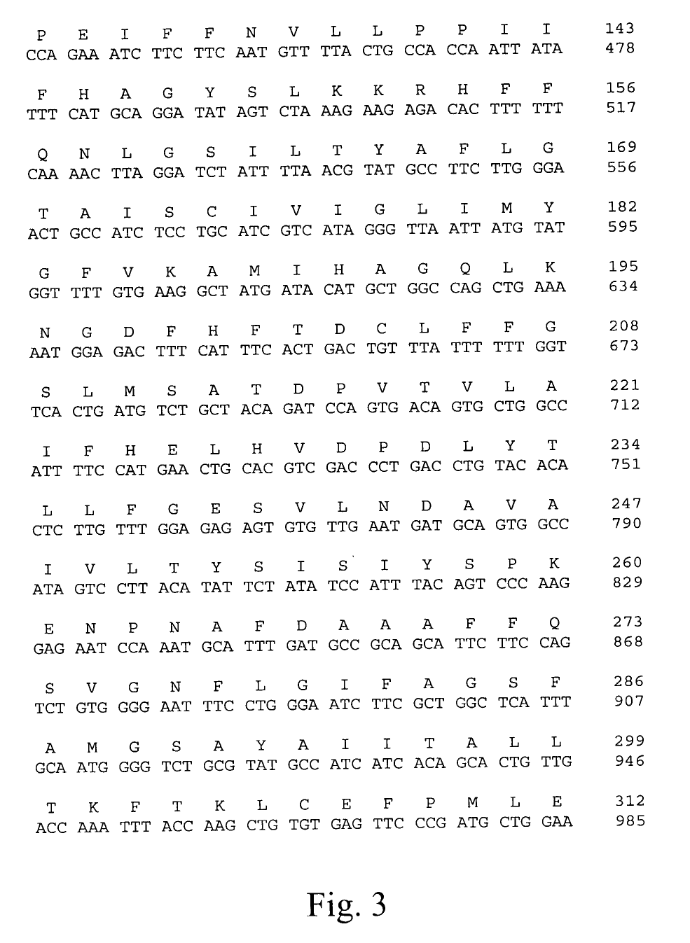

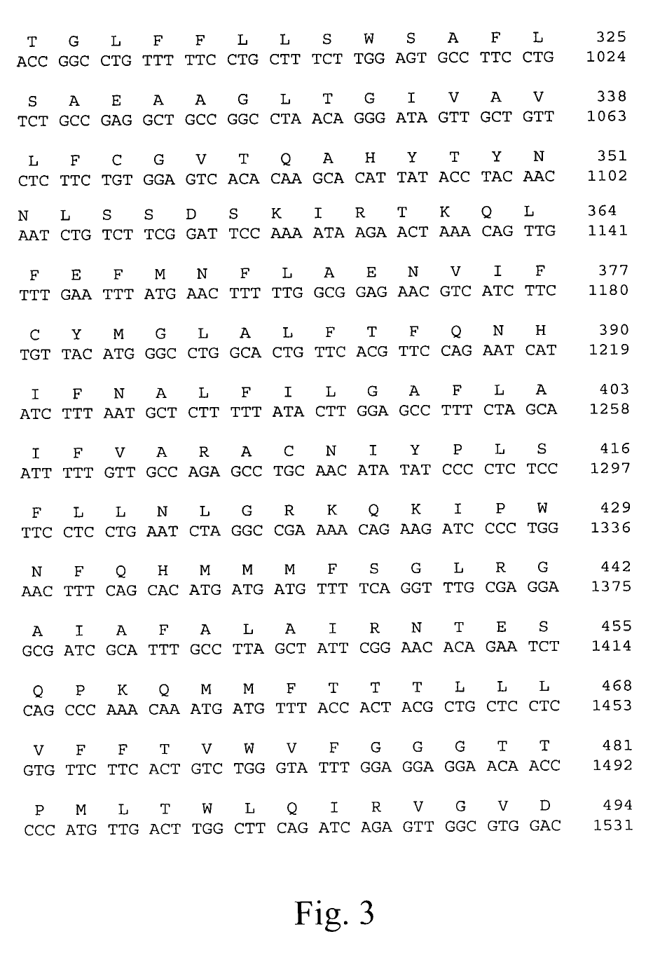

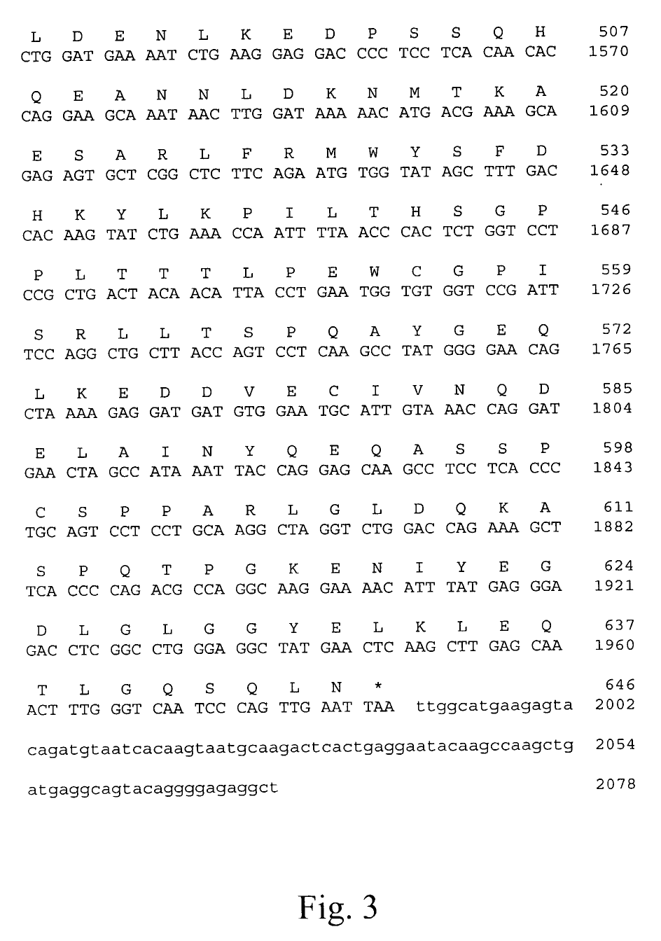

FIG. 3 presents the nucleotide sequence of the NHELP1 cDNA clone, with predicted amino acid translation; the sequences are further presented in the Sequence Listing, incorporated herein by reference in its entirety, in SEQ ID NOs: 1 (full length nucleotide sequence of human NHELP1 cDNA) and 3 (full length amino acid coding sequence of human NHELP1).

Unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Unless otherwise indicated, nucleotide sequences of the isolated nucleic acids of the present invention were determined by sequencing a DNA molecule that had resulted, directly or indirectly, from at least one enzymatic polymerization reaction (e.g., reverse transcription and/or polymerase chain reaction) using an automated sequencer (such as the MegaBACE™ 1000, Amersham Biosciences, Sunnyvale, Calif., USA), or by reliance upon such sequence or upon genomic sequence prior-accessioned into a public database. Unless otherwise indicated, all amino acid sequences of the polypeptides of the present invention were predicted by translation from the nucleic acid sequences so determined.

As a consequence, any nucleic acid sequence presented herein may contain errors introduced by erroneous incorporation of nucleotides during polymerization, by erroneous base calling by the automated sequencer (although such sequencing errors have been minimized for the nucleic acids directly determined herein, unless otherwise indicated, by the sequencing of each of the complementary strands of a duplex DNA), or by similar errors accessioned into the public database. Such errors can readily be identified and corrected by resequencing of the genomic locus using standard techniques.

Single nucleotide polymorphisms (SNPs) occur frequently in eukaryotic genomes—more than 1.4 million SNPs have already identified in the human genome, International Human Genome Sequencing Consortium, Nature 409:860-921-(2001)—and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is an aspect of the present invention to provide nucleic acids not only identical in sequence to those described with particularity herein, but also to provide isolated nucleic acids at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

For purposes herein, percent identity of two nucleic acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at

http://www.ncbi.nlm.nih.gov/blast/bl2seq/bl2.html.

To assess percent identity of nucleic acids, the BLASTN module of BLAST 2 SEQUENCES is used with default values of (i) reward for a match: 1; (ii) penalty for a mismatch: −2; (iii) open gap 5 and extension gap 2 penalties; (iv) gap X_dropoff 50 expect 10 word size 11 filter, and both sequences are entered in their entireties.

As is well known, the genetic code is degenerate, with each amino acid except methionine translated from a plurality of codons, thus permitting a plurality of nucleic acids of disparate sequence to encode the identical protein. As is also well known, codon choice for optimal expression varies from species to species. The isolated nucleic acids of the present invention being useful for expression of NHELP1 proteins and protein fragments, it is, therefore, another aspect of the present invention to provide isolated nucleic acids that encode NHELP1 proteins and portions thereof not only identical in sequence to those described with particularity herein, but degenerate variants thereof as well.

As is also well known, amino acid substitutions occur frequently among natural allelic variants, with conservative substitutions often occasioning only de minimis change in protein function.

Accordingly, it is an aspect of the present invention to provide nucleic acids not only identical in sequence to those described with particularity herein, but also to provide isolated nucleic acids that encode NHELP1, and portions thereof, having conservative amino acid substitutions, and also to provide isolated nucleic acids that encode NHELP1, and portions thereof, having moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of nucleic acids can also be characterized using a functional test, the ability of the two nucleic acids to base-pair to one another at defined hybridization stringencies.

It is, therefore, another aspect of the invention to provide isolated nucleic acids not only identical in sequence to those described with particularity herein, but also to provide isolated nucleic acids (“cross-hybridizing nucleic acids”) that hybridize under high stringency conditions (as defined herein below) to all or to a portion of various of the isolated NHELP1 nucleic acids of the present invention (“reference nucleic acids”), as well as cross-hybridizing nucleic acids that hybridize under moderate stringency conditions to all or to a portion of various of the isolated NHELP1 nucleic acids of the present invention.

Such cross-hybridizing nucleic acids are useful, inter alia, as probes for, and to drive expression of, proteins related to the proteins of the present invention as alternative isoforms, homologues, paralogues, and orthologues. Particularly useful orthologues are those from other primate species, such as chimpanzee, rhesus macaque, monkey, baboon, orangutan, and gorilla; from rodents, such as rats, mice, guinea pigs; from lagomorphs, such as rabbits; and from domestic livestock, such as cow, pig, sheep, horse, goat and chicken.

For purposes herein, high stringency conditions are defined as aqueous hybridization (i.e., free of formamide) in 6×SSC (where 20×SSC contains 3.0 M NaCl and 0.3 M sodium citrate), 1% SDS at 65° C. for at least 8 hours, followed by one or more washes in 0.2×SSC, 0.1% SDS at 65° C. For purposes herein, moderate stringency conditions are defined as aqueous hybridization (i.e., free of formamide) in 6×SSC, 1% SDS at 65° C. for at least 8 hours, followed by one or more washes in 2×SSC, 0.1% SDS at room temperature.

The hybridizing portion of the reference nucleic acid is typically at least 15 nucleotides in length, often at least 17 nucleotides in length. Often, however, the hybridizing portion of the reference nucleic acid is at least 20 nucleotides in length, 25 nucleotides in length, and even 30 nucleotides, 35 nucleotides, 40 nucleotides, and 50 nucleotides in length. Of course, cross-hybridizing nucleic acids that hybridize to a larger portion of the reference nucleic acid—for example, to a portion of at least 50 nt, at least 100 nt, at least 150 nt, 200 nt, 250 nt, 300 nt, 350 nt, 400 nt, 450 nt, or 500 nt or more—or even to the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the isolated nucleic acids of the present invention.

By “fragments” of a reference nucleic acid is here intended isolated nucleic acids, however obtained, that have a nucleotide sequence identical to a portion of the reference nucleic acid sequence, which portion is at least 17 nucleotides and less than the entirety of the reference nucleic acid. As so defined, “fragments” need not be obtained by physical fragmentation of the reference nucleic acid, although such provenance is not thereby precluded.

In theory, an oligonucleotide of 17 nucleotides is of sufficient length as to occur at random less frequently than once in the three gigabase human genome, and thus to provide a nucleic acid probe that can uniquely identify the reference sequence in a nucleic acid mixture of genomic complexity. As is well known, further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

As further described herein below, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., “Use of peptide synthesis to probe viral antigens for epitopes to a resolution of a single amino acid,” Proc. Natl. Acad. Sci. USA 81:3998-4002 (1984); and U.S. Pat. Nos. 4,708,871 and 5,595,915, the disclosures of which are incorporated herein by reference in their entireties.

As further described herein below, fragments that encode at least 8 contiguous amino acids (i.e., fragments of 24 nucleotides or more) are useful in directing the expression or the synthesis of peptides that have utility as immunogens. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties.

The nucleic acid fragment of the present invention is thus at least 17 nucleotides in length, typically at least 18 nucleotides in length, and often at least 24 nucleotides in length. Often, the nucleic acid of the present invention is at least 25 nucleotides in length, and even 30 nucleotides, 35 nucleotides, 40 nucleotides, or 45 nucleotides in length. Of course, larger fragments having at least 50 nt, at least 100 nt, at least 150 nt, 200 nt, 250 nt, 300 nt, 350 nt, 400 nt, 450 nt, or 500 nt or more are also useful, and at times preferred.

Having been based upon the mining of genomic sequence, rather than upon surveillance of expressed message, the present invention further provides isolated genome-derived nucleic acids that include portions of the NHELP1 gene.

The invention particularly provides genome-derived single exon probes.

As further described in commonly owned and copending U.S. patent application Ser. Nos. 09/864,761, filed May 23, 2001; 09/774,203, filed Jan. 29, 2001; and 09/632,366, filed Aug. 3, 2000, the disclosures of which are incorporated herein by reference in their entireties, “a single exon probe,, comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and instead consist of one or more exons that are found adjacent to the reference exon in the genome.

Genome-derived single exon probes typically further comprise, contiguous to a first end of the exon portion, a first intronic and/or intergenic sequence that is identically contiguous to the exon in the genome. Often, the genome-derived single exon probe further comprises, contiguous to a second end of the exonic portion, a second intronic and/or intergenic sequence that is identically contiguous to the exon in the genome.

The minimum length of genome-derived single exon probes is defined by the requirement that the exonic portion be of sufficient length to hybridize under high stringency conditions to transcript-derived nucleic acids. Accordingly, the exon portion is at least 17 nucleotides, typically at least 18 nucleotides, 20 nucleotides, 24 nucleotides, 25 nucleotides or even 30, 35, 40, 45, or 50 nucleotides in length, and can usefully include the entirety of the exon, up to 100 nt, 150 nt, 200 nt, 250 nt, 300 nt, 350 nt, 400 nt or even 500 nt or more in length.

The maximum length of genome-derived single exon probes is defined by the requirement that the probes contain portions of no more than one exon, that is, be unable to hybridize detectably under high stringency conditions to nucleic acids that lack the reference exon but include one or more exons that are found adjacent to the reference exon the genome.

Given variable spacing of exons through eukaryotic genomes, the maximum length of single exon probes of the present invention is typically no more than 25 kb, often no more than 20 kb, 15 kb, 10 kb or 7.5 kb, or even no more than 5 kb, 4 kb, 3 kb, or even no more than about 2.5 kb in length.

The genome-derived single exon probes of the present invention can usefully include at least a first terminal priming sequence not found in contiguity with the rest of the probe sequence in the genome, and often will contain a second terminal priming sequence not found in contiguity with the rest of the probe sequence in the genome.

The present invention also provides isolated genome-derived nucleic acids that include nucleic acid sequence elements that control transcription of the NHELP1 gene.

With a complete draft of the human genome now available, genomic sequences that are within the vicinity of the NHELP1 coding region (and that are additional to those described with particularity herein) can readily be obtained by PCR amplification.

The isolated nucleic acids of the present invention can be composed of natural nucleotides in native 5′-3′ phosphodiester internucleoside linkage—e.g., DNA or RNA—or can contain any or all of nonnatural nucleotide analogues, nonnative internucleoside bonds, or post-synthesis modifications, either throughout the length of the nucleic acid or localized to one or more portions thereof.

As is well known in the art, when the isolated nucleic acid is used as a hybridization probe, the range of such nonnatural analogues, nonnative internucleoside bonds, or post-synthesis modifications will be limited to those that permit sequence-discriminating basepairing of the resulting nucleic acid. When used to direct expression or RNA or protein in vitro or in vivo, the range of such nonnatural analogues, nonnative internucleoside bonds, or post-synthesis modifications will be limited to those that permit the nucleic acid to function properly as a polymerization substrate. When the isolated nucleic acid is used as a therapeutic agent, the range of such changes will be limited to those that do not confer toxicity upon the isolated nucleic acid.

For example, when desired to be used as probes, the isolated nucleic acids of the present invention can usefully include nucleotide analogues that incorporate labels that are directly detectable, such as radiolabels or fluorophores, or nucleotide analogues that incorporate labels that can be visualized in a subsequent reaction, such as biotin or various haptens.

Common radiolabeled analogues include those labeled with ³³p, ³²p, and ³⁵S, such as α-³²P-dATP, α-³²P-dCTP, α-³²P-dGTP, α-³²P-dTTP, α-³²P-3′dATP, α-³²P-ATP, α-³²P-CTP, α-³²P-GTP, α-32P-UTP, α-³⁵S-DATP, γ-³⁵S-GTP, γ-³³P-DATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Biosciences, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA).

Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000), the disclosure of which is incorporated herein by reference in its entirety.

Haptens that are commonly conjugated to nucleotides for subsequent labeling include biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

As another example, when desired to be used for antisense inhibition of transcription or translation, the isolated nucleic acids of the present invention can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties. Such altered internucloside bonds are often desired also when the isolated nucleic acid of the present invention is to be used for targeted gene correction, Gamper et al., Nucl. Acids Res. 28(21):4332-4339 (2000), the disclosures of which are incorporated herein by reference in its entirety.

Modified oligonucleotide backbones often preferred when the nucleic acid is to be used for antisense purposes are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Preferred modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

In other preferred oligonucleotide mimetics, both the sugar and the internucleoside linkage are replaced with novel groups, such as peptide nucleic acids (PNA).

In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages.

The uncharged nature of the PNA backbone provides PNA/DNA and PNA/RNA duplexes with a higher thermal stability than is found in DNA/DNA and DNA/RNA duplexes, resulting from the lack of charge repulsion between the PNA and DNA or RNA strand. In general, the Tm of a PNA/DNA or PNA/RNA duplex is 1° C. higher per base pair than the Tm of the corresponding DNA/DNA or DNA/RNA duplex (in 100 mM NaCl).

The neutral backbone also allows PNA to form stable DNA duplexes largely independent of salt concentration. At low ionic strength, PNA can be hybridized to a target sequence at temperatures that make DNA hybridization problematic or impossible. And unlike DNA/DNA duplex formation, PNA hybridization is possible in the absence of magnesium. Adjusting the ionic strength, therefore, is useful if competing DNA or RNA is present in the sample, or if the nucleic acid being probed contains a high level of secondary structure.

PNA also demonstrates greater specificity in binding to complementary DNA. A PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater.

Additionally, nucleases and proteases do not recognize the PNA polyamide backbone with nucleobase sidechains. As a result, PNA oligomers are resistant to degradation by enzymes, and the lifetime of these compounds is extended both in vivo and in vitro. In addition, PNA is stable over a wide pH range.

Because its backbone is formed from amide bonds, PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference; automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.).

PNA chemistry and applications are reviewed, inter alia, in Ray et al., FASEB J. 14(9):1041-60 (2000); Nielsen et al., Pharmacol Toxicol. 86(1):3-7 (2000); Larsen et al., Biochim Biophys Acta. 1489(1):159-66 (1999); Nielsen, Curr. Opin. Struct. Biol. 9(3):353-7 (1999), and Nielsen, Curr. Opin. Biotechnol. 10(1):71-5 (1999), the disclosures of which are incorporated herein by reference in their entireties.

Differences from nucleic acid compositions found in nature—e.g., nonnative bases, altered internucleoside linkages, post-synthesis modification—can be present throughout the length of the nucleic acid or can, instead, usefully be localized to discrete portions thereof. As an example of the latter, chimeric nucleic acids can be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. As another example, chimeric nucleic acids comprising both DNA and PNA have been demonstrated to have utility in modified PCR reactions. See Misra et al., Biochem. 37: 1917-1925 (1998); see also Finn et al., Nucl. Acids Res. 24: 3357-3363 (1996), incorporated herein by reference.

Unless otherwise specified, nucleic acids of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Banér et al., Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994), the disclosures of which are incorporated herein by reference in their entireties. Triplex and quadruplex conformations, and their utilities, are reviewed in Praseuth et al., Biochim. Biophys. Acta. 1489(1):181-206 (1999); Fox, Curr. Med. Chem. 7(1):17-37 (2000); Kochetkova et al., Methods Mol. Biol. 130:189-201 (2000); Chan et al., J. Mol. Med. 75(4):267-82 (1997), the disclosures of which are incorporated herein by reference in their entireties.

The nucleic acids of the present invention can be detectably labeled.

Commonly-used labels include radionuclides, such as ³²P, ³³P, ³⁵S, ³H (and for NMR detection, ¹³C and ¹⁵N), haptens that can be detected by specific antibody or high affinity binding partner (such as avidin), and fluorophores.

As noted above, detectable labels can be incorporated by inclusion of labeled nucleotide analogues in the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach.

Analogues can also be incorporated during automated solid phase chemical synthesis.

As is well known, labels can also be incorporated after nucleic acid synthesis, with the 5′ phosphate and 3′ hydroxyl providing convenient sites for post-synthetic covalent attachment of detectable labels.

Various other post-synthetic approaches permit internal labeling of nucleic acids.

For example, fluorophores can be attached using a cisplatin reagent that reacts with the N7 of guanine residues (and, to a lesser extent, adenine bases) in DNA, RNA, and PNA to provide a stable coordination complex between the nucleic acid and fluorophore label (Universal Linkage System) (available from Molecular Probes, Inc., Eugene, Oreg., USA and Amersham Biosciences, Piscataway, N.J., USA); see Alers et al., Genes, Chromosomes & Cancer, Vol. 25, pp. 301-305 (1999); Jelsma et al., J. NIH Res. 5:82 (1994); Van Belkum et al., BioTechniques 16:148-153 (1994), incorporated herein by reference. As another example, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

Multiple independent or interacting labels can be incorporated into the nucleic acids of the present invention.

For example, both a fluorophore and a moiety that in proximity thereto acts to quench fluorescence can be included to report specific hybridization through release of fluorescence quenching, Tyagi et al., Nature Biotechnol. 14: 303-308 (1996); Tyagi et al., Nature Biotechnol. 16, 49-53 (1998); Sokol et al., Proc. Natl. Acad. Sci. USA 95: 11538-11543 (1998); Kostrikis et al., Science 279:1228-1229 (1998); Marras et al., Genet. Anal. 14: 151-156 (1999); U.S. Pat. Nos. 5,846,726, 5,925,517, 5925517, or to report exonucleotidic excision, U.S. Pat. No. 5,538,848; Holland et al., Proc. Natl. Acad. Sci. USA 88:7276-7280 (1991); Heid et al., Genome Res. 6(10):986-94 (1996); Kuimelis et al., Nucleic Acids Symp Ser. (37):255-6 (1997); U.S. Pat. No. 5,723,591, the disclosures of which are incorporated herein by reference in their entireties.

So labeled, the isolated nucleic acids of the present invention can be used as probes, as further described below.

Nucleic acids of the present invention can also usefully be bound to a substrate. The substrate can porous or solid, planar or non-planar, unitary or distributed; the bond can be covalent or noncovalent. Bound to a substrate, nucleic acids of the present invention can be used as probes in their unlabeled state.

For example, the nucleic acids of the present invention can usefully be bound to a porous substrate, commonly a membrane, typically comprising nitrocellulose, nylon, or positively-charged derivatized nylon; so attached, the nucleic acids of the present invention can be used to detect NHELP1 nucleic acids present within a labeled nucleic acid sample, either a sample of genomic nucleic acids or a sample of transcript-derived nucleic acids, e.g. by reverse dot blot.

The nucleic acids of the present invention can also usefully be bound to a solid substrate, such as glass, although other solid materials, such as amorphous silicon, crystalline silicon, or plastics, can also be used. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof.

Typically, the solid substrate will be rectangular, although other shapes, particularly disks and even spheres, present certain advantages. Particularly advantageous alternatives to glass slides as support substrates for array of nucleic acids are optical discs, as described in Demers, “Spatially Addressable Combinatorial Chemical Arrays in CD-ROM Format,” international patent publication WO 98/12559, incorporated herein by reference in its entirety.

The nucleic acids of the present invention can be attached covalently to a surface of the support substrate or applied to a derivatized surface in a chaotropic agent that facilitates denaturation and adherence by presumed noncovalent interactions, or some combination thereof.

The nucleic acids of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that include the nucleic acids of the present invention.

The isolated nucleic acids of the present invention can be used as hybridization probes to detect, characterize, and quantify NHELP1 nucleic acids in, and isolate NHELP1 nucleic acids from, both genomic and transcript-derived nucleic acid samples. When free in solution, such probes are typically, but not invariably, detectably labeled; bound to a substrate, as in a microarray, such probes are typically, but not invariably unlabeled.

For example, the isolated nucleic acids of the present invention can be used as probes to detect and characterize gross alterations in the NHELP1 genomic locus, such as deletions, insertions, translocations, and duplications of the NHELP1 genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acids of the present invention can be used as probes to isolate genomic clones that include the nucleic acids of the present invention, which thereafter can be restriction mapped and sequenced to identify deletions, insertions, translocations, and substitutions (single nucleotide polymorphisms, SNPs) at the sequence level.

The isolated nucleic acids of the present invention can also be used as probes to detect, characterize, and quantify NHELP1 nucleic acids in, and isolate NHELP1 nucleic acids from, transcript-derived nucleic acid samples.

For example, the isolated nucleic acids of the present invention can be used as hybridization probes to detect, characterize by length, and quantify NHELP1 mRNA by northern blot of total or poly-A⁺-selected RNA samples. For example, the isolated nucleic acids of the present invention can be used as hybridization probes to detect, characterize by location, and quantify NHELP1 message by in situ hybridization to tissue sections (see, e.g., Schwarchzacher et al., In Situ Hybridization, Springer-Verlag New York (2000) (ISBN: 0387915966), the disclosure of which is incorporated herein by reference in its entirety). For example, the isolated nucleic acids of the present invention can be used as hybridization probes to measure the representation of NHELP1 clones in a cDNA library. For example, the isolated nucleic acids of the present invention can be used as hybridization probes to isolate NHELP1 nucleic acids from cDNA libraries, permitting sequence level characterization of NHELP1 messages, including identification of deletions, insertions, truncations—including deletions, insertions, and truncations of exons in alternatively spliced forms—and single nucleotide polymorphisms.

All of the aforementioned probe techniques are well within the skill in the art, and are described at greater length in standard texts such as Sambrook et al., Molecular Cloning: A Laboratory Manual (3^{rd} ed.), Cold Spring Harbor Laboratory Press (2001) (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X); and Walker et al. (eds.), The Nucleic Acids Protocols Handbook, Humana Press (2000) (ISBN: 0896034593), the disclosures of which are incorporated herein by reference in their entirety.

As described in the Examples herein below, the nucleic acids of the present invention can also be used to detect and quantify NHELP1 nucleic acids in transcript-derived samples—that is, to measure expression of the NHELP1 gene—when included in a microarray. Measurement of NHELP1 expression has particular utility in the diagnosis and treatmnet of cancer and AIDS, as further described in the Examples herein below.

As would be readily apparent to one of skill in the art, each NHELP1 nucleic acid probe—whether labeled, substrate-bound, or both—is thus currently available for use as a tool for measuring the level of NHELP1 expression in each of the tissues in which expression has already been confirmed, notably brain, adult liver, adrenal, bone marrow, fetal liver, testis and prostate, as well as a cell line, hela. The utility is specific to the probe: under high stringency conditions, the probe reports the level of expression of message specifically containing that portion of the NHELP1 gene included within the probe.

Measuring tools are well known in many arts, not just in molecular biology, and are known to possess credible, specific, and substantial utility. For example, U.S. Pat. No. 6,016,191 describes and claims a tool for measuring characteristics of fluid flow in a hydrocarbon well; U.S. Pat. No. 6,042,549 describes and claims a device for measuring exercise intensity; U.S. Pat. No. 5,889,351 describes and claims a device for measuring viscosity and for measuring characteristics of a fluid; U.S. Pat. No. 5,570,694 describes and claims a device for measuring blood pressure; U.S. Pat. No. 5,930,143 describes and claims a device for measuring the dimensions of machine tools; U.S. Pat. No. 5,279,044 describes and claims a measuring device for determining an absolute position of a movable element; U.S. Pat. No. 5,186,042 describes and claims a device for measuring action force of a wheel; and U.S. Pat. No. 4,246,774 describes and claims a device for measuring the draft of smoking articles such as cigarettes.

As for tissues not yet demonstrated to express NHELP1, the NHELP1 nucleic acid probes of the present invention are currently available as tools for surveying such tissues to detect the presence of NHELP1 nucleic acids.

Survey tools—i.e., tools for determining the presence and/or location of a desired object by search of an area—are well known in many arts, not just in molecular biology, and are known to possess credible, specific, and substantial utility. For example, U.S. Pat. No. 6,046,800 describes and claims a device for surveying an area for objects that move; U.S. Pat. No. 6,025,201 describes and claims an apparatus for locating and discriminating platelets from non-platelet particles or cells on a cell-by-cell basis in a whole blood sample; U.S. Pat. No. 5,990,689 describes and claims a device for detecting and locating anomalies in the electromagnetic protection of a system; U.S. Pat. No. 5,984,175 describes and claims a device for detecting and identifying wearable user identification units; U.S. Pat. No. 3,980,986 (“Oil well survey tool”), describes and claims a tool for finding the position of a drill bit working at the bottom of a borehole.

As noted above, the nucleic acid probes of the present invention are useful in constructing microarrays; the microarrays, in turn, are products of manufacture that are useful for measuring and for surveying gene expression.

When included on a microarray, each NHELP1 nucleic acid probe makes the microarray specifically useful for detecting that portion of the NHELP1 gene included within the probe, thus imparting upon the microarray device the ability to detect a signal where, absent such probe, it would have reported no signal. This utility makes each individual probe on such microarray akin to an antenna, circuit, firmware or software element included in an electronic apparatus, where the antenna, circuit, firmware or software element imparts upon the apparatus the ability newly and additionally to detect signal in a portion of the radio-frequency spectrum where previously it could not; such devices are known to have specific, substantial, and credible utility.

Changes in the level of expression need not be observed for the measurement of expression to have utility.

For example, where gene expression analysis is used to assess toxicity of chemical agents on cells, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part.

WO 99/58720, incorporated herein by reference in its entirety, provides methods for quantifying the relatedness of a first and second gene expression profile and for ordering the relatedness of a plurality of gene expression profiles, without regard to the identity or function of the genes whose expression is used in the calculation.

Gene expression analysis, including gene expression analysis by microarray hybridization, is, of course, principally a laboratory-based art. Devices and apparatus used principally in laboratories to facilitate laboratory research are well-established to possess specific, substantial, and credible utility. For example, U.S. Pat. No. 6,001,233 describes and claims a gel electrophoresis apparatus having a cam-activated clamp; for example, U.S. Pat. No. 6,051,831 describes and claims a high mass detector for use in time-of-flight mass spectrometers; for example, U.S. Pat. No. 5,824,269 describes and claims a flow cytometer—as is well known, few gel electrophoresis apparatuses, TOF-MS devices, or flow cytometers are sold for consumer use.

Indeed, and in particular, nucleic acid microarrays, as devices intended for laboratory use in measuring gene expression, are well-established to have specific, substantial and credible utility. Thus, the microarrays of the present invention have at least the specific, substantial and credible utilities of the microarrays claimed as devices and articles of manufacture in the following U.S. patents, the disclosures of each of which is incorporated herein by reference: U.S. Pat. Nos. 5,445,934 (“Array of oligonucleotides on a solid substrate”); 5,744,305 (“Arrays of materials attached to a substrate”); and 6,004,752 (“Solid support with attached molecules”).

Genome-derived single exon probes and genome-derived single exon probe microarrays have the additional utility, inter alia, of permitting high-throughput detection of splice variants of the nucleic acids of the present invention, as further described in copending and commonly owned U.S. patent application Ser. No. 09/632,366, filed Aug. 3, 2000, the disclosure of which is incorporated herein by reference in its entirety.

The isolated nucleic acids of the present invention can also be used to prime synthesis of nucleic acid, for purpose of either analysis or isolation, using mRNA, cDNA, or genomic DNA as template.

For use as primers, at least 17 contiguous nucleotides of the isolated nucleic acids of the present invention will be used. Often, at least 18, 19, or 20 contiguous nucleotides of the nucleic acids of the present invention will be used, and on occasion at least 20, 22, 24, or 25 contiguous nucleotides of the nucleic acids of the present invention will be used, and even 30 nucleotides or more of the nucleic acids of the present invention can be used to prime specific synthesis.

The nucleic acid primers of the present invention can be used, for example, to prime first strand cDNA synthesis on an mRNA template.

Such primer extension can be done directly to analyze the message. Alternatively, synthesis on an mRNA template can be done to produce first strand cDNA. The first strand cDNA can thereafter be used, inter alia, directly as a single-stranded probe, as above-described, as a template for sequencing—permitting identification of alterations, including deletions, insertions, and substitutions, both normal allelic variants and mutations associated with abnormal phenotypes—or as a template, either for second strand cDNA synthesis (e.g., as an antecedent to insertion into a cloning or expression vector), or for amplification.

The nucleic acid primers of the present invention can also be used, for example, to prime single base extension (SBE) for SNP detection (see, e.g., U.S. Pat. No. 6,004,744, the disclosure of which is incorporated herein by reference in its entirety).

As another example, the nucleic acid primers of the present invention can be used to prime amplification of NHELP1 nucleic acids, using transcript-derived or genomic DNA as template.

Primer-directed amplification methods are now well-established in the art. Methods for performing the polymerase chain reaction (PCR) are compiled, inter alia, in McPherson, PCR (Basics: From Background to Bench), Springer Verlag (2000) (ISBN: 0387916008); Innis et al. (eds.), PCR Applications: Protocols for Functional Genomics, Academic Press (1999) (ISBN: 0123721857); Gelfand et al. (eds.), PCR Strategies, Academic Press (1998) (ISBN: 0123721822); Newton et al., PCR, Springer-Verlag New York (1997) (ISBN: 0387915060); Burke (ed.), PCR: Essential Techniques, John Wiley & Son Ltd (1996) (ISBN: 047195697X); White (ed.), PCR Cloning Protocols: From Molecular Cloning to Genetic Engineering, Vol. 67, Humana Press (1996) (ISBN: 0896033430); McPherson et al. (eds.), PCR 2: A Practical Approach, Oxford University Press, Inc. (1995) (ISBN: 0199634254), the disclosures of which are incorporated herein by reference in their entireties. Methods for performing RT-PCR are collected, e.g., in Siebert et al. (eds.), Gene Cloning and Analysis by RT-PCR, Eaton Publishing Company/Bio Techniques Books Division, 1998 (ISBN: 1881299147); Siebert (ed.), PCR Technique:RT-PCR, Eaton Publishing Company/BioTechniques Books (1995) (ISBN:1881299139), the disclosure of which is incorporated herein by reference in its entirety.

Isothermal amplification approaches, such as rolling circle amplification, are also now well-described. See, e.g., Schweitzer et al., Curr. Opin. Biotechnol. 12(1):21-7 (2001); U.S. Pat. Nos. 6,235,502, 6,221,603, 6,210,884, 6,183,960, 5,854,033, 5,714,320, 5,648,245, and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

As further described below, nucleic acids of the present invention, inserted into vectors that flank the nucleic acid insert with a phage promoter, such as T7, T3, or SP6 promoter, can be used to drive in vitro expression of RNA complementary to either strand of the nucleic acid of the present invention. The RNA can be used, inter alia, as a single-stranded probe, in cDNA-mRNA subtraction, or for in vitro translation.

As will be further discussed herein below, nucleic acids of the present invention that encode NHELP1 protein or portions thereof can be used, inter alia, to express the NHELP1 proteins or protein fragments, either alone, or as part of fusion proteins.

Expression can be from genomic nucleic acids of the present invention, or from transcript-derived nucleic acids of the present invention.

Where protein expression is effected from genomic DNA, expression will typically be effected in eukaryotic, typically mammalian, cells capable of splicing introns from the initial RNA transcript. Expression can be driven from episomal vectors, such as EBV-based vectors, or can be effected from genomic DNA integrated into a host cell chromosome. As will be more fully described below, where expression is from transcript-derived (or otherwise intron-less) nucleic acids of the present invention, expression can be effected in wide variety of prokaryotic or eukaryotic cells.

Expressed in vitro, the protein, protein fragment, or protein fusion can thereafter be isolated, to be used, inter alia, as a standard in immunoassays specific for the proteins, or protein isoforms, of the present invention; to be used as a therapeutic agent, e.g., to be administered as passive replacement therapy in individuals deficient in the proteins of the present invention, or to be administered as a vaccine; to be used for in vitro production of specific antibody, the antibody thereafter to be used, e.g., as an analytical reagent for detection and quantitation of the proteins of the present invention or to be used as an immunotherapeutic agent.

The isolated nucleic acids of the present invention can also be used to drive in vivo expression of the proteins of the present invention. In vivo expression can be driven from a vector—typically a viral vector, often a vector based upon a replication incompetent retrovirus, an adenovirus, or an adeno-associated virus (AAV)—for purpose of gene therapy. In vivo expression can also be driven from signals endogenous to the nucleic acid or from a vector, often a plasmid vector, such as pVAX1 (Invitrogen, Carlsbad Calif., USA), for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; 6,204,250, the disclosures of which are incorporated herein by reference in their entireties.

The nucleic acids of the present invention can also be used for antisense inhibition of transcription or translation. See Phillips (ed.), Antisense Technology, Part B, Methods in Enzymology Vol. 314, Academic Press, Inc. (1999) (ISBN: 012182215X); Phillips (ed.), Antisense Technology, Part A, Methods in Enzymology Vol. 313, Academic Press, Inc. (1999) (ISBN: 0121822141); Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Agrawal et al. (eds.), Antisense Research and Application, Springer-Verlag New York, Inc. (1998) (ISBN: 3540638334); Lichtenstein et al. (eds.), Antisense Technology: A Practical Approach, Vol. 185, Oxford University Press, INC. (1998) (ISBN: 0199635838); Gibson (ed.), Antisense and Ribozyme Methodology: Laboratory Companion, Chapman & Hall (1997) (ISBN: 3826100794); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties.

Nucleic acids of the present invention, particularly cDNAs of the present invention, that encode full-length human NHELP1 protein isoforms, have additional, well-recognized, immediate, real world utility as commercial products of manufacture suitable for sale.

For example, Invitrogen Corp. (Carlsbad, Calif., USA), through its Research Genetics subsidiary, sells full length human cDNAs cloned into one of a selection of expression vectors as GeneStorm® expression-ready clones; utility is specific for the gene, since each gene is capable of being ordered separately and has a distinct catalogue number, and utility is substantial, each clone selling for $650.00 US. Similarly, Incyte Genomics (Palo Alto, Calif., USA) sells clones from public and proprietary sources in multi-well plates or individual tubes.

Nucleic acids of the present invention that include genomic regions encoding the human NHELP1 protein, or portions thereof, have yet further utilities.

For example, genomic nucleic acids of the present invention can be used as amplification substrates, e.g. for preparation of genome-derived single exon probes of the present invention, as described above and in copending and commonly-owned U.S. patent application Ser. Nos. 09/864,761, filed May 23, 2001, 09/774,203, filed Jan. 29, 2001, and 09/632,366, filed Aug. 3, 2000, the disclosures of which are incorporated herein by reference in their entireties.

As another example, genomic nucleic acids of the present invention can be integrated non-homologously into the genome of somatic cells, e.g. CHO cells, COS cells, or 293 cells, with or without amplification of the insertional locus, in order, e.g., to create stable cell lines capable of producing the proteins of the present invention.

As another example, more fully described herein below, genomic nucleic acids of the present invention can be integrated nonhomologously into embryonic stem (ES) cells to create transgenic non-human animals capable of producing the proteins of the present invention.

Genomic nucleic acids of the present invention can also be used to target homologous recombination to the human NHELP1 locus. See, e.g., U.S. Pat. Nos. 6,187,305; 6,204,061; 5,631,153; 5,627,059; 5,487,992; 5,464,764; 5,614,396; 5,527,695 and 6,063,630; and Kmiec et al. (eds.), Gene Targeting Protocols, Vol. 133, Humana Press (2000) (ISBN: 0896033600); Joyner (ed.), Gene Targeting: A Practical Approach, Oxford University Press, Inc. (2000) (ISBN: 0199637938); Sedivy et al., Gene Targeting, Oxford University Press (1998) (ISBN: 071677013X); Tymms et al. (eds.), Gene Knockout Protocols, Humana Press (2000) (ISBN: 0896035727); Mak et al. (eds.), The Gene Knockout FactsBook, Vol. 2, Academic Press, Inc. (1998) (ISBN: 0124660444); Torres et al., Laboratory Protocols for Conditional Gene Targeting, Oxford University Press (1997) (ISBN: 019963677X); Vega (ed.), Gene Targeting, CRC Press, LLC (1994) (ISBN: 084938950X), the disclosures of which are incorporated herein by reference in their entireties.

Where the genomic region includes transcription regulatory elements, homologous recombination can be used to alter the expression of NHELP1, both for purpose of in vitro production of NHELP1 protein from human cells, and for purpose of gene therapy. See, e.g., U.S. Pat. Nos. 5,981,214, 6,048,524; 5,272,071.

Fragments of the nucleic acids of the present invention smaller than those typically used for homologous recombination can also be used for targeted gene correction or alteration, possibly by cellular mechanisms different from those engaged during homologous recombination.

For example, partially duplexed RNA/DNA chimeras have been shown to have utility in targeted gene correction, U.S. Pat. Nos. 5,945,339, 5,888,983, 5,871,984, 5,795,972, 5,780,296, 5,760,012, 5,756,325, 5,731,181, the disclosures of which are incorporated herein by reference in their entireties. So too have small oligonucleotides fused to triplexing domains have been shown to have utility in targeted gene correction, Culver et al., “Correction of chromosomal point mutations in human cells with bifunctional oligonucleotides,” Nature Biotechnol. 17(10):989-93 (1999), as have oligonucleotides having modified terminal bases or modified terminal internucleoside bonds, Gamper et al., Nucl. Acids Res. 28(21):4332-9 (2000), the disclosures of which are incorporated herein by reference.

The isolated nucleic acids of the present invention can also be used to provide the initial substrate for recombinant engineering of NHELP1 protein variants having desired phenotypic improvements. Such engineering includes, for example, site-directed mutagenesis, random mutagenesis with subsequent functional screening, and more elegant schemes for recombinant evolution of proteins, as are described, inter alia, in U.S. Pat. Nos. 6,180,406; 6,165,793; 6,117,679; and 6,096,548, the disclosures of which are incorporated herein by reference in their entireties.

Nucleic acids of the present invention can be obtained by using the labeled probes of the present invention to probe nucleic acid samples, such as genomic libraries, cDNA libraries, and mRNA samples, by standard techniques. Nucleic acids of the present invention can also be obtained by amplification, using the nucleic acid primers of the present invention, as further demonstrated in Example 1, herein below. Nucleic acids of the present invention of fewer than about 100 nt can also be synthesized chemically, typically by solid phase synthesis using commercially available automated synthesizers.

### “Full Length” NHELP1 Nucleic Acids

In a first series of nucleic acid embodiments, the invention provides isolated nucleic acids that encode the entirety of the NHELP1 protein. As discussed above, the “full-length” nucleic acids of the present invention can be used, inter alia, to express full length NHELP1 protein. The full-length nucleic acids can also be used as nucleic acid probes; used as probes, the isolated nucleic acids of these embodiments will hybridize to NHELP1.

In a first such embodiment, the invention provides an isolated nucleic acid comprising (i) the nucleotide sequence of SEQ ID NO: 1, or (ii) the complement of (i). The SEQ ID NO: 1 presents the entire cDNA of NHELP1, including the 5′ untranslated (UT) region and 3′ UT.

In a second embodiment, the invention provides an isolated nucleic acid comprising (i) the nucleotide sequence of SEQ ID NO: 2, (ii) a degenerate variant of the nucleotide sequence of SEQ ID NO: 2, or (iii) the complement of (i) or (ii). SEQ ID NO: 2 presents the open reading frame (ORF) from SEQ ID NO: 1.

In a third embodiment, the invention provides an isolated nucleic acid comprising (i) a nucleotide sequence that encodes a polypeptide with the amino acid sequence of SEQ ID NO: 3 or (ii) the complement of a nucleotide sequence that encodes a polypeptide with the amino acid sequence of SEQ ID NO: 3. SEQ ID NO: 3 provides the amino acid sequence of NHELP1.

In a fourth embodiment, the invention provides an isolated nucleic acid having a nucleotide sequence that (i) encodes a polypeptide having the sequence of SEQ ID NO: 3, (ii) encodes a polypeptide having the sequence of SEQ ID NO: 3 with conservative amino acid substitutions, or (iii) that is the complement of (i) or (ii), where SEQ ID NO: 3 provides the amino acid sequence of NHELP1.

### Selected Partial Nucleic Acids

In a second series of nucleic acid embodiments, the invention provides isolated nucleic acids that encode select portions of NHELP1. As will be further discussed herein below, these “partial” nucleic acids can be used, inter alia, to express specific portions of the NHELP1. These “partial” nucleic acids can also be used, inter alia, as nucleic probes.

In a first such embodiment, the invention provides an isolated nucleic acid comprising (i) the nucleotide sequence of SEQ ID NO: 4, (ii) a degenerate variant of SEQ ID NO: 6, or (iii) the complement of (i) or (ii), wherein the isolated nucleic acid is no more than about 100 kb in length, typically no more than about 75 kb in length, more typically no more than about 50 kb length. SEQ ID NO: 6 encodes a novel portion of NHELP1. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 7 or (ii) the complement of a nucleotide sequence that encodes SEQ ID NO: 7, wherein the isolated nucleic acid is no more than about 100 kb in length, typically no more than about 75 kb in length, frequently no more than about 50 kb in length. SEQ ID NO: 7 is the amino acid sequence encoded by the portion of NHELP1 not found in any EST fragments. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 7, (ii) a nucleotide sequence that encodes SEQ ID NO: 7 with conservative substitutions, or (iii) the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

### Cross-Hybridizing Nucleic Acids

In another series of nucleic acid embodiments, the invention provides isolated nucleic acids that hybridize to various of the NHELP1 nucleic acids of the present invention. These cross-hybridizing nucleic acids can be used, inter alia, as probes for, and to drive expression of, proteins that are related to NHELP1 of the present invention as further isoforms, homologues, paralogues, or orthologues.

In a first such embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a hybridization probe the nucleotide sequence of which (i) encodes a polypeptide having the sequence of SEQ ID NO: 7, (ii) encodes a polypeptide having the sequence of SEQ ID NO: 7 with conservative amino acid substitutions, or (iii) is the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

### Particularly Useful Nucleic Acids

Particularly useful among the above-described nucleic acids are those that are expressed, or the complement of which are expressed, in brain, adult liver, adrenal, bone marrow, fetal liver, testis and prostate, as well as hela cell line.

Also particularly useful among the above-described nucleic acids are those that encode, or the complement of which encode, a polypeptide having Na⁺/H⁺ exchange activity.

Other particularly useful embodiments of the nucleic acids above-described are those that encode, or the complement of which encode, a polypeptide having a Na_H_Exchanger domain.

### Nucleic Acid Fragments

In another series of nucleic acid embodiments, the invention provides fragments of various of the isolated nucleic acids of the present invention which prove useful, inter alia, as nucleic acid probes, as amplification primers, and to direct expression or synthesis of epitopic or immunogenic protein fragments.

In a first such embodiment, the invention provides an isolated nucleic acid comprising at least 17 nucleotides, 18 nucleotides, 20 nucleotides, 24 nucleotides, or 25 nucleotides of (i) SEQ ID NO: 4, (ii) a degenerate variant of SEQ ID NO: 6, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising (i) a nucleotide sequence that encodes a peptide of at least 8 contiguous amino acids of SEQ ID NO: 7, (ii) a nucleotide sequence that encodes a peptide of at least 15 contiguous amino acids of SEQ ID NO: 7, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising a nucleotide sequence that encodes (i) a polypeptide having the sequence of at least 8 contiguous amino acids of SEQ ID NO: 7 with conservative amino acid substitutions, (ii) a polypeptide having the sequence of at least 15 contiguous amino acids of SEQ ID NO: 7 with conservative amino acid substitutions, (iii) a polypeptide having the sequence of at least 8 contiguous amino acids of SEQ ID NO: 7 with moderately conservative substitutions, (iv) a polypeptide having the sequence of at least 15 contiguous amino acids of SEQ ID NO: 7 with moderately conservative substitutions, or (v) the complement of any of (i)-(iv), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

### Single Exon Probes

The invention further provides genome-derived single exon probes having portions of no more than one exon of the NHELP1 gene. As further described in commonly owned and copending U.S. patent application Ser. No. 09/632,366, filed Aug. 3, 2000 (“Methods and Apparatus for High Throughput Detection and Characterization of alternatively Spliced Genes”), the disclosure of which is incorporated herein by reference in its entirety, such single exon probes have particular utility in identifying and characterizing splice variants. In particular, such single exon probes are useful for identifying and discriminating the expression of distinct isoforms of NHELP1.

In a first embodiment, the invention provides an isolated nucleic acid comprising a nucleotide sequence of no more than one portion of SEQ ID NOs: 8-23 or the complement of SEQ ID NOs: 8-23, wherein the portion comprises at least 17 contiguous nucleotides, 18 contiguous nucleotides, 20 contiguous nucleotides, 24 contiguous nucleotides, 25 contiguous nucleotides, or 50 contiguous nucleotides of any one of SEQ ID NOs: 8-23, or their complement. In a further embodiment, the exonic portion comprises the entirety of the referenced SEQ ID NO: or its complement.

In other embodiments, the invention provides isolated single exon probes having the nucleotide sequence of any one of SEQ ID NOs: 24-39.

### Transcription Control Nucleic Acids

In another aspect, the present invention provides genome-derived isolated nucleic acids that include nucleic acid sequence elements that control transcription of the NHELP1 gene. These nucleic acids can be used, inter alia, to drive expression of heterologous coding regions in recombinant constructs, thus conferring upon such heterologous coding regions the expression pattern of the native NHELP1 gene. These nucleic acids can also be used, conversely, to target heterologous transcription control elements to the NHELP1 genomic locus, altering the expression pattern of the NHELP1 gene itself.

In a first such embodiment, the invention provides an isolated nucleic acid comprising the nucleotide sequence of SEQ ID NO: 40 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another embodiment, the invention provides an isolated nucleic acid comprising at least 17, 18, 20, 24, or 25 nucleotides of the sequence of SEQ ID NO: 40 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

### VECTORS AND HOST CELLS

In another aspect, the present invention provides vectors that comprise one or more of the isolated nucleic acids of the present invention, and host cells in which such vectors have been introduced.

The vectors can be used, inter alia, for propagating the nucleic acids of the present invention in host cells (cloning vectors), for shuttling the nucleic acids of the present invention between host cells derived from disparate organisms (shuttle vectors), for inserting the nucleic acids of the present invention into host cell chromosomes (insertion vectors), for expressing sense or antisense RNA transcripts of the nucleic acids of the present invention in vitro or within a host cell, and for expressing polypeptides encoded by the nucleic acids of the present invention, alone or as fusions to heterologous polypeptides. Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems : Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. Furthermore, an enormous variety of vectors are available commercially. Use of existing vectors and modifications thereof being well within the skill in the art, only basic features need be described here.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the present invention will further include at least one nucleic acid of the present invention inserted into the vector in at least one location.

Where present, the origin of replication and selectable markers are chosen based upon the desired host cell or host cells; the host cells, in turn, are selected based upon the desired application.

For example, prokaryotic cells, typically E. coli, are typically chosen for cloning. In such case, vector replication is predicated on the replication strategies of coliform-infecting phage—such as phage lambda, M13, T7, T3 and P1—or on the replication origin of autonomously replicating episomes, notably the ColE1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin, zeocin; auxotrophic markers can also be used.

As another example, yeast cells, typically S. cerevisiae, are chosen, inter alia, for eukaryotic genetic studies, due to the ease of targeting genetic changes by homologous recombination and to the ready ability to complement genetic defects using recombinantly expressed proteins, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast.

Integrative YIp vectors do not replicate autonomously, but integrate, typically in single copy, into the yeast genome at low frequencies and thus replicate as part of the host cell chromosome; these vectors lack an origin of replication that is functional in yeast, although they typically have at least one origin of replication suitable for propagation of the vector in bacterial cells. YEp vectors, in contrast, replicate episomally and autonomously due to presence of the yeast 2 micron plasmid origin (2 μm ori). The YCp yeast centromere plasmid vectors are autonomously replicating vectors containing centromere sequences, CEN, and autonomously replicating sequences, ARS; the ARS sequences are believed to correspond to the natural replication origins of yeast chromosomes. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trpl-D1 and lys2-201. The URA3 and LYS2 yeast genes further permit negative selection based on specific inhibitors, 5-fluoro-orotic acid (FOA) and α-aminoadipic acid (αAA), respectively, that prevent growth of the prototrophic strains but allows growth of the ura3 and lys2 mutants, respectively. Other selectable markers confer resistance to, e.g., zeocin.

As yet another example, insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

As yet another example, mammalian cells are often chosen for expression of proteins intended as pharmaceutical agents, and are also chosen as host cells for screening of potential agonist and antagonists of a protein or a physiological pathway.

Where mammalian cells are chosen as host cells, vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Plant cells can also be used for expression, with the vector replicon typically derived from a plant virus (e.g., cauliflower mosaic virus, CaMV; tobacco mosaic virus, TMV) and selectable markers chosen for suitability in plants.

For propagation of nucleic acids of the present invention that are larger than can readily be accomodated in vectors derived from plasmids or virus, the invention further provides artificial chromosomes—BACs, YACs, PACs, and HACs—that comprise NHELP1 nucleic acids, often genomic nucleic acids.

The BAC system is based on the well-characterized E. coli F-factor, a low copy plasmid that exists in a supercoiled circular form in host cells. The structural features of the F-factor allow stable maintenance of individual human DNA clones as well as easy manipulation of the cloned DNA. See Shizuya et al., Keio J. Med. 50(1):26-30 (2001); Shizuya et al., Proc. Natl. Acad. Sci. USA 89(18):8794-7 (1992).

YACs are based on yeast linear plasmids, denoted YLp, containing homologous or heterologous DNA sequences that function as telomeres (TEL) in vivo, as well as containing yeast ARS (origins of replication) and CEN (centromeres) segments.

HACs are human artifical chromosomes. Kuroiwa et al., Nature Biotechnol. 18(10):1086-90 (2000); Henning et al., Proc. Natl. Acad. Sci. USA 96(2):592-7 (1999); Harrington et al., Nature Genet. 15(4):345-55 (1997). In one version, long synthetic arrays of alpha satellite DNA are combined with telomeric DNA and genomic DNA to generate linear microchromosomes that are mitotically and cytogenetically stable in the absence of selection.

PACs are P1-derived artificial chromosomes. Sternberg, Proc. Natl. Acad. Sci. USA 87(1):103-7 (1990); Sternberg et al., New Biol. 2(2):151-62 (1990); Pierce et al., Proc. Natl Acad. Sci. USA 89(6):2056-60 (1992).

Vectors of the present invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Expression vectors of the present invention—that is, those vectors that will drive expression of polypeptides from the inserted heterologous nucleic acid—will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences.

For example, vectors for expressing proteins of the present invention in prokaryotic cells, typically E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), or the araBAD operon. Often, such prokaryotic expression vectors will further include transcription terminators, such as the aspA terminator, and elements that facilitate translation, such as a consensus ribosome binding site and translation termination codon, Schomer et al., Proc. Natl. Acad. Sci. USA 83:8506-8510 (1986).

As another example, vectors for expressing proteins of the present invention in yeast cells, typically S. cerevisiae, will include a yeast promoter, such as the CYC1 promoter, the GAL1 promoter, ADH1 promoter, or the GPD promoter, and will typically have elements that facilitate transcription termination, such as the transcription termination signals from the CYC1 or ADH1 gene.

As another example, vectors for expressing proteins of the present invention in mammalian cells will include a promoter active in mammalian cells. Such promoters are often drawn from mammalian viruses—such as the enhancer-promoter sequences from the immediate early gene of the human cytomegalovirus (CMV), the enhancer-promoter sequences from the Rous sarcoma virus long terminal repeat (RSV LTR), and the enhancer-promoter from SV40. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Vector-drive protein expression can be constitutive or inducible.

Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline.

As another example of inducible elements, hormone response elements, such as the glucocorticoid response element (GRE) and the estrogen response element (ERE), can confer hormone inducibility where vectors are used for expression in cells having the respective hormone receptors. To reduce background levels of expression, elements responsive to ecdysone, an insect hormone, can be used instead, with coexpression of the ecdysone receptor.

Expression vectors can be designed to fuse the expressed polypeptide to small protein tags that facilitate purification and/or visualization.

For example, proteins of the present invention can be expressed with a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). As another example, the fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the proteins of the present invention can be expressed as a fusion to glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione.

Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG® antibody (Stratagene, La Jolla, Calif., USA), and the HA epitope.

For secretion of expressed proteins, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Vectors for phage display fuse the encoded polypeptide to, e.g., the gene III protein (pIII) or gene VIII protein (pVIII) for display on the surface of filamentous phage, such as M13. See Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, San Diego: Academic Press, Inc., 1996; Abelson et al. (eds.), Combinatorial Chemistry, Methods in Enzymology vol. 267, Academic Press (May 1996).

Vectors for yeast display, e.g. the pYD1 yeast display vector (Invitrogen, Carlsbad, Calif., USA), use the α-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea victoria (“GFP”) and its variants. These proteins are intrinsically fluorescent: the GFP-like chromophore is entirely encoded by its amino acid sequence and can fluoresce without requirement for cofactor or substrate.

Structurally, the GFP-like chromophore comprises an 11-stranded β-barrel (β-can) with a central α-helix, the central α-helix having a conjugated π-resonance system that includes two aromatic ring systems and the bridge between them. The π-resonance system is created by autocatalytic cyclization among amino acids; cyclization proceeds through an imidazolinone intermediate, with subsequent dehydrogenation by molecular oxygen at the Cα-Cβ bond of a participating tyrosine.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al.,“Deletions of the Aequorea victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety. A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormö et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in the fusion proteins of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein, Methods in Enzymol. Vol. 302, pp 378-394 (1999), incorporated herein by reference in its entirety. A variety of such modified chromophores are now commercially available and can readily be used in the fusion proteins of the present invention.

Fusions to the IgG Fc region increase serum half life of protein pharmaceutical products through interaction with the FcRn receptor (also denominated the FcRp receptor and the Brambell receptor, FcRb), further described in international patent application nos. WO 97/43316, WO 97/34631, WO 96/32478, WO 96/18412.

For long-term, high-yield recombinant production of the proteins, protein fusions, and protein fragments of the present invention, stable expression is particularly useful.

Stable expression is readily achieved by integration into the host cell genome of vectors having selectable markers, followed by selection for integrants.

For example, the pUB6/V5-His A, B, and C vectors (Invitrogen, Carlsbad, Calif., USA) are designed for high-level stable expression of heterologous proteins in a wide range of mammalian tissue types and cell lines. pUB6/V5-His uses the promoter/enhancer sequence from the human ubiquitin C gene to drive expression of recombinant proteins: expression levels in 293, CHO, and NIH3T3 cells are comparable to levels from the CMV and human EF-la promoters. The bsd gene permits rapid selection of stably transfected mammalian cells with the potent antibiotic blasticidin.

Replication incompetent retroviral vectors, typically derived from Moloney murine leukemia virus, prove particularly useful for creating stable transfectants having integrated provirus. The highly efficient transduction machinery of retroviruses, coupled with the availability of a variety of packaging cell lines—such as RetroPack™ PT 67, EcoPack2™-293, AmphoPack-293, GP2-293 cell lines (all available from Clontech Laboratories, Palo Alto, Calif., USA)—allow a wide host range to be infected with high efficiency; varying the multiplicity of infection readily adjusts the copy number of the integrated provirus. Retroviral vectors are available with a variety of selectable markers, such as resistance to neomycin, hygromycin, and puromycin, permitting ready selection of stable integrants.

The present invention further includes host cells comprising the vectors of the present invention, either present episomally within the cell or integrated, in whole or in part, into the host cell chromosome.

Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide NHELP1 proteins with such post-translational modifications.

As noted earlier, host cells can be prokaryotic or eukaryotic. Representative examples of appropriate host cells include, but are not limited to, bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF™ cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, murine ES cell lines (e.g., from strains 129/SV, C57/BL6, DBA-1, 129/SVJ), K562, Jurkat cells, and BW5147. Other mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

For example, phage lambda vectors will typically be packaged using a packaging extract (e.g., Gigapack® packaging extract, Stratagene, La Jolla, Calif., USA), and the packaged virus used to infect E. coli. Plasmid vectors will typically be introduced into chemically competent or electrocompetent bacterial cells.

E. coli cells can be rendered chemically competent by treatment, e.g., with CaCl₂, or a solution of Mg²⁺, Mn²⁺, Ca²⁺, Rb⁺ or K⁺, dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian Coli® XL10-Gold® Ultracompetent Cells (Stratagene, La Jolla, Calif., USA); DH5a competent cells (Clontech Laboratories, Palo Alto, Calif., USA); TOP10 Chemically Competent E. coli Kit (Invitrogen, Carlsbad, Calif., USA)).

Bacterial cells can be rendered electrocompetent—that is, competent to take up exogenous DNA by electroporation—by various pre-pulse treatments; vectors are introduced by electroporation followed by subsequent outgrowth in selected media. An extensive series of protocols is provided online in Electroprotocols (BioRad, Richmond, Calif., USA) (http://www.bio-rad.com/LifeScience/pdf/New_Gene_Pulser.pdf).

Vectors can be introduced into yeast cells by spheroplasting, treatment with lithium salts, electroporation, or protoplast fusion.

Spheroplasts are prepared by the action of hydrolytic enzymes—a snail-gut extract, usually denoted Glusulase, or Zymolyase, an enzyme from Arthrobacter luteus—to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol. For lithium-mediated transformation, yeast cells are treated with lithium acetate, which apparently permeabilizes the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6):339-46 (1989). For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194:182-7 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means.

For chemical transfection, DNA can be coprecipitated with CaPO₄ or introduced using liposomal and nonliposomal lipid-based agents. Commercial kits are available for CaPO₄ transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Protocols for electroporating mammalian cells can be found online in Electroprotocols (Bio-Rad, Richmond, Calif., USA) (http://www.bio-rad.com/LifeScience/pdf/New_Gene_Pulser.pdf). See also, Norton et al. (eds.), Gene Transfer Methods: Introducing DNA into Living Cells and Organisms, BioTechniques Books, Eaton Publishing Co. (2000) (ISBN 1-881299-34-1), incorporated herein by reference in its entirety.

Other transfection techniques include transfection by particle embardment. See, e.g., Cheng et al., Proc. Natl. Acad. Sci. USA 90(10):4455-9 (1993); Yang et al., Proc. Natl. Acad. Sci. USA 87(24):9568-72 (1990).

### PROTEINS

In another aspect, the present invention provides NHELP1 proteins, various fragments thereof suitable for use as antigens (e.g., for epitope mapping) and for use as immunogens (e.g., for raising antibodies or as vaccines), fusions of NHELP1 polypeptides and fragments to heterologous polypeptides, and conjugates of the proteins, fragments, and fusions of the present invention to other moieties (e.g., to carrier proteins, to fluorophores).

FIG. 3 presents the predicted amino acid sequences encoded by the NHELP1 cDNA clone. The amino acid sequence is further presented in SEQ ID NO: 3.

Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Small deletions and insertions can often be found that do not alter the function of the protein.

Accordingly, it is an aspect of the present invention to provide proteins not only identical in sequence to those described with particularity herein, but also to provide isolated proteins at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at

http://www.ncbi.nlm.nih.gov/blast/bl2seq/bl2.html,

To assess percent identity of amino acid sequences, the BLASTP module of BLAST 2 SEQUENCES is used with default values of (i) BLOSUM62 matrix, Henikoff et al., Proc. Natl. Acad. Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function.

Accordingly, it is an aspect of the present invention to provide proteins not only identical in sequence to those described with particularity herein, but also to provide isolated proteins having the sequence of NHELP1 proteins, or portions thereof, with conservative amino acid substitutions. It is a further aspect to provide isolated proteins having the sequence of NHELP1 proteins, and portions thereof, with moderately conservative amino acid substitutions. These conservatively-substituted and moderately conservatively-substituted variants can be naturally occurring or can result from human intervention.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of proteins can also be characterized using a functional test, the ability of the encoding nucleic acids to base-pair to one another at defined hybridization stringencies.

It is, therefore, another aspect of the invention to provide isolated proteins not only identical in sequence to those described with particularity herein, but also to provide isolated proteins (“hybridization related proteins”) that are encoded by nucleic acids that hybridize under high stringency conditions (as defined herein above) to all or to a portion of various of the isolated nucleic acids of the present invention (“reference nucleic acids”). It is a further aspect of the invention to provide isolated proteins (“hybridization related proteins”) that are encoded by nucleic acids that hybridize under moderate stringency conditions (as defined herein above) to all or to a portion of various of the isolated nucleic acids of the present invention (“reference nucleic acids”).

The hybridization related proteins can be alternative isoforms, homologues, paralogues, and orthologues of the NHELP1 protein of the present invention. Particularly useful orthologues are those from other primate species, such as chimpanzee, rhesus macaque monkey, baboon, orangutan, and gorilla, from rodents, such as rats, mice, guinea pigs; from lagomorphs, such as rabbits, and from domestic livestock, such as cow, pig, sheep, horse, and goat.

Relatedness of proteins can also be characterized using a second functional test, the ability of a first protein competitively to inhibit the binding of a second protein to an antibody.

It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated NHELP1 proteins of the present invention (“reference proteins”). Such competitive inhibition can readily be determined using immunoassays well known in the art.

Among the proteins of the present invention that differ in amino acid sequence from those described with particularity herein—including those that have deletions and insertions causing up to 10% non-identity, those having conservative or moderately conservative substitutions, hybridization related proteins, and cross-reactive proteins—those that substantially retain one or more NHELP1 activities are particularly useful. As described above, those activities include Na+/H+ exchange.

Residues that are tolerant of change while retaining function can be identified by altering the protein at known residues using methods known in the art, such as alanine scanning mutagenesis, Cunningham et al., Science 244(4908):1081-5 (1989); transposon linker scanning mutagenesis, Chen et al., Gene 263(1-2):39-48 (2001); combinations of homolog- and alanine-scanning mutagenesis, Jin et al., J. Mol. Biol. 226(3):851-65 (1992); combinatorial alanine scanning, Weiss et al., Proc. Natl. Acad. Sci USA 97(16):8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, Epicentre Technologies Corporation, Madison, Wis., USA).

As further described below, the isolated proteins of the present invention can readily be used as specific immunogens to raise antibodies that specifically recognize NHELP1 proteins, their isoforms, homologues, paralogues, and/or orthologues. The antibodies, in turn, can be used, inter alia, specifically to assay for the NHELP1 proteins of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of NHELP1 proteins, as for example by immunoprecipitation, and for use as specific agonists or antagonists of NHELP1 action.

The isolated proteins of the present invention are also immediately available for use as specific standards in assays used to determine the concentration and/or amount specifically of the NHELP1 proteins of the present invention. As is well known, ELISA kits for detection and quantitation of protein analytes typically include isolated and purified protein of known concentration for use as a measurement standard (e.g., the human interferon-γ OptEIA kit, catalog no. 555142, Pharmingen, San Diego, Calif., USA includes human recombinant gamma interferon, baculovirus produced).

The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al., Electrophoresis 21(6):1164-77 (2000), the disclosures of which are incorporated herein by reference in their entireties. Analogously, the isolated proteins of the present invention are also immediately available for use as specific biomolecule capture probes on BIACORE surface plasmon resonance probes. . See Weinberger et al., Pharmacogenomics 1(4):395-416 (2000); Malmqvist, Biochem. Soc. Trans. 27(2):335-40 (1999).

The isolated proteins of the present invention are also useful as a therapeutic supplement in patients having a specific deficiency in NHELP1 production.

In another aspect, the invention also provides fragments of various of the proteins of the present invention. The protein fragments are useful, inter alia, as antigenic and immunogenic fragments of NHELP1.

By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least 8 contiguous amino acids, often at least 15 contiguous amino acids, have utility as immunogens for raising antibodies that recognize the proteins of the present invention. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20 amino acids in length, even 25 amino acids, 30 amino acids, 35 amino acids, or 50 amino acids or more in length. Of course, larger fragments having at least 75 amino acids, 100 amino acids, or even 150 amino acids are also useful, and at times preferred.

The present invention further provides fusions of each of the proteins and protein fragments of the present invention to heterologous polypeptides.

By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the NHELP1 protein in altered arrangement; in such case, any of the NHELP1 fragments can be considered heterologous to the other NHELP1 fragments in the fusion protein. More typically, however, the heterologous polypeptide is not drawn from the NHELP1 protein itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25 amino acids long, at least 50 amino acids long, and can be at least 75, 100, or even 150 amino acids long. Fusions that include the entirety of the proteins of the present invention have particular utility.

The heterologous polypeptide included within the fusion protein of the present invention is at least 6 amino acids in length, often at least 8 amino acids in length, and usefully at least 15, 20, and 25 amino acids in length. Fusions that include larger polypeptides, such as the IgG Fc region, and even entire proteins (such as GFP chromophore-containing proteins), have particular utility.

As described above in the description of vectors and expression vectors of the present invention, which discussion is incorporated herein by reference in its entirety, heterologous polypeptides to be included in the fusion proteins of the present invention can usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of NHELP1 presence.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al., Yeast Hybrid Technologies, Eaton Publishing, (2000) (ISBN 1-881299-15-5); Fields et al., Trends Genet. 10(8):286-92 (1994); Mendelsohn et al., Curr. Opin. Biotechnol. 5(5):482-6 (1994); Luban et al., Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al., Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful protein fusions include those that permit display of the encoded protein on the surface of a phage or cell, fusions to intrinsically fluorescent proteins, such as green fluorescent protein (GFP), and fusions to the IgG Fc region, as described above, which discussion is incorporated here by reference in its entirety.

The proteins and protein fragments of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, nonnative bonds, and post-synthetic (post translational) modifications, either throughout the length of the protein or localized to one or more portions thereof.

As is well known in the art, when the isolated protein is used, e.g., for epitope mapping, the range of such nonnatural analogues, nonnative inter-residue bonds, or post-synthesis modifications will be limited to those that permit binding of the peptide to antibodies. When used as an immunogen for the preparation of antibodies in a non-human host, such as a mouse, the range of such nonnatural analogues, nonnative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Non-natural amino acids can be incorporated during solid phase chemical synthesis or by recombinant techniques, although the former is typically more common.

Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

For example, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide.

Biotin, for example (indirectly detectable through interaction with avidin, streptavidin, neutravidin, captavidin, or anti-biotin antibody), can be added using biotinoyl—(9-fluorenylmethoxycarbonyl)-L-lysine (FMOC biocytin) (Molecular Probes, Eugene, Oreg., USA). (Biotin can also be added enzymatically by incorporation into a fusion protein of a E. coli BirA substrate peptide.) The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)—TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylithio)butyric acid, Fmoc-L-2-amino-4-(ethylthio)butyric acid, Fmoc-L-buthionine, Fmoc-S-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-b-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-?-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronoraharman-3-carboxylic acid, Fmoc-L-thiazolidine-4-carboxylic acid, all available from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl Acad. Sci. USA 96(9):4780-5 (1999); Wang et al., Science 292(5516):498-500 (2001).

The isolated proteins, protein fragments and fusion proteins of the present invention can also include nonnative inter-residue bonds, including bonds that lead to circular and branched forms.

The isolated proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or O-linked glycosylation, the pattern of which will reflect both the availability of glycosylation sites on the protein sequence and the identity of the host cell. Further modification of glycosylation pattern can be performed enzymatically.

As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide.

Useful post-synthetic (and post-translational) modifications include conjugation to detectable labels, such as fluorophores.

A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents.

Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive.

Other labels that usefully can be conjugated to the proteins, protein fragments, and fusion proteins of the present invention include radioactive labels, echosonographic contrast reagents, and MRI contrast agents.

The proteins, protein fragments, and protein fusions of the present invention can also usefully be conjugated using cross-linking agents to carrier proteins, such as KLH, bovine thyroglobulin, and even bovine serum albumin (BSA), to increase immunogenicity for raising anti-NHELP1 antibodies.

The proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

The isolated proteins of the present invention, including fusions thereof, can be produced by recombinant expression, typically using the expression vectors of the present invention as above-described or, if fewer than about 100 amino acids, by chemical synthesis (typically, solid phase synthesis), and, on occasion, by in vitro translation.

Production of the isolated proteins of the present invention can optionally be followed by purification. Purification of recombinantly expressed proteins is now well within the skill in the art. See, e.g., Thorner et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Expression and Protein Purification : Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Purification of chemically-synthesized peptides can readily be effected, e.g., by HPLC.

Accordingly, it is an aspect of the present invention to provide the isolated proteins of the present invention in pure or substantially pure form.

A purified protein of the present invention is an isolated protein, as above described, that is present at a concentration of at least 95%, as measured on a weight basis (w/w) with respect to total protein in a composition. Such purities can often be obtained during chemical synthesis without further purification, as, e.g., by HPLC. Purified proteins of the present invention can be present at a concentration (measured on a weight basis with respect to total protein in a composition) of 96%, 97%, 98%, and even 99%. The proteins of the present invention can even be present at levels of 99.5%, 99.6%, and even 99.7%, 99.8%, or even 99.9% following purification, as by HPLC.

Although high levels of purity are particularly useful when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, in another aspect, the present invention provides the isolated proteins of the present invention in substantially purified form. A “substantially purified protein” of the present invention is an isolated protein, as above described, present at a concentration of at least 70%, measured on a weight basis with respect to total protein in a composition. Usefully, the substantially purified protein is present at a concentration, measured on a weight basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The proteins, fragments, and fusions of the present invention can usefully be attached to a substrate. The substrate can porous or solid, planar or non-planar; the bond can be covalent or noncovalent.

For example, the proteins, fragments, and fusions of the present invention can usefully be bound to a porous substrate, commonly a membrane, typically comprising nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention.

As another example, the proteins, fragments, and fusions of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in standard microtiter dish, the plastic is typically polystyrene.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween. The proteins, fragments, and fusions of the present invention can also be attached to a substrate suitable for use in surface plasmon resonance detection; so attached, the protein, fragment, or fusion of the present invention is useful for binding and then detecting secondary proteins that bind with sufficient affinity or avidity to the surface-bound protein to indicate biological interaction therebetween.

### NHELP1 Proteins

In a first series of protein embodiments, the invention provides an isolated NHELP1 polypeptide having an amino acid sequence in SEQ ID NO: 3, which are full length NHELP1 proteins. When used as immunogens, the full length proteins of the present invention can be used, inter alia, to elicit antibodies that bind to a variety of epitopes of the NHELP1 protein.

The invention further provides fragments of the above-described polypeptides, particularly fragments having at least 6 amino acids, typically at least 8 amino acids, often at least 15 amino acids, and even the entirety of the sequence given in SEQ ID NO: 3.

The invention further provides fragments of at least 6 amino acids, typically at least 8 amino acids, often at least 15 amino acids, and even the entirety of the sequence given in SEQ ID NO: 7.

As described above, the invention further provides proteins that differ in sequence from those described with particularity in the above-referenced SEQ ID NOs., whether by way of insertion or deletion, by way of conservative or moderately conservative substitutions, as hybridization related proteins, or as cross-hybridizing proteins, with those that substantially retain a NHELP1 activity particularly useful.

The invention further provides fusions of the proteins and protein fragments herein described to heterologous polypeptides.

### ANTIBODIES AND ANTIBODY-PRODUCING CELLS

In another aspect, the invention provides antibodies, including fragments and derivatives thereof, that bind specifically to NHELP1 proteins and protein fragments of the present invention or to one or more of the proteins and protein fragments encoded by the isolated NHELP1 nucleic acids of the present invention. The antibodies of the present invention can be specific for all of linear epitopes, discontinuous epitopes, or conformational epitopes of such proteins or protein fragments, either as present on the protein in its native conformation or, in some cases, as present on the proteins as denatured, as, e.g., by solubilization in SDS.

In other embodiments, the invention provides antibodies, including fragments and derivatives thereof, the binding of which can be competitively inhibited by one or more of the NHELP1 proteins and protein fragments of the present invention, or by one or more of the proteins and protein fragments encoded by the isolated NHELP1 nucleic acids of the present invention.

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize,! a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to non-NHELP1 proteins by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human brain, adult liver, adrenal, bone marrow, fetal liver, testis and prostate.

Typically, the affinity or avidity of an antibody (or antibody multimer, as in the case of an IgM pentamer) of the present invention for a protein or protein fragment of the present invention will be at least about 1×10⁻⁶ molar (M) typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of 8 or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties.

For example, peptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies : Preparation and Use of Monoclonal Antibodies and Engineered Antibody Derivatives (Basics: From Background to Bench), Springer Verlag (2000) (ISBN: 0387915907), the disclosures of which are incorporated herein by reference, and often include multiple immunizations, either with or without adjuvants such as Freund's complete adjuvant and Freund's incomplete adjuvant.

Antibodies from nonhuman mammals can be polyclonal or monoclonal, with polyclonal antibodies having certain advantages in immunohistochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907); Howard et al. (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Techniques, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Briefly, however, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the proteins or protein fragments of the present invention can be cloned from hybridomas and thereafter expressed in other host cells. Nor need the two necessarily be performed together: e.g., genes encoding antibodies specific for the proteins and protein fragments of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production—either whole antibodies, antibody fragments, or antibody derivatives—can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1):102-8 (1998); Hoogenboom et al., Immunotechnology, 4(l):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the present invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(1):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention can also be produced in plant cells, Giddings et al., Nature Biotechnol. 18(11):1151-5 (2000); Gavilondo et al., Biotechniques 29(1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2):165-81 (1998), review and compare bacterial, yeast, insect and mammalian expression systems for expression of antibodies.

Antibodies of the present invention can also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998).

It is also an aspect of the present invention to provide antibody derivatives that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA.81(21):6851-5 (1984); Sharon et al., Nature 309(5966):364-7 (1984); Takeda et al., Nature 314(6010):452-4 (1985), the disclosures of which are incorporated herein by reference in their entireties. Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

The choice of label depends, in part, upon the desired use.

For example, when the antibodies of the present invention are used for immunohistochemical staining of tissue samples, the label can usefully be an enzyme that catalyzes production and local deposition of a detectable product.

Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as ³³P, ³²P, ³⁵S, ³H, and ¹²⁵I.

As another example, when the antibodies of the present invention are used for radioimmunotherapy, the label can usefully be ²²⁸Th, ²²⁷Ac, ²²⁵Ac, ²²³Ra, ²¹³Bi , ²¹²Pb, ²¹²Bi, ²¹¹At, ²⁰³Pb, ¹⁹⁴Os, ¹⁸⁸Re, ¹⁸⁶Re, ¹⁵³Sm, ¹⁴⁹Tb, ¹³¹I, ¹²⁵I, ¹¹¹In, ¹⁰⁵ Rh, ^{99m}Tc, ⁹⁷Ru, ⁹⁰Y, ⁹⁰Sr, ⁸⁸Y, ⁷²Se, ⁶⁷Cu, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling

As would be understood, use of the labels described above is not restricted to the application as for which they were mentioned.

The antibodies of the present invention, including fragments and derivatives thereof, can also be conjugated to toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate, and it is, therefore, another aspect of the invention to provide antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention, attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

### NHELP1 Antibodies

In a first series of antibody embodiments, the invention provides antibodies, both polyclonal and monoclonal, and fragments and derivatives thereof, that bind specifically to a polypeptide having an amino acid sequence in SEQ ID NO: 3, which are full length NHELP1 proteins.

Such antibodies are useful in in vitro immunoassays, such as ELISA, western blot or immunohistochemical assay of disease tissue or cells. Such antibodies are also useful in isolating and purifying NHELP1 proteins, including related cross-reactive proteins, by immunoprecipitation, immunoaffinity chromatography, or magnetic bead-mediated purification.

In another series of antibody embodiments, the invention provides antibodies, both polyclonal and monoclonal, and fragments and derivatives thereof, the specific binding of which can be competitively inhibited by the isolated proteins and polypeptides of the present invention.

In other embodiments, the invention further provides the above-described antibodies detectably labeled, and in yet other embodiments, provides the above-described antibodies attached to a substrate.

### PHARMACEUTICAL COMPOSITIONS

NHELP1 is important for Na⁺/H⁺ exchange; defects in NHELP1 expression, activity, distribution, localization, and/or solubility are a cause of human disease, which disease can manifest as a disorder of brain, adrenal, bone marrow, liver, testis or prostate function. Accordingly, pharmaceutical compositions comprising nucleic acids, proteins, and antibodies of the present invention, as well as mimetics, agonists, antagonists, or inhibitors of NHELP1 activity, can be administered as therapeutics for treatment of NHELP1 defects.

Thus, in another aspect, the invention provides pharmaceutical compositions comprising the nucleic acids, nucleic acid fragments, proteins, protein fusions, protein fragments, antibodies, antibody derivatives, antibody fragments, mimetics, agonists, antagonists, and inhibitors of the present invention.

Such a composition typically contains from about 0.1 to 90% by weight of a therapeutic agent of the invention formulated in and/or with a pharmaceutically acceptable carrier or excipient.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties, and thus need not be described in detail herein.

Briefly, however, formulation of the pharmaceutical compositions of the present invention will depend upon the route chosen for administration. The pharmaceutical compositions utilized in this invention can be administered by various routes including both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

Oral dosage forms can be formulated as tablets, pills, dragees, capsules, liquids, gels, syrups, slurries, suspensions, and the like, for ingestion by the patient.

Solid formulations of the compositions for oral administration can contain suitable carriers or excipients, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid.

Agents that facilitate disintegration and/or solubilization can be added, such as the cross-linked polyvinyl pyrrolidone, agar, alginic acid, or a salt thereof, such as sodium alginate, microcrystalline cellulose, corn starch, sodium starch glycolate, and alginic acid.

Tablet binders that can be used include acacia, methylcellulose, sodium carboxymethylcellulose, polyvinylpyrrolidone (Povidone™), hydroxypropyl methylcellulose, sucrose, starch and ethylcellulose.

Lubricants that can be used include magnesium stearates, stearic acid, silicone fluid, talc, waxes, oils, and colloidal silica.

Fillers, agents that facilitate disintegration and/or solubilization, tablet binders and lubricants, including the aforementioned, can be used singly or in combination.

Solid oral dosage forms need not be uniform throughout.

For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Oral dosage forms of the present invention include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Additionally, dyestuffs or pigments can be added to the tablets or dragee coatings for product identification or to characterize the quantity of active compound, i.e., dosage.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents.

The pharmaceutical compositions of the present invention can also be formulated for parenteral administration.

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution.

Intramuscular preparations, e.g. a sterile formulation of a suitable soluble salt form of the compounds of the present invention, can be dissolved and administered in a pharmaceutical excipient such as Water-for-Injection, 0.9% saline, or 5% glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Parenteral formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical compositions of the present invention can also be formulated to permit injectable, long-term, deposition.

The pharmaceutical compositions of the present invention can be administered topically.

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. In other transdermal formulations, typically in patch-delivered formulations, the pharmaceutically active compound is formulated with one or more skin penetrants, such as 2-N-methyl-pyrrolidone (NMP) or Azone.

Inhalation formulations can also readily be formulated. For inhalation, various powder and liquid formulations can be prepared.

The pharmaceutically active compound in the pharmaceutical compositions of the present inention can be provided as the salt of a variety of acids, including but not limited to hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art.

A “therapeutically effective dose” refers to that amount of active ingredient—for example NHELP1 protein, fusion protein, or fragments thereof, antibodies specific for NHELP1, agonists, antagonists or inhibitors of NHELP1—which ameliorates the signs or symptoms of the disease or prevents progression thereof; as would be understood in the medical arts, cure, although desired, is not required.

The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial useful concentration range and route of administration.

For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are particularly useful.

The data obtained from cell culture assays and animal studies is used in formulating an initial dosage range for human use, and preferably provides a range of circulating concentrations that includes the ED50 with little or no toxicity. After administration, or between successive administrations, the circulating concentration of active agent varies within this range depending upon pharmacokinetic factors well known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions.

### THERAPEUTIC METHODS

The present invention further provides methods of treating subjects having defects in NHELP1—e.g., in expression, activity, distribution, localization, and/or solubility of NHELP1—which can manifest as a disorder of brain, adrenal, bone marrow, liver, testis or prostate function. As used herein, “treating” includes all medically-acceptable types of therapeutic intervention, including palliation and prophylaxis (prevention) of disease.

In one embodiment of the therapeutic methods of the present invention, a therapeutically effective amount of a pharmaceutical composition comprising NHELP1 protein, fusion, fragment or derivative thereof is administered to a subject with a clinically-significant NHELP1 defect.

Protein compositions are administered, for example, to complement a deficiency in native NHELP1. In other embodiments, protein compositions are administered as a vaccine to elicit a humoral and/or cellular immune response to NHELP1. The immune response can be used to modulate activity of NHELP1 or, depending on the immunogen, to immunize against aberrant or aberrantly expressed forms, such as mutant or inappropriately expressed isoforms. In yet other embodiments, protein fusions having a toxic moiety are administered to ablate cells that aberrantly accumulate NHELP1.

In another embodiment of the therapeutic methods of the present invention, a therapeutically effective amount of a pharmaceutical composition comprising nucleic acid of the present invention is administered. The nucleic acid can be delivered in a vector that drives expression of NHELP1 protein, fusion, or fragment thereof, or without such vector.

Nucleic acid compositions that can drive expression of NHELP1 are administered, for example, to complement a deficiency in native NHELP1, or as DNA vaccines. Expression vectors derived from virus, replication deficient retroviruses, adenovirus, adeno-associated (AAV) virus, herpes virus, or vaccinia virus can be used—see, e.g., Cid-Arregui (ed.), Viral Vectors: Basic Science and Gene Therapy, Eaton Publishing Co., 2000 (ISBN: 188129935X)—as can plasmids.

Antisense nucleic acid compositions, or vectors that drive expression of NHELP1 antisense nucleic acids, are administered to downregulate transcription and/or translation of NHELP1 in circumstances in which excessive production, or production of aberrant protein, is the pathophysiologic basis of disease.

Antisense compositions useful in therapy can have sequence that is complementary to coding or to noncoding regions of the NHELP1 gene. For example, oligonucleotides derived from the transcription initiation site, e.g., between positions −10 and +10 from the start site, are particularly useful.

Catalytic antisense compositions, such as ribozymes, that are capable of sequence-specific hybridization to NHELP1 transcripts, are also useful in therapy. See, e.g., Phylactou, Adv. Drug Deliv. Rev. 44(2-3):97-108 (2000); Phylactou et al., Hum. Mol. Genet. 7(10):1649-53 (1998); Rossi, Ciba Found. Symp. 209:195-204 (1997); and Sigurdsson et al., Trends Biotechnol. 13(8):286-9 (1995), the disclosures of which are incorporated herein by reference in their entireties.

Other nucleic acids useful in the therapeutic methods of the present invention are those that are capable of triplex helix formation in or near the NHELP1 genomic locus. Such triplexing oligonucleotides are able to inhibit transcription, Intody et al., Nucleic Acids Res. 28(21):4283-90 (2000); McGuffie et al., Cancer Res. 60(14):3790-9 (2000), the disclosures of which are incorporated herein by reference, and pharmaceutical compositions comprising such triplex forming oligos (TFOs) are administered in circumstances in which excessive production, or production of aberrant protein, is a pathophysiologic basis of disease.

In another embodiment of the therapeutic methods of the present invention, a therapeutically effective amount of a pharmaceutical composition comprising an antibody (including fragment or derivative thereof) of the present invention is administered. As is well known, antibody compositions are administered, for example, to antagonize activity of NHELP1, or to target therapeutic agents to sites of NHELP1 presence and/or accumulation.

In another embodiment of the therapeutic methods of the present invention, a pharmaceutical composition comprising a non-antibody antagonist of NHELP1 is administered. Antagonists of NHELP1 can be produced using methods generally known in the art. In particular, purified NHELP1 can be used to screen libraries of pharmaceutical agents, often combinatorial libraries of small molecules, to identify those that specifically bind and antagonize at least one activity of NHELP1.

In other embodiments a pharmaceutical composition comprising an agonist of NHELP1 is administered. Agonists can be identified using methods analogous to those used to identify antagonists.

In still other therapeutic methods of the present invention, pharmaceutical compositions comprising host cells that express NHELP1, fusions, or fragments thereof can be administered. In such cases, the cells are typically autologous, so as to circumvent xenogeneic or allotypic rejection, and are administered to complement defects in NHELP1 production or activity.

In other embodiments, pharmaceutical compositions comprising the NHELP1 proteins, nucleic acids, antibodies, antagonists, and agonists of the present invention can be administered in combination with other appropriate therapeutic agents. Selection of the appropriate agents for use in combination therapy can be made by one of ordinary skill in the art according to conventional pharmaceutical principles. The combination of therapeutic agents or approaches can act additively or synergistically to effect the treatment or prevention of the various disorders described above, providing greater therapeutic efficacy and/or permitting use of the pharmaceutical compositions of the present invention using lower dosages, reducing the potential for adverse side effects.

### TRANSGENIC ANIMALS AND CELLS

In another aspect, the invention provides transgenic cells and non-human organisms comprising NHELP1 isoform nucleic acids, and transgenic cells and non-human organisms with targeted disruption of the endogenous orthologue of the human NHELP1 gene.

The cells can be embryonic stem cells or somatic cells. The transgenic non-human organisms can be chimeric, nonchimeric heterozygotes, and nonchimeric homozygotes.

### DIAGNOSTIC METHODS

The nucleic acids of the present invention can be used as nucleic acid probes to assess the levels of NHELP1 mRNA in brain, adrenal, bone marrow, liver, testis and prostate, and antibodies of the present invention can be used to assess the expression levels of NHELP1 proteins in brain, adrenal, bone marrow, liver, testis and prostate to diagnose cancer.

The following examples are offered for purpose of illustration, not limitation.

### EXAMPLE 1

### Identification and Characterization of cDNAs Encoding NHELP1 Proteins

Predicating our gene discovery efforts on use of genome-derived single exon probes and hybridization to genome-derived single exon microarrays—an approach that we have previously demonstrated will readily identify novel genes that have proven refractory to mRNA-based identification efforts—we identified an exon in raw human genomic sequence that is particularly expressed in human brain, adrenal, bone marrow, liver, testis and prostate.

Briefly, bioinformatic algorithms were applied to human genomic sequence data to identify putative exons. Each of the predicted exons was amplified from genomic DNA, typically centering the putative coding sequence within a larger amplicon that included flanking noncoding sequence. These genome-derived single exon probes were arrayed on a support and expression of the bioinformatically predicted exons assessed through a series of simultaneous two-color hybridizations to the genome-derived single exon microarrays.

The approach and procedures are further described in detail in Penn et al., “Mining the Human Genome using Microarrays of Open Reading Frames,” Nature Genetics 26:315-318 (2000); commonly owned and copending U.S. patent application Ser Nos. 09/864,761, filed May 23, 2001, 09/774,203, filed Jan. 29, 2001, and 09/632,366, filed Aug. 3, 2000, the disclosures of which are incorporated herein by reference in their entireties.

Using a graphical display particularly designed to facilitate computerized query of the resulting exon-specific expression data, as further described in commonly owned and copending U.S. patent application Ser. Nos. 09/864,761, filed May 23, 2001, 09/774,203, filed Jan. 29, 2001 and 09/632,366, filed Aug. 3, 2000, the disclosures of which are incorporated herein by reference in their entireties, one exon was identified that is expressed in all the human tissues tested; subsequent analysis revealed that the exon belong to a novel gene.

Table 1 summarizes the microarray expression data obtained using genome-derived single exon probe corresponding to exon 9. The probe was completely sequenced on both strands prior to its use on a genome-derived single exon microarray; sequencing confirmed the exact chemical structure of the probe. An added benefit of sequencing is that it placed us in possession of a set of single base-incremented fragments of the sequenced nucleic acid, starting from the sequencing primer's 3′ OH. (Since the single exon probes were first obtained by PCR amplification from genomic DNA, we were of course additionally in possession of an even larger set of single base incremented fragments of each of the single exon probes, each fragment corresponding to an extension product from one of the two amplification primers.)

Signals are normalized values measured and calculated as further described in commonly owned and copending U.S. patent application Ser. Nos. 09/864,761, filed May 23, 2001, 09/774,203, filed Jan. 29, 2001, 09/632,366, filed Aug. 3, 2000, and U.S. provisional patent application No. 60/207,456, filed May 26, 2000, the disclosures of which are incorporated herein by reference in their entireties.

TABLE 1
Expression Analysis
Genome-Derived Single Exon Microarray

| (TISSUE) | Amplicon 26945, Exon 9 |
|---|---|
| adrenal | 0.56 |
| adult liver | 0.46 |
| bone marrow | 0.46 |
| brain | 0.38 |
| fetal liver | 0.40 |
| hela | 0.50 |
| prostate | 0.46 |
| testis | 0.46 |

As shown in Table 1, significant expression of exon 9 was seen in brain, adrenal, bone marrow, liver, testis and prostate, as well as a cell line, hela.

Marathon-Ready™ brain cDNA (Clontech Laboratories, Palo Alto, Calif., USA) was used as a substrate for standard RACE (rapid amplification of cDNA ends) to obtain a cDNA clone that spans 2.1 kilobases and appears to contain the entire coding region of the gene to which the exon contributes; for reasons described below, we termed this cDNA NHELP1. Marathon-Ready™ cDNAs are adaptor-ligated double stranded cDNAs suitable for 3′ and 5′ RACE. Chenchik et al., BioTechniques 21:526-532 (1996); Chenchik et al., CLONTECHniques X(1):5-8 (January 1995). RACE techniques are described, inter alia, in the Marathon-Ready™ cDNA User Manual (Clontech Labs., Palo Alto, Calif., USA, Mar. 30, 2000, Part No. PT1156-1 (PR03517)), Ausubel et al. (eds.), Short Protocols in Molecular Biology : A Compendium of Methods from Current Protocols in Molecular Biology, 4^{th} edition (April 1999), John Wiley & Sons (ISBN: 047132938X) and Sambrook et al. (eds.), Molecular Cloning: A Laboratory Manual (3rd ed.), Cold Spring Harbor Laboratory Press (2000) (ISBN: 0879695773), the disclosures of which are incorporated herein by reference in their entireties.

The NHELP1 cDNA was sequenced on both strands using a MegaBACE™ 1000 sequencer (Amersham Biosciences, Sunnyvale, Calif., USA). Sequencing both strands provided us with the exact chemical structure of the cDNA, which is shown in FIG. 3 and further presented in the SEQUENCE LISTING as SEQ ID NO: 1, and placed us in actual physical possession of the entire set of single-base incremented fragments of the sequenced clone, starting at the 5′ and 3′ termini.

As shown in FIG. 3, the NHELP1 cDNA spans 2078 nucleotides and contains an open reading frame from nucleotide 50 through and including nt 1987 (inclusive of termination condon), predicting a protein of 645 amino acids with a (posttranslationally unmodified) molecular weight of 72.6 kD. The clone appears full length, with the reading frame opening starting with a methionine and terminating with a stop codon.

BLAST query of genomic sequence identified 7 BACS, spanning at least 80k, that constitute the minimum set of clones encompassing the cDNA sequence. Based upon the known origin of the BACs (GenBank accession numbers AC013592.5, AC013641.4, AC013805.4, AC016934.18, AC026673.18, AC073242.4, AC073358.9), the NHELP1 gene can be mapped to human chromosome 3q23.

Comparison of the cDNA and genomic sequences identified 16 exons. Exon organization is listed in Table 2.

TABLE 2
NHELP1 Exon Structure

| Exon no. | cDNA range | genomic range | BAC accession |
|---|---|---|---|
| 1 | 1-224 | 7128-7351 | AC013592.5 |
| 2 | 225-427 | 74122-73920 | |
| 3 | 428-505 | 183669-183592 | AC016934.18 |
| 4 | 506-582 | 181843-181767 | |
| 5 | 583-698 | 105464-105349 | |
| 6 | 699-804 | 14356-14461 | AC073358.9 |
| 7 | 805-943 | 44158-44020 | |
| 8 | 944-1049 | 39628-39523 | |
| 9 | 1050-1138 | 93408-93496 | |
| 10 | 1139-1252 | 89740-89853 | AC073242.4 |
| 11 | 1253-1364 | 42080-41969 | AC013641.4 |
| 12 | 1365-1518 | 27019-26866 | AC073242.4 |
| 13 | 1519-1574 | 108149-108094 | AC026673.18 |
| 14 | 1575-1653 | 5421-5499 | |
| 15 | 1654-1759 | 89858-89753 | AC013805.4 |
| 16 | 1760-2078 | 87807-87489 | |

FIG. 2 schematizes the exon organization of the NHELP1 clone.

At the top are shown the seven bacterial artificial chromosomes (BACs), with GenBank accession numbers, that span the NHELP1 locus. The genome-derived single-exon probe first used to demonstrate expression from this locus is shown below the BACs and labeled “592”. The 592 bp probe includes sequence drawn from exon 9, with additional sequence from introns 8 and 9.

As shown in FIG. 2, NHELP1, encoding a protein of 645 amino acids, comprises exons 1-16. Predicted molecular weight, prior to any post translational modification, is 72.6 kD. The clone appears full length, with the reading frame opening starting with a methionine and terminating with a stop codon.

As further discussed in the examples herein, expression of NHELP1 was assessed using hybridization to genome-derived single exon microarrays. Microarray analysis of exon nine showed expression in all tissues tested, including fetal liver, adult liver, brain, prostate, adrenal gland, testis and bone marrow.

The sequence of the NHELP1 cDNA was used as a BLAST query into the GenBank nr and dbEst databases. The nr database includes all non-redundant GenBank coding sequence translations, sequences derived from the 3-dimensional structures in the Brookhaven Protein Data Bank (PDB), sequences from SwissProt, sequences from the protein information resource (PIR), and sequences from protein research foundation (PRF). The dbEst (database of expressed sequence tags) includes ESTs, short, single pass read cDNA (mRNA) sequences, and cDNA sequences from differential display experiments and RACE experiments.

BLAST search identified multiple human and mouse ESTs, one EST from rat (BF404144.1) and one from zebrafish (AW566665.1) as having sequence closely related to NHELP1.

Globally, the human NHELP1 protein resembles the human sodium/hydrogen exchanger isoform 7 (GenBank accession: AAK54508.1, the NHELP1 protein with 59% amino acid identity and 71% amino acid similarity over 653 amino acids). NHELP1 aslo resembles the human sodium/hydrogen exchanger isoform 6 (GenBank accession: AAC39643.1, the NHELP1 protein with 60% amino acid identity and 73% amino acid similarity over 636 amino acids).

Motif searches using Pfam (http://pfam.wustl.edu), SMART (http://smart.embl-heidelberg.de), and PROSITE pattern and profile databases (http://www.expasy.ch/prosite), identified one known domains shared with human sodium/hydrogen exchangers.

FIG. 1 shows the domain structure of NHELP1.

As schematized in FIG. 1, the newly isolated gene product shares certain protein domains and an overall structural organization with other human sodium/hydrogen exchangers. The shared structural features strongly imply that NHELP1 plays a role similar to that of other human sodium/hydrogen exchangers in maintaining cation ion homeostasis.

Like the other human sodium/hydrogen exchanger isoforms, NHELP1 contains a Na_H_Exchanger domain. In NHELP1, the Na_H_Exchanger motif ocurrs at amino acids 128-454 (http://www.ncbi.nlm.nih.gov/Structure/cdd/wrpsb.cgi/). Other signatures of the newly isolated NHELP1 proteins were identified by searching the PROSITE database (http://www.expasy.ch/tools/scnpsit1.html). These include three N-glycosylation sites (96-99, 352-355, 516-519), four protein kinase C phosphorylation sites (9-11, 149-151, 258-260 and 522-524), one tyrosine kinase phosphorylation site (6-14), a leucine zipper (311-332), a amidation site, seven Casein kinase II phosphorylation sites, and eleven N-myristoylation sites.

Possession of the genomic sequence permitted search for promoter and other control sequences for the NHELP1 gene. A putative transcriptional control region, inclusive of promoter and downstream elements, was defined as 1 kb around the transcription start site, itself defined as the first nucleotide of the NHELP1 cDNA clone. The region, drawn from sequence of BAC AC013592.5, has the sequence given in SEQ ID NO: 40, which lists 1000 nucleotides before the transcription start site.

Transcription factor binding sites were identified using a web based program (http://motif.genome.ad.jp/), including binding sites for CdxA (681-687 and 710-716), AP-1 (600-608), as well as c-Ets-1 (870-879, with numbering according to SEQ ID NO: 40), amongst others.

We have thus identified a newly described human gene, NHELP1, which shares certain protein domains and an overall structural organization with human sodium/hydrogen exchanger isoforms. The shared structural features strongly imply that the NHELP1 protein plays a role similar to other sodium/hydrogen exchangers, in maintaining cation ion homeostasis, making the NHELP1 proteins and nucleic acids clinically useful diagnostic markers and potential therapeutic agents for cancer and AIDS.

### EXAMPLE 2

### Preparation and Labeling of Useful Fragments of NHELP1

Useful fragments of NHELP1 are produced by PCR, using standard techniques, or solid phase chemical synthesis using an automated nucleic acid synthesizer. Each fragment is sequenced, confirming the exact chemical structure thereof.

The exact chemical structure of preferred fragments is provided in the attached SEQUENCE LISTING, the disclosure of which is incorporated herein by reference in its entirety. The following summary identifies the fragments whose structures are more fully described in the SEQUENCE LISTING:

SEQ ID NO: 1 (nt, full length NHELP1 cDNA)

SEQ ID NO: 2 (nt, cDNA ORF)

SEQ ID NO: 3 (aa, full length NHELP1 protein)

SEQ ID NO: 4 (nt, (nt 1-1528) portion of NHELP1)

SEQ ID NO: 5 (nt, 5′ UT portion of SEQ ID NO: 4)

SEQ ID NO: 6 (nt, coding region of SEQ ID NO: 4)

SEQ ID NO: 7 (aa, (aa 1-493) CDS entirely within portion of NHELP1)

SEQ ID NOs: 8-23 (nt, exons 1-16 (from genomic sequence))

SEQ ID NOs: 24-39 (nt, 500 bp genomic amplicon centered about exons 1-16)

SEQ ID NO: 40 (nt, 1000 bp putative promoter)

SEQ ID NOs: 41-1552 (nt, 17-mers scanning (nt 1-1528) portion of NHELP1)

SEQ ID NOs: 1553-3056 (nt, 25-mers scanning (nt 1-1528) portion of NHELP1)

Upon confirmation of the exact structure, each of the above-described nucleic acids of confirmed structure is recognized to be immediately useful as a NHELP1 -specific probe.

For use as labeled nucleic acid probes, the above-described NHELP1 nucleic acids are separately labeled by random priming. As is well known in the art of molecular biology, random priming places the investigator in possession of a near-complete set of labeled fragments of the template of varying length and varying starting nucleotide.

The labeled probes are used to identify the NHELP1 gene on a Southern blot, and are used to measure expression of NHELP1 mRNA on a northern blot and by RT-PCR, using standard techniques.

### EXAMPLE 3

### Production of NHELP1 Protein

The full length NHELP1 cDNA clone is cloned into the mammalian expression vector pcDNA3.1/HISA (Invitrogen, Carlsbad, Calif., USA), transfected into COS7 cells, transfectants selected with G418, and protein expression in transfectants confirmed by detection of the anti-Xpress™ epitope according to manufacturer's instructions. Protein is purified using immobilized metal affinity chromatography and vector-encoded protein sequence is then removed with enterokinase, per manufacturer's instructions, followed by gel filtration and/or HPLC.

Following epitope tag removal, NHELP1 protein is present at a concentration of at least 70%, measured on a weight basis with respect to total protein (i.e., w/w), and is free of acrylamide monomers, bis acrylamide monomers, polyacrylamide and ampholytes. Further HPLC purification provides NHELP1 protein at a concentration of at least 95%, measured on a weight basis with respect to total protein (i.e., w/w).

### EXAMPLE 4

### Production of Anti-NHELP1 Antibody

Purified proteins prepared as in Example 3 are conjugated to carrier proteins and used to prepare murine monoclonal antibodies by standard techniques. Initial screening with the unconjugated purified proteins, followed by competitive inhibition screening using peptide fragments of the NHELP1, identifies monoclonal antibodies with specificity for NHELP1.

### EXAMPLE 5

### Use of NHELP1 Probes and Antibodies for Diagnosis of Cancer

After informed consent is obtained, samples are drawn from disease tissue or cells and tested for NHELP1 mRNA levels by standard techniques and tested additionally for NHELP1 protein levels using anti-NHELP1 antibodies in a standard ELISA.

### EXAMPLE 6

### Use of NHELP1 Nucleic Acids, Proteins, and Antibodies in Therapy

Once over-expression of NHELP1 is detected in patients, NHELP1 antisense RNA or NHELP1 -specific antibody is introduced into disease cells to reduce the amount of the protein.

Once mutations of NHELP1 have been detected in patients, normal NHELP1 is reintroduced into the patient's disease cells by introduction of expression vectors that drive NHELP1 expression or by introducing NHELP1 proteins into cells. Antibodies for the mutated forms of NHELP1 are used to block the function of the abnormal forms of the protein.

### EXAMPLE 7

### NHELP1 Disease Associations

Diseases that map to the NHELP1 chromosomal region are shown in Table 3. Mutations of NHELP1 might lead to the disease(s) listed below. Alternatively, mutations of NHELP1 might lead to some other human disorder(s) as well.

TABLE 3
Diseases mapped to human chromosome 3q23 (NHELP1 region)

| | | chromosomal |
|---|---|---|
| Mim_num | Disease | location |
| 601682 | Primary open angle glaucoma | 3q21-q24 |
| 127550 | Dyskeratosis congenita | 3q21-q28 |

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. An isolated nucleic acid that encodes a Na+/H+ exchanger protein, comprising:
(a) a nucleotide sequence selected from the group consisting of:
(i) SEQ ID NO: 1;
(ii) the complement of the sequences set forth in (i);
(iii) the nucleotide sequence of SEQ ID NO: 2;
(iv) a degenerate variant of the sequences set forth in (iii); and
(v) the complement of the sequences set forth in (iii) and (iv); or
(b) a nucleotide sequence selected from the group consisting of:
(i) a nucleotide sequence that encodes a polypeptide having the sequence of SEQ ID No: 3;
(ii) a nucleotide sequence that encodes a polypeptide having the sequence of SEQ ID No: 3, with conservative amino acid substitutions; and
(iii) the complement of the sequences set forth in (i) and (ii),
wherein said isolated nucleic acid comprising a nucleotide sequence selected from group (b) is no more than about 100 kb in length.

2. The isolated nucleic acid of claim 1 wherein said nucleic acid, or the complement of said nucleic acid, encodes a polypeptide.

3. The isolated nucleic acid of claim 1, wherein said nucleic acid, or the complement of said nucleic acid, is expressed in brain, adrenal, bone marrow, liver, testis and prostate, as well as a cell line, hela.

4. A nucleic acid probe, comprising:
(a) the nucleic acid of claim 1; or
(b) at least 17 contiguous nucleotides of SEQ ID NO: 4,
wherein said probe according to (b) is no longer than about 100 kb in length.

5. The probe of claim 4, wherein said probe is detectably labeled.

6. The probe of claim 4, attached to a substrate.

7. A microarray, wherein at least one probe of said array is a probe according to claim 4.

8. The isolated nucleic acid molecule of claim 1, wherein said nucleic acid molecule is operably linked to one or more expression control elements.

9. A replicable vector comprising a nucleic acid molecule of claim 1.

10. A replicable vector comprising an isolated nucleic acid molecule of claim 8.

11. A host cell transformed to contain the nucleic acid molecule of any one of claims 1 or 8-10, or the progeny thereof.

12. A method for producing a polypeptide, the method comprising: culturing the host cell of claim 11 under conditions in which the protein encoded by said nucleic acid molecule is expressed.

13. An isolated polypeptide produced by the method of claim 12.

14. An isolated polypeptide, comprising:
(a) an amino acid sequence of SEQ ID NO: 3;
(b) an amino acid sequence having at least 65% amino acid sequence identity to that of (a);
(c) an amino acid sequence according to (a) in which at least 95% of deviations from the sequence of (a) are conservative substitutions; or
(d) a fragment of at least 8 contiguous amino acids of any of (a)-(c).

15. A fusion protein, said fusion protein comprising a polypeptide of claim 14 fused to a heterologous amino acid sequence.

16. The fusion protein of claim 15, wherein said heterologous amino acid sequence is a detectable moiety.

17. The fusion protein of claim 16, wherein said detectable moiety is fluorescent.

18. The fusion protein of claim 15, wherein said heterologous amino acid sequence is an Ig Fc region.

19. An isolated antibody, or antigen-binding fragment or derivative thereof, the binding of which can be competitively inhibited by a polypeptide of claim 14.

20. A transgenic non-human animal modified to contain the nucleic acid molecule of any one of claims 1 or 8-10.

21. A transgenic non-human animal unable to express the endogenous orthologue of the nucleic acid molecule of claim 1.

22. A method of identifying agents that modulate the expression of NHELP1, the method comprising:
contacting a cell or tissue sample believed to express NHELP1 with a chemical or biological agent, and then comparing the amount of NHELP1 expression in said cell or tissue sample with that of a control,
changes in the amount relative to control identifying an agent that modulates expression of NHELP1.

23. A method of identifying agonists and antagonists of NHELP1, the method comprising:
contacting a cell or tissue sample believed to express NHELP1 with a chemical or biological agent, and then comparing the activity of NHELP1 with that of a control,
increased activity relative to a control identifying an agonist, decreased activity relative to a control identifying an antagonist.

24. A purified agonist of the polypeptide of claim 14.

25. A purified antagonist of the polypeptide of claim 14.

26. A method of identifying a specific binding partner for a polypeptide according to claim 14, the method comprising:
contacting said polypeptide to a potential binding partner; and
determining if the potential binding partner binds to said polypeptide.

27. The method of claim 26, wherein said contacting is performed in vivo.

28. A purified binding partner of the polypeptide of claim 14.

29. A method for detecting a target nucleic acid in a sample, said target being a nucleic acid according to claim 1, the method comprising:
(a) hybridizing the sample with a probe comprising at least 17 contiguous nucleotides of a sequence complementary to said target nucleic acid in said sample under high stringency hybridization conditions, and
(b) detecting the presence or absence, and optionally the amount, of said binding.

30. A method of diagnosing a disease caused by mutation in NHELP1, comprising:
detecting said mutation in a sample of nucleic acids that derives from a subject suspected to have said disease.

31. A method of diagnosing or monitoring a disease caused by altered expression of NHELP1, comprising:
determining the level of expression of NHELP1 in a sample of nucleic acids or proteins that derives from a subject suspected to have said disease,
alterations from a normal level of expression providing diagnostic and/or monitoring information.

32. A diagnostic composition comprising the nucleic acid of claim 1, said nucleic acid being detectably labeled.

33. The diagnostic composition of claim 32, wherein said composition is further suitable for in vivo administration.

34. A diagnostic composition comprising the polypeptide of claim 14, said polypeptide being detectably labeled.

35. The diagnostic composition of claim 34, wherein said composition is further suitable for in vivo administration.

36. A diagnostic composition comprising the antibody, or antigen-binding fragment or derivative thereof, of claim 19.

37. The diagnostic composition of claim 36, wherein said antibody or antigen-binding fragment or derivative thereof is detectably labeled.

38. The diagnostic composition of claim 37, wherein said composition is further suitable for in vivo administration.

39. A pharmaceutical composition comprising the nucleic acid of claim 1 and a pharmaceutically acceptable excipient.

40. A pharmaceutical composition comprising the polypeptide of claim 14 and a pharmaceutically acceptable excipient.

41. A pharmaceutical composition comprising the antibody or antigen-binding fragment or derivative thereof of claim 19 and a pharmaceutically acceptable excipient.

42. A pharmaceutical composition comprising the agonist of claim 24 and a pharmaceutically acceptable excipient.

43. A pharmaceutical composition comprising the antagonist of claim 25 and a pharmaceutically acceptable excipient.

44. A method for treating or preventing a disorder associated with decreased expression or activity of NHELP1, the method comprising administering to a subject in need of such treatment an effective amount of the pharmaceutical composition of any of claims 39, 40 or 42.

45. A method for treating or preventing a disorder associated with increased expression or activity of NHELP1, the method comprising administering to a subject in need of such treatment an effective amount of the pharmaceutical composition of claim 41 or 43.

46. A method of modulating the expression of a nucleic acid according to claim 1, the method comprising:
administering an effective amount of an agent which modulates the expression of a nucleic acid according to claim 1.

47. A method of modulating at least one activity of a polypeptide according to claim 14, the method comprising:
administering an effective amount of an agent which modulates at least one activity of a polypeptide according to claim 14.


SEQUENCE LISTING (3056 sequences)

SEQ ID NO: 1 - DNA, 2078 bp, Homo sapiens
atagcccttatccaggtttttatctaaggaatcccaagaagactggggaatggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatggccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaagatgacatttgatccagaaatcttcttcaatgttttactgccaccaattatatttcatgcaggatatagtctaaagaagagacacttttttcaaaacttaggatctattttaacgtatgccttcttgggaactgccatctcctgcatcgtcatagggttaattatgtatggttttgtgaaggctatgatacatgctggccagctgaaaaatggagactttcatttcactgactgtttattttttggttcactgatgtctgctacagatccagtgacagtgctggccattttccatgaactgcacgtcgaccctgacctgtacacactcttgtttggagagagtgtgttgaatgatgcagtggccatagtccttacatattctatatccatttacagtcccaaggagaatccaaatgcatttgatgccgcagcattcttccagtctgtggggaatttcctgggaatcttcgctggctcatttgcaatggggtctgcgtatgccatcatcacagcactgttgaccaaatttaccaagctgtgtgagttcccgatgctggaaaccggcctgtttttcctgctttcttggagtgccttcctgtctgccgaggctgccggcctaacagggatagttgctgttctcttctgtggagtcacacaagcacattatacctacaacaatctgtcttcggattccaaaataagaactaaacagttgtttgaatttatgaactttttggcggagaacgtcatcttctgttacatgggcctggcactgttcacgttccagaatcatatctttaatgctctttttatacttggagcctttctagcaatttttgttgccagagcctgcaacatatatcccctctccttcctcctgaatctaggccgaaaacagaagatcccctggaactttcagcacatgatgatgttttcaggtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcagagttggcgtggacctggatgaaaatctgaaggaggacccctcctcacaacaccaggaagcaaataacttggataaaaacatgacgaaagcagagagtgctcggctcttcagaatgtggtatagctttgaccacaagtatctgaaaccaattttaacccactctggtcctccgctgactacaacattacctgaatggtgtggtccgatttccaggctgcttaccagtcctcaagcctatggggaacagctaaaagaggatgatgtggaatgcattgtaaaccaggatgaactagccataaattaccaggagcaagcctcctcaccctgcagtcctcctgcaaggctaggtctggaccagaaagcttcaccccagacgccaggcaaggaaaacatttatgagggagacctcggcctgggaggctatgaactcaagcttgagcaaactttgggtcaatcccagttgaattaattggcatgaagagtacagatgtaatcacaagtaatgcaagactcactgaggaatacaagccaagctgatgaggcagtacaggggagaggct

SEQ ID NO: 2 - DNA, 1938 bp, Homo sapiens
atggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatggccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaagatgacatttgatccagaaatcttcttcaatgttttactgccaccaattatatttcatgcaggatatagtctaaagaagagacacttttttcaaaacttaggatctattttaacgtatgccttcttgggaactgccatctcctgcatcgtcatagggttaattatgtatggttttgtgaaggctatgatacatgctggccagctgaaaaatggagactttcatttcactgactgtttattttttggttcactgatgtctgctacagatccagtgacagtgctggccattttccatgaactgcacgtcgaccctgacctgtacacactcttgtttggagagagtgtgttgaatgatgcagtggccatagtccttacatattctatatccatttacagtcccaaggagaatccaaatgcatttgatgccgcagcattcttccagtctgtggggaatttcctgggaatcttcgctggctcatttgcaatggggtctgcgtatgccatcatcacagcactgttgaccaaatttaccaagctgtgtgagttcccgatgctggaaaccggcctgtttttcctgctttcttggagtgccttcctgtctgccgaggctgccggcctaacagggatagttgctgttctcttctgtggagtcacacaagcacattatacctacaacaatctgtcttcggattccaaaataagaactaaacagttgtttgaatttatgaactttttggcggagaacgtcatcttctgttacatgggcctggcactgttcacgttccagaatcatatctttaatgctctttttatacttggagcctttctagcaatttttgttgccagagcctgcaacatatatcccctctccttcctcctgaatctaggccgaaaacagaagatcccctggaactttcagcacatgatgatgttttcaggtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcagagttggcgtggacctggatgaaaatctgaaggaggacccctcctcacaacaccaggaagcaaataacttggataaaaacatgacgaaagcagagagtgctcggctcttcagaatgtggtatagctttgaccacaagtatctgaaaccaattttaacccactctggtcctccgctgactacaacattacctgaatggtgtggtccgatttccaggctgcttaccagtcctcaagcctatggggaacagctaaaagaggatgatgtggaatgcattgtaaaccaggatgaactagccataaattaccaggagcaagcctcctcaccctgcagtcctcctgcaaggctaggtctggaccagaaagcttcaccccagacgccaggcaaggaaaacatttatgagggagacctcggcctgggaggctatgaactcaagcttgagcaaactttgggtcaatcccagttgaattaa

SEQ ID NO: 3 - protein, 645 aa, Homo sapiens
MERQSRVMSEKDEYQFQHQGAVELLVFNFLLILTILTIWLFKNHRFRFLHETGGAMVYGLIMGLILRYATAPTDIESGTVYDCVKLTFSPSTLLVNITDQVYEYKYKREISQHNINPHQGNAILEKMTFDPEIFFNVLLPPIIFHAGYSLKKRHFFQNLGSILTYAFLGTAISCIVIGLIMYGFVKAMIHAGQLKNGDFHFTDCLFFGSLMSATDPVTVLAIFHELHVDPDLYTLLFGESVLNDAVAIVLTYSISIYSPKENPNAFDAAAFFQSVGNFLGIFAGSFAMGSAYAIITALLTKFTKLCEFPMLETGLFFLLSWSAFLSAEAAGLTGIVAVLFCGVTQAHYTYNNLSSDSKIRTKQLFEFMNFLAENVIFCYMGLALFTFQNHIFNALFILGAFLAIFVARACNIYPLSFLLNLGRKQKIPWNFQHMMMFSGLRGAIAFALAIRNTESQPKQMMFTTTLLLVFFTVWVFGGGTTPMLTWLQIRVGVDLDENLKEDPSSQHQEANNLDKNMTKAESARLFRMWYSFDHKYLKPILTHSGPPLTTTLPEWCGPISRLLTSPQAYGEQLKEDDVECIVNQDELAINYQEQASSPCSPPARLGLDQKASPQTPGKENIYEGDLGLGGYELKLEQTLGQSQLN

SEQ ID NO: 4 - DNA, 1528 bp, Homo sapiens
atagcccttatccaggtttttatctaaggaatcccaagaagactggggaatggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatggccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaagatgacatttgatccagaaatcttcttcaatgttttactgccaccaattatatttcatgcaggatatagtctaaagaagagacacttttttcaaaacttaggatctattttaacgtatgccttcttgggaactgccatctcctgcatcgtcatagggttaattatgtatggttttgtgaaggctatgatacatgctggccagctgaaaaatggagactttcatttcactgactgtttattttttggttcactgatgtctgctacagatccagtgacagtgctggccattttccatgaactgcacgtcgaccctgacctgtacacactcttgtttggagagagtgtgttgaatgatgcagtggccatagtccttacatattctatatccatttacagtcccaaggagaatccaaatgcatttgatgccgcagcattcttccagtctgtggggaatttcctgggaatcttcgctggctcatttgcaatggggtctgcgtatgccatcatcacagcactgttgaccaaatttaccaagctgtgtgagttcccgatgctggaaaccggcctgtttttcctgctttcttggagtgccttcctgtctgccgaggctgccggcctaacagggatagttgctgttctcttctgtggagtcacacaagcacattatacctacaacaatctgtcttcggattccaaaataagaactaaacagttgtttgaatttatgaactttttggcggagaacgtcatcttctgttacatgggcctggcactgttcacgttccagaatcatatctttaatgctctttttatacttggagcctttctagcaatttttgttgccagagcctgcaacatatatcccctctccttcctcctgaatctaggccgaaaacagaagatcccctggaactttcagcacatgatgatgttttcaggtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcagagttggcgtg

SEQ ID NO: 5 - DNA, 49 bp, Homo sapiens
atagcccttatccaggtttttatctaaggaatcccaagaagactgggga

SEQ ID NO: 6 - DNA, 1479 bp, Homo sapiens
atggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatggccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaagatgacatttgatccagaaatcttcttcaatgttttactgccaccaattatatttcatgcaggatatagtctaaagaagagacacttttttcaaaacttaggatctattttaacgtatgccttcttgggaactgccatctcctgcatcgtcatagggttaattatgtatggttttgtgaaggctatgatacatgctggccagctgaaaaatggagactttcatttcactgactgtttattttttggttcactgatgtctgctacagatccagtgacagtgctggccattttccatgaactgcacgtcgaccctgacctgtacacactcttgtttggagagagtgtgttgaatgatgcagtggccatagtccttacatattctatatccatttacagtcccaaggagaatccaaatgcatttgatgccgcagcattcttccagtctgtggggaatttcctgggaatcttcgctggctcatttgcaatggggtctgcgtatgccatcatcacagcactgttgaccaaatttaccaagctgtgtgagttcccgatgctggaaaccggcctgtttttcctgctttcttggagtgccttcctgtctgccgaggctgccggcctaacagggatagttgctgttctcttctgtggagtcacacaagcacattatacctacaacaatctgtcttcggattccaaaataagaactaaacagttgtttgaatttatgaactttttggcggagaacgtcatcttctgttacatgggcctggcactgttcacgttccagaatcatatctttaatgctctttttatacttggagcctttctagcaatttttgttgccagagcctgcaacatatatcccctctccttcctcctgaatctaggccgaaaacagaagatcccctggaactttcagcacatgatgatgttttcaggtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcagagttggcgtg

SEQ ID NO: 7 - protein, 493 aa, Homo sapiens
MERQSRVMSEKDEYQFQHQGAVELLVFNFLLILTILTIWLFKNHRFRFLHETGGAMVYGLIMGLILRYATAPTDIESGTVYDCVKLTFSPSTLLVNITDQVYEYKYKREISQHNINPHQGNAILEKMTFDPEIFFNVLLPPIIFHAGYSLKKRHFFQNLGSILTYAFLGTAISCIVIGLIMYGFVKAMIHAGQLKNGDFHFTDCLFFGSLMSATDPVTVLAIFHELHVDPDLYTLLFGESVLNDAVAIVLTYSISIYSPKENPNAFDAAAFFQSVGNFLGIFAGSFAMGSAYAIITALLTKFTKLCEFPMLETGLFFLLSWSAFLSAEAAGLTGIVAVLFCGVTQAHYTYNNLSSDSKIRTKQLFEFMNFLAENVIFCYMGLALFTFQNHIFNALFILGAFLAIFVARACNIYPLSFLLNLGRKQKIPWNFQHMMMFSGLRGAIAFALAIRNTESQPKQMMFTTTLLLVFFTVWVFGGGTTPMLTWLQIRVGV

SEQ ID NO: 8 - DNA, 224 bp, Homo sapiens
atagcccttatccaggtttttatctaaggaatcccaagaagactggggaatggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatg

SEQ ID NO: 9 - DNA, 203 bp, Homo sapiens
gccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaag

SEQ ID NO: 10 - DNA, 78 bp, Homo sapiens
atgacatttgatccagaaatcttcttcaatgttttactgccaccaattatatttcatgcaggatatagtctaaagaag

SEQ ID NO: 11 - DNA, 77 bp, Homo sapiens
agacacttttttcaaaacttaggatctattttaacgtatgccttcttgggaactgccatctcctgcatcgtcatagg

SEQ ID NO: 12 - DNA, 116 bp, Homo sapiens
gttaattatgtatggttttgtgaaggctatgatacatgctggccagctgaaaaatggagactttcatttcactgactgtttattttttggttcactgatgtctgctacagatccag

SEQ ID NO: 13 - DNA, 106 bp, Homo sapiens
tgacagtgctggccattttccatgaactgcacgtcgaccctgacctgtacacactcttgtttggagagagtgtgttgaatgatgcagtggccatagtccttacata

SEQ ID NO: 14 - DNA, 139 bp, Homo sapiens
ttctatatccatttacagtcccaaggagaatccaaatgcatttgatgccgcagcattcttccagtctgtggggaatttcctgggaatcttcgctggctcatttgcaatggggtctgcgtatgccatcatcacagcactg

SEQ ID NO: 15 - DNA, 106 bp, Homo sapiens
ttgaccaaatttaccaagctgtgtgagttcccgatgctggaaaccggcctgtttttcctgctttcttggagtgccttcctgtctgccgaggctgccggcctaacag

SEQ ID NO: 16 - DNA, 89 bp, Homo sapiens
ggatagttgctgttctcttctgtggagtcacacaagcacattatacctacaacaatctgtcttcggattccaaaataagaactaaacag

SEQ ID NO: 17 - DNA, 114 bp, Homo sapiens
ttgtttgaatttatgaactttttggcggagaacgtcatcttctgttacatgggcctggcactgttcacgttccagaatcatatctttaatgctctttttatacttggagccttt

SEQ ID NO: 18 - DNA, 112 bp, Homo sapiens
ctagcaatttttgttgccagagcctgcaacatatatcccctctccttcctcctgaatctaggccgaaaacagaagatcccctggaactttcagcacatgatgatgttttcag

SEQ ID NO: 19 - DNA, 154 bp, Homo sapiens
gtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcag

SEQ ID NO: 20 - DNA, 56 bp, Homo sapiens
agttggcgtggacctggatgaaaatctgaaggaggacccctcctcacaacaccagg

SEQ ID NO: 21 - DNA, 79 bp, Homo sapiens
aagcaaataacttggataaaaacatgacgaaagcagagagtgctcggctcttcagaatgtggtatagctttgaccacaa

SEQ ID NO: 22 - DNA, 106 bp, Homo sapiens
gtatctgaaaccaattttaacccactctggtcctccgctgactacaacattacctgaatggtgtggtccgatttccaggctgcttaccagtcctcaagcctatggg

SEQ ID NO: 23 - DNA, 319 bp, Homo sapiens
gaacagctaaaagaggatgatgtggaatgcattgtaaaccaggatgaactagccataaattaccaggagcaagcctcctcaccctgcagtcctcctgcaaggctaggtctggaccagaaagcttcaccccagacgccaggcaaggaaaacatttatgagggagacctcggcctgggaggctatgaactcaagcttgagcaaactttgggtcaatcccagttgaattaattggcatgaagagtacagatgtaatcacaagtaatgcaagactcactgaggaatacaagccaagctgatgaggcagtacaggggagaggct

SEQ ID NO: 24 - DNA, 500 bp, Homo sapiens
agagagaaacaggaagttgtaactagaagccatctgaatactaagccagggcagaatgcttgtgaagtagcaactaaagtggcagtgtttcttctgaaattctcaggcagtcagactgtcttaggcaaatcttgataaaatagcctttatccaggtttttatctaaggaatcccaagaagactggggaatggagagacagtcaagggttatgtcagaaaaggatgagtatcagtttcaacatcagggagcggtggagctgcttgtcttcaattttttgctcatccttaccattttgacaatctggttatttaaaaatcatcgattccgcttcttgcatgaaactggaggagcaatggtgtatggtgagtgcataaattgtgagattgatgtgaaacttgttgcttgagcgtaattagagacttgtgctcagatagcaaagtaccgaaactggagaaaatgtgctgattgaaatgtcattagtttgatgaaaggtgctgca

SEQ ID NO: 25 - DNA, 500 bp, Homo sapiens
tccatgcaaaagtgaacacttttctagaccaggcaataacccaacactttatcctgtcacttctgaaggttccaaatcatataggtttccaatagtggcattttagattgccttcctccagtaccattatttattcttttttttttaggccttataatgggactaattttacgatatgctacagcaccaactgatattgaaagtggaactgtctatgactgtgtaaaactaactttcagtccatcaactctgctggttaatatcactgaccaagtttatgaatataaatacaaaagagaaataagtcagcacaacatcaatcctcatcaaggaaatgctatacttgaaaaggtaaggatcctgtctggtattatatatttgttttacagtataaataattgtatcatatttgagcctttatactttttatataatgcattcacacacacatatatgcctattttaacaccactcttttggagacgttaattgactaagga

SEQ ID NO: 26 - DNA, 500 bp, Homo sapiens
gtatgtagattttacctttagaagctacagttatactgtatttatgaatttccaaattttcattttttcctaccttcatctctgccaagttaatagttaatatgcctcacaaaggcttagcaaaagcaattacaactgttttttgaaacatgtttttttaatgacccaaaggagcatcattctaactatcctttttttcctttctggcagatgacatttgatccagaaatcttcttcaatgttttactgccaccaattatatttcatgcaggatatagtctaaagaaggtaaatatatagtgatcattttcttcttttatcattaagtagagaattttaccagcactaatgagaaactgttatttttatgggcttctctatttggtaagagtatcattatcttaaatttgtgatgcagattcaaacctgacatgcttagaattctagacttcttctaaacctgttctgttgaatacaggccattaaccacatgtaactat

SEQ ID NO: 27 - DNA, 500 bp, Homo sapiens
tttttttctgcttcttgacttttccctccagtcctttgcaatgataatcagttaagtccctattagcacaataaggccaggaacgaggctccaaaaccaaaatttgcctaaacagtgaggggtctgtgaactgcatacatttatttttattttcttttgcactaagaaaaaattctgatgtactctattaaatatctttttttctccccagagacacttttttcaaaacttaggatctattttaacgtatgccttcttgggaactgccatctcctgcatcgtcatagggtaagtgacattcggagctcaagttgcaggtggctgtggggtctgtgatctgtgtgagggatctaacacttccaggattcttgctggctgggaaaattgtcttttttttagtatatcacatatttgtatgttttttctgacttaattccacggcttctgacaaatacaaggcttcaaatcaaagcaaactagaggattgctggactttctct

SEQ ID NO: 28 - DNA, 500 bp, Homo sapiens
tttggtaaaattgtctttcatagccaagagattccagtgtacagagacctagcacggctgtcatttggcaggatggtggcaatttgtgcatgatacgtattcaggaaaaacattagcatgtggcttatgatagcatgggtttacacagggttcatcttgatgtttgaaggtcatgtttttctcttcaacaggttaattatgtatggttttgtgaaggctatgatacatgctggccagctgaaaaatggagactttcatttcactgactgtttattttttggttcactgatgtctgctacagatccaggtaattgctcaaataatatattttcttcttttaagaatcaaacatttaaattgaatgtttctccccaagaccatcacctgcgtctacataataataatttctatttttcttcacctgcagttgacatatctgttagtaattacaaaagaaaagaaaaggggaatattttgatagtgctaaagaaattggttag

SEQ ID NO: 29 - DNA, 500 bp, Homo sapiens
attttatctttaagcagtagggagccaagagaagcctcttaagcatcagatattttttgaataaaggtctggtgttagtatagtcattggatttgagaagatgagcatttcattaatgtttttgagtggtgaccatagccaaggaaaactccatgcctgcatccaagctaacctgcatgtttgtgtgtgtcttcctagtgacagtgctggccattttccatgaactgcacgtcgaccctgacctgtacacactcttgtttggagagagtgtgttgaatgatgcagtggccatagtccttacatagtaagtaccagaacttggcctatgtttctttaatcatgtgaaatatgctttcacttgtatcttttctcttatgtcacagagatacgggcagtctctagcttttttcaaggaattgttatcacagaaaatttccataatgggcacaattcattaggcatctaatttctaatactattaatgggttttatgtttccag

SEQ ID NO: 30 - DNA, 500 bp, Homo sapiens
aggaagcagaaagatttctgcattctccaactctccccttctgtatctgttttcccctaactcttccaaccagcacccaacattacagggaaagatacctctccccaaagtgggtgcgaaatcctatctgggctatgagatgaaagtcactaactaacctccaccctcttctcttttcagttctatatccatttacagtcccaaggagaatccaaatgcatttgatgccgcagcattcttccagtctgtggggaatttcctgggaatcttcgctggctcatttgcaatggggtctgcgtatgccatcatcacagcactgatatcctttgtctgtatgtggaaagctgggactgtcaagaacagtcagtatccatggaatatttctgatgatggacaacctccaggcccataaaaggctagatagtctggtccaagtttcatacggcaccactggtgccatcttggttctcagtcacccctggaggcataggttggtacca

SEQ ID NO: 31 - DNA, 500 bp, Homo sapiens
cccccggtgaagcatggaactcgtgctggccttcaaagtaggtcccagtgagaattatggccttccagtcaggtctgaatatcagttcttttactttgctctagagctatcatcaccagatcagtttttattctcagccaagaaggagatgtagctgtaaaagctgttgttgtttcccttaacttatcttcctcacttgaccaaatttaccaagctgtgtgagttcccgatgctggaaaccggcctgtttttcctgctttcttggagtgccttcctgtctgccgaggctgccggcctaacaggtcagtgcttcatgctgcacagccaactggatgtgtgaatatgtgtgtggctctggtgtgtgcccctccctgacctagcaggaggtgaaatgaagtctctcttaaatagtgcatgtgaatggtttgggtcagaatcagcctctcttgggtacgctggagagaaaaaataatagtaattcatccattttgtaccaaaca

SEQ ID NO: 32 - DNA, 500 bp, Homo sapiens
tttttctttaggtttaatccttcaggaccctgtttaaaccctgtttaaagcaagtgcaagatttacctaggttgcattatcatggtaactactagcagttttaactctactgacaccaagtctattttccaactggaatagccctaacctttgacttggatggaaaagaaaaattggttttgactgatctgtttttttaatttcagggatagttgctgttctcttctgtggagtcacacaagcacattatacctacaacaatctgtcttcggattccaaaataagaactaaacaggtaaaagaaaaaatttactacagacttggtctcttttctcagtgaaatggttatgtagcaattctggcagtatagtcagaaagagtttcattagtaaaggaagaatgtaagcttaaggcttcattacgagttggatatttatagatttttttccctccaggataaccattaacaattctcttggtaagcaaaaatatagttataa

SEQ ID NO: 33 - DNA, 500 bp, Homo sapiens
tgatgtggctgtggcatctgtccctgggtcaagagccagggcctccatcctccttcagaggctaagcacacggccaaaggggtggccttccagataaaggagacccagtcagttgtcaaatacagttaccagaaagtaagtgcactcaaacctgagttataattaatggttttttgttttatgttttgtttcagttgtttgaatttatgaactttttggcggagaacgtcatcttctgttacatgggcctggcactgttcacgttccagaatcatatctttaatgctctttttatacttggagcctttgtatcctttgaaatacagaacatggggtgattagagaagataacgaatgacttacttttacggattattctgttaaatcattaaagcactttcctaaagtccactgcaaggctcagacacaaggcagatacgtgatttctagttaggggaggaaaacatcctaagaaattaggacctccattaaataattat

SEQ ID NO: 34 - DNA, 500 bp, Homo sapiens
tattatttctgggaagaacttgactttgaaatataaattttggaagcaagttggacaaatgtgaattgacatgtttatttctggttttggggtcataatgaagagaagcttgaggacataatatttaagctcaaggattcaaaccatcattgcagcaacactttcctcaatgtttccctgtgcttatctacagctagcaatttttgttgccagagcctgcaacatatatcccctctccttcctcctgaatctaggccgaaaacagaagatcccctggaactttcagcacatgatgatgttttcaggtatgtgaactatgttatatgtaaagtgctgctggttttctcacaattttacagaacatgaaaaaccatgagaaaatttagaacccttttggggaaacaacttctctttatcccattgtacctaacactatgctgattctccgtaagtgctgagcaacagcaggatttctctttccaagtgggaaatgaagttag

SEQ ID NO: 35 - DNA, 500 bp, Homo sapiens
gtacacagtgtctttctgttttatttcttacaactctttatgaatctataattatctcaaaataaaaagattaatttaaaaaaattgtgtcagcaaattgtaaagatgaatccattgaaatgaaaaagacacctgcaaggcatttattttctcctttggtttttcctgcaaggtttgcgaggagcgatcgcatttgccttagctattcggaacacagaatctcagcccaaacaaatgatgtttaccactacgctgctcctcgtgttcttcactgtctgggtatttggaggaggaacaacccccatgttgacttggcttcagatcaggtgagtgacaccgtctagcaaaggaaatcattatgaggcatggctgcgctgttcaatttctggtattgctgacttagtcaatagtacagtggcaggtcctaccctaactttcccagcagtcaagttgaccttcactcataaggtgaaacaatatgacatagtaccaaccacact

SEQ ID NO: 36 - DNA, 500 bp, Homo sapiens
tgctgttgatgcttaatttctccagcatcctgaggcccagctgttcaagttcttattttggtataaatctgaggttcttttgattcaagaagacgtaggatagttttctgccaaatcttttctcagattcacattgtgtttgggttaggtcatctgacacacaagactgtctcattccttctgcagcaggggattgaccagtttttggttttgtttaacagagttggcgtggacctggatgaaaatctgaaggaggacccctcctcacaacaccaggcaagtcaaagaaaccaatgcacattatatagagatatgattgttctgttccataggctgttccatgtttgtccagcatgtgaccaaggagctatttctaaacggagcttcctctgctgataaaacaaatgagattacaataaaatacaagggaattcagggtgttgataacttctagcatatgaatgcttagaactagcatgtacttgcaagcctctctatgg

SEQ ID NO: 37 - DNA, 500 bp, Homo sapiens
aatgttgaaatactccaaaagggtctcatccacatagcaagatttatgagctaatttgcctggaaactgcttcaccatttagatgaaaatgcctatacttagaaaggttttttttaaatgtagttaaatttaacttggtttgacatgtatttttatattaaaatctttttgactatttacccaatgcctttttctattgtttctccccaggaagcaaataacttggataaaaacatgacgaaagcagagagtgctcggctcttcagaatgtggtatagctttgaccacaagtatcctttgataataacgatctcacagatcctgctgcaggcagagaattactttaagctcttaatcattgaagtgtgcactcctgctcttcaagaaacaccaaaaggtaaagtgaattcacagccaaacttataaaaaggaatgatttcataaaataaaaagcagggtagtccatacttaagtgtttcagagagctaaagtgaaacttc

SEQ ID NO: 38 - DNA, 500 bp, Homo sapiens
ggctatcaggaaggctggaaaagtatgtgtaacaaagattataaacagcaatttttgcagggcaaatttcttcctaagtgagggagtaggggatttaaaggcaaacgtagctgtctcagcatacaaaggtttgtgcagcgtaagattcctaaatcctaactccctgttgacaagtattccttaacaggcctcccaggtatctgaaaccaattttaacccactctggtcctccgctgactacaacattacctgaatggtgtggtccgatttccaggctgcttaccagtcctcaagcctatggggtaagtaaatctcaggcctctagggtgagggaatatcccttgtgggacagacttggtgaatatcgatgagcccaggaacctcaaaaagcctgctacttgcaggattgaggaggaagctttttttttttttttttttttttttgagacggagtcttgctctgttgcccaggctggagtgcagtggcgtgatctcagctc

SEQ ID NO: 39 - DNA, 500 bp, Homo sapiens
attctgtttaaaaaaaaaaaaaaattgtaggactgctattgttttctgccttcatcatgaagtgacacctctctctcttttcccaaccaggaacagctaaaagaggatgatgtggaatgcattgtaaaccaggatgaactagccataaattaccaggagcaagcctcctcaccctgcagtcctcctgcaaggctaggtctggaccagaaagcttcaccccagacgccaggcaaggaaaacatttatgagggagacctcggcctgggaggctatgaactcaagcttgagcaaactttgggtcaatcccagttgaattaattggcatgaagagtacagatgtaatcacaagtaatgcaagactcactgaggaatacaagccaagctgatgaggcagtacaggggagaggctggaaaacatattaagagcataaattggagagaatcaaagccttgtcacatggatcctctggtgcctgaagaaatgagattttattatccct

SEQ ID NO: 40 - DNA, 1000 bp, Homo sapiens
tctgtgggtcctggatgccgagaccaggaaccctacaaagagaaatccatgcatgtggcgttgctgaggatggctgcgtgagtttctctttcgaagtaatgtgttttgttttttttcttttttgtctcaacgtatccaaacagtacagttgtagagatggggatggaattttatttgtcttttgctttcattgttttctcttgttaaaagaaaaacaattgatttagagtttaaaggctgctctgcaactatcaattatataaagccaaatagagtcatatactaaatcctttcttgcttctcaactaaaaaaaaaaaaaaaaaatcctgtgaaatcactttaggtttgcttaaccaatgtacagtcatgatttgagagcataagaaaccctataagaaaccctatgccttaagtgaatcccattgtgagatacttgagctatgaggcggaagtttcaggctaagcagtgtaatatcttttcaggaggctaatatgctgatccatttagagcacagaggctgattgataactgtgctatcaatccccatcactttggcaggtcttggttagagaggaaagatcttgcatttaggtgaagttgagtcaggaattggctttttgaacttagaaaacgttgctagtgtaaggctaccttttttattattaatctacagttcatatttatgcagcttaagaagacttatttggaataataagaccgtgattcaagatcttagcatcattgcttgcagatttgtccagtatgaaagatattttcatgagatgaatcacttaaagacattaagagtcagtgatgaaaccacacactctcgagtttgaaaagagacagggctgctttaagagagaaacaggaagttgtaactagaagccatctgaatactaagccagggcagaatgcttgtgaagtagcaactaaagtggcagtgtttcttctgaaattctcaggcagtcagactgtcttaggcaaatcttgataaa

SEQ ID NO: 41 - DNA, 17 bp, Homo sapiens
atagcccttatccaggt

SEQ ID NO: 42 - DNA, 17 bp, Homo sapiens
tagcccttatccaggtt

SEQ ID NO: 43 - DNA, 17 bp, Homo sapiens
agcccttatccaggttt

SEQ ID NO: 44 - DNA, 17 bp, Homo sapiens
gcccttatccaggtttt

SEQ ID NO: 45 - DNA, 17 bp, Homo sapiens
cccttatccaggttttt

SEQ ID NO: 46 - DNA, 17 bp, Homo sapiens
ccttatccaggttttta

SEQ ID NO: 47 - DNA, 17 bp, Homo sapiens
cttatccaggtttttat

SEQ ID NO: 48 - DNA, 17 bp, Homo sapiens
ttatccaggtttttatc

SEQ ID NO: 49 - DNA, 17 bp, Homo sapiens
tatccaggtttttatct

SEQ ID NO: 50 - DNA, 17 bp, Homo sapiens
atccaggtttttatcta

SEQ ID NO: 51 - DNA, 17 bp, Homo sapiens
tccaggtttttatctaa

SEQ ID NO: 52 - DNA, 17 bp, Homo sapiens
ccaggtttttatctaag

SEQ ID NO: 53 - DNA, 17 bp, Homo sapiens
caggtttttatctaagg

SEQ ID NO: 54 - DNA, 17 bp, Homo sapiens
aggtttttatctaagga

SEQ ID NO: 55 - DNA, 17 bp, Homo sapiens
ggtttttatctaaggaa

SEQ ID NO: 56 - DNA, 17 bp, Homo sapiens
gtttttatctaaggaat

SEQ ID NO: 57 - DNA, 17 bp, Homo sapiens
tttttatctaaggaatc

SEQ ID NO: 58 - DNA, 17 bp, Homo sapiens
ttttatctaaggaatcc

SEQ ID NO: 59 - DNA, 17 bp, Homo sapiens
tttatctaaggaatccc

SEQ ID NO: 60 - DNA, 17 bp, Homo sapiens
ttatctaaggaatccca

SEQ ID NO: 61 - DNA, 17 bp, Homo sapiens
tatctaaggaatcccaa

SEQ ID NO: 62 - DNA, 17 bp, Homo sapiens
atctaaggaatcccaag

SEQ ID NO: 63 - DNA, 17 bp, Homo sapiens
tctaaggaatcccaaga

SEQ ID NO: 64 - DNA, 17 bp, Homo sapiens
ctaaggaatcccaagaa

SEQ ID NO: 65 - DNA, 17 bp, Homo sapiens
taaggaatcccaagaag

SEQ ID NO: 66 - DNA, 17 bp, Homo sapiens
aaggaatcccaagaaga

SEQ ID NO: 67 - DNA, 17 bp, Homo sapiens
aggaatcccaagaagac

SEQ ID NO: 68 - DNA, 17 bp, Homo sapiens
ggaatcccaagaagact

SEQ ID NO: 69 - DNA, 17 bp, Homo sapiens
gaatcccaagaagactg

SEQ ID NO: 70 - DNA, 17 bp, Homo sapiens
aatcccaagaagactgg

SEQ ID NO: 71 - DNA, 17 bp, Homo sapiens
atcccaagaagactggg

SEQ ID NO: 72 - DNA, 17 bp, Homo sapiens
tcccaagaagactgggg

SEQ ID NO: 73 - DNA, 17 bp, Homo sapiens
cccaagaagactgggga

SEQ ID NO: 74 - DNA, 17 bp, Homo sapiens
ccaagaagactggggaa

SEQ ID NO: 75 - DNA, 17 bp, Homo sapiens
caagaagactggggaat

SEQ ID NO: 76 - DNA, 17 bp, Homo sapiens
aagaagactggggaatg

SEQ ID NO: 77 - DNA, 17 bp, Homo sapiens
agaagactggggaatgg

SEQ ID NO: 78 - DNA, 17 bp, Homo sapiens
gaagactggggaatgga

SEQ ID NO: 79 - DNA, 17 bp, Homo sapiens
aagactggggaatggag

SEQ ID NO: 80 - DNA, 17 bp, Homo sapiens
agactggggaatggaga

SEQ ID NO: 81 - DNA, 17 bp, Homo sapiens
gactggggaatggagag

SEQ ID NO: 82 - DNA, 17 bp, Homo sapiens
actggggaatggagaga

SEQ ID NO: 83 - DNA, 17 bp, Homo sapiens
ctggggaatggagagac

SEQ ID NO: 84 - DNA, 17 bp, Homo sapiens
tggggaatggagagaca

SEQ ID NO: 85 - DNA, 17 bp, Homo sapiens
ggggaatggagagacag

SEQ ID NO: 86 - DNA, 17 bp, Homo sapiens
gggaatggagagacagt

SEQ ID NO: 87 - DNA, 17 bp, Homo sapiens
ggaatggagagacagtc

SEQ ID NO: 88 - DNA, 17 bp, Homo sapiens
gaatggagagacagtca

SEQ ID NO: 89 - DNA, 17 bp, Homo sapiens
aatggagagacagtcaa

SEQ ID NO: 90 - DNA, 17 bp, Homo sapiens
atggagagacagtcaag

SEQ ID NO: 91 - DNA, 17 bp, Homo sapiens
tggagagacagtcaagg

SEQ ID NO: 92 - DNA, 17 bp, Homo sapiens
ggagagacagtcaaggg

SEQ ID NO: 93 - DNA, 17 bp, Homo sapiens
gagagacagtcaagggt

SEQ ID NO: 94 - DNA, 17 bp, Homo sapiens
agagacagtcaagggtt

SEQ ID NO: 95 - DNA, 17 bp, Homo sapiens
gagacagtcaagggtta

SEQ ID NO: 96 - DNA, 17 bp, Homo sapiens
agacagtcaagggttat

SEQ ID NO: 97 - DNA, 17 bp, Homo sapiens
gacagtcaagggttatg

SEQ ID NO: 98 - DNA, 17 bp, Homo sapiens
acagtcaagggttatgt

SEQ ID NO: 99 - DNA, 17 bp, Homo sapiens
cagtcaagggttatgtc

SEQ ID NO: 100 - DNA, 17 bp, Homo sapiens
agtcaagggttatgtca

SEQ ID NO: 101 - DNA, 17 bp, Homo sapiens
gtcaagggttatgtcag

SEQ ID NO: 102 - DNA, 17 bp, Homo sapiens
tcaagggttatgtcaga

SEQ ID NO: 103 - DNA, 17 bp, Homo sapiens
caagggttatgtcagaa

SEQ ID NO: 104 - DNA, 17 bp, Homo sapiens
aagggttatgtcagaaa

SEQ ID NO: 105 - DNA, 17 bp, Homo sapiens
agggttatgtcagaaaa

SEQ ID NO: 106 - DNA, 17 bp, Homo sapiens
gggttatgtcagaaaag

SEQ ID NO: 107 - DNA, 17 bp, Homo sapiens
ggttatgtcagaaaagg

SEQ ID NO: 108 - DNA, 17 bp, Homo sapiens
gttatgtcagaaaagga

SEQ ID NO: 109 - DNA, 17 bp, Homo sapiens
ttatgtcagaaaaggat

SEQ ID NO: 110 - DNA, 17 bp, Homo sapiens
tatgtcagaaaaggatg

SEQ ID NO: 111 - DNA, 17 bp, Homo sapiens
atgtcagaaaaggatga

SEQ ID NO: 112 - DNA, 17 bp, Homo sapiens
tgtcagaaaaggatgag

SEQ ID NO: 113 - DNA, 17 bp, Homo sapiens
gtcagaaaaggatgagt

SEQ ID NO: 114 - DNA, 17 bp, Homo sapiens
tcagaaaaggatgagta

SEQ ID NO: 115 - DNA, 17 bp, Homo sapiens
cagaaaaggatgagtat

SEQ ID NO: 116 - DNA, 17 bp, Homo sapiens
agaaaaggatgagtatc

SEQ ID NO: 117 - DNA, 17 bp, Homo sapiens
gaaaaggatgagtatca

SEQ ID NO: 118 - DNA, 17 bp, Homo sapiens
aaaaggatgagtatcag

SEQ ID NO: 119 - DNA, 17 bp, Homo sapiens
aaaggatgagtatcagt

SEQ ID NO: 120 - DNA, 17 bp, Homo sapiens
aaggatgagtatcagtt

SEQ ID NO: 121 - DNA, 17 bp, Homo sapiens
aggatgagtatcagttt

SEQ ID NO: 122 - DNA, 17 bp, Homo sapiens
ggatgagtatcagtttc

SEQ ID NO: 123 - DNA, 17 bp, Homo sapiens
gatgagtatcagtttca

SEQ ID NO: 124 - DNA, 17 bp, Homo sapiens
atgagtatcagtttcaa

SEQ ID NO: 125 - DNA, 17 bp, Homo sapiens
tgagtatcagtttcaac

SEQ ID NO: 126 - DNA, 17 bp, Homo sapiens
gagtatcagtttcaaca

SEQ ID NO: 127 - DNA, 17 bp, Homo sapiens
agtatcagtttcaacat

SEQ ID NO: 128 - DNA, 17 bp, Homo sapiens
gtatcagtttcaacatc

SEQ ID NO: 129 - DNA, 17 bp, Homo sapiens
tatcagtttcaacatca

SEQ ID NO: 130 - DNA, 17 bp, Homo sapiens
atcagtttcaacatcag

SEQ ID NO: 131 - DNA, 17 bp, Homo sapiens
tcagtttcaacatcagg

SEQ ID NO: 132 - DNA, 17 bp, Homo sapiens
cagtttcaacatcaggg

SEQ ID NO: 133 - DNA, 17 bp, Homo sapiens
agtttcaacatcaggga

SEQ ID NO: 134 - DNA, 17 bp, Homo sapiens
gtttcaacatcagggag

SEQ ID NO: 135 - DNA, 17 bp, Homo sapiens
tttcaacatcagggagc

SEQ ID NO: 136 - DNA, 17 bp, Homo sapiens
ttcaacatcagggagcg

SEQ ID NO: 137 - DNA, 17 bp, Homo sapiens
tcaacatcagggagcgg

SEQ ID NO: 138 - DNA, 17 bp, Homo sapiens
caacatcagggagcggt

SEQ ID NO: 139 - DNA, 17 bp, Homo sapiens
aacatcagggagcggtg

SEQ ID NO: 140 - DNA, 17 bp, Homo sapiens
acatcagggagcggtgg

SEQ ID NO: 141 - DNA, 17 bp, Homo sapiens
catcagggagcggtgga

SEQ ID NO: 142 - DNA, 17 bp, Homo sapiens
atcagggagcggtggag

SEQ ID NO: 143 - DNA, 17 bp, Homo sapiens
tcagggagcggtggagc

SEQ ID NO: 144 - DNA, 17 bp, Homo sapiens
cagggagcggtggagct

SEQ ID NO: 145 - DNA, 17 bp, Homo sapiens
agggagcggtggagctg

SEQ ID NO: 146 - DNA, 17 bp, Homo sapiens
gggagcggtggagctgc

SEQ ID NO: 147 - DNA, 17 bp, Homo sapiens
ggagcggtggagctgct

SEQ ID NO: 148 - DNA, 17 bp, Homo sapiens
gagcggtggagctgctt

SEQ ID NO: 149 - DNA, 17 bp, Homo sapiens
agcggtggagctgcttg

SEQ ID NO: 150 - DNA, 17 bp, Homo sapiens
gcggtggagctgcttgt

SEQ ID NO: 151 - DNA, 17 bp, Homo sapiens
cggtggagctgcttgtc

SEQ ID NO: 152 - DNA, 17 bp, Homo sapiens
ggtggagctgcttgtct

SEQ ID NO: 153 - DNA, 17 bp, Homo sapiens
gtggagctgcttgtctt

SEQ ID NO: 154 - DNA, 17 bp, Homo sapiens
tggagctgcttgtcttc

SEQ ID NO: 155 - DNA, 17 bp, Homo sapiens
ggagctgcttgtcttca

SEQ ID NO: 156 - DNA, 17 bp, Homo sapiens
gagctgcttgtcttcaa

SEQ ID NO: 157 - DNA, 17 bp, Homo sapiens
agctgcttgtcttcaat

SEQ ID NO: 158 - DNA, 17 bp, Homo sapiens
gctgcttgtcttcaatt

SEQ ID NO: 159 - DNA, 17 bp, Homo sapiens
ctgcttgtcttcaattt

SEQ ID NO: 160 - DNA, 17 bp, Homo sapiens
tgcttgtcttcaatttt

SEQ ID NO: 161 - DNA, 17 bp, Homo sapiens
gcttgtcttcaattttt

SEQ ID NO: 162 - DNA, 17 bp, Homo sapiens
cttgtcttcaatttttt

SEQ ID NO: 163 - DNA, 17 bp, Homo sapiens
ttgtcttcaattttttg

SEQ ID NO: 164 - DNA, 17 bp, Homo sapiens
tgtcttcaattttttgc

SEQ ID NO: 165 - DNA, 17 bp, Homo sapiens
gtcttcaattttttgct

SEQ ID NO: 166 - DNA, 17 bp, Homo sapiens
tcttcaattttttgctc

SEQ ID NO: 167 - DNA, 17 bp, Homo sapiens
cttcaattttttgctca

SEQ ID NO: 168 - DNA, 17 bp, Homo sapiens
ttcaattttttgctcat

SEQ ID NO: 169 - DNA, 17 bp, Homo sapiens
tcaattttttgctcatc

SEQ ID NO: 170 - DNA, 17 bp, Homo sapiens
caattttttgctcatcc

SEQ ID NO: 171 - DNA, 17 bp, Homo sapiens
aattttttgctcatcct

SEQ ID NO: 172 - DNA, 17 bp, Homo sapiens
attttttgctcatcctt

SEQ ID NO: 173 - DNA, 17 bp, Homo sapiens
ttttttgctcatcctta

SEQ ID NO: 174 - DNA, 17 bp, Homo sapiens
tttttgctcatccttac

SEQ ID NO: 175 - DNA, 17 bp, Homo sapiens
ttttgctcatccttacc

SEQ ID NO: 176 - DNA, 17 bp, Homo sapiens
tttgctcatccttacca

SEQ ID NO: 177 - DNA, 17 bp, Homo sapiens
ttgctcatccttaccat

SEQ ID NO: 178 - DNA, 17 bp, Homo sapiens
tgctcatccttaccatt

SEQ ID NO: 179 - DNA, 17 bp, Homo sapiens
gctcatccttaccattt

SEQ ID NO: 180 - DNA, 17 bp, Homo sapiens
ctcatccttaccatttt

SEQ ID NO: 181 - DNA, 17 bp, Homo sapiens
tcatccttaccattttg

SEQ ID NO: 182 - DNA, 17 bp, Homo sapiens
catccttaccattttga

SEQ ID NO: 183 - DNA, 17 bp, Homo sapiens
atccttaccattttgac

SEQ ID NO: 184 - DNA, 17 bp, Homo sapiens
tccttaccattttgaca

SEQ ID NO: 185 - DNA, 17 bp, Homo sapiens
ccttaccattttgacaa

SEQ ID NO: 186 - DNA, 17 bp, Homo sapiens
cttaccattttgacaat

SEQ ID NO: 187 - DNA, 17 bp, Homo sapiens
ttaccattttgacaatc

SEQ ID NO: 188 - DNA, 17 bp, Homo sapiens
taccattttgacaatct

SEQ ID NO: 189 - DNA, 17 bp, Homo sapiens
accattttgacaatctg

SEQ ID NO: 190 - DNA, 17 bp, Homo sapiens
ccattttgacaatctgg

SEQ ID NO: 191 - DNA, 17 bp, Homo sapiens
cattttgacaatctggt

SEQ ID NO: 192 - DNA, 17 bp, Homo sapiens
attttgacaatctggtt

SEQ ID NO: 193 - DNA, 17 bp, Homo sapiens
ttttgacaatctggtta

SEQ ID NO: 194 - DNA, 17 bp, Homo sapiens
tttgacaatctggttat

SEQ ID NO: 195 - DNA, 17 bp, Homo sapiens
ttgacaatctggttatt

SEQ ID NO: 196 - DNA, 17 bp, Homo sapiens
tgacaatctggttattt

SEQ ID NO: 197 - DNA, 17 bp, Homo sapiens
gacaatctggttattta

SEQ ID NO: 198 - DNA, 17 bp, Homo sapiens
acaatctggttatttaa

SEQ ID NO: 199 - DNA, 17 bp, Homo sapiens
caatctggttatttaaa

SEQ ID NO: 200 - DNA, 17 bp, Homo sapiens
aatctggttatttaaaa

SEQ ID NO: 201 - DNA, 17 bp, Homo sapiens
atctggttatttaaaaa

SEQ ID NO: 202 - DNA, 17 bp, Homo sapiens
tctggttatttaaaaat

SEQ ID NO: 203 - DNA, 17 bp, Homo sapiens
ctggttatttaaaaatc

SEQ ID NO: 204 - DNA, 17 bp, Homo sapiens
tggttatttaaaaatca

SEQ ID NO: 205 - DNA, 17 bp, Homo sapiens
ggttatttaaaaatcat

SEQ ID NO: 206 - DNA, 17 bp, Homo sapiens
gttatttaaaaatcatc

SEQ ID NO: 207 - DNA, 17 bp, Homo sapiens
ttatttaaaaatcatcg

SEQ ID NO: 208 - DNA, 17 bp, Homo sapiens
tatttaaaaatcatcga

SEQ ID NO: 209 - DNA, 17 bp, Homo sapiens
atttaaaaatcatcgat

SEQ ID NO: 210 - DNA, 17 bp, Homo sapiens
tttaaaaatcatcgatt

SEQ ID NO: 211 - DNA, 17 bp, Homo sapiens
ttaaaaatcatcgattc

SEQ ID NO: 212 - DNA, 17 bp, Homo sapiens
taaaaatcatcgattcc

SEQ ID NO: 213 - DNA, 17 bp, Homo sapiens
aaaaatcatcgattccg

SEQ ID NO: 214 - DNA, 17 bp, Homo sapiens
aaaatcatcgattccgc

SEQ ID NO: 215 - DNA, 17 bp, Homo sapiens
aaatcatcgattccgct

SEQ ID NO: 216 - DNA, 17 bp, Homo sapiens
aatcatcgattccgctt

SEQ ID NO: 217 - DNA, 17 bp, Homo sapiens
atcatcgattccgcttc

SEQ ID NO: 218 - DNA, 17 bp, Homo sapiens
tcatcgattccgcttct

SEQ ID NO: 219 - DNA, 17 bp, Homo sapiens
catcgattccgcttctt

SEQ ID NO: 220 - DNA, 17 bp, Homo sapiens
atcgattccgcttcttg

SEQ ID NO: 221 - DNA, 17 bp, Homo sapiens
tcgattccgcttcttgc

SEQ ID NO: 222 - DNA, 17 bp, Homo sapiens
cgattccgcttcttgca

SEQ ID NO: 223 - DNA, 17 bp, Homo sapiens
gattccgcttcttgcat

SEQ ID NO: 224 - DNA, 17 bp, Homo sapiens
attccgcttcttgcatg

SEQ ID NO: 225 - DNA, 17 bp, Homo sapiens
ttccgcttcttgcatga

SEQ ID NO: 226 - DNA, 17 bp, Homo sapiens
tccgcttcttgcatgaa

SEQ ID NO: 227 - DNA, 17 bp, Homo sapiens
ccgcttcttgcatgaaa

SEQ ID NO: 228 - DNA, 17 bp, Homo sapiens
cgcttcttgcatgaaac

SEQ ID NO: 229 - DNA, 17 bp, Homo sapiens
gcttcttgcatgaaact

SEQ ID NO: 230 - DNA, 17 bp, Homo sapiens
cttcttgcatgaaactg

SEQ ID NO: 231 - DNA, 17 bp, Homo sapiens
ttcttgcatgaaactgg

SEQ ID NO: 232 - DNA, 17 bp, Homo sapiens
tcttgcatgaaactgga

SEQ ID NO: 233 - DNA, 17 bp, Homo sapiens
cttgcatgaaactggag

SEQ ID NO: 234 - DNA, 17 bp, Homo sapiens
ttgcatgaaactggagg

SEQ ID NO: 235 - DNA, 17 bp, Homo sapiens
tgcatgaaactggagga

SEQ ID NO: 236 - DNA, 17 bp, Homo sapiens
gcatgaaactggaggag

SEQ ID NO: 237 - DNA, 17 bp, Homo sapiens
catgaaactggaggagc

SEQ ID NO: 238 - DNA, 17 bp, Homo sapiens
atgaaactggaggagca

SEQ ID NO: 239 - DNA, 17 bp, Homo sapiens
tgaaactggaggagcaa

SEQ ID NO: 240 - DNA, 17 bp, Homo sapiens
gaaactggaggagcaat

SEQ ID NO: 241 - DNA, 17 bp, Homo sapiens
aaactggaggagcaatg

SEQ ID NO: 242 - DNA, 17 bp, Homo sapiens
aactggaggagcaatgg

SEQ ID NO: 243 - DNA, 17 bp, Homo sapiens
actggaggagcaatggt

SEQ ID NO: 244 - DNA, 17 bp, Homo sapiens
ctggaggagcaatggtg

SEQ ID NO: 245 - DNA, 17 bp, Homo sapiens
tggaggagcaatggtgt

SEQ ID NO: 246 - DNA, 17 bp, Homo sapiens
ggaggagcaatggtgta

SEQ ID NO: 247 - DNA, 17 bp, Homo sapiens
gaggagcaatggtgtat

SEQ ID NO: 248 - DNA, 17 bp, Homo sapiens
aggagcaatggtgtatg

SEQ ID NO: 249 - DNA, 17 bp, Homo sapiens
ggagcaatggtgtatgg

SEQ ID NO: 250 - DNA, 17 bp, Homo sapiens
gagcaatggtgtatggc

SEQ ID NO: 251 - DNA, 17 bp, Homo sapiens
agcaatggtgtatggcc

SEQ ID NO: 252 - DNA, 17 bp, Homo sapiens
gcaatggtgtatggcct

SEQ ID NO: 253 - DNA, 17 bp, Homo sapiens
caatggtgtatggcctt

SEQ ID NO: 254 - DNA, 17 bp, Homo sapiens
aatggtgtatggcctta

SEQ ID NO: 255 - DNA, 17 bp, Homo sapiens
atggtgtatggccttat

SEQ ID NO: 256 - DNA, 17 bp, Homo sapiens
tggtgtatggccttata

SEQ ID NO: 257 - DNA, 17 bp, Homo sapiens
ggtgtatggccttataa

SEQ ID NO: 258 - DNA, 17 bp, Homo sapiens
gtgtatggccttataat

SEQ ID NO: 259 - DNA, 17 bp, Homo sapiens
tgtatggccttataatg

SEQ ID NO: 260 - DNA, 17 bp, Homo sapiens
gtatggccttataatgg

SEQ ID NO: 261 - DNA, 17 bp, Homo sapiens
tatggccttataatggg

SEQ ID NO: 262 - DNA, 17 bp, Homo sapiens
atggccttataatggga

SEQ ID NO: 263 - DNA, 17 bp, Homo sapiens
tggccttataatgggac

SEQ ID NO: 264 - DNA, 17 bp, Homo sapiens
ggccttataatgggact

SEQ ID NO: 265 - DNA, 17 bp, Homo sapiens
gccttataatgggacta

SEQ ID NO: 266 - DNA, 17 bp, Homo sapiens
ccttataatgggactaa

SEQ ID NO: 267 - DNA, 17 bp, Homo sapiens
cttataatgggactaat

SEQ ID NO: 268 - DNA, 17 bp, Homo sapiens
ttataatgggactaatt

SEQ ID NO: 269 - DNA, 17 bp, Homo sapiens
tataatgggactaattt

SEQ ID NO: 270 - DNA, 17 bp, Homo sapiens
ataatgggactaatttt

SEQ ID NO: 271 - DNA, 17 bp, Homo sapiens
taatgggactaatttta

SEQ ID NO: 272 - DNA, 17 bp, Homo sapiens
aatgggactaattttac

SEQ ID NO: 273 - DNA, 17 bp, Homo sapiens
atgggactaattttacg

SEQ ID NO: 274 - DNA, 17 bp, Homo sapiens
tgggactaattttacga

SEQ ID NO: 275 - DNA, 17 bp, Homo sapiens
gggactaattttacgat

SEQ ID NO: 276 - DNA, 17 bp, Homo sapiens
ggactaattttacgata

SEQ ID NO: 277 - DNA, 17 bp, Homo sapiens
gactaattttacgatat

SEQ ID NO: 278 - DNA, 17 bp, Homo sapiens
actaattttacgatatg

SEQ ID NO: 279 - DNA, 17 bp, Homo sapiens
ctaattttacgatatgc

SEQ ID NO: 280 - DNA, 17 bp, Homo sapiens
taattttacgatatgct

SEQ ID NO: 281 - DNA, 17 bp, Homo sapiens
aattttacgatatgcta

SEQ ID NO: 282 - DNA, 17 bp, Homo sapiens
attttacgatatgctac

SEQ ID NO: 283 - DNA, 17 bp, Homo sapiens
ttttacgatatgctaca

SEQ ID NO: 284 - DNA, 17 bp, Homo sapiens
tttacgatatgctacag

SEQ ID NO: 285 - DNA, 17 bp, Homo sapiens
ttacgatatgctacagc

SEQ ID NO: 286 - DNA, 17 bp, Homo sapiens
tacgatatgctacagca

SEQ ID NO: 287 - DNA, 17 bp, Homo sapiens
acgatatgctacagcac

SEQ ID NO: 288 - DNA, 17 bp, Homo sapiens
cgatatgctacagcacc

SEQ ID NO: 289 - DNA, 17 bp, Homo sapiens
gatatgctacagcacca

SEQ ID NO: 290 - DNA, 17 bp, Homo sapiens
atatgctacagcaccaa

SEQ ID NO: 291 - DNA, 17 bp, Homo sapiens
tatgctacagcaccaac

SEQ ID NO: 292 - DNA, 17 bp, Homo sapiens
atgctacagcaccaact

SEQ ID NO: 293 - DNA, 17 bp, Homo sapiens
tgctacagcaccaactg

SEQ ID NO: 294 - DNA, 17 bp, Homo sapiens
gctacagcaccaactga

SEQ ID NO: 295 - DNA, 17 bp, Homo sapiens
ctacagcaccaactgat

SEQ ID NO: 296 - DNA, 17 bp, Homo sapiens
tacagcaccaactgata

SEQ ID NO: 297 - DNA, 17 bp, Homo sapiens
acagcaccaactgatat

SEQ ID NO: 298 - DNA, 17 bp, Homo sapiens
cagcaccaactgatatt

SEQ ID NO: 299 - DNA, 17 bp, Homo sapiens
agcaccaactgatattg

SEQ ID NO: 300 - DNA, 17 bp, Homo sapiens
gcaccaactgatattga

SEQ ID NO: 301 - DNA, 17 bp, Homo sapiens
caccaactgatattgaa

SEQ ID NO: 302 - DNA, 17 bp, Homo sapiens
accaactgatattgaaa

SEQ ID NO: 303 - DNA, 17 bp, Homo sapiens
ccaactgatattgaaag

SEQ ID NO: 304 - DNA, 17 bp, Homo sapiens
caactgatattgaaagt

SEQ ID NO: 305 - DNA, 17 bp, Homo sapiens
aactgatattgaaagtg

SEQ ID NO: 306 - DNA, 17 bp, Homo sapiens
actgatattgaaagtgg

SEQ ID NO: 307 - DNA, 17 bp, Homo sapiens
ctgatattgaaagtgga

SEQ ID NO: 308 - DNA, 17 bp, Homo sapiens
tgatattgaaagtggaa

SEQ ID NO: 309 - DNA, 17 bp, Homo sapiens
gatattgaaagtggaac

SEQ ID NO: 310 - DNA, 17 bp, Homo sapiens
atattgaaagtggaact

SEQ ID NO: 311 - DNA, 17 bp, Homo sapiens
tattgaaagtggaactg

SEQ ID NO: 312 - DNA, 17 bp, Homo sapiens
attgaaagtggaactgt

SEQ ID NO: 313 - DNA, 17 bp, Homo sapiens
ttgaaagtggaactgtc

SEQ ID NO: 314 - DNA, 17 bp, Homo sapiens
tgaaagtggaactgtct

SEQ ID NO: 315 - DNA, 17 bp, Homo sapiens
gaaagtggaactgtcta

SEQ ID NO: 316 - DNA, 17 bp, Homo sapiens
aaagtggaactgtctat

SEQ ID NO: 317 - DNA, 17 bp, Homo sapiens
aagtggaactgtctatg

SEQ ID NO: 318 - DNA, 17 bp, Homo sapiens
agtggaactgtctatga

SEQ ID NO: 319 - DNA, 17 bp, Homo sapiens
gtggaactgtctatgac

SEQ ID NO: 320 - DNA, 17 bp, Homo sapiens
tggaactgtctatgact

SEQ ID NO: 321 - DNA, 17 bp, Homo sapiens
ggaactgtctatgactg

SEQ ID NO: 322 - DNA, 17 bp, Homo sapiens
gaactgtctatgactgt

SEQ ID NO: 323 - DNA, 17 bp, Homo sapiens
aactgtctatgactgtg

SEQ ID NO: 324 - DNA, 17 bp, Homo sapiens
actgtctatgactgtgt

SEQ ID NO: 325 - DNA, 17 bp, Homo sapiens
ctgtctatgactgtgta

SEQ ID NO: 326 - DNA, 17 bp, Homo sapiens
tgtctatgactgtgtaa

SEQ ID NO: 327 - DNA, 17 bp, Homo sapiens
gtctatgactgtgtaaa

SEQ ID NO: 328 - DNA, 17 bp, Homo sapiens
tctatgactgtgtaaaa

SEQ ID NO: 329 - DNA, 17 bp, Homo sapiens
ctatgactgtgtaaaac

SEQ ID NO: 330 - DNA, 17 bp, Homo sapiens
tatgactgtgtaaaact

SEQ ID NO: 331 - DNA, 17 bp, Homo sapiens
atgactgtgtaaaacta

SEQ ID NO: 332 - DNA, 17 bp, Homo sapiens
tgactgtgtaaaactaa

SEQ ID NO: 333 - DNA, 17 bp, Homo sapiens
gactgtgtaaaactaac

SEQ ID NO: 334 - DNA, 17 bp, Homo sapiens
actgtgtaaaactaact

SEQ ID NO: 335 - DNA, 17 bp, Homo sapiens
ctgtgtaaaactaactt

SEQ ID NO: 336 - DNA, 17 bp, Homo sapiens
tgtgtaaaactaacttt

SEQ ID NO: 337 - DNA, 17 bp, Homo sapiens
gtgtaaaactaactttc

SEQ ID NO: 338 - DNA, 17 bp, Homo sapiens
tgtaaaactaactttca

SEQ ID NO: 339 - DNA, 17 bp, Homo sapiens
gtaaaactaactttcag

SEQ ID NO: 340 - DNA, 17 bp, Homo sapiens
taaaactaactttcagt

SEQ ID NO: 341 - DNA, 17 bp, Homo sapiens
aaaactaactttcagtc

SEQ ID NO: 342 - DNA, 17 bp, Homo sapiens
aaactaactttcagtcc

SEQ ID NO: 343 - DNA, 17 bp, Homo sapiens
aactaactttcagtcca

SEQ ID NO: 344 - DNA, 17 bp, Homo sapiens
actaactttcagtccat

SEQ ID NO: 345 - DNA, 17 bp, Homo sapiens
ctaactttcagtccatc

SEQ ID NO: 346 - DNA, 17 bp, Homo sapiens
taactttcagtccatca

SEQ ID NO: 347 - DNA, 17 bp, Homo sapiens
aactttcagtccatcaa

SEQ ID NO: 348 - DNA, 17 bp, Homo sapiens
actttcagtccatcaac

SEQ ID NO: 349 - DNA, 17 bp, Homo sapiens
ctttcagtccatcaact

SEQ ID NO: 350 - DNA, 17 bp, Homo sapiens
tttcagtccatcaactc

SEQ ID NO: 351 - DNA, 17 bp, Homo sapiens
ttcagtccatcaactct

SEQ ID NO: 352 - DNA, 17 bp, Homo sapiens
tcagtccatcaactctg

SEQ ID NO: 353 - DNA, 17 bp, Homo sapiens
cagtccatcaactctgc

SEQ ID NO: 354 - DNA, 17 bp, Homo sapiens
agtccatcaactctgct

SEQ ID NO: 355 - DNA, 17 bp, Homo sapiens
gtccatcaactctgctg

SEQ ID NO: 356 - DNA, 17 bp, Homo sapiens
tccatcaactctgctgg

SEQ ID NO: 357 - DNA, 17 bp, Homo sapiens
ccatcaactctgctggt

SEQ ID NO: 358 - DNA, 17 bp, Homo sapiens
catcaactctgctggtt

SEQ ID NO: 359 - DNA, 17 bp, Homo sapiens
atcaactctgctggtta

SEQ ID NO: 360 - DNA, 17 bp, Homo sapiens
tcaactctgctggttaa

SEQ ID NO: 361 - DNA, 17 bp, Homo sapiens
caactctgctggttaat

SEQ ID NO: 362 - DNA, 17 bp, Homo sapiens
aactctgctggttaata

SEQ ID NO: 363 - DNA, 17 bp, Homo sapiens
actctgctggttaatat

SEQ ID NO: 364 - DNA, 17 bp, Homo sapiens
ctctgctggttaatatc

SEQ ID NO: 365 - DNA, 17 bp, Homo sapiens
tctgctggttaatatca

SEQ ID NO: 366 - DNA, 17 bp, Homo sapiens
ctgctggttaatatcac

SEQ ID NO: 367 - DNA, 17 bp, Homo sapiens
tgctggttaatatcact

SEQ ID NO: 368 - DNA, 17 bp, Homo sapiens
gctggttaatatcactg

SEQ ID NO: 369 - DNA, 17 bp, Homo sapiens
ctggttaatatcactga

SEQ ID NO: 370 - DNA, 17 bp, Homo sapiens
tggttaatatcactgac

SEQ ID NO: 371 - DNA, 17 bp, Homo sapiens
ggttaatatcactgacc

SEQ ID NO: 372 - DNA, 17 bp, Homo sapiens
gttaatatcactgacca

SEQ ID NO: 373 - DNA, 17 bp, Homo sapiens
ttaatatcactgaccaa

SEQ ID NO: 374 - DNA, 17 bp, Homo sapiens
taatatcactgaccaag

SEQ ID NO: 375 - DNA, 17 bp, Homo sapiens
aatatcactgaccaagt

SEQ ID NO: 376 - DNA, 17 bp, Homo sapiens
atatcactgaccaagtt

SEQ ID NO: 377 - DNA, 17 bp, Homo sapiens
tatcactgaccaagttt

SEQ ID NO: 378 - DNA, 17 bp, Homo sapiens
atcactgaccaagttta

SEQ ID NO: 379 - DNA, 17 bp, Homo sapiens
tcactgaccaagtttat

SEQ ID NO: 380 - DNA, 17 bp, Homo sapiens
cactgaccaagtttatg

SEQ ID NO: 381 - DNA, 17 bp, Homo sapiens
actgaccaagtttatga

SEQ ID NO: 382 - DNA, 17 bp, Homo sapiens
ctgaccaagtttatgaa

SEQ ID NO: 383 - DNA, 17 bp, Homo sapiens
tgaccaagtttatgaat

SEQ ID NO: 384 - DNA, 17 bp, Homo sapiens
gaccaagtttatgaata

SEQ ID NO: 385 - DNA, 17 bp, Homo sapiens
accaagtttatgaatat

SEQ ID NO: 386 - DNA, 17 bp, Homo sapiens
ccaagtttatgaatata

SEQ ID NO: 387 - DNA, 17 bp, Homo sapiens
caagtttatgaatataa

SEQ ID NO: 388 - DNA, 17 bp, Homo sapiens
aagtttatgaatataaa

SEQ ID NO: 389 - DNA, 17 bp, Homo sapiens
agtttatgaatataaat

SEQ ID NO: 390 - DNA, 17 bp, Homo sapiens
gtttatgaatataaata

SEQ ID NO: 391 - DNA, 17 bp, Homo sapiens
tttatgaatataaatac

SEQ ID NO: 392 - DNA, 17 bp, Homo sapiens
ttatgaatataaataca

SEQ ID NO: 393 - DNA, 17 bp, Homo sapiens
tatgaatataaatacaa

SEQ ID NO: 394 - DNA, 17 bp, Homo sapiens
atgaatataaatacaaa

SEQ ID NO: 395 - DNA, 17 bp, Homo sapiens
tgaatataaatacaaaa

SEQ ID NO: 396 - DNA, 17 bp, Homo sapiens
gaatataaatacaaaag

SEQ ID NO: 397 - DNA, 17 bp, Homo sapiens
aatataaatacaaaaga

SEQ ID NO: 398 - DNA, 17 bp, Homo sapiens
atataaatacaaaagag

SEQ ID NO: 399 - DNA, 17 bp, Homo sapiens
tataaatacaaaagaga

SEQ ID NO: 400 - DNA, 17 bp, Homo sapiens
ataaatacaaaagagaa

SEQ ID NO: 401 - DNA, 17 bp, Homo sapiens
taaatacaaaagagaaa

SEQ ID NO: 402 - DNA, 17 bp, Homo sapiens
aaatacaaaagagaaat

SEQ ID NO: 403 - DNA, 17 bp, Homo sapiens
aatacaaaagagaaata

SEQ ID NO: 404 - DNA, 17 bp, Homo sapiens
atacaaaagagaaataa

SEQ ID NO: 405 - DNA, 17 bp, Homo sapiens
tacaaaagagaaataag

SEQ ID NO: 406 - DNA, 17 bp, Homo sapiens
acaaaagagaaataagt

SEQ ID NO: 407 - DNA, 17 bp, Homo sapiens
caaaagagaaataagtc

SEQ ID NO: 408 - DNA, 17 bp, Homo sapiens
aaaagagaaataagtca

SEQ ID NO: 409 - DNA, 17 bp, Homo sapiens
aaagagaaataagtcag

SEQ ID NO: 410 - DNA, 17 bp, Homo sapiens
aagagaaataagtcagc

SEQ ID NO: 411 - DNA, 17 bp, Homo sapiens
agagaaataagtcagca

SEQ ID NO: 412 - DNA, 17 bp, Homo sapiens
gagaaataagtcagcac

SEQ ID NO: 413 - DNA, 17 bp, Homo sapiens
agaaataagtcagcaca

SEQ ID NO: 414 - DNA, 17 bp, Homo sapiens
gaaataagtcagcacaa

SEQ ID NO: 415 - DNA, 17 bp, Homo sapiens
aaataagtcagcacaac

SEQ ID NO: 416 - DNA, 17 bp, Homo sapiens
aataagtcagcacaaca

SEQ ID NO: 417 - DNA, 17 bp, Homo sapiens
ataagtcagcacaacat

SEQ ID NO: 418 - DNA, 17 bp, Homo sapiens
taagtcagcacaacatc

SEQ ID NO: 419 - DNA, 17 bp, Homo sapiens
aagtcagcacaacatca

SEQ ID NO: 420 - DNA, 17 bp, Homo sapiens
agtcagcacaacatcaa

SEQ ID NO: 421 - DNA, 17 bp, Homo sapiens
gtcagcacaacatcaat

SEQ ID NO: 422 - DNA, 17 bp, Homo sapiens
tcagcacaacatcaatc

SEQ ID NO: 423 - DNA, 17 bp, Homo sapiens
cagcacaacatcaatcc

SEQ ID NO: 424 - DNA, 17 bp, Homo sapiens
agcacaacatcaatcct

SEQ ID NO: 425 - DNA, 17 bp, Homo sapiens
gcacaacatcaatcctc

SEQ ID NO: 426 - DNA, 17 bp, Homo sapiens
cacaacatcaatcctca

SEQ ID NO: 427 - DNA, 17 bp, Homo sapiens
acaacatcaatcctcat

SEQ ID NO: 428 - DNA, 17 bp, Homo sapiens
caacatcaatcctcatc

SEQ ID NO: 429 - DNA, 17 bp, Homo sapiens
aacatcaatcctcatca

SEQ ID NO: 430 - DNA, 17 bp, Homo sapiens
acatcaatcctcatcaa

SEQ ID NO: 431 - DNA, 17 bp, Homo sapiens
catcaatcctcatcaag

SEQ ID NO: 432 - DNA, 17 bp, Homo sapiens
atcaatcctcatcaagg

SEQ ID NO: 433 - DNA, 17 bp, Homo sapiens
tcaatcctcatcaagga

SEQ ID NO: 434 - DNA, 17 bp, Homo sapiens
caatcctcatcaaggaa

SEQ ID NO: 435 - DNA, 17 bp, Homo sapiens
aatcctcatcaaggaaa

SEQ ID NO: 436 - DNA, 17 bp, Homo sapiens
atcctcatcaaggaaat

SEQ ID NO: 437 - DNA, 17 bp, Homo sapiens
tcctcatcaaggaaatg

SEQ ID NO: 438 - DNA, 17 bp, Homo sapiens
cctcatcaaggaaatgc

SEQ ID NO: 439 - DNA, 17 bp, Homo sapiens
ctcatcaaggaaatgct

SEQ ID NO: 440 - DNA, 17 bp, Homo sapiens
tcatcaaggaaatgcta

SEQ ID NO: 441 - DNA, 17 bp, Homo sapiens
catcaaggaaatgctat

SEQ ID NO: 442 - DNA, 17 bp, Homo sapiens
atcaaggaaatgctata

SEQ ID NO: 443 - DNA, 17 bp, Homo sapiens
tcaaggaaatgctatac

SEQ ID NO: 444 - DNA, 17 bp, Homo sapiens
caaggaaatgctatact

SEQ ID NO: 445 - DNA, 17 bp, Homo sapiens
aaggaaatgctatactt

SEQ ID NO: 446 - DNA, 17 bp, Homo sapiens
aggaaatgctatacttg

SEQ ID NO: 447 - DNA, 17 bp, Homo sapiens
ggaaatgctatacttga

SEQ ID NO: 448 - DNA, 17 bp, Homo sapiens
gaaatgctatacttgaa

SEQ ID NO: 449 - DNA, 17 bp, Homo sapiens
aaatgctatacttgaaa

SEQ ID NO: 450 - DNA, 17 bp, Homo sapiens
aatgctatacttgaaaa

SEQ ID NO: 451 - DNA, 17 bp, Homo sapiens
atgctatacttgaaaag

SEQ ID NO: 452 - DNA, 17 bp, Homo sapiens
tgctatacttgaaaaga

SEQ ID NO: 453 - DNA, 17 bp, Homo sapiens
gctatacttgaaaagat

SEQ ID NO: 454 - DNA, 17 bp, Homo sapiens
ctatacttgaaaagatg

SEQ ID NO: 455 - DNA, 17 bp, Homo sapiens
tatacttgaaaagatga

SEQ ID NO: 456 - DNA, 17 bp, Homo sapiens
atacttgaaaagatgac

SEQ ID NO: 457 - DNA, 17 bp, Homo sapiens
tacttgaaaagatgaca

SEQ ID NO: 458 - DNA, 17 bp, Homo sapiens
acttgaaaagatgacat

SEQ ID NO: 459 - DNA, 17 bp, Homo sapiens
cttgaaaagatgacatt

SEQ ID NO: 460 - DNA, 17 bp, Homo sapiens
ttgaaaagatgacattt

SEQ ID NO: 461 - DNA, 17 bp, Homo sapiens
tgaaaagatgacatttg

SEQ ID NO: 462 - DNA, 17 bp, Homo sapiens
gaaaagatgacatttga

SEQ ID NO: 463 - DNA, 17 bp, Homo sapiens
aaaagatgacatttgat

SEQ ID NO: 464 - DNA, 17 bp, Homo sapiens
aaagatgacatttgatc

SEQ ID NO: 465 - DNA, 17 bp, Homo sapiens
aagatgacatttgatcc

SEQ ID NO: 466 - DNA, 17 bp, Homo sapiens
agatgacatttgatcca

SEQ ID NO: 467 - DNA, 17 bp, Homo sapiens
gatgacatttgatccag

SEQ ID NO: 468 - DNA, 17 bp, Homo sapiens
atgacatttgatccaga

SEQ ID NO: 469 - DNA, 17 bp, Homo sapiens
tgacatttgatccagaa

SEQ ID NO: 470 - DNA, 17 bp, Homo sapiens
gacatttgatccagaaa

SEQ ID NO: 471 - DNA, 17 bp, Homo sapiens
acatttgatccagaaat

SEQ ID NO: 472 - DNA, 17 bp, Homo sapiens
catttgatccagaaatc

SEQ ID NO: 473 - DNA, 17 bp, Homo sapiens
atttgatccagaaatct

SEQ ID NO: 474 - DNA, 17 bp, Homo sapiens
tttgatccagaaatctt

SEQ ID NO: 475 - DNA, 17 bp, Homo sapiens
ttgatccagaaatcttc

SEQ ID NO: 476 - DNA, 17 bp, Homo sapiens
tgatccagaaatcttct

SEQ ID NO: 477 - DNA, 17 bp, Homo sapiens
gatccagaaatcttctt

SEQ ID NO: 478 - DNA, 17 bp, Homo sapiens
atccagaaatcttcttc

SEQ ID NO: 479 - DNA, 17 bp, Homo sapiens
tccagaaatcttcttca

SEQ ID NO: 480 - DNA, 17 bp, Homo sapiens
ccagaaatcttcttcaa

SEQ ID NO: 481 - DNA, 17 bp, Homo sapiens
cagaaatcttcttcaat

SEQ ID NO: 482 - DNA, 17 bp, Homo sapiens
agaaatcttcttcaatg

SEQ ID NO: 483 - DNA, 17 bp, Homo sapiens
gaaatcttcttcaatgt

SEQ ID NO: 484 - DNA, 17 bp, Homo sapiens
aaatcttcttcaatgtt

SEQ ID NO: 485 - DNA, 17 bp, Homo sapiens
aatcttcttcaatgttt

SEQ ID NO: 486 - DNA, 17 bp, Homo sapiens
atcttcttcaatgtttt

SEQ ID NO: 487 - DNA, 17 bp, Homo sapiens
tcttcttcaatgtttta

SEQ ID NO: 488 - DNA, 17 bp, Homo sapiens
cttcttcaatgttttac

SEQ ID NO: 489 - DNA, 17 bp, Homo sapiens
ttcttcaatgttttact

SEQ ID NO: 490 - DNA, 17 bp, Homo sapiens
tcttcaatgttttactg

SEQ ID NO: 491 - DNA, 17 bp, Homo sapiens
cttcaatgttttactgc

SEQ ID NO: 492 - DNA, 17 bp, Homo sapiens
ttcaatgttttactgcc

SEQ ID NO: 493 - DNA, 17 bp, Homo sapiens
tcaatgttttactgcca

SEQ ID NO: 494 - DNA, 17 bp, Homo sapiens
caatgttttactgccac

SEQ ID NO: 495 - DNA, 17 bp, Homo sapiens
aatgttttactgccacc

SEQ ID NO: 496 - DNA, 17 bp, Homo sapiens
atgttttactgccacca

SEQ ID NO: 497 - DNA, 17 bp, Homo sapiens
tgttttactgccaccaa

SEQ ID NO: 498 - DNA, 17 bp, Homo sapiens
gttttactgccaccaat

SEQ ID NO: 499 - DNA, 17 bp, Homo sapiens
ttttactgccaccaatt

SEQ ID NO: 500 - DNA, 17 bp, Homo sapiens
tttactgccaccaatta

SEQ ID NO: 501 - DNA, 17 bp, Homo sapiens
ttactgccaccaattat

SEQ ID NO: 502 - DNA, 17 bp, Homo sapiens
tactgccaccaattata

SEQ ID NO: 503 - DNA, 17 bp, Homo sapiens
actgccaccaattatat

SEQ ID NO: 504 - DNA, 17 bp, Homo sapiens
ctgccaccaattatatt

SEQ ID NO: 505 - DNA, 17 bp, Homo sapiens
tgccaccaattatattt

SEQ ID NO: 506 - DNA, 17 bp, Homo sapiens
gccaccaattatatttc

SEQ ID NO: 507 - DNA, 17 bp, Homo sapiens
ccaccaattatatttca

SEQ ID NO: 508 - DNA, 17 bp, Homo sapiens
caccaattatatttcat

SEQ ID NO: 509 - DNA, 17 bp, Homo sapiens
accaattatatttcatg

SEQ ID NO: 510 - DNA, 17 bp, Homo sapiens
ccaattatatttcatgc

SEQ ID NO: 511 - DNA, 17 bp, Homo sapiens
caattatatttcatgca

SEQ ID NO: 512 - DNA, 17 bp, Homo sapiens
aattatatttcatgcag

SEQ ID NO: 513 - DNA, 17 bp, Homo sapiens
attatatttcatgcagg

SEQ ID NO: 514 - DNA, 17 bp, Homo sapiens
ttatatttcatgcagga

SEQ ID NO: 515 - DNA, 17 bp, Homo sapiens
tatatttcatgcaggat

SEQ ID NO: 516 - DNA, 17 bp, Homo sapiens
atatttcatgcaggata

SEQ ID NO: 517 - DNA, 17 bp, Homo sapiens
tatttcatgcaggatat

SEQ ID NO: 518 - DNA, 17 bp, Homo sapiens
atttcatgcaggatata

SEQ ID NO: 519 - DNA, 17 bp, Homo sapiens
tttcatgcaggatatag

SEQ ID NO: 520 - DNA, 17 bp, Homo sapiens
ttcatgcaggatatagt

SEQ ID NO: 521 - DNA, 17 bp, Homo sapiens
tcatgcaggatatagtc

SEQ ID NO: 522 - DNA, 17 bp, Homo sapiens
catgcaggatatagtct

SEQ ID NO: 523 - DNA, 17 bp, Homo sapiens
atgcaggatatagtcta

SEQ ID NO: 524 - DNA, 17 bp, Homo sapiens
tgcaggatatagtctaa

SEQ ID NO: 525 - DNA, 17 bp, Homo sapiens
gcaggatatagtctaaa

SEQ ID NO: 526 - DNA, 17 bp, Homo sapiens
caggatatagtctaaag

SEQ ID NO: 527 - DNA, 17 bp, Homo sapiens
aggatatagtctaaaga

SEQ ID NO: 528 - DNA, 17 bp, Homo sapiens
ggatatagtctaaagaa

SEQ ID NO: 529 - DNA, 17 bp, Homo sapiens
gatatagtctaaagaag

SEQ ID NO: 530 - DNA, 17 bp, Homo sapiens
atatagtctaaagaaga

SEQ ID NO: 531 - DNA, 17 bp, Homo sapiens
tatagtctaaagaagag

SEQ ID NO: 532 - DNA, 17 bp, Homo sapiens
atagtctaaagaagaga

SEQ ID NO: 533 - DNA, 17 bp, Homo sapiens
tagtctaaagaagagac

SEQ ID NO: 534 - DNA, 17 bp, Homo sapiens
agtctaaagaagagaca

SEQ ID NO: 535 - DNA, 17 bp, Homo sapiens
gtctaaagaagagacac

SEQ ID NO: 536 - DNA, 17 bp, Homo sapiens
tctaaagaagagacact

SEQ ID NO: 537 - DNA, 17 bp, Homo sapiens
ctaaagaagagacactt

SEQ ID NO: 538 - DNA, 17 bp, Homo sapiens
taaagaagagacacttt

SEQ ID NO: 539 - DNA, 17 bp, Homo sapiens
aaagaagagacactttt

SEQ ID NO: 540 - DNA, 17 bp, Homo sapiens
aagaagagacacttttt

SEQ ID NO: 541 - DNA, 17 bp, Homo sapiens
agaagagacactttttt

SEQ ID NO: 542 - DNA, 17 bp, Homo sapiens
gaagagacacttttttc

SEQ ID NO: 543 - DNA, 17 bp, Homo sapiens
aagagacacttttttca

SEQ ID NO: 544 - DNA, 17 bp, Homo sapiens
agagacacttttttcaa

SEQ ID NO: 545 - DNA, 17 bp, Homo sapiens
gagacacttttttcaaa

SEQ ID NO: 546 - DNA, 17 bp, Homo sapiens
agacacttttttcaaaa

SEQ ID NO: 547 - DNA, 17 bp, Homo sapiens
gacacttttttcaaaac

SEQ ID NO: 548 - DNA, 17 bp, Homo sapiens
acacttttttcaaaact

SEQ ID NO: 549 - DNA, 17 bp, Homo sapiens
cacttttttcaaaactt

SEQ ID NO: 550 - DNA, 17 bp, Homo sapiens
acttttttcaaaactta

SEQ ID NO: 551 - DNA, 17 bp, Homo sapiens
cttttttcaaaacttag

SEQ ID NO: 552 - DNA, 17 bp, Homo sapiens
ttttttcaaaacttagg

SEQ ID NO: 553 - DNA, 17 bp, Homo sapiens
tttttcaaaacttagga

SEQ ID NO: 554 - DNA, 17 bp, Homo sapiens
ttttcaaaacttaggat

SEQ ID NO: 555 - DNA, 17 bp, Homo sapiens
tttcaaaacttaggatc

SEQ ID NO: 556 - DNA, 17 bp, Homo sapiens
ttcaaaacttaggatct

SEQ ID NO: 557 - DNA, 17 bp, Homo sapiens
tcaaaacttaggatcta

SEQ ID NO: 558 - DNA, 17 bp, Homo sapiens
caaaacttaggatctat

SEQ ID NO: 559 - DNA, 17 bp, Homo sapiens
aaaacttaggatctatt

SEQ ID NO: 560 - DNA, 17 bp, Homo sapiens
aaacttaggatctattt

SEQ ID NO: 561 - DNA, 17 bp, Homo sapiens
aacttaggatctatttt

SEQ ID NO: 562 - DNA, 17 bp, Homo sapiens
acttaggatctatttta

SEQ ID NO: 563 - DNA, 17 bp, Homo sapiens
cttaggatctattttaa

SEQ ID NO: 564 - DNA, 17 bp, Homo sapiens
ttaggatctattttaac

SEQ ID NO: 565 - DNA, 17 bp, Homo sapiens
taggatctattttaacg

SEQ ID NO: 566 - DNA, 17 bp, Homo sapiens
aggatctattttaacgt

SEQ ID NO: 567 - DNA, 17 bp, Homo sapiens
ggatctattttaacgta

SEQ ID NO: 568 - DNA, 17 bp, Homo sapiens
gatctattttaacgtat

SEQ ID NO: 569 - DNA, 17 bp, Homo sapiens
atctattttaacgtatg

SEQ ID NO: 570 - DNA, 17 bp, Homo sapiens
tctattttaacgtatgc

SEQ ID NO: 571 - DNA, 17 bp, Homo sapiens
ctattttaacgtatgcc

SEQ ID NO: 572 - DNA, 17 bp, Homo sapiens
tattttaacgtatgcct

SEQ ID NO: 573 - DNA, 17 bp, Homo sapiens
attttaacgtatgcctt

SEQ ID NO: 574 - DNA, 17 bp, Homo sapiens
ttttaacgtatgccttc

SEQ ID NO: 575 - DNA, 17 bp, Homo sapiens
tttaacgtatgccttct

SEQ ID NO: 576 - DNA, 17 bp, Homo sapiens
ttaacgtatgccttctt

SEQ ID NO: 577 - DNA, 17 bp, Homo sapiens
taacgtatgccttcttg

SEQ ID NO: 578 - DNA, 17 bp, Homo sapiens
aacgtatgccttcttgg

SEQ ID NO: 579 - DNA, 17 bp, Homo sapiens
acgtatgccttcttggg

SEQ ID NO: 580 - DNA, 17 bp, Homo sapiens
cgtatgccttcttggga

SEQ ID NO: 581 - DNA, 17 bp, Homo sapiens
gtatgccttcttgggaa

SEQ ID NO: 582 - DNA, 17 bp, Homo sapiens
tatgccttcttgggaac

SEQ ID NO: 583 - DNA, 17 bp, Homo sapiens
atgccttcttgggaact

SEQ ID NO: 584 - DNA, 17 bp, Homo sapiens
tgccttcttgggaactg

SEQ ID NO: 585 - DNA, 17 bp, Homo sapiens
gccttcttgggaactgc

SEQ ID NO: 586 - DNA, 17 bp, Homo sapiens
ccttcttgggaactgcc

SEQ ID NO: 587 - DNA, 17 bp, Homo sapiens
cttcttgggaactgcca

SEQ ID NO: 588 - DNA, 17 bp, Homo sapiens
ttcttgggaactgccat

SEQ ID NO: 589 - DNA, 17 bp, Homo sapiens
tcttgggaactgccatc

SEQ ID NO: 590 - DNA, 17 bp, Homo sapiens
cttgggaactgccatct

SEQ ID NO: 591 - DNA, 17 bp, Homo sapiens
ttgggaactgccatctc

SEQ ID NO: 592 - DNA, 17 bp, Homo sapiens
tgggaactgccatctcc

SEQ ID NO: 593 - DNA, 17 bp, Homo sapiens
gggaactgccatctcct

SEQ ID NO: 594 - DNA, 17 bp, Homo sapiens
ggaactgccatctcctg

SEQ ID NO: 595 - DNA, 17 bp, Homo sapiens
gaactgccatctcctgc

SEQ ID NO: 596 - DNA, 17 bp, Homo sapiens
aactgccatctcctgca

SEQ ID NO: 597 - DNA, 17 bp, Homo sapiens
actgccatctcctgcat

SEQ ID NO: 598 - DNA, 17 bp, Homo sapiens
ctgccatctcctgcatc

SEQ ID NO: 599 - DNA, 17 bp, Homo sapiens
tgccatctcctgcatcg

SEQ ID NO: 600 - DNA, 17 bp, Homo sapiens
gccatctcctgcatcgt

SEQ ID NO: 601 - DNA, 17 bp, Homo sapiens
ccatctcctgcatcgtc

SEQ ID NO: 602 - DNA, 17 bp, Homo sapiens
catctcctgcatcgtca

SEQ ID NO: 603 - DNA, 17 bp, Homo sapiens
atctcctgcatcgtcat

SEQ ID NO: 604 - DNA, 17 bp, Homo sapiens
tctcctgcatcgtcata

SEQ ID NO: 605 - DNA, 17 bp, Homo sapiens
ctcctgcatcgtcatag

SEQ ID NO: 606 - DNA, 17 bp, Homo sapiens
tcctgcatcgtcatagg

SEQ ID NO: 607 - DNA, 17 bp, Homo sapiens
cctgcatcgtcataggg

SEQ ID NO: 608 - DNA, 17 bp, Homo sapiens
ctgcatcgtcatagggt

SEQ ID NO: 609 - DNA, 17 bp, Homo sapiens
tgcatcgtcatagggtt

SEQ ID NO: 610 - DNA, 17 bp, Homo sapiens
gcatcgtcatagggtta

SEQ ID NO: 611 - DNA, 17 bp, Homo sapiens
catcgtcatagggttaa

SEQ ID NO: 612 - DNA, 17 bp, Homo sapiens
atcgtcatagggttaat

SEQ ID NO: 613 - DNA, 17 bp, Homo sapiens
tcgtcatagggttaatt

SEQ ID NO: 614 - DNA, 17 bp, Homo sapiens
cgtcatagggttaatta

SEQ ID NO: 615 - DNA, 17 bp, Homo sapiens
gtcatagggttaattat

SEQ ID NO: 616 - DNA, 17 bp, Homo sapiens
tcatagggttaattatg

SEQ ID NO: 617 - DNA, 17 bp, Homo sapiens
catagggttaattatgt

SEQ ID NO: 618 - DNA, 17 bp, Homo sapiens
atagggttaattatgta

SEQ ID NO: 619 - DNA, 17 bp, Homo sapiens
tagggttaattatgtat

SEQ ID NO: 620 - DNA, 17 bp, Homo sapiens
agggttaattatgtatg

SEQ ID NO: 621 - DNA, 17 bp, Homo sapiens
gggttaattatgtatgg

SEQ ID NO: 622 - DNA, 17 bp, Homo sapiens
ggttaattatgtatggt

SEQ ID NO: 623 - DNA, 17 bp, Homo sapiens
gttaattatgtatggtt

SEQ ID NO: 624 - DNA, 17 bp, Homo sapiens
ttaattatgtatggttt

SEQ ID NO: 625 - DNA, 17 bp, Homo sapiens
taattatgtatggtttt

SEQ ID NO: 626 - DNA, 17 bp, Homo sapiens
aattatgtatggttttg

SEQ ID NO: 627 - DNA, 17 bp, Homo sapiens
attatgtatggttttgt

SEQ ID NO: 628 - DNA, 17 bp, Homo sapiens
ttatgtatggttttgtg

SEQ ID NO: 629 - DNA, 17 bp, Homo sapiens
tatgtatggttttgtga

SEQ ID NO: 630 - DNA, 17 bp, Homo sapiens
atgtatggttttgtgaa

SEQ ID NO: 631 - DNA, 17 bp, Homo sapiens
tgtatggttttgtgaag

SEQ ID NO: 632 - DNA, 17 bp, Homo sapiens
gtatggttttgtgaagg

SEQ ID NO: 633 - DNA, 17 bp, Homo sapiens
tatggttttgtgaaggc

SEQ ID NO: 634 - DNA, 17 bp, Homo sapiens
atggttttgtgaaggct

SEQ ID NO: 635 - DNA, 17 bp, Homo sapiens
tggttttgtgaaggcta

SEQ ID NO: 636 - DNA, 17 bp, Homo sapiens
ggttttgtgaaggctat

SEQ ID NO: 637 - DNA, 17 bp, Homo sapiens
gttttgtgaaggctatg

SEQ ID NO: 638 - DNA, 17 bp, Homo sapiens
ttttgtgaaggctatga

SEQ ID NO: 639 - DNA, 17 bp, Homo sapiens
tttgtgaaggctatgat

SEQ ID NO: 640 - DNA, 17 bp, Homo sapiens
ttgtgaaggctatgata

SEQ ID NO: 641 - DNA, 17 bp, Homo sapiens
tgtgaaggctatgatac

SEQ ID NO: 642 - DNA, 17 bp, Homo sapiens
gtgaaggctatgataca

SEQ ID NO: 643 - DNA, 17 bp, Homo sapiens
tgaaggctatgatacat

SEQ ID NO: 644 - DNA, 17 bp, Homo sapiens
gaaggctatgatacatg

SEQ ID NO: 645 - DNA, 17 bp, Homo sapiens
aaggctatgatacatgc

SEQ ID NO: 646 - DNA, 17 bp, Homo sapiens
aggctatgatacatgct

SEQ ID NO: 647 - DNA, 17 bp, Homo sapiens
ggctatgatacatgctg

SEQ ID NO: 648 - DNA, 17 bp, Homo sapiens
gctatgatacatgctgg

SEQ ID NO: 649 - DNA, 17 bp, Homo sapiens
ctatgatacatgctggc

SEQ ID NO: 650 - DNA, 17 bp, Homo sapiens
tatgatacatgctggcc

SEQ ID NO: 651 - DNA, 17 bp, Homo sapiens
atgatacatgctggcca

SEQ ID NO: 652 - DNA, 17 bp, Homo sapiens
tgatacatgctggccag

SEQ ID NO: 653 - DNA, 17 bp, Homo sapiens
gatacatgctggccagc

SEQ ID NO: 654 - DNA, 17 bp, Homo sapiens
atacatgctggccagct

SEQ ID NO: 655 - DNA, 17 bp, Homo sapiens
tacatgctggccagctg

SEQ ID NO: 656 - DNA, 17 bp, Homo sapiens
acatgctggccagctga

SEQ ID NO: 657 - DNA, 17 bp, Homo sapiens
catgctggccagctgaa

SEQ ID NO: 658 - DNA, 17 bp, Homo sapiens
atgctggccagctgaaa

SEQ ID NO: 659 - DNA, 17 bp, Homo sapiens
tgctggccagctgaaaa

SEQ ID NO: 660 - DNA, 17 bp, Homo sapiens
gctggccagctgaaaaa

SEQ ID NO: 661 - DNA, 17 bp, Homo sapiens
ctggccagctgaaaaat

SEQ ID NO: 662 - DNA, 17 bp, Homo sapiens
tggccagctgaaaaatg

SEQ ID NO: 663 - DNA, 17 bp, Homo sapiens
ggccagctgaaaaatgg

SEQ ID NO: 664 - DNA, 17 bp, Homo sapiens
gccagctgaaaaatgga

SEQ ID NO: 665 - DNA, 17 bp, Homo sapiens
ccagctgaaaaatggag

SEQ ID NO: 666 - DNA, 17 bp, Homo sapiens
cagctgaaaaatggaga

SEQ ID NO: 667 - DNA, 17 bp, Homo sapiens
agctgaaaaatggagac

SEQ ID NO: 668 - DNA, 17 bp, Homo sapiens
gctgaaaaatggagact

SEQ ID NO: 669 - DNA, 17 bp, Homo sapiens
ctgaaaaatggagactt

SEQ ID NO: 670 - DNA, 17 bp, Homo sapiens
tgaaaaatggagacttt

SEQ ID NO: 671 - DNA, 17 bp, Homo sapiens
gaaaaatggagactttc

SEQ ID NO: 672 - DNA, 17 bp, Homo sapiens
aaaaatggagactttca

SEQ ID NO: 673 - DNA, 17 bp, Homo sapiens
aaaatggagactttcat

SEQ ID NO: 674 - DNA, 17 bp, Homo sapiens
aaatggagactttcatt

SEQ ID NO: 675 - DNA, 17 bp, Homo sapiens
aatggagactttcattt

SEQ ID NO: 676 - DNA, 17 bp, Homo sapiens
atggagactttcatttc

SEQ ID NO: 677 - DNA, 17 bp, Homo sapiens
tggagactttcatttca

SEQ ID NO: 678 - DNA, 17 bp, Homo sapiens
ggagactttcatttcac

SEQ ID NO: 679 - DNA, 17 bp, Homo sapiens
gagactttcatttcact

SEQ ID NO: 680 - DNA, 17 bp, Homo sapiens
agactttcatttcactg

SEQ ID NO: 681 - DNA, 17 bp, Homo sapiens
gactttcatttcactga

SEQ ID NO: 682 - DNA, 17 bp, Homo sapiens
actttcatttcactgac

SEQ ID NO: 683 - DNA, 17 bp, Homo sapiens
ctttcatttcactgact

SEQ ID NO: 684 - DNA, 17 bp, Homo sapiens
tttcatttcactgactg

SEQ ID NO: 685 - DNA, 17 bp, Homo sapiens
ttcatttcactgactgt

SEQ ID NO: 686 - DNA, 17 bp, Homo sapiens
tcatttcactgactgtt

SEQ ID NO: 687 - DNA, 17 bp, Homo sapiens
catttcactgactgttt

SEQ ID NO: 688 - DNA, 17 bp, Homo sapiens
atttcactgactgttta

SEQ ID NO: 689 - DNA, 17 bp, Homo sapiens
tttcactgactgtttat

SEQ ID NO: 690 - DNA, 17 bp, Homo sapiens
ttcactgactgtttatt

SEQ ID NO: 691 - DNA, 17 bp, Homo sapiens
tcactgactgtttattt

SEQ ID NO: 692 - DNA, 17 bp, Homo sapiens
cactgactgtttatttt

SEQ ID NO: 693 - DNA, 17 bp, Homo sapiens
actgactgtttattttt

SEQ ID NO: 694 - DNA, 17 bp, Homo sapiens
ctgactgtttatttttt

SEQ ID NO: 695 - DNA, 17 bp, Homo sapiens
tgactgtttattttttg

SEQ ID NO: 696 - DNA, 17 bp, Homo sapiens
gactgtttattttttgg

SEQ ID NO: 697 - DNA, 17 bp, Homo sapiens
actgtttattttttggt

SEQ ID NO: 698 - DNA, 17 bp, Homo sapiens
ctgtttattttttggtt

SEQ ID NO: 699 - DNA, 17 bp, Homo sapiens
tgtttattttttggttc

SEQ ID NO: 700 - DNA, 17 bp, Homo sapiens
gtttattttttggttca

SEQ ID NO: 701 - DNA, 17 bp, Homo sapiens
tttattttttggttcac

SEQ ID NO: 702 - DNA, 17 bp, Homo sapiens
ttattttttggttcact

SEQ ID NO: 703 - DNA, 17 bp, Homo sapiens
tattttttggttcactg

SEQ ID NO: 704 - DNA, 17 bp, Homo sapiens
attttttggttcactga

SEQ ID NO: 705 - DNA, 17 bp, Homo sapiens
ttttttggttcactgat

SEQ ID NO: 706 - DNA, 17 bp, Homo sapiens
tttttggttcactgatg

SEQ ID NO: 707 - DNA, 17 bp, Homo sapiens
ttttggttcactgatgt

SEQ ID NO: 708 - DNA, 17 bp, Homo sapiens
tttggttcactgatgtc

SEQ ID NO: 709 - DNA, 17 bp, Homo sapiens
ttggttcactgatgtct

SEQ ID NO: 710 - DNA, 17 bp, Homo sapiens
tggttcactgatgtctg

SEQ ID NO: 711 - DNA, 17 bp, Homo sapiens
ggttcactgatgtctgc

SEQ ID NO: 712 - DNA, 17 bp, Homo sapiens
gttcactgatgtctgct

SEQ ID NO: 713 - DNA, 17 bp, Homo sapiens
ttcactgatgtctgcta

SEQ ID NO: 714 - DNA, 17 bp, Homo sapiens
tcactgatgtctgctac

SEQ ID NO: 715 - DNA, 17 bp, Homo sapiens
cactgatgtctgctaca

SEQ ID NO: 716 - DNA, 17 bp, Homo sapiens
actgatgtctgctacag

SEQ ID NO: 717 - DNA, 17 bp, Homo sapiens
ctgatgtctgctacaga

SEQ ID NO: 718 - DNA, 17 bp, Homo sapiens
tgatgtctgctacagat

SEQ ID NO: 719 - DNA, 17 bp, Homo sapiens
gatgtctgctacagatc

SEQ ID NO: 720 - DNA, 17 bp, Homo sapiens
atgtctgctacagatcc

SEQ ID NO: 721 - DNA, 17 bp, Homo sapiens
tgtctgctacagatcca

SEQ ID NO: 722 - DNA, 17 bp, Homo sapiens
gtctgctacagatccag

SEQ ID NO: 723 - DNA, 17 bp, Homo sapiens
tctgctacagatccagt

SEQ ID NO: 724 - DNA, 17 bp, Homo sapiens
ctgctacagatccagtg

SEQ ID NO: 725 - DNA, 17 bp, Homo sapiens
tgctacagatccagtga

SEQ ID NO: 726 - DNA, 17 bp, Homo sapiens
gctacagatccagtgac

SEQ ID NO: 727 - DNA, 17 bp, Homo sapiens
ctacagatccagtgaca

SEQ ID NO: 728 - DNA, 17 bp, Homo sapiens
tacagatccagtgacag

SEQ ID NO: 729 - DNA, 17 bp, Homo sapiens
acagatccagtgacagt

SEQ ID NO: 730 - DNA, 17 bp, Homo sapiens
cagatccagtgacagtg

SEQ ID NO: 731 - DNA, 17 bp, Homo sapiens
agatccagtgacagtgc

SEQ ID NO: 732 - DNA, 17 bp, Homo sapiens
gatccagtgacagtgct

SEQ ID NO: 733 - DNA, 17 bp, Homo sapiens
atccagtgacagtgctg

SEQ ID NO: 734 - DNA, 17 bp, Homo sapiens
tccagtgacagtgctgg

SEQ ID NO: 735 - DNA, 17 bp, Homo sapiens
ccagtgacagtgctggc

SEQ ID NO: 736 - DNA, 17 bp, Homo sapiens
cagtgacagtgctggcc

SEQ ID NO: 737 - DNA, 17 bp, Homo sapiens
agtgacagtgctggcca

SEQ ID NO: 738 - DNA, 17 bp, Homo sapiens
gtgacagtgctggccat

SEQ ID NO: 739 - DNA, 17 bp, Homo sapiens
tgacagtgctggccatt

SEQ ID NO: 740 - DNA, 17 bp, Homo sapiens
gacagtgctggccattt

SEQ ID NO: 741 - DNA, 17 bp, Homo sapiens
acagtgctggccatttt

SEQ ID NO: 742 - DNA, 17 bp, Homo sapiens
cagtgctggccattttc

SEQ ID NO: 743 - DNA, 17 bp, Homo sapiens
agtgctggccattttcc

SEQ ID NO: 744 - DNA, 17 bp, Homo sapiens
gtgctggccattttcca

SEQ ID NO: 745 - DNA, 17 bp, Homo sapiens
tgctggccattttccat

SEQ ID NO: 746 - DNA, 17 bp, Homo sapiens
gctggccattttccatg

SEQ ID NO: 747 - DNA, 17 bp, Homo sapiens
ctggccattttccatga

SEQ ID NO: 748 - DNA, 17 bp, Homo sapiens
tggccattttccatgaa

SEQ ID NO: 749 - DNA, 17 bp, Homo sapiens
ggccattttccatgaac

SEQ ID NO: 750 - DNA, 17 bp, Homo sapiens
gccattttccatgaact

SEQ ID NO: 751 - DNA, 17 bp, Homo sapiens
ccattttccatgaactg

SEQ ID NO: 752 - DNA, 17 bp, Homo sapiens
cattttccatgaactgc

SEQ ID NO: 753 - DNA, 17 bp, Homo sapiens
attttccatgaactgca

SEQ ID NO: 754 - DNA, 17 bp, Homo sapiens
ttttccatgaactgcac

SEQ ID NO: 755 - DNA, 17 bp, Homo sapiens
tttccatgaactgcacg

SEQ ID NO: 756 - DNA, 17 bp, Homo sapiens
ttccatgaactgcacgt

SEQ ID NO: 757 - DNA, 17 bp, Homo sapiens
tccatgaactgcacgtc

SEQ ID NO: 758 - DNA, 17 bp, Homo sapiens
ccatgaactgcacgtcg

SEQ ID NO: 759 - DNA, 17 bp, Homo sapiens
catgaactgcacgtcga

SEQ ID NO: 760 - DNA, 17 bp, Homo sapiens
atgaactgcacgtcgac

SEQ ID NO: 761 - DNA, 17 bp, Homo sapiens
tgaactgcacgtcgacc

SEQ ID NO: 762 - DNA, 17 bp, Homo sapiens
gaactgcacgtcgaccc

SEQ ID NO: 763 - DNA, 17 bp, Homo sapiens
aactgcacgtcgaccct

SEQ ID NO: 764 - DNA, 17 bp, Homo sapiens
actgcacgtcgaccctg

SEQ ID NO: 765 - DNA, 17 bp, Homo sapiens
ctgcacgtcgaccctga

SEQ ID NO: 766 - DNA, 17 bp, Homo sapiens
tgcacgtcgaccctgac

SEQ ID NO: 767 - DNA, 17 bp, Homo sapiens
gcacgtcgaccctgacc

SEQ ID NO: 768 - DNA, 17 bp, Homo sapiens
cacgtcgaccctgacct

SEQ ID NO: 769 - DNA, 17 bp, Homo sapiens
acgtcgaccctgacctg

SEQ ID NO: 770 - DNA, 17 bp, Homo sapiens
cgtcgaccctgacctgt

SEQ ID NO: 771 - DNA, 17 bp, Homo sapiens
gtcgaccctgacctgta

SEQ ID NO: 772 - DNA, 17 bp, Homo sapiens
tcgaccctgacctgtac

SEQ ID NO: 773 - DNA, 17 bp, Homo sapiens
cgaccctgacctgtaca

SEQ ID NO: 774 - DNA, 17 bp, Homo sapiens
gaccctgacctgtacac

SEQ ID NO: 775 - DNA, 17 bp, Homo sapiens
accctgacctgtacaca

SEQ ID NO: 776 - DNA, 17 bp, Homo sapiens
ccctgacctgtacacac

SEQ ID NO: 777 - DNA, 17 bp, Homo sapiens
cctgacctgtacacact

SEQ ID NO: 778 - DNA, 17 bp, Homo sapiens
ctgacctgtacacactc

SEQ ID NO: 779 - DNA, 17 bp, Homo sapiens
tgacctgtacacactct

SEQ ID NO: 780 - DNA, 17 bp, Homo sapiens
gacctgtacacactctt

SEQ ID NO: 781 - DNA, 17 bp, Homo sapiens
acctgtacacactcttg

SEQ ID NO: 782 - DNA, 17 bp, Homo sapiens
cctgtacacactcttgt

SEQ ID NO: 783 - DNA, 17 bp, Homo sapiens
ctgtacacactcttgtt

SEQ ID NO: 784 - DNA, 17 bp, Homo sapiens
tgtacacactcttgttt

SEQ ID NO: 785 - DNA, 17 bp, Homo sapiens
gtacacactcttgtttg

SEQ ID NO: 786 - DNA, 17 bp, Homo sapiens
tacacactcttgtttgg

SEQ ID NO: 787 - DNA, 17 bp, Homo sapiens
acacactcttgtttgga

SEQ ID NO: 788 - DNA, 17 bp, Homo sapiens
cacactcttgtttggag

SEQ ID NO: 789 - DNA, 17 bp, Homo sapiens
acactcttgtttggaga

SEQ ID NO: 790 - DNA, 17 bp, Homo sapiens
cactcttgtttggagag

SEQ ID NO: 791 - DNA, 17 bp, Homo sapiens
actcttgtttggagaga

SEQ ID NO: 792 - DNA, 17 bp, Homo sapiens
ctcttgtttggagagag

SEQ ID NO: 793 - DNA, 17 bp, Homo sapiens
tcttgtttggagagagt

SEQ ID NO: 794 - DNA, 17 bp, Homo sapiens
cttgtttggagagagtg

SEQ ID NO: 795 - DNA, 17 bp, Homo sapiens
ttgtttggagagagtgt

SEQ ID NO: 796 - DNA, 17 bp, Homo sapiens
tgtttggagagagtgtg

SEQ ID NO: 797 - DNA, 17 bp, Homo sapiens
gtttggagagagtgtgt

SEQ ID NO: 798 - DNA, 17 bp, Homo sapiens
tttggagagagtgtgtt

SEQ ID NO: 799 - DNA, 17 bp, Homo sapiens
ttggagagagtgtgttg

SEQ ID NO: 800 - DNA, 17 bp, Homo sapiens
tggagagagtgtgttga

SEQ ID NO: 801 - DNA, 17 bp, Homo sapiens
ggagagagtgtgttgaa

SEQ ID NO: 802 - DNA, 17 bp, Homo sapiens
gagagagtgtgttgaat

SEQ ID NO: 803 - DNA, 17 bp, Homo sapiens
agagagtgtgttgaatg

SEQ ID NO: 804 - DNA, 17 bp, Homo sapiens
gagagtgtgttgaatga

SEQ ID NO: 805 - DNA, 17 bp, Homo sapiens
agagtgtgttgaatgat

SEQ ID NO: 806 - DNA, 17 bp, Homo sapiens
gagtgtgttgaatgatg

SEQ ID NO: 807 - DNA, 17 bp, Homo sapiens
agtgtgttgaatgatgc

SEQ ID NO: 808 - DNA, 17 bp, Homo sapiens
gtgtgttgaatgatgca

SEQ ID NO: 809 - DNA, 17 bp, Homo sapiens
tgtgttgaatgatgcag

SEQ ID NO: 810 - DNA, 17 bp, Homo sapiens
gtgttgaatgatgcagt

SEQ ID NO: 811 - DNA, 17 bp, Homo sapiens
tgttgaatgatgcagtg

SEQ ID NO: 812 - DNA, 17 bp, Homo sapiens
gttgaatgatgcagtgg

SEQ ID NO: 813 - DNA, 17 bp, Homo sapiens
ttgaatgatgcagtggc

SEQ ID NO: 814 - DNA, 17 bp, Homo sapiens
tgaatgatgcagtggcc

SEQ ID NO: 815 - DNA, 17 bp, Homo sapiens
gaatgatgcagtggcca

SEQ ID NO: 816 - DNA, 17 bp, Homo sapiens
aatgatgcagtggccat

SEQ ID NO: 817 - DNA, 17 bp, Homo sapiens
atgatgcagtggccata

SEQ ID NO: 818 - DNA, 17 bp, Homo sapiens
tgatgcagtggccatag

SEQ ID NO: 819 - DNA, 17 bp, Homo sapiens
gatgcagtggccatagt

SEQ ID NO: 820 - DNA, 17 bp, Homo sapiens
atgcagtggccatagtc

SEQ ID NO: 821 - DNA, 17 bp, Homo sapiens
tgcagtggccatagtcc

SEQ ID NO: 822 - DNA, 17 bp, Homo sapiens
gcagtggccatagtcct

SEQ ID NO: 823 - DNA, 17 bp, Homo sapiens
cagtggccatagtcctt

SEQ ID NO: 824 - DNA, 17 bp, Homo sapiens
agtggccatagtcctta

SEQ ID NO: 825 - DNA, 17 bp, Homo sapiens
gtggccatagtccttac

SEQ ID NO: 826 - DNA, 17 bp, Homo sapiens
tggccatagtccttaca

SEQ ID NO: 827 - DNA, 17 bp, Homo sapiens
ggccatagtccttacat

SEQ ID NO: 828 - DNA, 17 bp, Homo sapiens
gccatagtccttacata

SEQ ID NO: 829 - DNA, 17 bp, Homo sapiens
ccatagtccttacatat

SEQ ID NO: 830 - DNA, 17 bp, Homo sapiens
catagtccttacatatt

SEQ ID NO: 831 - DNA, 17 bp, Homo sapiens
atagtccttacatattc

SEQ ID NO: 832 - DNA, 17 bp, Homo sapiens
tagtccttacatattct

SEQ ID NO: 833 - DNA, 17 bp, Homo sapiens
agtccttacatattcta

SEQ ID NO: 834 - DNA, 17 bp, Homo sapiens
gtccttacatattctat

SEQ ID NO: 835 - DNA, 17 bp, Homo sapiens
tccttacatattctata

SEQ ID NO: 836 - DNA, 17 bp, Homo sapiens
ccttacatattctatat

SEQ ID NO: 837 - DNA, 17 bp, Homo sapiens
cttacatattctatatc

SEQ ID NO: 838 - DNA, 17 bp, Homo sapiens
ttacatattctatatcc

SEQ ID NO: 839 - DNA, 17 bp, Homo sapiens
tacatattctatatcca

SEQ ID NO: 840 - DNA, 17 bp, Homo sapiens
acatattctatatccat

SEQ ID NO: 841 - DNA, 17 bp, Homo sapiens
catattctatatccatt

SEQ ID NO: 842 - DNA, 17 bp, Homo sapiens
atattctatatccattt

SEQ ID NO: 843 - DNA, 17 bp, Homo sapiens
tattctatatccattta

SEQ ID NO: 844 - DNA, 17 bp, Homo sapiens
attctatatccatttac

SEQ ID NO: 845 - DNA, 17 bp, Homo sapiens
ttctatatccatttaca

SEQ ID NO: 846 - DNA, 17 bp, Homo sapiens
tctatatccatttacag

SEQ ID NO: 847 - DNA, 17 bp, Homo sapiens
ctatatccatttacagt

SEQ ID NO: 848 - DNA, 17 bp, Homo sapiens
tatatccatttacagtc

SEQ ID NO: 849 - DNA, 17 bp, Homo sapiens
atatccatttacagtcc

SEQ ID NO: 850 - DNA, 17 bp, Homo sapiens
tatccatttacagtccc

SEQ ID NO: 851 - DNA, 17 bp, Homo sapiens
atccatttacagtccca

SEQ ID NO: 852 - DNA, 17 bp, Homo sapiens
tccatttacagtcccaa

SEQ ID NO: 853 - DNA, 17 bp, Homo sapiens
ccatttacagtcccaag

SEQ ID NO: 854 - DNA, 17 bp, Homo sapiens
catttacagtcccaagg

SEQ ID NO: 855 - DNA, 17 bp, Homo sapiens
atttacagtcccaagga

SEQ ID NO: 856 - DNA, 17 bp, Homo sapiens
tttacagtcccaaggag

SEQ ID NO: 857 - DNA, 17 bp, Homo sapiens
ttacagtcccaaggaga

SEQ ID NO: 858 - DNA, 17 bp, Homo sapiens
tacagtcccaaggagaa

SEQ ID NO: 859 - DNA, 17 bp, Homo sapiens
acagtcccaaggagaat

SEQ ID NO: 860 - DNA, 17 bp, Homo sapiens
cagtcccaaggagaatc

SEQ ID NO: 861 - DNA, 17 bp, Homo sapiens
agtcccaaggagaatcc

SEQ ID NO: 862 - DNA, 17 bp, Homo sapiens
gtcccaaggagaatcca

SEQ ID NO: 863 - DNA, 17 bp, Homo sapiens
tcccaaggagaatccaa

SEQ ID NO: 864 - DNA, 17 bp, Homo sapiens
cccaaggagaatccaaa

SEQ ID NO: 865 - DNA, 17 bp, Homo sapiens
ccaaggagaatccaaat

SEQ ID NO: 866 - DNA, 17 bp, Homo sapiens
caaggagaatccaaatg

SEQ ID NO: 867 - DNA, 17 bp, Homo sapiens
aaggagaatccaaatgc

SEQ ID NO: 868 - DNA, 17 bp, Homo sapiens
aggagaatccaaatgca

SEQ ID NO: 869 - DNA, 17 bp, Homo sapiens
ggagaatccaaatgcat

SEQ ID NO: 870 - DNA, 17 bp, Homo sapiens
gagaatccaaatgcatt

SEQ ID NO: 871 - DNA, 17 bp, Homo sapiens
agaatccaaatgcattt

SEQ ID NO: 872 - DNA, 17 bp, Homo sapiens
gaatccaaatgcatttg

SEQ ID NO: 873 - DNA, 17 bp, Homo sapiens
aatccaaatgcatttga

SEQ ID NO: 874 - DNA, 17 bp, Homo sapiens
atccaaatgcatttgat

SEQ ID NO: 875 - DNA, 17 bp, Homo sapiens
tccaaatgcatttgatg

SEQ ID NO: 876 - DNA, 17 bp, Homo sapiens
ccaaatgcatttgatgc

SEQ ID NO: 877 - DNA, 17 bp, Homo sapiens
caaatgcatttgatgcc

SEQ ID NO: 878 - DNA, 17 bp, Homo sapiens
aaatgcatttgatgccg

SEQ ID NO: 879 - DNA, 17 bp, Homo sapiens
aatgcatttgatgccgc

SEQ ID NO: 880 - DNA, 17 bp, Homo sapiens
atgcatttgatgccgca

SEQ ID NO: 881 - DNA, 17 bp, Homo sapiens
tgcatttgatgccgcag

SEQ ID NO: 882 - DNA, 17 bp, Homo sapiens
gcatttgatgccgcagc

SEQ ID NO: 883 - DNA, 17 bp, Homo sapiens
catttgatgccgcagca

SEQ ID NO: 884 - DNA, 17 bp, Homo sapiens
atttgatgccgcagcat

SEQ ID NO: 885 - DNA, 17 bp, Homo sapiens
tttgatgccgcagcatt

SEQ ID NO: 886 - DNA, 17 bp, Homo sapiens
ttgatgccgcagcattc

SEQ ID NO: 887 - DNA, 17 bp, Homo sapiens
tgatgccgcagcattct

SEQ ID NO: 888 - DNA, 17 bp, Homo sapiens
gatgccgcagcattctt

SEQ ID NO: 889 - DNA, 17 bp, Homo sapiens
atgccgcagcattcttc

SEQ ID NO: 890 - DNA, 17 bp, Homo sapiens
tgccgcagcattcttcc

SEQ ID NO: 891 - DNA, 17 bp, Homo sapiens
gccgcagcattcttcca

SEQ ID NO: 892 - DNA, 17 bp, Homo sapiens
ccgcagcattcttccag

SEQ ID NO: 893 - DNA, 17 bp, Homo sapiens
cgcagcattcttccagt

SEQ ID NO: 894 - DNA, 17 bp, Homo sapiens
gcagcattcttccagtc

SEQ ID NO: 895 - DNA, 17 bp, Homo sapiens
cagcattcttccagtct

SEQ ID NO: 896 - DNA, 17 bp, Homo sapiens
agcattcttccagtctg

SEQ ID NO: 897 - DNA, 17 bp, Homo sapiens
gcattcttccagtctgt

SEQ ID NO: 898 - DNA, 17 bp, Homo sapiens
cattcttccagtctgtg

SEQ ID NO: 899 - DNA, 17 bp, Homo sapiens
attcttccagtctgtgg

SEQ ID NO: 900 - DNA, 17 bp, Homo sapiens
ttcttccagtctgtggg

SEQ ID NO: 901 - DNA, 17 bp, Homo sapiens
tcttccagtctgtgggg

SEQ ID NO: 902 - DNA, 17 bp, Homo sapiens
cttccagtctgtgggga

SEQ ID NO: 903 - DNA, 17 bp, Homo sapiens
ttccagtctgtggggaa

SEQ ID NO: 904 - DNA, 17 bp, Homo sapiens
tccagtctgtggggaat

SEQ ID NO: 905 - DNA, 17 bp, Homo sapiens
ccagtctgtggggaatt

SEQ ID NO: 906 - DNA, 17 bp, Homo sapiens
cagtctgtggggaattt

SEQ ID NO: 907 - DNA, 17 bp, Homo sapiens
agtctgtggggaatttc

SEQ ID NO: 908 - DNA, 17 bp, Homo sapiens
gtctgtggggaatttcc

SEQ ID NO: 909 - DNA, 17 bp, Homo sapiens
tctgtggggaatttcct

SEQ ID NO: 910 - DNA, 17 bp, Homo sapiens
ctgtggggaatttcctg

SEQ ID NO: 911 - DNA, 17 bp, Homo sapiens
tgtggggaatttcctgg

SEQ ID NO: 912 - DNA, 17 bp, Homo sapiens
gtggggaatttcctggg

SEQ ID NO: 913 - DNA, 17 bp, Homo sapiens
tggggaatttcctggga

SEQ ID NO: 914 - DNA, 17 bp, Homo sapiens
ggggaatttcctgggaa

SEQ ID NO: 915 - DNA, 17 bp, Homo sapiens
gggaatttcctgggaat

SEQ ID NO: 916 - DNA, 17 bp, Homo sapiens
ggaatttcctgggaatc

SEQ ID NO: 917 - DNA, 17 bp, Homo sapiens
gaatttcctgggaatct

SEQ ID NO: 918 - DNA, 17 bp, Homo sapiens
aatttcctgggaatctt

SEQ ID NO: 919 - DNA, 17 bp, Homo sapiens
atttcctgggaatcttc

SEQ ID NO: 920 - DNA, 17 bp, Homo sapiens
tttcctgggaatcttcg

SEQ ID NO: 921 - DNA, 17 bp, Homo sapiens
ttcctgggaatcttcgc

SEQ ID NO: 922 - DNA, 17 bp, Homo sapiens
tcctgggaatcttcgct

SEQ ID NO: 923 - DNA, 17 bp, Homo sapiens
cctgggaatcttcgctg

SEQ ID NO: 924 - DNA, 17 bp, Homo sapiens
ctgggaatcttcgctgg

SEQ ID NO: 925 - DNA, 17 bp, Homo sapiens
tgggaatcttcgctggc

SEQ ID NO: 926 - DNA, 17 bp, Homo sapiens
gggaatcttcgctggct

SEQ ID NO: 927 - DNA, 17 bp, Homo sapiens
ggaatcttcgctggctc

SEQ ID NO: 928 - DNA, 17 bp, Homo sapiens
gaatcttcgctggctca

SEQ ID NO: 929 - DNA, 17 bp, Homo sapiens
aatcttcgctggctcat

SEQ ID NO: 930 - DNA, 17 bp, Homo sapiens
atcttcgctggctcatt

SEQ ID NO: 931 - DNA, 17 bp, Homo sapiens
tcttcgctggctcattt

SEQ ID NO: 932 - DNA, 17 bp, Homo sapiens
cttcgctggctcatttg

SEQ ID NO: 933 - DNA, 17 bp, Homo sapiens
ttcgctggctcatttgc

SEQ ID NO: 934 - DNA, 17 bp, Homo sapiens
tcgctggctcatttgca

SEQ ID NO: 935 - DNA, 17 bp, Homo sapiens
cgctggctcatttgcaa

SEQ ID NO: 936 - DNA, 17 bp, Homo sapiens
gctggctcatttgcaat

SEQ ID NO: 937 - DNA, 17 bp, Homo sapiens
ctggctcatttgcaatg

SEQ ID NO: 938 - DNA, 17 bp, Homo sapiens
tggctcatttgcaatgg

SEQ ID NO: 939 - DNA, 17 bp, Homo sapiens
ggctcatttgcaatggg

SEQ ID NO: 940 - DNA, 17 bp, Homo sapiens
gctcatttgcaatgggg

SEQ ID NO: 941 - DNA, 17 bp, Homo sapiens
ctcatttgcaatggggt

SEQ ID NO: 942 - DNA, 17 bp, Homo sapiens
tcatttgcaatggggtc

SEQ ID NO: 943 - DNA, 17 bp, Homo sapiens
catttgcaatggggtct

SEQ ID NO: 944 - DNA, 17 bp, Homo sapiens
atttgcaatggggtctg

SEQ ID NO: 945 - DNA, 17 bp, Homo sapiens
tttgcaatggggtctgc

SEQ ID NO: 946 - DNA, 17 bp, Homo sapiens
ttgcaatggggtctgcg

SEQ ID NO: 947 - DNA, 17 bp, Homo sapiens
tgcaatggggtctgcgt

SEQ ID NO: 948 - DNA, 17 bp, Homo sapiens
gcaatggggtctgcgta

SEQ ID NO: 949 - DNA, 17 bp, Homo sapiens
caatggggtctgcgtat

SEQ ID NO: 950 - DNA, 17 bp, Homo sapiens
aatggggtctgcgtatg

SEQ ID NO: 951 - DNA, 17 bp, Homo sapiens
atggggtctgcgtatgc

SEQ ID NO: 952 - DNA, 17 bp, Homo sapiens
tggggtctgcgtatgcc

SEQ ID NO: 953 - DNA, 17 bp, Homo sapiens
ggggtctgcgtatgcca

SEQ ID NO: 954 - DNA, 17 bp, Homo sapiens
gggtctgcgtatgccat

SEQ ID NO: 955 - DNA, 17 bp, Homo sapiens
ggtctgcgtatgccatc

SEQ ID NO: 956 - DNA, 17 bp, Homo sapiens
gtctgcgtatgccatca

SEQ ID NO: 957 - DNA, 17 bp, Homo sapiens
tctgcgtatgccatcat

SEQ ID NO: 958 - DNA, 17 bp, Homo sapiens
ctgcgtatgccatcatc

SEQ ID NO: 959 - DNA, 17 bp, Homo sapiens
tgcgtatgccatcatca

SEQ ID NO: 960 - DNA, 17 bp, Homo sapiens
gcgtatgccatcatcac

SEQ ID NO: 961 - DNA, 17 bp, Homo sapiens
cgtatgccatcatcaca

SEQ ID NO: 962 - DNA, 17 bp, Homo sapiens
gtatgccatcatcacag

SEQ ID NO: 963 - DNA, 17 bp, Homo sapiens
tatgccatcatcacagc

SEQ ID NO: 964 - DNA, 17 bp, Homo sapiens
atgccatcatcacagca

SEQ ID NO: 965 - DNA, 17 bp, Homo sapiens
tgccatcatcacagcac

SEQ ID NO: 966 - DNA, 17 bp, Homo sapiens
gccatcatcacagcact

SEQ ID NO: 967 - DNA, 17 bp, Homo sapiens
ccatcatcacagcactg

SEQ ID NO: 968 - DNA, 17 bp, Homo sapiens
catcatcacagcactgt

SEQ ID NO: 969 - DNA, 17 bp, Homo sapiens
atcatcacagcactgtt

SEQ ID NO: 970 - DNA, 17 bp, Homo sapiens
tcatcacagcactgttg

SEQ ID NO: 971 - DNA, 17 bp, Homo sapiens
catcacagcactgttga

SEQ ID NO: 972 - DNA, 17 bp, Homo sapiens
atcacagcactgttgac

SEQ ID NO: 973 - DNA, 17 bp, Homo sapiens
tcacagcactgttgacc

SEQ ID NO: 974 - DNA, 17 bp, Homo sapiens
cacagcactgttgacca

SEQ ID NO: 975 - DNA, 17 bp, Homo sapiens
acagcactgttgaccaa

SEQ ID NO: 976 - DNA, 17 bp, Homo sapiens
cagcactgttgaccaaa

SEQ ID NO: 977 - DNA, 17 bp, Homo sapiens
agcactgttgaccaaat

SEQ ID NO: 978 - DNA, 17 bp, Homo sapiens
gcactgttgaccaaatt

SEQ ID NO: 979 - DNA, 17 bp, Homo sapiens
cactgttgaccaaattt

SEQ ID NO: 980 - DNA, 17 bp, Homo sapiens
actgttgaccaaattta

SEQ ID NO: 981 - DNA, 17 bp, Homo sapiens
ctgttgaccaaatttac

SEQ ID NO: 982 - DNA, 17 bp, Homo sapiens
tgttgaccaaatttacc

SEQ ID NO: 983 - DNA, 17 bp, Homo sapiens
gttgaccaaatttacca

SEQ ID NO: 984 - DNA, 17 bp, Homo sapiens
ttgaccaaatttaccaa

SEQ ID NO: 985 - DNA, 17 bp, Homo sapiens
tgaccaaatttaccaag

SEQ ID NO: 986 - DNA, 17 bp, Homo sapiens
gaccaaatttaccaagc

SEQ ID NO: 987 - DNA, 17 bp, Homo sapiens
accaaatttaccaagct

SEQ ID NO: 988 - DNA, 17 bp, Homo sapiens
ccaaatttaccaagctg

SEQ ID NO: 989 - DNA, 17 bp, Homo sapiens
caaatttaccaagctgt

SEQ ID NO: 990 - DNA, 17 bp, Homo sapiens
aaatttaccaagctgtg

SEQ ID NO: 991 - DNA, 17 bp, Homo sapiens
aatttaccaagctgtgt

SEQ ID NO: 992 - DNA, 17 bp, Homo sapiens
atttaccaagctgtgtg

SEQ ID NO: 993 - DNA, 17 bp, Homo sapiens
tttaccaagctgtgtga

SEQ ID NO: 994 - DNA, 17 bp, Homo sapiens
ttaccaagctgtgtgag

SEQ ID NO: 995 - DNA, 17 bp, Homo sapiens
taccaagctgtgtgagt

SEQ ID NO: 996 - DNA, 17 bp, Homo sapiens
accaagctgtgtgagtt

SEQ ID NO: 997 - DNA, 17 bp, Homo sapiens
ccaagctgtgtgagttc

SEQ ID NO: 998 - DNA, 17 bp, Homo sapiens
caagctgtgtgagttcc

SEQ ID NO: 999 - DNA, 17 bp, Homo sapiens
aagctgtgtgagttccc

SEQ ID NO: 1000 - DNA, 17 bp, Homo sapiens
agctgtgtgagttcccg

SEQ ID NO: 1001 - DNA, 17 bp, Homo sapiens
gctgtgtgagttcccga

SEQ ID NO: 1002 - DNA, 17 bp, Homo sapiens
ctgtgtgagttcccgat

SEQ ID NO: 1003 - DNA, 17 bp, Homo sapiens
tgtgtgagttcccgatg

SEQ ID NO: 1004 - DNA, 17 bp, Homo sapiens
gtgtgagttcccgatgc

SEQ ID NO: 1005 - DNA, 17 bp, Homo sapiens
tgtgagttcccgatgct

SEQ ID NO: 1006 - DNA, 17 bp, Homo sapiens
gtgagttcccgatgctg

SEQ ID NO: 1007 - DNA, 17 bp, Homo sapiens
tgagttcccgatgctgg

SEQ ID NO: 1008 - DNA, 17 bp, Homo sapiens
gagttcccgatgctgga

SEQ ID NO: 1009 - DNA, 17 bp, Homo sapiens
agttcccgatgctggaa

SEQ ID NO: 1010 - DNA, 17 bp, Homo sapiens
gttcccgatgctggaaa

SEQ ID NO: 1011 - DNA, 17 bp, Homo sapiens
ttcccgatgctggaaac

SEQ ID NO: 1012 - DNA, 17 bp, Homo sapiens
tcccgatgctggaaacc

SEQ ID NO: 1013 - DNA, 17 bp, Homo sapiens
cccgatgctggaaaccg

SEQ ID NO: 1014 - DNA, 17 bp, Homo sapiens
ccgatgctggaaaccgg

SEQ ID NO: 1015 - DNA, 17 bp, Homo sapiens
cgatgctggaaaccggc

SEQ ID NO: 1016 - DNA, 17 bp, Homo sapiens
gatgctggaaaccggcc

SEQ ID NO: 1017 - DNA, 17 bp, Homo sapiens
atgctggaaaccggcct

SEQ ID NO: 1018 - DNA, 17 bp, Homo sapiens
tgctggaaaccggcctg

SEQ ID NO: 1019 - DNA, 17 bp, Homo sapiens
gctggaaaccggcctgt

SEQ ID NO: 1020 - DNA, 17 bp, Homo sapiens
ctggaaaccggcctgtt

SEQ ID NO: 1021 - DNA, 17 bp, Homo sapiens
tggaaaccggcctgttt

SEQ ID NO: 1022 - DNA, 17 bp, Homo sapiens
ggaaaccggcctgtttt

SEQ ID NO: 1023 - DNA, 17 bp, Homo sapiens
gaaaccggcctgttttt

SEQ ID NO: 1024 - DNA, 17 bp, Homo sapiens
aaaccggcctgtttttc

SEQ ID NO: 1025 - DNA, 17 bp, Homo sapiens
aaccggcctgtttttcc

SEQ ID NO: 1026 - DNA, 17 bp, Homo sapiens
accggcctgtttttcct

SEQ ID NO: 1027 - DNA, 17 bp, Homo sapiens
ccggcctgtttttcctg

SEQ ID NO: 1028 - DNA, 17 bp, Homo sapiens
cggcctgtttttcctgc

SEQ ID NO: 1029 - DNA, 17 bp, Homo sapiens
ggcctgtttttcctgct

SEQ ID NO: 1030 - DNA, 17 bp, Homo sapiens
gcctgtttttcctgctt

SEQ ID NO: 1031 - DNA, 17 bp, Homo sapiens
cctgtttttcctgcttt

SEQ ID NO: 1032 - DNA, 17 bp, Homo sapiens
ctgtttttcctgctttc

SEQ ID NO: 1033 - DNA, 17 bp, Homo sapiens
tgtttttcctgctttct

SEQ ID NO: 1034 - DNA, 17 bp, Homo sapiens
gtttttcctgctttctt

SEQ ID NO: 1035 - DNA, 17 bp, Homo sapiens
tttttcctgctttcttg

SEQ ID NO: 1036 - DNA, 17 bp, Homo sapiens
ttttcctgctttcttgg

SEQ ID NO: 1037 - DNA, 17 bp, Homo sapiens
tttcctgctttcttgga

SEQ ID NO: 1038 - DNA, 17 bp, Homo sapiens
ttcctgctttcttggag

SEQ ID NO: 1039 - DNA, 17 bp, Homo sapiens
tcctgctttcttggagt

SEQ ID NO: 1040 - DNA, 17 bp, Homo sapiens
cctgctttcttggagtg

SEQ ID NO: 1041 - DNA, 17 bp, Homo sapiens
ctgctttcttggagtgc

SEQ ID NO: 1042 - DNA, 17 bp, Homo sapiens
tgctttcttggagtgcc

SEQ ID NO: 1043 - DNA, 17 bp, Homo sapiens
gctttcttggagtgcct

SEQ ID NO: 1044 - DNA, 17 bp, Homo sapiens
ctttcttggagtgcctt

SEQ ID NO: 1045 - DNA, 17 bp, Homo sapiens
tttcttggagtgccttc

SEQ ID NO: 1046 - DNA, 17 bp, Homo sapiens
ttcttggagtgccttcc

SEQ ID NO: 1047 - DNA, 17 bp, Homo sapiens
tcttggagtgccttcct

SEQ ID NO: 1048 - DNA, 17 bp, Homo sapiens
cttggagtgccttcctg

SEQ ID NO: 1049 - DNA, 17 bp, Homo sapiens
ttggagtgccttcctgt

SEQ ID NO: 1050 - DNA, 17 bp, Homo sapiens
tggagtgccttcctgtc

SEQ ID NO: 1051 - DNA, 17 bp, Homo sapiens
ggagtgccttcctgtct

SEQ ID NO: 1052 - DNA, 17 bp, Homo sapiens
gagtgccttcctgtctg

SEQ ID NO: 1053 - DNA, 17 bp, Homo sapiens
agtgccttcctgtctgc

SEQ ID NO: 1054 - DNA, 17 bp, Homo sapiens
gtgccttcctgtctgcc

SEQ ID NO: 1055 - DNA, 17 bp, Homo sapiens
tgccttcctgtctgccg

SEQ ID NO: 1056 - DNA, 17 bp, Homo sapiens
gccttcctgtctgccga

SEQ ID NO: 1057 - DNA, 17 bp, Homo sapiens
ccttcctgtctgccgag

SEQ ID NO: 1058 - DNA, 17 bp, Homo sapiens
cttcctgtctgccgagg

SEQ ID NO: 1059 - DNA, 17 bp, Homo sapiens
ttcctgtctgccgaggc

SEQ ID NO: 1060 - DNA, 17 bp, Homo sapiens
tcctgtctgccgaggct

SEQ ID NO: 1061 - DNA, 17 bp, Homo sapiens
cctgtctgccgaggctg

SEQ ID NO: 1062 - DNA, 17 bp, Homo sapiens
ctgtctgccgaggctgc

SEQ ID NO: 1063 - DNA, 17 bp, Homo sapiens
tgtctgccgaggctgcc

SEQ ID NO: 1064 - DNA, 17 bp, Homo sapiens
gtctgccgaggctgccg

SEQ ID NO: 1065 - DNA, 17 bp, Homo sapiens
tctgccgaggctgccgg

SEQ ID NO: 1066 - DNA, 17 bp, Homo sapiens
ctgccgaggctgccggc

SEQ ID NO: 1067 - DNA, 17 bp, Homo sapiens
tgccgaggctgccggcc

SEQ ID NO: 1068 - DNA, 17 bp, Homo sapiens
gccgaggctgccggcct

SEQ ID NO: 1069 - DNA, 17 bp, Homo sapiens
ccgaggctgccggccta

SEQ ID NO: 1070 - DNA, 17 bp, Homo sapiens
cgaggctgccggcctaa

SEQ ID NO: 1071 - DNA, 17 bp, Homo sapiens
gaggctgccggcctaac

SEQ ID NO: 1072 - DNA, 17 bp, Homo sapiens
aggctgccggcctaaca

SEQ ID NO: 1073 - DNA, 17 bp, Homo sapiens
ggctgccggcctaacag

SEQ ID NO: 1074 - DNA, 17 bp, Homo sapiens
gctgccggcctaacagg

SEQ ID NO: 1075 - DNA, 17 bp, Homo sapiens
ctgccggcctaacaggg

SEQ ID NO: 1076 - DNA, 17 bp, Homo sapiens
tgccggcctaacaggga

SEQ ID NO: 1077 - DNA, 17 bp, Homo sapiens
gccggcctaacagggat

SEQ ID NO: 1078 - DNA, 17 bp, Homo sapiens
ccggcctaacagggata

SEQ ID NO: 1079 - DNA, 17 bp, Homo sapiens
cggcctaacagggatag

SEQ ID NO: 1080 - DNA, 17 bp, Homo sapiens
ggcctaacagggatagt

SEQ ID NO: 1081 - DNA, 17 bp, Homo sapiens
gcctaacagggatagtt

SEQ ID NO: 1082 - DNA, 17 bp, Homo sapiens
cctaacagggatagttg

SEQ ID NO: 1083 - DNA, 17 bp, Homo sapiens
ctaacagggatagttgc

SEQ ID NO: 1084 - DNA, 17 bp, Homo sapiens
taacagggatagttgct

SEQ ID NO: 1085 - DNA, 17 bp, Homo sapiens
aacagggatagttgctg

SEQ ID NO: 1086 - DNA, 17 bp, Homo sapiens
acagggatagttgctgt

SEQ ID NO: 1087 - DNA, 17 bp, Homo sapiens
cagggatagttgctgtt

SEQ ID NO: 1088 - DNA, 17 bp, Homo sapiens
agggatagttgctgttc

SEQ ID NO: 1089 - DNA, 17 bp, Homo sapiens
gggatagttgctgttct

SEQ ID NO: 1090 - DNA, 17 bp, Homo sapiens
ggatagttgctgttctc

SEQ ID NO: 1091 - DNA, 17 bp, Homo sapiens
gatagttgctgttctct

SEQ ID NO: 1092 - DNA, 17 bp, Homo sapiens
atagttgctgttctctt

SEQ ID NO: 1093 - DNA, 17 bp, Homo sapiens
tagttgctgttctcttc

SEQ ID NO: 1094 - DNA, 17 bp, Homo sapiens
agttgctgttctcttct

SEQ ID NO: 1095 - DNA, 17 bp, Homo sapiens
gttgctgttctcttctg

SEQ ID NO: 1096 - DNA, 17 bp, Homo sapiens
ttgctgttctcttctgt

SEQ ID NO: 1097 - DNA, 17 bp, Homo sapiens
tgctgttctcttctgtg

SEQ ID NO: 1098 - DNA, 17 bp, Homo sapiens
gctgttctcttctgtgg

SEQ ID NO: 1099 - DNA, 17 bp, Homo sapiens
ctgttctcttctgtgga

SEQ ID NO: 1100 - DNA, 17 bp, Homo sapiens
tgttctcttctgtggag

SEQ ID NO: 1101 - DNA, 17 bp, Homo sapiens
gttctcttctgtggagt

SEQ ID NO: 1102 - DNA, 17 bp, Homo sapiens
ttctcttctgtggagtc

SEQ ID NO: 1103 - DNA, 17 bp, Homo sapiens
tctcttctgtggagtca

SEQ ID NO: 1104 - DNA, 17 bp, Homo sapiens
ctcttctgtggagtcac

SEQ ID NO: 1105 - DNA, 17 bp, Homo sapiens
tcttctgtggagtcaca

SEQ ID NO: 1106 - DNA, 17 bp, Homo sapiens
cttctgtggagtcacac

SEQ ID NO: 1107 - DNA, 17 bp, Homo sapiens
ttctgtggagtcacaca

SEQ ID NO: 1108 - DNA, 17 bp, Homo sapiens
tctgtggagtcacacaa

SEQ ID NO: 1109 - DNA, 17 bp, Homo sapiens
ctgtggagtcacacaag

SEQ ID NO: 1110 - DNA, 17 bp, Homo sapiens
tgtggagtcacacaagc

SEQ ID NO: 1111 - DNA, 17 bp, Homo sapiens
gtggagtcacacaagca

SEQ ID NO: 1112 - DNA, 17 bp, Homo sapiens
tggagtcacacaagcac

SEQ ID NO: 1113 - DNA, 17 bp, Homo sapiens
ggagtcacacaagcaca

SEQ ID NO: 1114 - DNA, 17 bp, Homo sapiens
gagtcacacaagcacat

SEQ ID NO: 1115 - DNA, 17 bp, Homo sapiens
agtcacacaagcacatt

SEQ ID NO: 1116 - DNA, 17 bp, Homo sapiens
gtcacacaagcacatta

SEQ ID NO: 1117 - DNA, 17 bp, Homo sapiens
tcacacaagcacattat

SEQ ID NO: 1118 - DNA, 17 bp, Homo sapiens
cacacaagcacattata

SEQ ID NO: 1119 - DNA, 17 bp, Homo sapiens
acacaagcacattatac

SEQ ID NO: 1120 - DNA, 17 bp, Homo sapiens
cacaagcacattatacc

SEQ ID NO: 1121 - DNA, 17 bp, Homo sapiens
acaagcacattatacct

SEQ ID NO: 1122 - DNA, 17 bp, Homo sapiens
caagcacattataccta

SEQ ID NO: 1123 - DNA, 17 bp, Homo sapiens
aagcacattatacctac

SEQ ID NO: 1124 - DNA, 17 bp, Homo sapiens
agcacattatacctaca

SEQ ID NO: 1125 - DNA, 17 bp, Homo sapiens
gcacattatacctacaa

SEQ ID NO: 1126 - DNA, 17 bp, Homo sapiens
cacattatacctacaac

SEQ ID NO: 1127 - DNA, 17 bp, Homo sapiens
acattatacctacaaca

SEQ ID NO: 1128 - DNA, 17 bp, Homo sapiens
cattatacctacaacaa

SEQ ID NO: 1129 - DNA, 17 bp, Homo sapiens
attatacctacaacaat

SEQ ID NO: 1130 - DNA, 17 bp, Homo sapiens
ttatacctacaacaatc

SEQ ID NO: 1131 - DNA, 17 bp, Homo sapiens
tatacctacaacaatct

SEQ ID NO: 1132 - DNA, 17 bp, Homo sapiens
atacctacaacaatctg

SEQ ID NO: 1133 - DNA, 17 bp, Homo sapiens
tacctacaacaatctgt

SEQ ID NO: 1134 - DNA, 17 bp, Homo sapiens
acctacaacaatctgtc

SEQ ID NO: 1135 - DNA, 17 bp, Homo sapiens
cctacaacaatctgtct

SEQ ID NO: 1136 - DNA, 17 bp, Homo sapiens
ctacaacaatctgtctt

SEQ ID NO: 1137 - DNA, 17 bp, Homo sapiens
tacaacaatctgtcttc

SEQ ID NO: 1138 - DNA, 17 bp, Homo sapiens
acaacaatctgtcttcg

SEQ ID NO: 1139 - DNA, 17 bp, Homo sapiens
caacaatctgtcttcgg

SEQ ID NO: 1140 - DNA, 17 bp, Homo sapiens
aacaatctgtcttcgga

SEQ ID NO: 1141 - DNA, 17 bp, Homo sapiens
acaatctgtcttcggat

SEQ ID NO: 1142 - DNA, 17 bp, Homo sapiens
caatctgtcttcggatt

SEQ ID NO: 1143 - DNA, 17 bp, Homo sapiens
aatctgtcttcggattc

SEQ ID NO: 1144 - DNA, 17 bp, Homo sapiens
atctgtcttcggattcc

SEQ ID NO: 1145 - DNA, 17 bp, Homo sapiens
tctgtcttcggattcca

SEQ ID NO: 1146 - DNA, 17 bp, Homo sapiens
ctgtcttcggattccaa

SEQ ID NO: 1147 - DNA, 17 bp, Homo sapiens
tgtcttcggattccaaa

SEQ ID NO: 1148 - DNA, 17 bp, Homo sapiens
gtcttcggattccaaaa

SEQ ID NO: 1149 - DNA, 17 bp, Homo sapiens
tcttcggattccaaaat

SEQ ID NO: 1150 - DNA, 17 bp, Homo sapiens
cttcggattccaaaata

SEQ ID NO: 1151 - DNA, 17 bp, Homo sapiens
ttcggattccaaaataa

SEQ ID NO: 1152 - DNA, 17 bp, Homo sapiens
tcggattccaaaataag

SEQ ID NO: 1153 - DNA, 17 bp, Homo sapiens
cggattccaaaataaga

SEQ ID NO: 1154 - DNA, 17 bp, Homo sapiens
ggattccaaaataagaa

SEQ ID NO: 1155 - DNA, 17 bp, Homo sapiens
gattccaaaataagaac

SEQ ID NO: 1156 - DNA, 17 bp, Homo sapiens
attccaaaataagaact

SEQ ID NO: 1157 - DNA, 17 bp, Homo sapiens
ttccaaaataagaacta

SEQ ID NO: 1158 - DNA, 17 bp, Homo sapiens
tccaaaataagaactaa

SEQ ID NO: 1159 - DNA, 17 bp, Homo sapiens
ccaaaataagaactaaa

SEQ ID NO: 1160 - DNA, 17 bp, Homo sapiens
caaaataagaactaaac

SEQ ID NO: 1161 - DNA, 17 bp, Homo sapiens
aaaataagaactaaaca

SEQ ID NO: 1162 - DNA, 17 bp, Homo sapiens
aaataagaactaaacag

SEQ ID NO: 1163 - DNA, 17 bp, Homo sapiens
aataagaactaaacagt

SEQ ID NO: 1164 - DNA, 17 bp, Homo sapiens
ataagaactaaacagtt

SEQ ID NO: 1165 - DNA, 17 bp, Homo sapiens
taagaactaaacagttg

SEQ ID NO: 1166 - DNA, 17 bp, Homo sapiens
aagaactaaacagttgt

SEQ ID NO: 1167 - DNA, 17 bp, Homo sapiens
agaactaaacagttgtt

SEQ ID NO: 1168 - DNA, 17 bp, Homo sapiens
gaactaaacagttgttt

SEQ ID NO: 1169 - DNA, 17 bp, Homo sapiens
aactaaacagttgtttg

SEQ ID NO: 1170 - DNA, 17 bp, Homo sapiens
actaaacagttgtttga

SEQ ID NO: 1171 - DNA, 17 bp, Homo sapiens
ctaaacagttgtttgaa

SEQ ID NO: 1172 - DNA, 17 bp, Homo sapiens
taaacagttgtttgaat

SEQ ID NO: 1173 - DNA, 17 bp, Homo sapiens
aaacagttgtttgaatt

SEQ ID NO: 1174 - DNA, 17 bp, Homo sapiens
aacagttgtttgaattt

SEQ ID NO: 1175 - DNA, 17 bp, Homo sapiens
acagttgtttgaattta

SEQ ID NO: 1176 - DNA, 17 bp, Homo sapiens
cagttgtttgaatttat

SEQ ID NO: 1177 - DNA, 17 bp, Homo sapiens
agttgtttgaatttatg

SEQ ID NO: 1178 - DNA, 17 bp, Homo sapiens
gttgtttgaatttatga

SEQ ID NO: 1179 - DNA, 17 bp, Homo sapiens
ttgtttgaatttatgaa

SEQ ID NO: 1180 - DNA, 17 bp, Homo sapiens
tgtttgaatttatgaac

SEQ ID NO: 1181 - DNA, 17 bp, Homo sapiens
gtttgaatttatgaact

SEQ ID NO: 1182 - DNA, 17 bp, Homo sapiens
tttgaatttatgaactt

SEQ ID NO: 1183 - DNA, 17 bp, Homo sapiens
ttgaatttatgaacttt

SEQ ID NO: 1184 - DNA, 17 bp, Homo sapiens
tgaatttatgaactttt

SEQ ID NO: 1185 - DNA, 17 bp, Homo sapiens
gaatttatgaacttttt

SEQ ID NO: 1186 - DNA, 17 bp, Homo sapiens
aatttatgaactttttg

SEQ ID NO: 1187 - DNA, 17 bp, Homo sapiens
atttatgaactttttgg

SEQ ID NO: 1188 - DNA, 17 bp, Homo sapiens
tttatgaactttttggc

SEQ ID NO: 1189 - DNA, 17 bp, Homo sapiens
ttatgaactttttggcg

SEQ ID NO: 1190 - DNA, 17 bp, Homo sapiens
tatgaactttttggcgg

SEQ ID NO: 1191 - DNA, 17 bp, Homo sapiens
atgaactttttggcgga

SEQ ID NO: 1192 - DNA, 17 bp, Homo sapiens
tgaactttttggcggag

SEQ ID NO: 1193 - DNA, 17 bp, Homo sapiens
gaactttttggcggaga

SEQ ID NO: 1194 - DNA, 17 bp, Homo sapiens
aactttttggcggagaa

SEQ ID NO: 1195 - DNA, 17 bp, Homo sapiens
actttttggcggagaac

SEQ ID NO: 1196 - DNA, 17 bp, Homo sapiens
ctttttggcggagaacg

SEQ ID NO: 1197 - DNA, 17 bp, Homo sapiens
tttttggcggagaacgt

SEQ ID NO: 1198 - DNA, 17 bp, Homo sapiens
ttttggcggagaacgtc

SEQ ID NO: 1199 - DNA, 17 bp, Homo sapiens
tttggcggagaacgtca

SEQ ID NO: 1200 - DNA, 17 bp, Homo sapiens
ttggcggagaacgtcat

SEQ ID NO: 1201 - DNA, 17 bp, Homo sapiens
tggcggagaacgtcatc

SEQ ID NO: 1202 - DNA, 17 bp, Homo sapiens
ggcggagaacgtcatct

SEQ ID NO: 1203 - DNA, 17 bp, Homo sapiens
gcggagaacgtcatctt

SEQ ID NO: 1204 - DNA, 17 bp, Homo sapiens
cggagaacgtcatcttc

SEQ ID NO: 1205 - DNA, 17 bp, Homo sapiens
ggagaacgtcatcttct

SEQ ID NO: 1206 - DNA, 17 bp, Homo sapiens
gagaacgtcatcttctg

SEQ ID NO: 1207 - DNA, 17 bp, Homo sapiens
agaacgtcatcttctgt

SEQ ID NO: 1208 - DNA, 17 bp, Homo sapiens
gaacgtcatcttctgtt

SEQ ID NO: 1209 - DNA, 17 bp, Homo sapiens
aacgtcatcttctgtta

SEQ ID NO: 1210 - DNA, 17 bp, Homo sapiens
acgtcatcttctgttac

SEQ ID NO: 1211 - DNA, 17 bp, Homo sapiens
cgtcatcttctgttaca

SEQ ID NO: 1212 - DNA, 17 bp, Homo sapiens
gtcatcttctgttacat

SEQ ID NO: 1213 - DNA, 17 bp, Homo sapiens
tcatcttctgttacatg

SEQ ID NO: 1214 - DNA, 17 bp, Homo sapiens
catcttctgttacatgg

SEQ ID NO: 1215 - DNA, 17 bp, Homo sapiens
atcttctgttacatggg

SEQ ID NO: 1216 - DNA, 17 bp, Homo sapiens
tcttctgttacatgggc

SEQ ID NO: 1217 - DNA, 17 bp, Homo sapiens
cttctgttacatgggcc

SEQ ID NO: 1218 - DNA, 17 bp, Homo sapiens
ttctgttacatgggcct

SEQ ID NO: 1219 - DNA, 17 bp, Homo sapiens
tctgttacatgggcctg

SEQ ID NO: 1220 - DNA, 17 bp, Homo sapiens
ctgttacatgggcctgg

SEQ ID NO: 1221 - DNA, 17 bp, Homo sapiens
tgttacatgggcctggc

SEQ ID NO: 1222 - DNA, 17 bp, Homo sapiens
gttacatgggcctggca

SEQ ID NO: 1223 - DNA, 17 bp, Homo sapiens
ttacatgggcctggcac

SEQ ID NO: 1224 - DNA, 17 bp, Homo sapiens
tacatgggcctggcact

SEQ ID NO: 1225 - DNA, 17 bp, Homo sapiens
acatgggcctggcactg

SEQ ID NO: 1226 - DNA, 17 bp, Homo sapiens
catgggcctggcactgt

SEQ ID NO: 1227 - DNA, 17 bp, Homo sapiens
atgggcctggcactgtt

SEQ ID NO: 1228 - DNA, 17 bp, Homo sapiens
tgggcctggcactgttc

SEQ ID NO: 1229 - DNA, 17 bp, Homo sapiens
gggcctggcactgttca

SEQ ID NO: 1230 - DNA, 17 bp, Homo sapiens
ggcctggcactgttcac

SEQ ID NO: 1231 - DNA, 17 bp, Homo sapiens
gcctggcactgttcacg

SEQ ID NO: 1232 - DNA, 17 bp, Homo sapiens
cctggcactgttcacgt

SEQ ID NO: 1233 - DNA, 17 bp, Homo sapiens
ctggcactgttcacgtt

SEQ ID NO: 1234 - DNA, 17 bp, Homo sapiens
tggcactgttcacgttc

SEQ ID NO: 1235 - DNA, 17 bp, Homo sapiens
ggcactgttcacgttcc

SEQ ID NO: 1236 - DNA, 17 bp, Homo sapiens
gcactgttcacgttcca

SEQ ID NO: 1237 - DNA, 17 bp, Homo sapiens
cactgttcacgttccag

SEQ ID NO: 1238 - DNA, 17 bp, Homo sapiens
actgttcacgttccaga

SEQ ID NO: 1239 - DNA, 17 bp, Homo sapiens
ctgttcacgttccagaa

SEQ ID NO: 1240 - DNA, 17 bp, Homo sapiens
tgttcacgttccagaat

SEQ ID NO: 1241 - DNA, 17 bp, Homo sapiens
gttcacgttccagaatc

SEQ ID NO: 1242 - DNA, 17 bp, Homo sapiens
ttcacgttccagaatca

SEQ ID NO: 1243 - DNA, 17 bp, Homo sapiens
tcacgttccagaatcat

SEQ ID NO: 1244 - DNA, 17 bp, Homo sapiens
cacgttccagaatcata

SEQ ID NO: 1245 - DNA, 17 bp, Homo sapiens
acgttccagaatcatat

SEQ ID NO: 1246 - DNA, 17 bp, Homo sapiens
cgttccagaatcatatc

SEQ ID NO: 1247 - DNA, 17 bp, Homo sapiens
gttccagaatcatatct

SEQ ID NO: 1248 - DNA, 17 bp, Homo sapiens
ttccagaatcatatctt

SEQ ID NO: 1249 - DNA, 17 bp, Homo sapiens
tccagaatcatatcttt

SEQ ID NO: 1250 - DNA, 17 bp, Homo sapiens
ccagaatcatatcttta

SEQ ID NO: 1251 - DNA, 17 bp, Homo sapiens
cagaatcatatctttaa

SEQ ID NO: 1252 - DNA, 17 bp, Homo sapiens
agaatcatatctttaat

SEQ ID NO: 1253 - DNA, 17 bp, Homo sapiens
gaatcatatctttaatg

SEQ ID NO: 1254 - DNA, 17 bp, Homo sapiens
aatcatatctttaatgc

SEQ ID NO: 1255 - DNA, 17 bp, Homo sapiens
atcatatctttaatgct

SEQ ID NO: 1256 - DNA, 17 bp, Homo sapiens
tcatatctttaatgctc

SEQ ID NO: 1257 - DNA, 17 bp, Homo sapiens
catatctttaatgctct

SEQ ID NO: 1258 - DNA, 17 bp, Homo sapiens
atatctttaatgctctt

SEQ ID NO: 1259 - DNA, 17 bp, Homo sapiens
tatctttaatgctcttt

SEQ ID NO: 1260 - DNA, 17 bp, Homo sapiens
atctttaatgctctttt

SEQ ID NO: 1261 - DNA, 17 bp, Homo sapiens
tctttaatgctcttttt

SEQ ID NO: 1262 - DNA, 17 bp, Homo sapiens
ctttaatgctcttttta

SEQ ID NO: 1263 - DNA, 17 bp, Homo sapiens
tttaatgctctttttat

SEQ ID NO: 1264 - DNA, 17 bp, Homo sapiens
ttaatgctctttttata

SEQ ID NO: 1265 - DNA, 17 bp, Homo sapiens
taatgctctttttatac

SEQ ID NO: 1266 - DNA, 17 bp, Homo sapiens
aatgctctttttatact

SEQ ID NO: 1267 - DNA, 17 bp, Homo sapiens
atgctctttttatactt

SEQ ID NO: 1268 - DNA, 17 bp, Homo sapiens
tgctctttttatacttg

SEQ ID NO: 1269 - DNA, 17 bp, Homo sapiens
gctctttttatacttgg

SEQ ID NO: 1270 - DNA, 17 bp, Homo sapiens
ctctttttatacttgga

SEQ ID NO: 1271 - DNA, 17 bp, Homo sapiens
tctttttatacttggag

SEQ ID NO: 1272 - DNA, 17 bp, Homo sapiens
ctttttatacttggagc

SEQ ID NO: 1273 - DNA, 17 bp, Homo sapiens
tttttatacttggagcc

SEQ ID NO: 1274 - DNA, 17 bp, Homo sapiens
ttttatacttggagcct

SEQ ID NO: 1275 - DNA, 17 bp, Homo sapiens
tttatacttggagcctt

SEQ ID NO: 1276 - DNA, 17 bp, Homo sapiens
ttatacttggagccttt

SEQ ID NO: 1277 - DNA, 17 bp, Homo sapiens
tatacttggagcctttc

SEQ ID NO: 1278 - DNA, 17 bp, Homo sapiens
atacttggagcctttct

SEQ ID NO: 1279 - DNA, 17 bp, Homo sapiens
tacttggagcctttcta

SEQ ID NO: 1280 - DNA, 17 bp, Homo sapiens
acttggagcctttctag

SEQ ID NO: 1281 - DNA, 17 bp, Homo sapiens
cttggagcctttctagc

SEQ ID NO: 1282 - DNA, 17 bp, Homo sapiens
ttggagcctttctagca

SEQ ID NO: 1283 - DNA, 17 bp, Homo sapiens
tggagcctttctagcaa

SEQ ID NO: 1284 - DNA, 17 bp, Homo sapiens
ggagcctttctagcaat

SEQ ID NO: 1285 - DNA, 17 bp, Homo sapiens
gagcctttctagcaatt

SEQ ID NO: 1286 - DNA, 17 bp, Homo sapiens
agcctttctagcaattt

SEQ ID NO: 1287 - DNA, 17 bp, Homo sapiens
gcctttctagcaatttt

SEQ ID NO: 1288 - DNA, 17 bp, Homo sapiens
cctttctagcaattttt

SEQ ID NO: 1289 - DNA, 17 bp, Homo sapiens
ctttctagcaatttttg

SEQ ID NO: 1290 - DNA, 17 bp, Homo sapiens
tttctagcaatttttgt

SEQ ID NO: 1291 - DNA, 17 bp, Homo sapiens
ttctagcaatttttgtt

SEQ ID NO: 1292 - DNA, 17 bp, Homo sapiens
tctagcaatttttgttg

SEQ ID NO: 1293 - DNA, 17 bp, Homo sapiens
ctagcaatttttgttgc

SEQ ID NO: 1294 - DNA, 17 bp, Homo sapiens
tagcaatttttgttgcc

SEQ ID NO: 1295 - DNA, 17 bp, Homo sapiens
agcaatttttgttgcca

SEQ ID NO: 1296 - DNA, 17 bp, Homo sapiens
gcaatttttgttgccag

SEQ ID NO: 1297 - DNA, 17 bp, Homo sapiens
caatttttgttgccaga

SEQ ID NO: 1298 - DNA, 17 bp, Homo sapiens
aatttttgttgccagag

SEQ ID NO: 1299 - DNA, 17 bp, Homo sapiens
atttttgttgccagagc

SEQ ID NO: 1300 - DNA, 17 bp, Homo sapiens
tttttgttgccagagcc

SEQ ID NO: 1301 - DNA, 17 bp, Homo sapiens
ttttgttgccagagcct

SEQ ID NO: 1302 - DNA, 17 bp, Homo sapiens
tttgttgccagagcctg

SEQ ID NO: 1303 - DNA, 17 bp, Homo sapiens
ttgttgccagagcctgc

SEQ ID NO: 1304 - DNA, 17 bp, Homo sapiens
tgttgccagagcctgca

SEQ ID NO: 1305 - DNA, 17 bp, Homo sapiens
gttgccagagcctgcaa

SEQ ID NO: 1306 - DNA, 17 bp, Homo sapiens
ttgccagagcctgcaac

SEQ ID NO: 1307 - DNA, 17 bp, Homo sapiens
tgccagagcctgcaaca

SEQ ID NO: 1308 - DNA, 17 bp, Homo sapiens
gccagagcctgcaacat

SEQ ID NO: 1309 - DNA, 17 bp, Homo sapiens
ccagagcctgcaacata

SEQ ID NO: 1310 - DNA, 17 bp, Homo sapiens
cagagcctgcaacatat

SEQ ID NO: 1311 - DNA, 17 bp, Homo sapiens
agagcctgcaacatata

SEQ ID NO: 1312 - DNA, 17 bp, Homo sapiens
gagcctgcaacatatat

SEQ ID NO: 1313 - DNA, 17 bp, Homo sapiens
agcctgcaacatatatc

SEQ ID NO: 1314 - DNA, 17 bp, Homo sapiens
gcctgcaacatatatcc

SEQ ID NO: 1315 - DNA, 17 bp, Homo sapiens
cctgcaacatatatccc

SEQ ID NO: 1316 - DNA, 17 bp, Homo sapiens
ctgcaacatatatcccc

SEQ ID NO: 1317 - DNA, 17 bp, Homo sapiens
tgcaacatatatcccct

SEQ ID NO: 1318 - DNA, 17 bp, Homo sapiens
gcaacatatatcccctc

SEQ ID NO: 1319 - DNA, 17 bp, Homo sapiens
caacatatatcccctct

SEQ ID NO: 1320 - DNA, 17 bp, Homo sapiens
aacatatatcccctctc

SEQ ID NO: 1321 - DNA, 17 bp, Homo sapiens
acatatatcccctctcc

SEQ ID NO: 1322 - DNA, 17 bp, Homo sapiens
catatatcccctctcct

SEQ ID NO: 1323 - DNA, 17 bp, Homo sapiens
atatatcccctctcctt

SEQ ID NO: 1324 - DNA, 17 bp, Homo sapiens
tatatcccctctccttc

SEQ ID NO: 1325 - DNA, 17 bp, Homo sapiens
atatcccctctccttcc

SEQ ID NO: 1326 - DNA, 17 bp, Homo sapiens
tatcccctctccttcct

SEQ ID NO: 1327 - DNA, 17 bp, Homo sapiens
atcccctctccttcctc

SEQ ID NO: 1328 - DNA, 17 bp, Homo sapiens
tcccctctccttcctcc

SEQ ID NO: 1329 - DNA, 17 bp, Homo sapiens
cccctctccttcctcct

SEQ ID NO: 1330 - DNA, 17 bp, Homo sapiens
ccctctccttcctcctg

SEQ ID NO: 1331 - DNA, 17 bp, Homo sapiens
cctctccttcctcctga

SEQ ID NO: 1332 - DNA, 17 bp, Homo sapiens
ctctccttcctcctgaa

SEQ ID NO: 1333 - DNA, 17 bp, Homo sapiens
tctccttcctcctgaat

SEQ ID NO: 1334 - DNA, 17 bp, Homo sapiens
ctccttcctcctgaatc

SEQ ID NO: 1335 - DNA, 17 bp, Homo sapiens
tccttcctcctgaatct

SEQ ID NO: 1336 - DNA, 17 bp, Homo sapiens
ccttcctcctgaatcta

SEQ ID NO: 1337 - DNA, 17 bp, Homo sapiens
cttcctcctgaatctag

SEQ ID NO: 1338 - DNA, 17 bp, Homo sapiens
ttcctcctgaatctagg

SEQ ID NO: 1339 - DNA, 17 bp, Homo sapiens
tcctcctgaatctaggc

SEQ ID NO: 1340 - DNA, 17 bp, Homo sapiens
cctcctgaatctaggcc

SEQ ID NO: 1341 - DNA, 17 bp, Homo sapiens
ctcctgaatctaggccg

SEQ ID NO: 1342 - DNA, 17 bp, Homo sapiens
tcctgaatctaggccga

SEQ ID NO: 1343 - DNA, 17 bp, Homo sapiens
cctgaatctaggccgaa

SEQ ID NO: 1344 - DNA, 17 bp, Homo sapiens
ctgaatctaggccgaaa

SEQ ID NO: 1345 - DNA, 17 bp, Homo sapiens
tgaatctaggccgaaaa

SEQ ID NO: 1346 - DNA, 17 bp, Homo sapiens
gaatctaggccgaaaac

SEQ ID NO: 1347 - DNA, 17 bp, Homo sapiens
aatctaggccgaaaaca

SEQ ID NO: 1348 - DNA, 17 bp, Homo sapiens
atctaggccgaaaacag

SEQ ID NO: 1349 - DNA, 17 bp, Homo sapiens
tctaggccgaaaacaga

SEQ ID NO: 1350 - DNA, 17 bp, Homo sapiens
ctaggccgaaaacagaa

SEQ ID NO: 1351 - DNA, 17 bp, Homo sapiens
taggccgaaaacagaag

SEQ ID NO: 1352 - DNA, 17 bp, Homo sapiens
aggccgaaaacagaaga

SEQ ID NO: 1353 - DNA, 17 bp, Homo sapiens
ggccgaaaacagaagat

SEQ ID NO: 1354 - DNA, 17 bp, Homo sapiens
gccgaaaacagaagatc

SEQ ID NO: 1355 - DNA, 17 bp, Homo sapiens
ccgaaaacagaagatcc

SEQ ID NO: 1356 - DNA, 17 bp, Homo sapiens
cgaaaacagaagatccc

SEQ ID NO: 1357 - DNA, 17 bp, Homo sapiens
gaaaacagaagatcccc

SEQ ID NO: 1358 - DNA, 17 bp, Homo sapiens
aaaacagaagatcccct

SEQ ID NO: 1359 - DNA, 17 bp, Homo sapiens
aaacagaagatcccctg

SEQ ID NO: 1360 - DNA, 17 bp, Homo sapiens
aacagaagatcccctgg

SEQ ID NO: 1361 - DNA, 17 bp, Homo sapiens
acagaagatcccctgga

SEQ ID NO: 1362 - DNA, 17 bp, Homo sapiens
cagaagatcccctggaa

SEQ ID NO: 1363 - DNA, 17 bp, Homo sapiens
agaagatcccctggaac

SEQ ID NO: 1364 - DNA, 17 bp, Homo sapiens
gaagatcccctggaact

SEQ ID NO: 1365 - DNA, 17 bp, Homo sapiens
aagatcccctggaactt

SEQ ID NO: 1366 - DNA, 17 bp, Homo sapiens
agatcccctggaacttt

SEQ ID NO: 1367 - DNA, 17 bp, Homo sapiens
gatcccctggaactttc

SEQ ID NO: 1368 - DNA, 17 bp, Homo sapiens
atcccctggaactttca

SEQ ID NO: 1369 - DNA, 17 bp, Homo sapiens
tcccctggaactttcag

SEQ ID NO: 1370 - DNA, 17 bp, Homo sapiens
cccctggaactttcagc

SEQ ID NO: 1371 - DNA, 17 bp, Homo sapiens
ccctggaactttcagca

SEQ ID NO: 1372 - DNA, 17 bp, Homo sapiens
cctggaactttcagcac

SEQ ID NO: 1373 - DNA, 17 bp, Homo sapiens
ctggaactttcagcaca

SEQ ID NO: 1374 - DNA, 17 bp, Homo sapiens
tggaactttcagcacat

SEQ ID NO: 1375 - DNA, 17 bp, Homo sapiens
ggaactttcagcacatg

SEQ ID NO: 1376 - DNA, 17 bp, Homo sapiens
gaactttcagcacatga

SEQ ID NO: 1377 - DNA, 17 bp, Homo sapiens
aactttcagcacatgat

SEQ ID NO: 1378 - DNA, 17 bp, Homo sapiens
actttcagcacatgatg

SEQ ID NO: 1379 - DNA, 17 bp, Homo sapiens
ctttcagcacatgatga

SEQ ID NO: 1380 - DNA, 17 bp, Homo sapiens
tttcagcacatgatgat

SEQ ID NO: 1381 - DNA, 17 bp, Homo sapiens
ttcagcacatgatgatg

SEQ ID NO: 1382 - DNA, 17 bp, Homo sapiens
tcagcacatgatgatgt

SEQ ID NO: 1383 - DNA, 17 bp, Homo sapiens
cagcacatgatgatgtt

SEQ ID NO: 1384 - DNA, 17 bp, Homo sapiens
agcacatgatgatgttt

SEQ ID NO: 1385 - DNA, 17 bp, Homo sapiens
gcacatgatgatgtttt

SEQ ID NO: 1386 - DNA, 17 bp, Homo sapiens
cacatgatgatgttttc

SEQ ID NO: 1387 - DNA, 17 bp, Homo sapiens
acatgatgatgttttca

SEQ ID NO: 1388 - DNA, 17 bp, Homo sapiens
catgatgatgttttcag

SEQ ID NO: 1389 - DNA, 17 bp, Homo sapiens
atgatgatgttttcagg

SEQ ID NO: 1390 - DNA, 17 bp, Homo sapiens
tgatgatgttttcaggt

SEQ ID NO: 1391 - DNA, 17 bp, Homo sapiens
gatgatgttttcaggtt

SEQ ID NO: 1392 - DNA, 17 bp, Homo sapiens
atgatgttttcaggttt

SEQ ID NO: 1393 - DNA, 17 bp, Homo sapiens
tgatgttttcaggtttg

SEQ ID NO: 1394 - DNA, 17 bp, Homo sapiens
gatgttttcaggtttgc

SEQ ID NO: 1395 - DNA, 17 bp, Homo sapiens
atgttttcaggtttgcg

SEQ ID NO: 1396 - DNA, 17 bp, Homo sapiens
tgttttcaggtttgcga

SEQ ID NO: 1397 - DNA, 17 bp, Homo sapiens
gttttcaggtttgcgag

SEQ ID NO: 1398 - DNA, 17 bp, Homo sapiens
ttttcaggtttgcgagg

SEQ ID NO: 1399 - DNA, 17 bp, Homo sapiens
tttcaggtttgcgagga

SEQ ID NO: 1400 - DNA, 17 bp, Homo sapiens
ttcaggtttgcgaggag

SEQ ID NO: 1401 - DNA, 17 bp, Homo sapiens
tcaggtttgcgaggagc

SEQ ID NO: 1402 - DNA, 17 bp, Homo sapiens
caggtttgcgaggagcg

SEQ ID NO: 1403 - DNA, 17 bp, Homo sapiens
aggtttgcgaggagcga

SEQ ID NO: 1404 - DNA, 17 bp, Homo sapiens
ggtttgcgaggagcgat

SEQ ID NO: 1405 - DNA, 17 bp, Homo sapiens
gtttgcgaggagcgatc

SEQ ID NO: 1406 - DNA, 17 bp, Homo sapiens
tttgcgaggagcgatcg

SEQ ID NO: 1407 - DNA, 17 bp, Homo sapiens
ttgcgaggagcgatcgc

SEQ ID NO: 1408 - DNA, 17 bp, Homo sapiens
tgcgaggagcgatcgca

SEQ ID NO: 1409 - DNA, 17 bp, Homo sapiens
gcgaggagcgatcgcat

SEQ ID NO: 1410 - DNA, 17 bp, Homo sapiens
cgaggagcgatcgcatt

SEQ ID NO: 1411 - DNA, 17 bp, Homo sapiens
gaggagcgatcgcattt

SEQ ID NO: 1412 - DNA, 17 bp, Homo sapiens
aggagcgatcgcatttg

SEQ ID NO: 1413 - DNA, 17 bp, Homo sapiens
ggagcgatcgcatttgc

SEQ ID NO: 1414 - DNA, 17 bp, Homo sapiens
gagcgatcgcatttgcc

SEQ ID NO: 1415 - DNA, 17 bp, Homo sapiens
agcgatcgcatttgcct

SEQ ID NO: 1416 - DNA, 17 bp, Homo sapiens
gcgatcgcatttgcctt

SEQ ID NO: 1417 - DNA, 17 bp, Homo sapiens
cgatcgcatttgcctta

SEQ ID NO: 1418 - DNA, 17 bp, Homo sapiens
gatcgcatttgccttag

SEQ ID NO: 1419 - DNA, 17 bp, Homo sapiens
atcgcatttgccttagc

SEQ ID NO: 1420 - DNA, 17 bp, Homo sapiens
tcgcatttgccttagct

SEQ ID NO: 1421 - DNA, 17 bp, Homo sapiens
cgcatttgccttagcta

SEQ ID NO: 1422 - DNA, 17 bp, Homo sapiens
gcatttgccttagctat

SEQ ID NO: 1423 - DNA, 17 bp, Homo sapiens
catttgccttagctatt

SEQ ID NO: 1424 - DNA, 17 bp, Homo sapiens
atttgccttagctattc

SEQ ID NO: 1425 - DNA, 17 bp, Homo sapiens
tttgccttagctattcg

SEQ ID NO: 1426 - DNA, 17 bp, Homo sapiens
ttgccttagctattcgg

SEQ ID NO: 1427 - DNA, 17 bp, Homo sapiens
tgccttagctattcgga

SEQ ID NO: 1428 - DNA, 17 bp, Homo sapiens
gccttagctattcggaa

SEQ ID NO: 1429 - DNA, 17 bp, Homo sapiens
ccttagctattcggaac

SEQ ID NO: 1430 - DNA, 17 bp, Homo sapiens
cttagctattcggaaca

SEQ ID NO: 1431 - DNA, 17 bp, Homo sapiens
ttagctattcggaacac

SEQ ID NO: 1432 - DNA, 17 bp, Homo sapiens
tagctattcggaacaca

SEQ ID NO: 1433 - DNA, 17 bp, Homo sapiens
agctattcggaacacag

SEQ ID NO: 1434 - DNA, 17 bp, Homo sapiens
gctattcggaacacaga

SEQ ID NO: 1435 - DNA, 17 bp, Homo sapiens
ctattcggaacacagaa

SEQ ID NO: 1436 - DNA, 17 bp, Homo sapiens
tattcggaacacagaat

SEQ ID NO: 1437 - DNA, 17 bp, Homo sapiens
attcggaacacagaatc

SEQ ID NO: 1438 - DNA, 17 bp, Homo sapiens
ttcggaacacagaatct

SEQ ID NO: 1439 - DNA, 17 bp, Homo sapiens
tcggaacacagaatctc

SEQ ID NO: 1440 - DNA, 17 bp, Homo sapiens
cggaacacagaatctca

SEQ ID NO: 1441 - DNA, 17 bp, Homo sapiens
ggaacacagaatctcag

SEQ ID NO: 1442 - DNA, 17 bp, Homo sapiens
gaacacagaatctcagc

SEQ ID NO: 1443 - DNA, 17 bp, Homo sapiens
aacacagaatctcagcc

SEQ ID NO: 1444 - DNA, 17 bp, Homo sapiens
acacagaatctcagccc

SEQ ID NO: 1445 - DNA, 17 bp, Homo sapiens
cacagaatctcagccca

SEQ ID NO: 1446 - DNA, 17 bp, Homo sapiens
acagaatctcagcccaa

SEQ ID NO: 1447 - DNA, 17 bp, Homo sapiens
cagaatctcagcccaaa

SEQ ID NO: 1448 - DNA, 17 bp, Homo sapiens
agaatctcagcccaaac

SEQ ID NO: 1449 - DNA, 17 bp, Homo sapiens
gaatctcagcccaaaca

SEQ ID NO: 1450 - DNA, 17 bp, Homo sapiens
aatctcagcccaaacaa

SEQ ID NO: 1451 - DNA, 17 bp, Homo sapiens
atctcagcccaaacaaa

SEQ ID NO: 1452 - DNA, 17 bp, Homo sapiens
tctcagcccaaacaaat

SEQ ID NO: 1453 - DNA, 17 bp, Homo sapiens
ctcagcccaaacaaatg

SEQ ID NO: 1454 - DNA, 17 bp, Homo sapiens
tcagcccaaacaaatga

SEQ ID NO: 1455 - DNA, 17 bp, Homo sapiens
cagcccaaacaaatgat

SEQ ID NO: 1456 - DNA, 17 bp, Homo sapiens
agcccaaacaaatgatg

SEQ ID NO: 1457 - DNA, 17 bp, Homo sapiens
gcccaaacaaatgatgt

SEQ ID NO: 1458 - DNA, 17 bp, Homo sapiens
cccaaacaaatgatgtt

SEQ ID NO: 1459 - DNA, 17 bp, Homo sapiens
ccaaacaaatgatgttt

SEQ ID NO: 1460 - DNA, 17 bp, Homo sapiens
caaacaaatgatgttta

SEQ ID NO: 1461 - DNA, 17 bp, Homo sapiens
aaacaaatgatgtttac

SEQ ID NO: 1462 - DNA, 17 bp, Homo sapiens
aacaaatgatgtttacc

SEQ ID NO: 1463 - DNA, 17 bp, Homo sapiens
acaaatgatgtttacca

SEQ ID NO: 1464 - DNA, 17 bp, Homo sapiens
caaatgatgtttaccac

SEQ ID NO: 1465 - DNA, 17 bp, Homo sapiens
aaatgatgtttaccact

SEQ ID NO: 1466 - DNA, 17 bp, Homo sapiens
aatgatgtttaccacta

SEQ ID NO: 1467 - DNA, 17 bp, Homo sapiens
atgatgtttaccactac

SEQ ID NO: 1468 - DNA, 17 bp, Homo sapiens
tgatgtttaccactacg

SEQ ID NO: 1469 - DNA, 17 bp, Homo sapiens
gatgtttaccactacgc

SEQ ID NO: 1470 - DNA, 17 bp, Homo sapiens
atgtttaccactacgct

SEQ ID NO: 1471 - DNA, 17 bp, Homo sapiens
tgtttaccactacgctg

SEQ ID NO: 1472 - DNA, 17 bp, Homo sapiens
gtttaccactacgctgc

SEQ ID NO: 1473 - DNA, 17 bp, Homo sapiens
tttaccactacgctgct

SEQ ID NO: 1474 - DNA, 17 bp, Homo sapiens
ttaccactacgctgctc

SEQ ID NO: 1475 - DNA, 17 bp, Homo sapiens
taccactacgctgctcc

SEQ ID NO: 1476 - DNA, 17 bp, Homo sapiens
accactacgctgctcct

SEQ ID NO: 1477 - DNA, 17 bp, Homo sapiens
ccactacgctgctcctc

SEQ ID NO: 1478 - DNA, 17 bp, Homo sapiens
cactacgctgctcctcg

SEQ ID NO: 1479 - DNA, 17 bp, Homo sapiens
actacgctgctcctcgt

SEQ ID NO: 1480 - DNA, 17 bp, Homo sapiens
ctacgctgctcctcgtg

SEQ ID NO: 1481 - DNA, 17 bp, Homo sapiens
tacgctgctcctcgtgt

SEQ ID NO: 1482 - DNA, 17 bp, Homo sapiens
acgctgctcctcgtgtt

SEQ ID NO: 1483 - DNA, 17 bp, Homo sapiens
cgctgctcctcgtgttc

SEQ ID NO: 1484 - DNA, 17 bp, Homo sapiens
gctgctcctcgtgttct

SEQ ID NO: 1485 - DNA, 17 bp, Homo sapiens
ctgctcctcgtgttctt

SEQ ID NO: 1486 - DNA, 17 bp, Homo sapiens
tgctcctcgtgttcttc

SEQ ID NO: 1487 - DNA, 17 bp, Homo sapiens
gctcctcgtgttcttca

SEQ ID NO: 1488 - DNA, 17 bp, Homo sapiens
ctcctcgtgttcttcac

SEQ ID NO: 1489 - DNA, 17 bp, Homo sapiens
tcctcgtgttcttcact

SEQ ID NO: 1490 - DNA, 17 bp, Homo sapiens
cctcgtgttcttcactg

SEQ ID NO: 1491 - DNA, 17 bp, Homo sapiens
ctcgtgttcttcactgt

SEQ ID NO: 1492 - DNA, 17 bp, Homo sapiens
tcgtgttcttcactgtc

SEQ ID NO: 1493 - DNA, 17 bp, Homo sapiens
cgtgttcttcactgtct

SEQ ID NO: 1494 - DNA, 17 bp, Homo sapiens
gtgttcttcactgtctg

SEQ ID NO: 1495 - DNA, 17 bp, Homo sapiens
tgttcttcactgtctgg

SEQ ID NO: 1496 - DNA, 17 bp, Homo sapiens
gttcttcactgtctggg

SEQ ID NO: 1497 - DNA, 17 bp, Homo sapiens
ttcttcactgtctgggt

SEQ ID NO: 1498 - DNA, 17 bp, Homo sapiens
tcttcactgtctgggta

SEQ ID NO: 1499 - DNA, 17 bp, Homo sapiens
cttcactgtctgggtat

SEQ ID NO: 1500 - DNA, 17 bp, Homo sapiens
ttcactgtctgggtatt

SEQ ID NO: 1501 - DNA, 17 bp, Homo sapiens
tcactgtctgggtattt

SEQ ID NO: 1502 - DNA, 17 bp, Homo sapiens
cactgtctgggtatttg

SEQ ID NO: 1503 - DNA, 17 bp, Homo sapiens
actgtctgggtatttgg

SEQ ID NO: 1504 - DNA, 17 bp, Homo sapiens
ctgtctgggtatttgga

SEQ ID NO: 1505 - DNA, 17 bp, Homo sapiens
tgtctgggtatttggag

SEQ ID NO: 1506 - DNA, 17 bp, Homo sapiens
gtctgggtatttggagg

SEQ ID NO: 1507 - DNA, 17 bp, Homo sapiens
tctgggtatttggagga

SEQ ID NO: 1508 - DNA, 17 bp, Homo sapiens
ctgggtatttggaggag

SEQ ID NO: 1509 - DNA, 17 bp, Homo sapiens
tgggtatttggaggagg

SEQ ID NO: 1510 - DNA, 17 bp, Homo sapiens
gggtatttggaggagga

SEQ ID NO: 1511 - DNA, 17 bp, Homo sapiens
ggtatttggaggaggaa

SEQ ID NO: 1512 - DNA, 17 bp, Homo sapiens
gtatttggaggaggaac

SEQ ID NO: 1513 - DNA, 17 bp, Homo sapiens
tatttggaggaggaaca

SEQ ID NO: 1514 - DNA, 17 bp, Homo sapiens
atttggaggaggaacaa

SEQ ID NO: 1515 - DNA, 17 bp, Homo sapiens
tttggaggaggaacaac

SEQ ID NO: 1516 - DNA, 17 bp, Homo sapiens
ttggaggaggaacaacc

SEQ ID NO: 1517 - DNA, 17 bp, Homo sapiens
tggaggaggaacaaccc

SEQ ID NO: 1518 - DNA, 17 bp, Homo sapiens
ggaggaggaacaacccc

SEQ ID NO: 1519 - DNA, 17 bp, Homo sapiens
gaggaggaacaaccccc

SEQ ID NO: 1520 - DNA, 17 bp, Homo sapiens
aggaggaacaaccccca

SEQ ID NO: 1521 - DNA, 17 bp, Homo sapiens
ggaggaacaacccccat

SEQ ID NO: 1522 - DNA, 17 bp, Homo sapiens
gaggaacaacccccatg

SEQ ID NO: 1523 - DNA, 17 bp, Homo sapiens
aggaacaacccccatgt

SEQ ID NO: 1524 - DNA, 17 bp, Homo sapiens
ggaacaacccccatgtt

SEQ ID NO: 1525 - DNA, 17 bp, Homo sapiens
gaacaacccccatgttg

SEQ ID NO: 1526 - DNA, 17 bp, Homo sapiens
aacaacccccatgttga

SEQ ID NO: 1527 - DNA, 17 bp, Homo sapiens
acaacccccatgttgac

SEQ ID NO: 1528 - DNA, 17 bp, Homo sapiens
caacccccatgttgact

SEQ ID NO: 1529 - DNA, 17 bp, Homo sapiens
aacccccatgttgactt

SEQ ID NO: 1530 - DNA, 17 bp, Homo sapiens
acccccatgttgacttg

SEQ ID NO: 1531 - DNA, 17 bp, Homo sapiens
cccccatgttgacttgg

SEQ ID NO: 1532 - DNA, 17 bp, Homo sapiens
ccccatgttgacttggc

SEQ ID NO: 1533 - DNA, 17 bp, Homo sapiens
cccatgttgacttggct

SEQ ID NO: 1534 - DNA, 17 bp, Homo sapiens
ccatgttgacttggctt

SEQ ID NO: 1535 - DNA, 17 bp, Homo sapiens
catgttgacttggcttc

SEQ ID NO: 1536 - DNA, 17 bp, Homo sapiens
atgttgacttggcttca

SEQ ID NO: 1537 - DNA, 17 bp, Homo sapiens
tgttgacttggcttcag

SEQ ID NO: 1538 - DNA, 17 bp, Homo sapiens
gttgacttggcttcaga

SEQ ID NO: 1539 - DNA, 17 bp, Homo sapiens
ttgacttggcttcagat

SEQ ID NO: 1540 - DNA, 17 bp, Homo sapiens
tgacttggcttcagatc

SEQ ID NO: 1541 - DNA, 17 bp, Homo sapiens
gacttggcttcagatca

SEQ ID NO: 1542 - DNA, 17 bp, Homo sapiens
acttggcttcagatcag

SEQ ID NO: 1543 - DNA, 17 bp, Homo sapiens
cttggcttcagatcaga

SEQ ID NO: 1544 - DNA, 17 bp, Homo sapiens
ttggcttcagatcagag

SEQ ID NO: 1545 - DNA, 17 bp, Homo sapiens
tggcttcagatcagagt

SEQ ID NO: 1546 - DNA, 17 bp, Homo sapiens
ggcttcagatcagagtt

SEQ ID NO: 1547 - DNA, 17 bp, Homo sapiens
gcttcagatcagagttg

SEQ ID NO: 1548 - DNA, 17 bp, Homo sapiens
cttcagatcagagttgg

SEQ ID NO: 1549 - DNA, 17 bp, Homo sapiens
ttcagatcagagttggc

SEQ ID NO: 1550 - DNA, 17 bp, Homo sapiens
tcagatcagagttggcg

SEQ ID NO: 1551 - DNA, 17 bp, Homo sapiens
cagatcagagttggcgt

SEQ ID NO: 1552 - DNA, 17 bp, Homo sapiens
agatcagagttggcgtg

SEQ ID NO: 1553 - DNA, 25 bp, Homo sapiens
atagcccttatccaggtttttatct

SEQ ID NO: 1554 - DNA, 25 bp, Homo sapiens
tagcccttatccaggtttttatcta

SEQ ID NO: 1555 - DNA, 25 bp, Homo sapiens
agcccttatccaggtttttatctaa

SEQ ID NO: 1556 - DNA, 25 bp, Homo sapiens
gcccttatccaggtttttatctaag

SEQ ID NO: 1557 - DNA, 25 bp, Homo sapiens
cccttatccaggtttttatctaagg

SEQ ID NO: 1558 - DNA, 25 bp, Homo sapiens
ccttatccaggtttttatctaagga

SEQ ID NO: 1559 - DNA, 25 bp, Homo sapiens
cttatccaggtttttatctaaggaa

SEQ ID NO: 1560 - DNA, 25 bp, Homo sapiens
ttatccaggtttttatctaaggaat

SEQ ID NO: 1561 - DNA, 25 bp, Homo sapiens
tatccaggtttttatctaaggaatc

SEQ ID NO: 1562 - DNA, 25 bp, Homo sapiens
atccaggtttttatctaaggaatcc

SEQ ID NO: 1563 - DNA, 25 bp, Homo sapiens
tccaggtttttatctaaggaatccc

SEQ ID NO: 1564 - DNA, 25 bp, Homo sapiens
ccaggtttttatctaaggaatccca

SEQ ID NO: 1565 - DNA, 25 bp, Homo sapiens
caggtttttatctaaggaatcccaa

SEQ ID NO: 1566 - DNA, 25 bp, Homo sapiens
aggtttttatctaaggaatcccaag

SEQ ID NO: 1567 - DNA, 25 bp, Homo sapiens
ggtttttatctaaggaatcccaaga

SEQ ID NO: 1568 - DNA, 25 bp, Homo sapiens
gtttttatctaaggaatcccaagaa

SEQ ID NO: 1569 - DNA, 25 bp, Homo sapiens
tttttatctaaggaatcccaagaag

SEQ ID NO: 1570 - DNA, 25 bp, Homo sapiens
ttttatctaaggaatcccaagaaga

SEQ ID NO: 1571 - DNA, 25 bp, Homo sapiens
tttatctaaggaatcccaagaagac

SEQ ID NO: 1572 - DNA, 25 bp, Homo sapiens
ttatctaaggaatcccaagaagact

SEQ ID NO: 1573 - DNA, 25 bp, Homo sapiens
tatctaaggaatcccaagaagactg

SEQ ID NO: 1574 - DNA, 25 bp, Homo sapiens
atctaaggaatcccaagaagactgg

SEQ ID NO: 1575 - DNA, 25 bp, Homo sapiens
tctaaggaatcccaagaagactggg

SEQ ID NO: 1576 - DNA, 25 bp, Homo sapiens
ctaaggaatcccaagaagactgggg

SEQ ID NO: 1577 - DNA, 25 bp, Homo sapiens
taaggaatcccaagaagactgggga

SEQ ID NO: 1578 - DNA, 25 bp, Homo sapiens
aaggaatcccaagaagactggggaa

SEQ ID NO: 1579 - DNA, 25 bp, Homo sapiens
aggaatcccaagaagactggggaat

SEQ ID NO: 1580 - DNA, 25 bp, Homo sapiens
ggaatcccaagaagactggggaatg

SEQ ID NO: 1581 - DNA, 25 bp, Homo sapiens
gaatcccaagaagactggggaatgg

SEQ ID NO: 1582 - DNA, 25 bp, Homo sapiens
aatcccaagaagactggggaatgga

SEQ ID NO: 1583 - DNA, 25 bp, Homo sapiens
atcccaagaagactggggaatggag

SEQ ID NO: 1584 - DNA, 25 bp, Homo sapiens
tcccaagaagactggggaatggaga

SEQ ID NO: 1585 - DNA, 25 bp, Homo sapiens
cccaagaagactggggaatggagag

SEQ ID NO: 1586 - DNA, 25 bp, Homo sapiens
ccaagaagactggggaatggagaga

SEQ ID NO: 1587 - DNA, 25 bp, Homo sapiens
caagaagactggggaatggagagac

SEQ ID NO: 1588 - DNA, 25 bp, Homo sapiens
aagaagactggggaatggagagaca

SEQ ID NO: 1589 - DNA, 25 bp, Homo sapiens
agaagactggggaatggagagacag

SEQ ID NO: 1590 - DNA, 25 bp, Homo sapiens
gaagactggggaatggagagacagt

SEQ ID NO: 1591 - DNA, 25 bp, Homo sapiens
aagactggggaatggagagacagtc

SEQ ID NO: 1592 - DNA, 25 bp, Homo sapiens
agactggggaatggagagacagtca

SEQ ID NO: 1593 - DNA, 25 bp, Homo sapiens
gactggggaatggagagacagtcaa

SEQ ID NO: 1594 - DNA, 25 bp, Homo sapiens
actggggaatggagagacagtcaag

SEQ ID NO: 1595 - DNA, 25 bp, Homo sapiens
ctggggaatggagagacagtcaagg

SEQ ID NO: 1596 - DNA, 25 bp, Homo sapiens
tggggaatggagagacagtcaaggg

SEQ ID NO: 1597 - DNA, 25 bp, Homo sapiens
ggggaatggagagacagtcaagggt

SEQ ID NO: 1598 - DNA, 25 bp, Homo sapiens
gggaatggagagacagtcaagggtt

SEQ ID NO: 1599 - DNA, 25 bp, Homo sapiens
ggaatggagagacagtcaagggtta

SEQ ID NO: 1600 - DNA, 25 bp, Homo sapiens
gaatggagagacagtcaagggttat

SEQ ID NO: 1601 - DNA, 25 bp, Homo sapiens
aatggagagacagtcaagggttatg

SEQ ID NO: 1602 - DNA, 25 bp, Homo sapiens
atggagagacagtcaagggttatgt

SEQ ID NO: 1603 - DNA, 25 bp, Homo sapiens
tggagagacagtcaagggttatgtc

SEQ ID NO: 1604 - DNA, 25 bp, Homo sapiens
ggagagacagtcaagggttatgtca

SEQ ID NO: 1605 - DNA, 25 bp, Homo sapiens
gagagacagtcaagggttatgtcag

SEQ ID NO: 1606 - DNA, 25 bp, Homo sapiens
agagacagtcaagggttatgtcaga

SEQ ID NO: 1607 - DNA, 25 bp, Homo sapiens
gagacagtcaagggttatgtcagaa

SEQ ID NO: 1608 - DNA, 25 bp, Homo sapiens
agacagtcaagggttatgtcagaaa

SEQ ID NO: 1609 - DNA, 25 bp, Homo sapiens
gacagtcaagggttatgtcagaaaa

SEQ ID NO: 1610 - DNA, 25 bp, Homo sapiens
acagtcaagggttatgtcagaaaag

SEQ ID NO: 1611 - DNA, 25 bp, Homo sapiens
cagtcaagggttatgtcagaaaagg

SEQ ID NO: 1612 - DNA, 25 bp, Homo sapiens
agtcaagggttatgtcagaaaagga

SEQ ID NO: 1613 - DNA, 25 bp, Homo sapiens
gtcaagggttatgtcagaaaaggat

SEQ ID NO: 1614 - DNA, 25 bp, Homo sapiens
tcaagggttatgtcagaaaaggatg

SEQ ID NO: 1615 - DNA, 25 bp, Homo sapiens
caagggttatgtcagaaaaggatga

SEQ ID NO: 1616 - DNA, 25 bp, Homo sapiens
aagggttatgtcagaaaaggatgag

SEQ ID NO: 1617 - DNA, 25 bp, Homo sapiens
agggttatgtcagaaaaggatgagt

SEQ ID NO: 1618 - DNA, 25 bp, Homo sapiens
gggttatgtcagaaaaggatgagta

SEQ ID NO: 1619 - DNA, 25 bp, Homo sapiens
ggttatgtcagaaaaggatgagtat

SEQ ID NO: 1620 - DNA, 25 bp, Homo sapiens
gttatgtcagaaaaggatgagtatc

SEQ ID NO: 1621 - DNA, 25 bp, Homo sapiens
ttatgtcagaaaaggatgagtatca

SEQ ID NO: 1622 - DNA, 25 bp, Homo sapiens
tatgtcagaaaaggatgagtatcag

SEQ ID NO: 1623 - DNA, 25 bp, Homo sapiens
atgtcagaaaaggatgagtatcagt

SEQ ID NO: 1624 - DNA, 25 bp, Homo sapiens
tgtcagaaaaggatgagtatcagtt

SEQ ID NO: 1625 - DNA, 25 bp, Homo sapiens
gtcagaaaaggatgagtatcagttt

SEQ ID NO: 1626 - DNA, 25 bp, Homo sapiens
tcagaaaaggatgagtatcagtttc

SEQ ID NO: 1627 - DNA, 25 bp, Homo sapiens
cagaaaaggatgagtatcagtttca

SEQ ID NO: 1628 - DNA, 25 bp, Homo sapiens
agaaaaggatgagtatcagtttcaa

SEQ ID NO: 1629 - DNA, 25 bp, Homo sapiens
gaaaaggatgagtatcagtttcaac

SEQ ID NO: 1630 - DNA, 25 bp, Homo sapiens
aaaaggatgagtatcagtttcaaca

SEQ ID NO: 1631 - DNA, 25 bp, Homo sapiens
aaaggatgagtatcagtttcaacat

SEQ ID NO: 1632 - DNA, 25 bp, Homo sapiens
aaggatgagtatcagtttcaacatc

SEQ ID NO: 1633 - DNA, 25 bp, Homo sapiens
aggatgagtatcagtttcaacatca

SEQ ID NO: 1634 - DNA, 25 bp, Homo sapiens
ggatgagtatcagtttcaacatcag

SEQ ID NO: 1635 - DNA, 25 bp, Homo sapiens
gatgagtatcagtttcaacatcagg

SEQ ID NO: 1636 - DNA, 25 bp, Homo sapiens
atgagtatcagtttcaacatcaggg

SEQ ID NO: 1637 - DNA, 25 bp, Homo sapiens
tgagtatcagtttcaacatcaggga

SEQ ID NO: 1638 - DNA, 25 bp, Homo sapiens
gagtatcagtttcaacatcagggag

SEQ ID NO: 1639 - DNA, 25 bp, Homo sapiens
agtatcagtttcaacatcagggagc

SEQ ID NO: 1640 - DNA, 25 bp, Homo sapiens
gtatcagtttcaacatcagggagcg

SEQ ID NO: 1641 - DNA, 25 bp, Homo sapiens
tatcagtttcaacatcagggagcgg

SEQ ID NO: 1642 - DNA, 25 bp, Homo sapiens
atcagtttcaacatcagggagcggt

SEQ ID NO: 1643 - DNA, 25 bp, Homo sapiens
tcagtttcaacatcagggagcggtg

SEQ ID NO: 1644 - DNA, 25 bp, Homo sapiens
cagtttcaacatcagggagcggtgg

SEQ ID NO: 1645 - DNA, 25 bp, Homo sapiens
agtttcaacatcagggagcggtgga

SEQ ID NO: 1646 - DNA, 25 bp, Homo sapiens
gtttcaacatcagggagcggtggag

SEQ ID NO: 1647 - DNA, 25 bp, Homo sapiens
tttcaacatcagggagcggtggagc

SEQ ID NO: 1648 - DNA, 25 bp, Homo sapiens
ttcaacatcagggagcggtggagct

SEQ ID NO: 1649 - DNA, 25 bp, Homo sapiens
tcaacatcagggagcggtggagctg

SEQ ID NO: 1650 - DNA, 25 bp, Homo sapiens
caacatcagggagcggtggagctgc

SEQ ID NO: 1651 - DNA, 25 bp, Homo sapiens
aacatcagggagcggtggagctgct

SEQ ID NO: 1652 - DNA, 25 bp, Homo sapiens
acatcagggagcggtggagctgctt

SEQ ID NO: 1653 - DNA, 25 bp, Homo sapiens
catcagggagcggtggagctgcttg

SEQ ID NO: 1654 - DNA, 25 bp, Homo sapiens
atcagggagcggtggagctgcttgt

SEQ ID NO: 1655 - DNA, 25 bp, Homo sapiens
tcagggagcggtggagctgcttgtc

SEQ ID NO: 1656 - DNA, 25 bp, Homo sapiens
cagggagcggtggagctgcttgtct

SEQ ID NO: 1657 - DNA, 25 bp, Homo sapiens
agggagcggtggagctgcttgtctt

SEQ ID NO: 1658 - DNA, 25 bp, Homo sapiens
gggagcggtggagctgcttgtcttc

SEQ ID NO: 1659 - DNA, 25 bp, Homo sapiens
ggagcggtggagctgcttgtcttca

SEQ ID NO: 1660 - DNA, 25 bp, Homo sapiens
gagcggtggagctgcttgtcttcaa

SEQ ID NO: 1661 - DNA, 25 bp, Homo sapiens
agcggtggagctgcttgtcttcaat

SEQ ID NO: 1662 - DNA, 25 bp, Homo sapiens
gcggtggagctgcttgtcttcaatt

SEQ ID NO: 1663 - DNA, 25 bp, Homo sapiens
cggtggagctgcttgtcttcaattt

SEQ ID NO: 1664 - DNA, 25 bp, Homo sapiens
ggtggagctgcttgtcttcaatttt

SEQ ID NO: 1665 - DNA, 25 bp, Homo sapiens
gtggagctgcttgtcttcaattttt

SEQ ID NO: 1666 - DNA, 25 bp, Homo sapiens
tggagctgcttgtcttcaatttttt

SEQ ID NO: 1667 - DNA, 25 bp, Homo sapiens
ggagctgcttgtcttcaattttttg

SEQ ID NO: 1668 - DNA, 25 bp, Homo sapiens
gagctgcttgtcttcaattttttgc

SEQ ID NO: 1669 - DNA, 25 bp, Homo sapiens
agctgcttgtcttcaattttttgct

SEQ ID NO: 1670 - DNA, 25 bp, Homo sapiens
gctgcttgtcttcaattttttgctc

SEQ ID NO: 1671 - DNA, 25 bp, Homo sapiens
ctgcttgtcttcaattttttgctca

SEQ ID NO: 1672 - DNA, 25 bp, Homo sapiens
tgcttgtcttcaattttttgctcat

SEQ ID NO: 1673 - DNA, 25 bp, Homo sapiens
gcttgtcttcaattttttgctcatc

SEQ ID NO: 1674 - DNA, 25 bp, Homo sapiens
cttgtcttcaattttttgctcatcc

SEQ ID NO: 1675 - DNA, 25 bp, Homo sapiens
ttgtcttcaattttttgctcatcct

SEQ ID NO: 1676 - DNA, 25 bp, Homo sapiens
tgtcttcaattttttgctcatcctt

SEQ ID NO: 1677 - DNA, 25 bp, Homo sapiens
gtcttcaattttttgctcatcctta

SEQ ID NO: 1678 - DNA, 25 bp, Homo sapiens
tcttcaattttttgctcatccttac

SEQ ID NO: 1679 - DNA, 25 bp, Homo sapiens
cttcaattttttgctcatccttacc

SEQ ID NO: 1680 - DNA, 25 bp, Homo sapiens
ttcaattttttgctcatccttacca

SEQ ID NO: 1681 - DNA, 25 bp, Homo sapiens
tcaattttttgctcatccttaccat

SEQ ID NO: 1682 - DNA, 25 bp, Homo sapiens
caattttttgctcatccttaccatt

SEQ ID NO: 1683 - DNA, 25 bp, Homo sapiens
aattttttgctcatccttaccattt

SEQ ID NO: 1684 - DNA, 25 bp, Homo sapiens
attttttgctcatccttaccatttt

SEQ ID NO: 1685 - DNA, 25 bp, Homo sapiens
ttttttgctcatccttaccattttg

SEQ ID NO: 1686 - DNA, 25 bp, Homo sapiens
tttttgctcatccttaccattttga

SEQ ID NO: 1687 - DNA, 25 bp, Homo sapiens
ttttgctcatccttaccattttgac

SEQ ID NO: 1688 - DNA, 25 bp, Homo sapiens
tttgctcatccttaccattttgaca

SEQ ID NO: 1689 - DNA, 25 bp, Homo sapiens
ttgctcatccttaccattttgacaa

SEQ ID NO: 1690 - DNA, 25 bp, Homo sapiens
tgctcatccttaccattttgacaat

SEQ ID NO: 1691 - DNA, 25 bp, Homo sapiens
gctcatccttaccattttgacaatc

SEQ ID NO: 1692 - DNA, 25 bp, Homo sapiens
ctcatccttaccattttgacaatct

SEQ ID NO: 1693 - DNA, 25 bp, Homo sapiens
tcatccttaccattttgacaatctg

SEQ ID NO: 1694 - DNA, 25 bp, Homo sapiens
catccttaccattttgacaatctgg

SEQ ID NO: 1695 - DNA, 25 bp, Homo sapiens
atccttaccattttgacaatctggt

SEQ ID NO: 1696 - DNA, 25 bp, Homo sapiens
tccttaccattttgacaatctggtt

SEQ ID NO: 1697 - DNA, 25 bp, Homo sapiens
ccttaccattttgacaatctggtta

SEQ ID NO: 1698 - DNA, 25 bp, Homo sapiens
cttaccattttgacaatctggttat

SEQ ID NO: 1699 - DNA, 25 bp, Homo sapiens
ttaccattttgacaatctggttatt

SEQ ID NO: 1700 - DNA, 25 bp, Homo sapiens
taccattttgacaatctggttattt

SEQ ID NO: 1701 - DNA, 25 bp, Homo sapiens
accattttgacaatctggttattta

SEQ ID NO: 1702 - DNA, 25 bp, Homo sapiens
ccattttgacaatctggttatttaa

SEQ ID NO: 1703 - DNA, 25 bp, Homo sapiens
cattttgacaatctggttatttaaa

SEQ ID NO: 1704 - DNA, 25 bp, Homo sapiens
attttgacaatctggttatttaaaa

SEQ ID NO: 1705 - DNA, 25 bp, Homo sapiens
ttttgacaatctggttatttaaaaa

SEQ ID NO: 1706 - DNA, 25 bp, Homo sapiens
tttgacaatctggttatttaaaaat

SEQ ID NO: 1707 - DNA, 25 bp, Homo sapiens
ttgacaatctggttatttaaaaatc

SEQ ID NO: 1708 - DNA, 25 bp, Homo sapiens
tgacaatctggttatttaaaaatca

SEQ ID NO: 1709 - DNA, 25 bp, Homo sapiens
gacaatctggttatttaaaaatcat

SEQ ID NO: 1710 - DNA, 25 bp, Homo sapiens
acaatctggttatttaaaaatcatc

SEQ ID NO: 1711 - DNA, 25 bp, Homo sapiens
caatctggttatttaaaaatcatcg

SEQ ID NO: 1712 - DNA, 25 bp, Homo sapiens
aatctggttatttaaaaatcatcga

SEQ ID NO: 1713 - DNA, 25 bp, Homo sapiens
atctggttatttaaaaatcatcgat

SEQ ID NO: 1714 - DNA, 25 bp, Homo sapiens
tctggttatttaaaaatcatcgatt

SEQ ID NO: 1715 - DNA, 25 bp, Homo sapiens
ctggttatttaaaaatcatcgattc

SEQ ID NO: 1716 - DNA, 25 bp, Homo sapiens
tggttatttaaaaatcatcgattcc

SEQ ID NO: 1717 - DNA, 25 bp, Homo sapiens
ggttatttaaaaatcatcgattccg

SEQ ID NO: 1718 - DNA, 25 bp, Homo sapiens
gttatttaaaaatcatcgattccgc

SEQ ID NO: 1719 - DNA, 25 bp, Homo sapiens
ttatttaaaaatcatcgattccgct

SEQ ID NO: 1720 - DNA, 25 bp, Homo sapiens
tatttaaaaatcatcgattccgctt

SEQ ID NO: 1721 - DNA, 25 bp, Homo sapiens
atttaaaaatcatcgattccgcttc

SEQ ID NO: 1722 - DNA, 25 bp, Homo sapiens
tttaaaaatcatcgattccgcttct

SEQ ID NO: 1723 - DNA, 25 bp, Homo sapiens
ttaaaaatcatcgattccgcttctt

SEQ ID NO: 1724 - DNA, 25 bp, Homo sapiens
taaaaatcatcgattccgcttcttg

SEQ ID NO: 1725 - DNA, 25 bp, Homo sapiens
aaaaatcatcgattccgcttcttgc

SEQ ID NO: 1726 - DNA, 25 bp, Homo sapiens
aaaatcatcgattccgcttcttgca

SEQ ID NO: 1727 - DNA, 25 bp, Homo sapiens
aaatcatcgattccgcttcttgcat

SEQ ID NO: 1728 - DNA, 25 bp, Homo sapiens
aatcatcgattccgcttcttgcatg

SEQ ID NO: 1729 - DNA, 25 bp, Homo sapiens
atcatcgattccgcttcttgcatga

SEQ ID NO: 1730 - DNA, 25 bp, Homo sapiens
tcatcgattccgcttcttgcatgaa

SEQ ID NO: 1731 - DNA, 25 bp, Homo sapiens
catcgattccgcttcttgcatgaaa

SEQ ID NO: 1732 - DNA, 25 bp, Homo sapiens
atcgattccgcttcttgcatgaaac

SEQ ID NO: 1733 - DNA, 25 bp, Homo sapiens
tcgattccgcttcttgcatgaaact

SEQ ID NO: 1734 - DNA, 25 bp, Homo sapiens
cgattccgcttcttgcatgaaactg

SEQ ID NO: 1735 - DNA, 25 bp, Homo sapiens
gattccgcttcttgcatgaaactgg

SEQ ID NO: 1736 - DNA, 25 bp, Homo sapiens
attccgcttcttgcatgaaactgga

SEQ ID NO: 1737 - DNA, 25 bp, Homo sapiens
ttccgcttcttgcatgaaactggag

SEQ ID NO: 1738 - DNA, 25 bp, Homo sapiens
tccgcttcttgcatgaaactggagg

SEQ ID NO: 1739 - DNA, 25 bp, Homo sapiens
ccgcttcttgcatgaaactggagga

SEQ ID NO: 1740 - DNA, 25 bp, Homo sapiens
cgcttcttgcatgaaactggaggag

SEQ ID NO: 1741 - DNA, 25 bp, Homo sapiens
gcttcttgcatgaaactggaggagc

SEQ ID NO: 1742 - DNA, 25 bp, Homo sapiens
cttcttgcatgaaactggaggagca

SEQ ID NO: 1743 - DNA, 25 bp, Homo sapiens
ttcttgcatgaaactggaggagcaa

SEQ ID NO: 1744 - DNA, 25 bp, Homo sapiens
tcttgcatgaaactggaggagcaat

SEQ ID NO: 1745 - DNA, 25 bp, Homo sapiens
cttgcatgaaactggaggagcaatg

SEQ ID NO: 1746 - DNA, 25 bp, Homo sapiens
ttgcatgaaactggaggagcaatgg

SEQ ID NO: 1747 - DNA, 25 bp, Homo sapiens
tgcatgaaactggaggagcaatggt

SEQ ID NO: 1748 - DNA, 25 bp, Homo sapiens
gcatgaaactggaggagcaatggtg

SEQ ID NO: 1749 - DNA, 25 bp, Homo sapiens
catgaaactggaggagcaatggtgt

SEQ ID NO: 1750 - DNA, 25 bp, Homo sapiens
atgaaactggaggagcaatggtgta

SEQ ID NO: 1751 - DNA, 25 bp, Homo sapiens
tgaaactggaggagcaatggtgtat

SEQ ID NO: 1752 - DNA, 25 bp, Homo sapiens
gaaactggaggagcaatggtgtatg

SEQ ID NO: 1753 - DNA, 25 bp, Homo sapiens
aaactggaggagcaatggtgtatgg

SEQ ID NO: 1754 - DNA, 25 bp, Homo sapiens
aactggaggagcaatggtgtatggc

SEQ ID NO: 1755 - DNA, 25 bp, Homo sapiens
actggaggagcaatggtgtatggcc

SEQ ID NO: 1756 - DNA, 25 bp, Homo sapiens
ctggaggagcaatggtgtatggcct

SEQ ID NO: 1757 - DNA, 25 bp, Homo sapiens
tggaggagcaatggtgtatggcctt

SEQ ID NO: 1758 - DNA, 25 bp, Homo sapiens
ggaggagcaatggtgtatggcctta

SEQ ID NO: 1759 - DNA, 25 bp, Homo sapiens
gaggagcaatggtgtatggccttat

SEQ ID NO: 1760 - DNA, 25 bp, Homo sapiens
aggagcaatggtgtatggccttata

SEQ ID NO: 1761 - DNA, 25 bp, Homo sapiens
ggagcaatggtgtatggccttataa

SEQ ID NO: 1762 - DNA, 25 bp, Homo sapiens
gagcaatggtgtatggccttataat

SEQ ID NO: 1763 - DNA, 25 bp, Homo sapiens
agcaatggtgtatggccttataatg

SEQ ID NO: 1764 - DNA, 25 bp, Homo sapiens
gcaatggtgtatggccttataatgg

SEQ ID NO: 1765 - DNA, 25 bp, Homo sapiens
caatggtgtatggccttataatggg

SEQ ID NO: 1766 - DNA, 25 bp, Homo sapiens
aatggtgtatggccttataatggga

SEQ ID NO: 1767 - DNA, 25 bp, Homo sapiens
atggtgtatggccttataatgggac

SEQ ID NO: 1768 - DNA, 25 bp, Homo sapiens
tggtgtatggccttataatgggact

SEQ ID NO: 1769 - DNA, 25 bp, Homo sapiens
ggtgtatggccttataatgggacta

SEQ ID NO: 1770 - DNA, 25 bp, Homo sapiens
gtgtatggccttataatgggactaa

SEQ ID NO: 1771 - DNA, 25 bp, Homo sapiens
tgtatggccttataatgggactaat

SEQ ID NO: 1772 - DNA, 25 bp, Homo sapiens
gtatggccttataatgggactaatt

SEQ ID NO: 1773 - DNA, 25 bp, Homo sapiens
tatggccttataatgggactaattt

SEQ ID NO: 1774 - DNA, 25 bp, Homo sapiens
atggccttataatgggactaatttt

SEQ ID NO: 1775 - DNA, 25 bp, Homo sapiens
tggccttataatgggactaatttta

SEQ ID NO: 1776 - DNA, 25 bp, Homo sapiens
ggccttataatgggactaattttac

SEQ ID NO: 1777 - DNA, 25 bp, Homo sapiens
gccttataatgggactaattttacg

SEQ ID NO: 1778 - DNA, 25 bp, Homo sapiens
ccttataatgggactaattttacga

SEQ ID NO: 1779 - DNA, 25 bp, Homo sapiens
cttataatgggactaattttacgat

SEQ ID NO: 1780 - DNA, 25 bp, Homo sapiens
ttataatgggactaattttacgata

SEQ ID NO: 1781 - DNA, 25 bp, Homo sapiens
tataatgggactaattttacgatat

SEQ ID NO: 1782 - DNA, 25 bp, Homo sapiens
ataatgggactaattttacgatatg

SEQ ID NO: 1783 - DNA, 25 bp, Homo sapiens
taatgggactaattttacgatatgc

SEQ ID NO: 1784 - DNA, 25 bp, Homo sapiens
aatgggactaattttacgatatgct

SEQ ID NO: 1785 - DNA, 25 bp, Homo sapiens
atgggactaattttacgatatgcta

SEQ ID NO: 1786 - DNA, 25 bp, Homo sapiens
tgggactaattttacgatatgctac

SEQ ID NO: 1787 - DNA, 25 bp, Homo sapiens
gggactaattttacgatatgctaca

SEQ ID NO: 1788 - DNA, 25 bp, Homo sapiens
ggactaattttacgatatgctacag

SEQ ID NO: 1789 - DNA, 25 bp, Homo sapiens
gactaattttacgatatgctacagc

SEQ ID NO: 1790 - DNA, 25 bp, Homo sapiens
actaattttacgatatgctacagca

SEQ ID NO: 1791 - DNA, 25 bp, Homo sapiens
ctaattttacgatatgctacagcac

SEQ ID NO: 1792 - DNA, 25 bp, Homo sapiens
taattttacgatatgctacagcacc

SEQ ID NO: 1793 - DNA, 25 bp, Homo sapiens
aattttacgatatgctacagcacca

SEQ ID NO: 1794 - DNA, 25 bp, Homo sapiens
attttacgatatgctacagcaccaa

SEQ ID NO: 1795 - DNA, 25 bp, Homo sapiens
ttttacgatatgctacagcaccaac

SEQ ID NO: 1796 - DNA, 25 bp, Homo sapiens
tttacgatatgctacagcaccaact

SEQ ID NO: 1797 - DNA, 25 bp, Homo sapiens
ttacgatatgctacagcaccaactg

SEQ ID NO: 1798 - DNA, 25 bp, Homo sapiens
tacgatatgctacagcaccaactga

SEQ ID NO: 1799 - DNA, 25 bp, Homo sapiens
acgatatgctacagcaccaactgat

SEQ ID NO: 1800 - DNA, 25 bp, Homo sapiens
cgatatgctacagcaccaactgata

SEQ ID NO: 1801 - DNA, 25 bp, Homo sapiens
gatatgctacagcaccaactgatat

SEQ ID NO: 1802 - DNA, 25 bp, Homo sapiens
atatgctacagcaccaactgatatt

SEQ ID NO: 1803 - DNA, 25 bp, Homo sapiens
tatgctacagcaccaactgatattg

SEQ ID NO: 1804 - DNA, 25 bp, Homo sapiens
atgctacagcaccaactgatattga

SEQ ID NO: 1805 - DNA, 25 bp, Homo sapiens
tgctacagcaccaactgatattgaa

SEQ ID NO: 1806 - DNA, 25 bp, Homo sapiens
gctacagcaccaactgatattgaaa

SEQ ID NO: 1807 - DNA, 25 bp, Homo sapiens
ctacagcaccaactgatattgaaag

SEQ ID NO: 1808 - DNA, 25 bp, Homo sapiens
tacagcaccaactgatattgaaagt

SEQ ID NO: 1809 - DNA, 25 bp, Homo sapiens
acagcaccaactgatattgaaagtg

SEQ ID NO: 1810 - DNA, 25 bp, Homo sapiens
cagcaccaactgatattgaaagtgg

SEQ ID NO: 1811 - DNA, 25 bp, Homo sapiens
agcaccaactgatattgaaagtgga

SEQ ID NO: 1812 - DNA, 25 bp, Homo sapiens
gcaccaactgatattgaaagtggaa

SEQ ID NO: 1813 - DNA, 25 bp, Homo sapiens
caccaactgatattgaaagtggaac

SEQ ID NO: 1814 - DNA, 25 bp, Homo sapiens
accaactgatattgaaagtggaact

SEQ ID NO: 1815 - DNA, 25 bp, Homo sapiens
ccaactgatattgaaagtggaactg

SEQ ID NO: 1816 - DNA, 25 bp, Homo sapiens
caactgatattgaaagtggaactgt

SEQ ID NO: 1817 - DNA, 25 bp, Homo sapiens
aactgatattgaaagtggaactgtc

SEQ ID NO: 1818 - DNA, 25 bp, Homo sapiens
actgatattgaaagtggaactgtct

SEQ ID NO: 1819 - DNA, 25 bp, Homo sapiens
ctgatattgaaagtggaactgtcta

SEQ ID NO: 1820 - DNA, 25 bp, Homo sapiens
tgatattgaaagtggaactgtctat

SEQ ID NO: 1821 - DNA, 25 bp, Homo sapiens
gatattgaaagtggaactgtctatg

SEQ ID NO: 1822 - DNA, 25 bp, Homo sapiens
atattgaaagtggaactgtctatga

SEQ ID NO: 1823 - DNA, 25 bp, Homo sapiens
tattgaaagtggaactgtctatgac

SEQ ID NO: 1824 - DNA, 25 bp, Homo sapiens
attgaaagtggaactgtctatgact

SEQ ID NO: 1825 - DNA, 25 bp, Homo sapiens
ttgaaagtggaactgtctatgactg

SEQ ID NO: 1826 - DNA, 25 bp, Homo sapiens
tgaaagtggaactgtctatgactgt

SEQ ID NO: 1827 - DNA, 25 bp, Homo sapiens
gaaagtggaactgtctatgactgtg

SEQ ID NO: 1828 - DNA, 25 bp, Homo sapiens
aaagtggaactgtctatgactgtgt

SEQ ID NO: 1829 - DNA, 25 bp, Homo sapiens
aagtggaactgtctatgactgtgta

SEQ ID NO: 1830 - DNA, 25 bp, Homo sapiens
agtggaactgtctatgactgtgtaa

SEQ ID NO: 1831 - DNA, 25 bp, Homo sapiens
gtggaactgtctatgactgtgtaaa

SEQ ID NO: 1832 - DNA, 25 bp, Homo sapiens
tggaactgtctatgactgtgtaaaa

SEQ ID NO: 1833 - DNA, 25 bp, Homo sapiens
ggaactgtctatgactgtgtaaaac

SEQ ID NO: 1834 - DNA, 25 bp, Homo sapiens
gaactgtctatgactgtgtaaaact

SEQ ID NO: 1835 - DNA, 25 bp, Homo sapiens
aactgtctatgactgtgtaaaacta

SEQ ID NO: 1836 - DNA, 25 bp, Homo sapiens
actgtctatgactgtgtaaaactaa

SEQ ID NO: 1837 - DNA, 25 bp, Homo sapiens
ctgtctatgactgtgtaaaactaac

SEQ ID NO: 1838 - DNA, 25 bp, Homo sapiens
tgtctatgactgtgtaaaactaact

SEQ ID NO: 1839 - DNA, 25 bp, Homo sapiens
gtctatgactgtgtaaaactaactt

SEQ ID NO: 1840 - DNA, 25 bp, Homo sapiens
tctatgactgtgtaaaactaacttt

SEQ ID NO: 1841 - DNA, 25 bp, Homo sapiens
ctatgactgtgtaaaactaactttc

SEQ ID NO: 1842 - DNA, 25 bp, Homo sapiens
tatgactgtgtaaaactaactttca

SEQ ID NO: 1843 - DNA, 25 bp, Homo sapiens
atgactgtgtaaaactaactttcag

SEQ ID NO: 1844 - DNA, 25 bp, Homo sapiens
tgactgtgtaaaactaactttcagt

SEQ ID NO: 1845 - DNA, 25 bp, Homo sapiens
gactgtgtaaaactaactttcagtc

SEQ ID NO: 1846 - DNA, 25 bp, Homo sapiens
actgtgtaaaactaactttcagtcc

SEQ ID NO: 1847 - DNA, 25 bp, Homo sapiens
ctgtgtaaaactaactttcagtcca

SEQ ID NO: 1848 - DNA, 25 bp, Homo sapiens
tgtgtaaaactaactttcagtccat

SEQ ID NO: 1849 - DNA, 25 bp, Homo sapiens
gtgtaaaactaactttcagtccatc

SEQ ID NO: 1850 - DNA, 25 bp, Homo sapiens
tgtaaaactaactttcagtccatca

SEQ ID NO: 1851 - DNA, 25 bp, Homo sapiens
gtaaaactaactttcagtccatcaa

SEQ ID NO: 1852 - DNA, 25 bp, Homo sapiens
taaaactaactttcagtccatcaac

SEQ ID NO: 1853 - DNA, 25 bp, Homo sapiens
aaaactaactttcagtccatcaact

SEQ ID NO: 1854 - DNA, 25 bp, Homo sapiens
aaactaactttcagtccatcaactc

SEQ ID NO: 1855 - DNA, 25 bp, Homo sapiens
aactaactttcagtccatcaactct

SEQ ID NO: 1856 - DNA, 25 bp, Homo sapiens
actaactttcagtccatcaactctg

SEQ ID NO: 1857 - DNA, 25 bp, Homo sapiens
ctaactttcagtccatcaactctgc

SEQ ID NO: 1858 - DNA, 25 bp, Homo sapiens
taactttcagtccatcaactctgct

SEQ ID NO: 1859 - DNA, 25 bp, Homo sapiens
aactttcagtccatcaactctgctg

SEQ ID NO: 1860 - DNA, 25 bp, Homo sapiens
actttcagtccatcaactctgctgg

SEQ ID NO: 1861 - DNA, 25 bp, Homo sapiens
ctttcagtccatcaactctgctggt

SEQ ID NO: 1862 - DNA, 25 bp, Homo sapiens
tttcagtccatcaactctgctggtt

SEQ ID NO: 1863 - DNA, 25 bp, Homo sapiens
ttcagtccatcaactctgctggtta

SEQ ID NO: 1864 - DNA, 25 bp, Homo sapiens
tcagtccatcaactctgctggttaa

SEQ ID NO: 1865 - DNA, 25 bp, Homo sapiens
cagtccatcaactctgctggttaat

SEQ ID NO: 1866 - DNA, 25 bp, Homo sapiens
agtccatcaactctgctggttaata

SEQ ID NO: 1867 - DNA, 25 bp, Homo sapiens
gtccatcaactctgctggttaatat

SEQ ID NO: 1868 - DNA, 25 bp, Homo sapiens
tccatcaactctgctggttaatatc

SEQ ID NO: 1869 - DNA, 25 bp, Homo sapiens
ccatcaactctgctggttaatatca

SEQ ID NO: 1870 - DNA, 25 bp, Homo sapiens
catcaactctgctggttaatatcac

SEQ ID NO: 1871 - DNA, 25 bp, Homo sapiens
atcaactctgctggttaatatcact

SEQ ID NO: 1872 - DNA, 25 bp, Homo sapiens
tcaactctgctggttaatatcactg

SEQ ID NO: 1873 - DNA, 25 bp, Homo sapiens
caactctgctggttaatatcactga

SEQ ID NO: 1874 - DNA, 25 bp, Homo sapiens
aactctgctggttaatatcactgac

SEQ ID NO: 1875 - DNA, 25 bp, Homo sapiens
actctgctggttaatatcactgacc

SEQ ID NO: 1876 - DNA, 25 bp, Homo sapiens
ctctgctggttaatatcactgacca

SEQ ID NO: 1877 - DNA, 25 bp, Homo sapiens
tctgctggttaatatcactgaccaa

SEQ ID NO: 1878 - DNA, 25 bp, Homo sapiens
ctgctggttaatatcactgaccaag

SEQ ID NO: 1879 - DNA, 25 bp, Homo sapiens
tgctggttaatatcactgaccaagt

SEQ ID NO: 1880 - DNA, 25 bp, Homo sapiens
gctggttaatatcactgaccaagtt

SEQ ID NO: 1881 - DNA, 25 bp, Homo sapiens
ctggttaatatcactgaccaagttt

SEQ ID NO: 1882 - DNA, 25 bp, Homo sapiens
tggttaatatcactgaccaagttta

SEQ ID NO: 1883 - DNA, 25 bp, Homo sapiens
ggttaatatcactgaccaagtttat

SEQ ID NO: 1884 - DNA, 25 bp, Homo sapiens
gttaatatcactgaccaagtttatg

SEQ ID NO: 1885 - DNA, 25 bp, Homo sapiens
ttaatatcactgaccaagtttatga

SEQ ID NO: 1886 - DNA, 25 bp, Homo sapiens
taatatcactgaccaagtttatgaa

SEQ ID NO: 1887 - DNA, 25 bp, Homo sapiens
aatatcactgaccaagtttatgaat

SEQ ID NO: 1888 - DNA, 25 bp, Homo sapiens
atatcactgaccaagtttatgaata

SEQ ID NO: 1889 - DNA, 25 bp, Homo sapiens
tatcactgaccaagtttatgaatat

SEQ ID NO: 1890 - DNA, 25 bp, Homo sapiens
atcactgaccaagtttatgaatata

SEQ ID NO: 1891 - DNA, 25 bp, Homo sapiens
tcactgaccaagtttatgaatataa

SEQ ID NO: 1892 - DNA, 25 bp, Homo sapiens
cactgaccaagtttatgaatataaa

SEQ ID NO: 1893 - DNA, 25 bp, Homo sapiens
actgaccaagtttatgaatataaat

SEQ ID NO: 1894 - DNA, 25 bp, Homo sapiens
ctgaccaagtttatgaatataaata

SEQ ID NO: 1895 - DNA, 25 bp, Homo sapiens
tgaccaagtttatgaatataaatac

SEQ ID NO: 1896 - DNA, 25 bp, Homo sapiens
gaccaagtttatgaatataaataca

SEQ ID NO: 1897 - DNA, 25 bp, Homo sapiens
accaagtttatgaatataaatacaa

SEQ ID NO: 1898 - DNA, 25 bp, Homo sapiens
ccaagtttatgaatataaatacaaa

SEQ ID NO: 1899 - DNA, 25 bp, Homo sapiens
caagtttatgaatataaatacaaaa

SEQ ID NO: 1900 - DNA, 25 bp, Homo sapiens
aagtttatgaatataaatacaaaag

SEQ ID NO: 1901 - DNA, 25 bp, Homo sapiens
agtttatgaatataaatacaaaaga

SEQ ID NO: 1902 - DNA, 25 bp, Homo sapiens
gtttatgaatataaatacaaaagag

SEQ ID NO: 1903 - DNA, 25 bp, Homo sapiens
tttatgaatataaatacaaaagaga

SEQ ID NO: 1904 - DNA, 25 bp, Homo sapiens
ttatgaatataaatacaaaagagaa

SEQ ID NO: 1905 - DNA, 25 bp, Homo sapiens
tatgaatataaatacaaaagagaaa

SEQ ID NO: 1906 - DNA, 25 bp, Homo sapiens
atgaatataaatacaaaagagaaat

SEQ ID NO: 1907 - DNA, 25 bp, Homo sapiens
tgaatataaatacaaaagagaaata

SEQ ID NO: 1908 - DNA, 25 bp, Homo sapiens
gaatataaatacaaaagagaaataa

SEQ ID NO: 1909 - DNA, 25 bp, Homo sapiens
aatataaatacaaaagagaaataag

SEQ ID NO: 1910 - DNA, 25 bp, Homo sapiens
atataaatacaaaagagaaataagt

SEQ ID NO: 1911 - DNA, 25 bp, Homo sapiens
tataaatacaaaagagaaataagtc

SEQ ID NO: 1912 - DNA, 25 bp, Homo sapiens
ataaatacaaaagagaaataagtca

SEQ ID NO: 1913 - DNA, 25 bp, Homo sapiens
taaatacaaaagagaaataagtcag

SEQ ID NO: 1914 - DNA, 25 bp, Homo sapiens
aaatacaaaagagaaataagtcagc

SEQ ID NO: 1915 - DNA, 25 bp, Homo sapiens
aatacaaaagagaaataagtcagca

SEQ ID NO: 1916 - DNA, 25 bp, Homo sapiens
atacaaaagagaaataagtcagcac

SEQ ID NO: 1917 - DNA, 25 bp, Homo sapiens
tacaaaagagaaataagtcagcaca

SEQ ID NO: 1918 - DNA, 25 bp, Homo sapiens
acaaaagagaaataagtcagcacaa

SEQ ID NO: 1919 - DNA, 25 bp, Homo sapiens
caaaagagaaataagtcagcacaac

SEQ ID NO: 1920 - DNA, 25 bp, Homo sapiens
aaaagagaaataagtcagcacaaca

SEQ ID NO: 1921 - DNA, 25 bp, Homo sapiens
aaagagaaataagtcagcacaacat

SEQ ID NO: 1922 - DNA, 25 bp, Homo sapiens
aagagaaataagtcagcacaacatc

SEQ ID NO: 1923 - DNA, 25 bp, Homo sapiens
agagaaataagtcagcacaacatca

SEQ ID NO: 1924 - DNA, 25 bp, Homo sapiens
gagaaataagtcagcacaacatcaa

SEQ ID NO: 1925 - DNA, 25 bp, Homo sapiens
agaaataagtcagcacaacatcaat

SEQ ID NO: 1926 - DNA, 25 bp, Homo sapiens
gaaataagtcagcacaacatcaatc

SEQ ID NO: 1927 - DNA, 25 bp, Homo sapiens
aaataagtcagcacaacatcaatcc

SEQ ID NO: 1928 - DNA, 25 bp, Homo sapiens
aataagtcagcacaacatcaatcct

SEQ ID NO: 1929 - DNA, 25 bp, Homo sapiens
ataagtcagcacaacatcaatcctc

SEQ ID NO: 1930 - DNA, 25 bp, Homo sapiens
taagtcagcacaacatcaatcctca

SEQ ID NO: 1931 - DNA, 25 bp, Homo sapiens
aagtcagcacaacatcaatcctcat

SEQ ID NO: 1932 - DNA, 25 bp, Homo sapiens
agtcagcacaacatcaatcctcatc

SEQ ID NO: 1933 - DNA, 25 bp, Homo sapiens
gtcagcacaacatcaatcctcatca

SEQ ID NO: 1934 - DNA, 25 bp, Homo sapiens
tcagcacaacatcaatcctcatcaa

SEQ ID NO: 1935 - DNA, 25 bp, Homo sapiens
cagcacaacatcaatcctcatcaag

SEQ ID NO: 1936 - DNA, 25 bp, Homo sapiens
agcacaacatcaatcctcatcaagg

SEQ ID NO: 1937 - DNA, 25 bp, Homo sapiens
gcacaacatcaatcctcatcaagga

SEQ ID NO: 1938 - DNA, 25 bp, Homo sapiens
cacaacatcaatcctcatcaaggaa

SEQ ID NO: 1939 - DNA, 25 bp, Homo sapiens
acaacatcaatcctcatcaaggaaa

SEQ ID NO: 1940 - DNA, 25 bp, Homo sapiens
caacatcaatcctcatcaaggaaat

SEQ ID NO: 1941 - DNA, 25 bp, Homo sapiens
aacatcaatcctcatcaaggaaatg

SEQ ID NO: 1942 - DNA, 25 bp, Homo sapiens
acatcaatcctcatcaaggaaatgc

SEQ ID NO: 1943 - DNA, 25 bp, Homo sapiens
catcaatcctcatcaaggaaatgct

SEQ ID NO: 1944 - DNA, 25 bp, Homo sapiens
atcaatcctcatcaaggaaatgcta

SEQ ID NO: 1945 - DNA, 25 bp, Homo sapiens
tcaatcctcatcaaggaaatgctat

SEQ ID NO: 1946 - DNA, 25 bp, Homo sapiens
caatcctcatcaaggaaatgctata

SEQ ID NO: 1947 - DNA, 25 bp, Homo sapiens
aatcctcatcaaggaaatgctatac

SEQ ID NO: 1948 - DNA, 25 bp, Homo sapiens
atcctcatcaaggaaatgctatact

SEQ ID NO: 1949 - DNA, 25 bp, Homo sapiens
tcctcatcaaggaaatgctatactt

SEQ ID NO: 1950 - DNA, 25 bp, Homo sapiens
cctcatcaaggaaatgctatacttg

SEQ ID NO: 1951 - DNA, 25 bp, Homo sapiens
ctcatcaaggaaatgctatacttga

SEQ ID NO: 1952 - DNA, 25 bp, Homo sapiens
tcatcaaggaaatgctatacttgaa

SEQ ID NO: 1953 - DNA, 25 bp, Homo sapiens
catcaaggaaatgctatacttgaaa

SEQ ID NO: 1954 - DNA, 25 bp, Homo sapiens
atcaaggaaatgctatacttgaaaa

SEQ ID NO: 1955 - DNA, 25 bp, Homo sapiens
tcaaggaaatgctatacttgaaaag

SEQ ID NO: 1956 - DNA, 25 bp, Homo sapiens
caaggaaatgctatacttgaaaaga

SEQ ID NO: 1957 - DNA, 25 bp, Homo sapiens
aaggaaatgctatacttgaaaagat

SEQ ID NO: 1958 - DNA, 25 bp, Homo sapiens
aggaaatgctatacttgaaaagatg

SEQ ID NO: 1959 - DNA, 25 bp, Homo sapiens
ggaaatgctatacttgaaaagatga

SEQ ID NO: 1960 - DNA, 25 bp, Homo sapiens
gaaatgctatacttgaaaagatgac

SEQ ID NO: 1961 - DNA, 25 bp, Homo sapiens
aaatgctatacttgaaaagatgaca

SEQ ID NO: 1962 - DNA, 25 bp, Homo sapiens
aatgctatacttgaaaagatgacat

SEQ ID NO: 1963 - DNA, 25 bp, Homo sapiens
atgctatacttgaaaagatgacatt

SEQ ID NO: 1964 - DNA, 25 bp, Homo sapiens
tgctatacttgaaaagatgacattt

SEQ ID NO: 1965 - DNA, 25 bp, Homo sapiens
gctatacttgaaaagatgacatttg

SEQ ID NO: 1966 - DNA, 25 bp, Homo sapiens
ctatacttgaaaagatgacatttga

SEQ ID NO: 1967 - DNA, 25 bp, Homo sapiens
tatacttgaaaagatgacatttgat

SEQ ID NO: 1968 - DNA, 25 bp, Homo sapiens
atacttgaaaagatgacatttgatc

SEQ ID NO: 1969 - DNA, 25 bp, Homo sapiens
tacttgaaaagatgacatttgatcc

SEQ ID NO: 1970 - DNA, 25 bp, Homo sapiens
acttgaaaagatgacatttgatcca

SEQ ID NO: 1971 - DNA, 25 bp, Homo sapiens
cttgaaaagatgacatttgatccag

SEQ ID NO: 1972 - DNA, 25 bp, Homo sapiens
ttgaaaagatgacatttgatccaga

SEQ ID NO: 1973 - DNA, 25 bp, Homo sapiens
tgaaaagatgacatttgatccagaa

SEQ ID NO: 1974 - DNA, 25 bp, Homo sapiens
gaaaagatgacatttgatccagaaa

SEQ ID NO: 1975 - DNA, 25 bp, Homo sapiens
aaaagatgacatttgatccagaaat

SEQ ID NO: 1976 - DNA, 25 bp, Homo sapiens
aaagatgacatttgatccagaaatc

SEQ ID NO: 1977 - DNA, 25 bp, Homo sapiens
aagatgacatttgatccagaaatct

SEQ ID NO: 1978 - DNA, 25 bp, Homo sapiens
agatgacatttgatccagaaatctt

SEQ ID NO: 1979 - DNA, 25 bp, Homo sapiens
gatgacatttgatccagaaatcttc

SEQ ID NO: 1980 - DNA, 25 bp, Homo sapiens
atgacatttgatccagaaatcttct

SEQ ID NO: 1981 - DNA, 25 bp, Homo sapiens
tgacatttgatccagaaatcttctt

SEQ ID NO: 1982 - DNA, 25 bp, Homo sapiens
gacatttgatccagaaatcttcttc

SEQ ID NO: 1983 - DNA, 25 bp, Homo sapiens
acatttgatccagaaatcttcttca

SEQ ID NO: 1984 - DNA, 25 bp, Homo sapiens
catttgatccagaaatcttcttcaa

SEQ ID NO: 1985 - DNA, 25 bp, Homo sapiens
atttgatccagaaatcttcttcaat

SEQ ID NO: 1986 - DNA, 25 bp, Homo sapiens
tttgatccagaaatcttcttcaatg

SEQ ID NO: 1987 - DNA, 25 bp, Homo sapiens
ttgatccagaaatcttcttcaatgt

SEQ ID NO: 1988 - DNA, 25 bp, Homo sapiens
tgatccagaaatcttcttcaatgtt

SEQ ID NO: 1989 - DNA, 25 bp, Homo sapiens
gatccagaaatcttcttcaatgttt

SEQ ID NO: 1990 - DNA, 25 bp, Homo sapiens
atccagaaatcttcttcaatgtttt

SEQ ID NO: 1991 - DNA, 25 bp, Homo sapiens
tccagaaatcttcttcaatgtttta

SEQ ID NO: 1992 - DNA, 25 bp, Homo sapiens
ccagaaatcttcttcaatgttttac

SEQ ID NO: 1993 - DNA, 25 bp, Homo sapiens
cagaaatcttcttcaatgttttact

SEQ ID NO: 1994 - DNA, 25 bp, Homo sapiens
agaaatcttcttcaatgttttactg

SEQ ID NO: 1995 - DNA, 25 bp, Homo sapiens
gaaatcttcttcaatgttttactgc

SEQ ID NO: 1996 - DNA, 25 bp, Homo sapiens
aaatcttcttcaatgttttactgcc

SEQ ID NO: 1997 - DNA, 25 bp, Homo sapiens
aatcttcttcaatgttttactgcca

SEQ ID NO: 1998 - DNA, 25 bp, Homo sapiens
atcttcttcaatgttttactgccac

SEQ ID NO: 1999 - DNA, 25 bp, Homo sapiens
tcttcttcaatgttttactgccacc

SEQ ID NO: 2000 - DNA, 25 bp, Homo sapiens
cttcttcaatgttttactgccacca

SEQ ID NO: 2001 - DNA, 25 bp, Homo sapiens
ttcttcaatgttttactgccaccaa

SEQ ID NO: 2002 - DNA, 25 bp, Homo sapiens
tcttcaatgttttactgccaccaat

SEQ ID NO: 2003 - DNA, 25 bp, Homo sapiens
cttcaatgttttactgccaccaatt

SEQ ID NO: 2004 - DNA, 25 bp, Homo sapiens
ttcaatgttttactgccaccaatta

SEQ ID NO: 2005 - DNA, 25 bp, Homo sapiens
tcaatgttttactgccaccaattat

SEQ ID NO: 2006 - DNA, 25 bp, Homo sapiens
caatgttttactgccaccaattata

SEQ ID NO: 2007 - DNA, 25 bp, Homo sapiens
aatgttttactgccaccaattatat

SEQ ID NO: 2008 - DNA, 25 bp, Homo sapiens
atgttttactgccaccaattatatt

SEQ ID NO: 2009 - DNA, 25 bp, Homo sapiens
tgttttactgccaccaattatattt

SEQ ID NO: 2010 - DNA, 25 bp, Homo sapiens
gttttactgccaccaattatatttc

SEQ ID NO: 2011 - DNA, 25 bp, Homo sapiens
ttttactgccaccaattatatttca

SEQ ID NO: 2012 - DNA, 25 bp, Homo sapiens
tttactgccaccaattatatttcat

SEQ ID NO: 2013 - DNA, 25 bp, Homo sapiens
ttactgccaccaattatatttcatg

SEQ ID NO: 2014 - DNA, 25 bp, Homo sapiens
tactgccaccaattatatttcatgc

SEQ ID NO: 2015 - DNA, 25 bp, Homo sapiens
actgccaccaattatatttcatgca

SEQ ID NO: 2016 - DNA, 25 bp, Homo sapiens
ctgccaccaattatatttcatgcag

SEQ ID NO: 2017 - DNA, 25 bp, Homo sapiens
tgccaccaattatatttcatgcagg

SEQ ID NO: 2018 - DNA, 25 bp, Homo sapiens
gccaccaattatatttcatgcagga

SEQ ID NO: 2019 - DNA, 25 bp, Homo sapiens
ccaccaattatatttcatgcaggat

SEQ ID NO: 2020 - DNA, 25 bp, Homo sapiens
caccaattatatttcatgcaggata

SEQ ID NO: 2021 - DNA, 25 bp, Homo sapiens
accaattatatttcatgcaggatat

SEQ ID NO: 2022 - DNA, 25 bp, Homo sapiens
ccaattatatttcatgcaggatata

SEQ ID NO: 2023 - DNA, 25 bp, Homo sapiens
caattatatttcatgcaggatatag

SEQ ID NO: 2024 - DNA, 25 bp, Homo sapiens
aattatatttcatgcaggatatagt

SEQ ID NO: 2025 - DNA, 25 bp, Homo sapiens
attatatttcatgcaggatatagtc

SEQ ID NO: 2026 - DNA, 25 bp, Homo sapiens
ttatatttcatgcaggatatagtct

SEQ ID NO: 2027 - DNA, 25 bp, Homo sapiens
tatatttcatgcaggatatagtcta

SEQ ID NO: 2028 - DNA, 25 bp, Homo sapiens
atatttcatgcaggatatagtctaa

SEQ ID NO: 2029 - DNA, 25 bp, Homo sapiens
tatttcatgcaggatatagtctaaa

SEQ ID NO: 2030 - DNA, 25 bp, Homo sapiens
atttcatgcaggatatagtctaaag

SEQ ID NO: 2031 - DNA, 25 bp, Homo sapiens
tttcatgcaggatatagtctaaaga

SEQ ID NO: 2032 - DNA, 25 bp, Homo sapiens
ttcatgcaggatatagtctaaagaa

SEQ ID NO: 2033 - DNA, 25 bp, Homo sapiens
tcatgcaggatatagtctaaagaag

SEQ ID NO: 2034 - DNA, 25 bp, Homo sapiens
catgcaggatatagtctaaagaaga

SEQ ID NO: 2035 - DNA, 25 bp, Homo sapiens
atgcaggatatagtctaaagaagag

SEQ ID NO: 2036 - DNA, 25 bp, Homo sapiens
tgcaggatatagtctaaagaagaga

SEQ ID NO: 2037 - DNA, 25 bp, Homo sapiens
gcaggatatagtctaaagaagagac

SEQ ID NO: 2038 - DNA, 25 bp, Homo sapiens
caggatatagtctaaagaagagaca

SEQ ID NO: 2039 - DNA, 25 bp, Homo sapiens
aggatatagtctaaagaagagacac

SEQ ID NO: 2040 - DNA, 25 bp, Homo sapiens
ggatatagtctaaagaagagacact

SEQ ID NO: 2041 - DNA, 25 bp, Homo sapiens
gatatagtctaaagaagagacactt

SEQ ID NO: 2042 - DNA, 25 bp, Homo sapiens
atatagtctaaagaagagacacttt

SEQ ID NO: 2043 - DNA, 25 bp, Homo sapiens
tatagtctaaagaagagacactttt

SEQ ID NO: 2044 - DNA, 25 bp, Homo sapiens
atagtctaaagaagagacacttttt

SEQ ID NO: 2045 - DNA, 25 bp, Homo sapiens
tagtctaaagaagagacactttttt

SEQ ID NO: 2046 - DNA, 25 bp, Homo sapiens
agtctaaagaagagacacttttttc

SEQ ID NO: 2047 - DNA, 25 bp, Homo sapiens
gtctaaagaagagacacttttttca

SEQ ID NO: 2048 - DNA, 25 bp, Homo sapiens
tctaaagaagagacacttttttcaa

SEQ ID NO: 2049 - DNA, 25 bp, Homo sapiens
ctaaagaagagacacttttttcaaa

SEQ ID NO: 2050 - DNA, 25 bp, Homo sapiens
taaagaagagacacttttttcaaaa

SEQ ID NO: 2051 - DNA, 25 bp, Homo sapiens
aaagaagagacacttttttcaaaac

SEQ ID NO: 2052 - DNA, 25 bp, Homo sapiens
aagaagagacacttttttcaaaact

SEQ ID NO: 2053 - DNA, 25 bp, Homo sapiens
agaagagacacttttttcaaaactt

SEQ ID NO: 2054 - DNA, 25 bp, Homo sapiens
gaagagacacttttttcaaaactta

SEQ ID NO: 2055 - DNA, 25 bp, Homo sapiens
aagagacacttttttcaaaacttag

SEQ ID NO: 2056 - DNA, 25 bp, Homo sapiens
agagacacttttttcaaaacttagg

SEQ ID NO: 2057 - DNA, 25 bp, Homo sapiens
gagacacttttttcaaaacttagga

SEQ ID NO: 2058 - DNA, 25 bp, Homo sapiens
agacacttttttcaaaacttaggat

SEQ ID NO: 2059 - DNA, 25 bp, Homo sapiens
gacacttttttcaaaacttaggatc

SEQ ID NO: 2060 - DNA, 25 bp, Homo sapiens
acacttttttcaaaacttaggatct

SEQ ID NO: 2061 - DNA, 25 bp, Homo sapiens
cacttttttcaaaacttaggatcta

SEQ ID NO: 2062 - DNA, 25 bp, Homo sapiens
acttttttcaaaacttaggatctat

SEQ ID NO: 2063 - DNA, 25 bp, Homo sapiens
cttttttcaaaacttaggatctatt

SEQ ID NO: 2064 - DNA, 25 bp, Homo sapiens
ttttttcaaaacttaggatctattt

SEQ ID NO: 2065 - DNA, 25 bp, Homo sapiens
tttttcaaaacttaggatctatttt

SEQ ID NO: 2066 - DNA, 25 bp, Homo sapiens
ttttcaaaacttaggatctatttta

SEQ ID NO: 2067 - DNA, 25 bp, Homo sapiens
tttcaaaacttaggatctattttaa

SEQ ID NO: 2068 - DNA, 25 bp, Homo sapiens
ttcaaaacttaggatctattttaac

SEQ ID NO: 2069 - DNA, 25 bp, Homo sapiens
tcaaaacttaggatctattttaacg

SEQ ID NO: 2070 - DNA, 25 bp, Homo sapiens
caaaacttaggatctattttaacgt

SEQ ID NO: 2071 - DNA, 25 bp, Homo sapiens
aaaacttaggatctattttaacgta

SEQ ID NO: 2072 - DNA, 25 bp, Homo sapiens
aaacttaggatctattttaacgtat

SEQ ID NO: 2073 - DNA, 25 bp, Homo sapiens
aacttaggatctattttaacgtatg

SEQ ID NO: 2074 - DNA, 25 bp, Homo sapiens
acttaggatctattttaacgtatgc

SEQ ID NO: 2075 - DNA, 25 bp, Homo sapiens
cttaggatctattttaacgtatgcc

SEQ ID NO: 2076 - DNA, 25 bp, Homo sapiens
ttaggatctattttaacgtatgcct

SEQ ID NO: 2077 - DNA, 25 bp, Homo sapiens
taggatctattttaacgtatgcctt

SEQ ID NO: 2078 - DNA, 25 bp, Homo sapiens
aggatctattttaacgtatgccttc

SEQ ID NO: 2079 - DNA, 25 bp, Homo sapiens
ggatctattttaacgtatgccttct

SEQ ID NO: 2080 - DNA, 25 bp, Homo sapiens
gatctattttaacgtatgccttctt

SEQ ID NO: 2081 - DNA, 25 bp, Homo sapiens
atctattttaacgtatgccttcttg

SEQ ID NO: 2082 - DNA, 25 bp, Homo sapiens
tctattttaacgtatgccttcttgg

SEQ ID NO: 2083 - DNA, 25 bp, Homo sapiens
ctattttaacgtatgccttcttggg

SEQ ID NO: 2084 - DNA, 25 bp, Homo sapiens
tattttaacgtatgccttcttggga

SEQ ID NO: 2085 - DNA, 25 bp, Homo sapiens
attttaacgtatgccttcttgggaa

SEQ ID NO: 2086 - DNA, 25 bp, Homo sapiens
ttttaacgtatgccttcttgggaac

SEQ ID NO: 2087 - DNA, 25 bp, Homo sapiens
tttaacgtatgccttcttgggaact

SEQ ID NO: 2088 - DNA, 25 bp, Homo sapiens
ttaacgtatgccttcttgggaactg

SEQ ID NO: 2089 - DNA, 25 bp, Homo sapiens
taacgtatgccttcttgggaactgc

SEQ ID NO: 2090 - DNA, 25 bp, Homo sapiens
aacgtatgccttcttgggaactgcc

SEQ ID NO: 2091 - DNA, 25 bp, Homo sapiens
acgtatgccttcttgggaactgcca

SEQ ID NO: 2092 - DNA, 25 bp, Homo sapiens
cgtatgccttcttgggaactgccat

SEQ ID NO: 2093 - DNA, 25 bp, Homo sapiens
gtatgccttcttgggaactgccatc

SEQ ID NO: 2094 - DNA, 25 bp, Homo sapiens
tatgccttcttgggaactgccatct

SEQ ID NO: 2095 - DNA, 25 bp, Homo sapiens
atgccttcttgggaactgccatctc

SEQ ID NO: 2096 - DNA, 25 bp, Homo sapiens
tgccttcttgggaactgccatctcc

SEQ ID NO: 2097 - DNA, 25 bp, Homo sapiens
gccttcttgggaactgccatctcct

SEQ ID NO: 2098 - DNA, 25 bp, Homo sapiens
ccttcttgggaactgccatctcctg

SEQ ID NO: 2099 - DNA, 25 bp, Homo sapiens
cttcttgggaactgccatctcctgc

SEQ ID NO: 2100 - DNA, 25 bp, Homo sapiens
ttcttgggaactgccatctcctgca

SEQ ID NO: 2101 - DNA, 25 bp, Homo sapiens
tcttgggaactgccatctcctgcat

SEQ ID NO: 2102 - DNA, 25 bp, Homo sapiens
cttgggaactgccatctcctgcatc

SEQ ID NO: 2103 - DNA, 25 bp, Homo sapiens
ttgggaactgccatctcctgcatcg

SEQ ID NO: 2104 - DNA, 25 bp, Homo sapiens
tgggaactgccatctcctgcatcgt

SEQ ID NO: 2105 - DNA, 25 bp, Homo sapiens
gggaactgccatctcctgcatcgtc

SEQ ID NO: 2106 - DNA, 25 bp, Homo sapiens
ggaactgccatctcctgcatcgtca

SEQ ID NO: 2107 - DNA, 25 bp, Homo sapiens
gaactgccatctcctgcatcgtcat

SEQ ID NO: 2108 - DNA, 25 bp, Homo sapiens
aactgccatctcctgcatcgtcata

SEQ ID NO: 2109 - DNA, 25 bp, Homo sapiens
actgccatctcctgcatcgtcatag

SEQ ID NO: 2110 - DNA, 25 bp, Homo sapiens
ctgccatctcctgcatcgtcatagg

SEQ ID NO: 2111 - DNA, 25 bp, Homo sapiens
tgccatctcctgcatcgtcataggg

SEQ ID NO: 2112 - DNA, 25 bp, Homo sapiens
gccatctcctgcatcgtcatagggt

SEQ ID NO: 2113 - DNA, 25 bp, Homo sapiens
ccatctcctgcatcgtcatagggtt

SEQ ID NO: 2114 - DNA, 25 bp, Homo sapiens
catctcctgcatcgtcatagggtta

SEQ ID NO: 2115 - DNA, 25 bp, Homo sapiens
atctcctgcatcgtcatagggttaa

SEQ ID NO: 2116 - DNA, 25 bp, Homo sapiens
tctcctgcatcgtcatagggttaat

SEQ ID NO: 2117 - DNA, 25 bp, Homo sapiens
ctcctgcatcgtcatagggttaatt

SEQ ID NO: 2118 - DNA, 25 bp, Homo sapiens
tcctgcatcgtcatagggttaatta

SEQ ID NO: 2119 - DNA, 25 bp, Homo sapiens
cctgcatcgtcatagggttaattat

SEQ ID NO: 2120 - DNA, 25 bp, Homo sapiens
ctgcatcgtcatagggttaattatg

SEQ ID NO: 2121 - DNA, 25 bp, Homo sapiens
tgcatcgtcatagggttaattatgt

SEQ ID NO: 2122 - DNA, 25 bp, Homo sapiens
gcatcgtcatagggttaattatgta

SEQ ID NO: 2123 - DNA, 25 bp, Homo sapiens
catcgtcatagggttaattatgtat

SEQ ID NO: 2124 - DNA, 25 bp, Homo sapiens
atcgtcatagggttaattatgtatg

SEQ ID NO: 2125 - DNA, 25 bp, Homo sapiens
tcgtcatagggttaattatgtatgg

SEQ ID NO: 2126 - DNA, 25 bp, Homo sapiens
cgtcatagggttaattatgtatggt

SEQ ID NO: 2127 - DNA, 25 bp, Homo sapiens
gtcatagggttaattatgtatggtt

SEQ ID NO: 2128 - DNA, 25 bp, Homo sapiens
tcatagggttaattatgtatggttt

SEQ ID NO: 2129 - DNA, 25 bp, Homo sapiens
catagggttaattatgtatggtttt

SEQ ID NO: 2130 - DNA, 25 bp, Homo sapiens
atagggttaattatgtatggttttg

SEQ ID NO: 2131 - DNA, 25 bp, Homo sapiens
tagggttaattatgtatggttttgt

SEQ ID NO: 2132 - DNA, 25 bp, Homo sapiens
agggttaattatgtatggttttgtg

SEQ ID NO: 2133 - DNA, 25 bp, Homo sapiens
gggttaattatgtatggttttgtga

SEQ ID NO: 2134 - DNA, 25 bp, Homo sapiens
ggttaattatgtatggttttgtgaa

SEQ ID NO: 2135 - DNA, 25 bp, Homo sapiens
gttaattatgtatggttttgtgaag

SEQ ID NO: 2136 - DNA, 25 bp, Homo sapiens
ttaattatgtatggttttgtgaagg

SEQ ID NO: 2137 - DNA, 25 bp, Homo sapiens
taattatgtatggttttgtgaaggc

SEQ ID NO: 2138 - DNA, 25 bp, Homo sapiens
aattatgtatggttttgtgaaggct

SEQ ID NO: 2139 - DNA, 25 bp, Homo sapiens
attatgtatggttttgtgaaggcta

SEQ ID NO: 2140 - DNA, 25 bp, Homo sapiens
ttatgtatggttttgtgaaggctat

SEQ ID NO: 2141 - DNA, 25 bp, Homo sapiens
tatgtatggttttgtgaaggctatg

SEQ ID NO: 2142 - DNA, 25 bp, Homo sapiens
atgtatggttttgtgaaggctatga

SEQ ID NO: 2143 - DNA, 25 bp, Homo sapiens
tgtatggttttgtgaaggctatgat

SEQ ID NO: 2144 - DNA, 25 bp, Homo sapiens
gtatggttttgtgaaggctatgata

SEQ ID NO: 2145 - DNA, 25 bp, Homo sapiens
tatggttttgtgaaggctatgatac

SEQ ID NO: 2146 - DNA, 25 bp, Homo sapiens
atggttttgtgaaggctatgataca

SEQ ID NO: 2147 - DNA, 25 bp, Homo sapiens
tggttttgtgaaggctatgatacat

SEQ ID NO: 2148 - DNA, 25 bp, Homo sapiens
ggttttgtgaaggctatgatacatg

SEQ ID NO: 2149 - DNA, 25 bp, Homo sapiens
gttttgtgaaggctatgatacatgc

SEQ ID NO: 2150 - DNA, 25 bp, Homo sapiens
ttttgtgaaggctatgatacatgct

SEQ ID NO: 2151 - DNA, 25 bp, Homo sapiens
tttgtgaaggctatgatacatgctg

SEQ ID NO: 2152 - DNA, 25 bp, Homo sapiens
ttgtgaaggctatgatacatgctgg

SEQ ID NO: 2153 - DNA, 25 bp, Homo sapiens
tgtgaaggctatgatacatgctggc

SEQ ID NO: 2154 - DNA, 25 bp, Homo sapiens
gtgaaggctatgatacatgctggcc

SEQ ID NO: 2155 - DNA, 25 bp, Homo sapiens
tgaaggctatgatacatgctggcca

SEQ ID NO: 2156 - DNA, 25 bp, Homo sapiens
gaaggctatgatacatgctggccag

SEQ ID NO: 2157 - DNA, 25 bp, Homo sapiens
aaggctatgatacatgctggccagc

SEQ ID NO: 2158 - DNA, 25 bp, Homo sapiens
aggctatgatacatgctggccagct

SEQ ID NO: 2159 - DNA, 25 bp, Homo sapiens
ggctatgatacatgctggccagctg

SEQ ID NO: 2160 - DNA, 25 bp, Homo sapiens
gctatgatacatgctggccagctga

SEQ ID NO: 2161 - DNA, 25 bp, Homo sapiens
ctatgatacatgctggccagctgaa

SEQ ID NO: 2162 - DNA, 25 bp, Homo sapiens
tatgatacatgctggccagctgaaa

SEQ ID NO: 2163 - DNA, 25 bp, Homo sapiens
atgatacatgctggccagctgaaaa

SEQ ID NO: 2164 - DNA, 25 bp, Homo sapiens
tgatacatgctggccagctgaaaaa

SEQ ID NO: 2165 - DNA, 25 bp, Homo sapiens
gatacatgctggccagctgaaaaat

SEQ ID NO: 2166 - DNA, 25 bp, Homo sapiens
atacatgctggccagctgaaaaatg

SEQ ID NO: 2167 - DNA, 25 bp, Homo sapiens
tacatgctggccagctgaaaaatgg

SEQ ID NO: 2168 - DNA, 25 bp, Homo sapiens
acatgctggccagctgaaaaatgga

SEQ ID NO: 2169 - DNA, 25 bp, Homo sapiens
catgctggccagctgaaaaatggag

SEQ ID NO: 2170 - DNA, 25 bp, Homo sapiens
atgctggccagctgaaaaatggaga

SEQ ID NO: 2171 - DNA, 25 bp, Homo sapiens
tgctggccagctgaaaaatggagac

SEQ ID NO: 2172 - DNA, 25 bp, Homo sapiens
gctggccagctgaaaaatggagact

SEQ ID NO: 2173 - DNA, 25 bp, Homo sapiens
ctggccagctgaaaaatggagactt

SEQ ID NO: 2174 - DNA, 25 bp, Homo sapiens
tggccagctgaaaaatggagacttt

SEQ ID NO: 2175 - DNA, 25 bp, Homo sapiens
ggccagctgaaaaatggagactttc

SEQ ID NO: 2176 - DNA, 25 bp, Homo sapiens
gccagctgaaaaatggagactttca

SEQ ID NO: 2177 - DNA, 25 bp, Homo sapiens
ccagctgaaaaatggagactttcat

SEQ ID NO: 2178 - DNA, 25 bp, Homo sapiens
cagctgaaaaatggagactttcatt

SEQ ID NO: 2179 - DNA, 25 bp, Homo sapiens
agctgaaaaatggagactttcattt

SEQ ID NO: 2180 - DNA, 25 bp, Homo sapiens
gctgaaaaatggagactttcatttc

SEQ ID NO: 2181 - DNA, 25 bp, Homo sapiens
ctgaaaaatggagactttcatttca

SEQ ID NO: 2182 - DNA, 25 bp, Homo sapiens
tgaaaaatggagactttcatttcac

SEQ ID NO: 2183 - DNA, 25 bp, Homo sapiens
gaaaaatggagactttcatttcact

SEQ ID NO: 2184 - DNA, 25 bp, Homo sapiens
aaaaatggagactttcatttcactg

SEQ ID NO: 2185 - DNA, 25 bp, Homo sapiens
aaaatggagactttcatttcactga

SEQ ID NO: 2186 - DNA, 25 bp, Homo sapiens
aaatggagactttcatttcactgac

SEQ ID NO: 2187 - DNA, 25 bp, Homo sapiens
aatggagactttcatttcactgact

SEQ ID NO: 2188 - DNA, 25 bp, Homo sapiens
atggagactttcatttcactgactg

SEQ ID NO: 2189 - DNA, 25 bp, Homo sapiens
tggagactttcatttcactgactgt

SEQ ID NO: 2190 - DNA, 25 bp, Homo sapiens
ggagactttcatttcactgactgtt

SEQ ID NO: 2191 - DNA, 25 bp, Homo sapiens
gagactttcatttcactgactgttt

SEQ ID NO: 2192 - DNA, 25 bp, Homo sapiens
agactttcatttcactgactgttta

SEQ ID NO: 2193 - DNA, 25 bp, Homo sapiens
gactttcatttcactgactgtttat

SEQ ID NO: 2194 - DNA, 25 bp, Homo sapiens
actttcatttcactgactgtttatt

SEQ ID NO: 2195 - DNA, 25 bp, Homo sapiens
ctttcatttcactgactgtttattt

SEQ ID NO: 2196 - DNA, 25 bp, Homo sapiens
tttcatttcactgactgtttatttt

SEQ ID NO: 2197 - DNA, 25 bp, Homo sapiens
ttcatttcactgactgtttattttt

SEQ ID NO: 2198 - DNA, 25 bp, Homo sapiens
tcatttcactgactgtttatttttt

SEQ ID NO: 2199 - DNA, 25 bp, Homo sapiens
catttcactgactgtttattttttg

SEQ ID NO: 2200 - DNA, 25 bp, Homo sapiens
atttcactgactgtttattttttgg

SEQ ID NO: 2201 - DNA, 25 bp, Homo sapiens
tttcactgactgtttattttttggt

SEQ ID NO: 2202 - DNA, 25 bp, Homo sapiens
ttcactgactgtttattttttggtt

SEQ ID NO: 2203 - DNA, 25 bp, Homo sapiens
tcactgactgtttattttttggttc

SEQ ID NO: 2204 - DNA, 25 bp, Homo sapiens
cactgactgtttattttttggttca

SEQ ID NO: 2205 - DNA, 25 bp, Homo sapiens
actgactgtttattttttggttcac

SEQ ID NO: 2206 - DNA, 25 bp, Homo sapiens
ctgactgtttattttttggttcact

SEQ ID NO: 2207 - DNA, 25 bp, Homo sapiens
tgactgtttattttttggttcactg

SEQ ID NO: 2208 - DNA, 25 bp, Homo sapiens
gactgtttattttttggttcactga

SEQ ID NO: 2209 - DNA, 25 bp, Homo sapiens
actgtttattttttggttcactgat

SEQ ID NO: 2210 - DNA, 25 bp, Homo sapiens
ctgtttattttttggttcactgatg

SEQ ID NO: 2211 - DNA, 25 bp, Homo sapiens
tgtttattttttggttcactgatgt

SEQ ID NO: 2212 - DNA, 25 bp, Homo sapiens
gtttattttttggttcactgatgtc

SEQ ID NO: 2213 - DNA, 25 bp, Homo sapiens
tttattttttggttcactgatgtct

SEQ ID NO: 2214 - DNA, 25 bp, Homo sapiens
ttattttttggttcactgatgtctg

SEQ ID NO: 2215 - DNA, 25 bp, Homo sapiens
tattttttggttcactgatgtctgc

SEQ ID NO: 2216 - DNA, 25 bp, Homo sapiens
attttttggttcactgatgtctgct

SEQ ID NO: 2217 - DNA, 25 bp, Homo sapiens
ttttttggttcactgatgtctgcta

SEQ ID NO: 2218 - DNA, 25 bp, Homo sapiens
tttttggttcactgatgtctgctac

SEQ ID NO: 2219 - DNA, 25 bp, Homo sapiens
ttttggttcactgatgtctgctaca

SEQ ID NO: 2220 - DNA, 25 bp, Homo sapiens
tttggttcactgatgtctgctacag

SEQ ID NO: 2221 - DNA, 25 bp, Homo sapiens
ttggttcactgatgtctgctacaga

SEQ ID NO: 2222 - DNA, 25 bp, Homo sapiens
tggttcactgatgtctgctacagat

SEQ ID NO: 2223 - DNA, 25 bp, Homo sapiens
ggttcactgatgtctgctacagatc

SEQ ID NO: 2224 - DNA, 25 bp, Homo sapiens
gttcactgatgtctgctacagatcc

SEQ ID NO: 2225 - DNA, 25 bp, Homo sapiens
ttcactgatgtctgctacagatcca

SEQ ID NO: 2226 - DNA, 25 bp, Homo sapiens
tcactgatgtctgctacagatccag

SEQ ID NO: 2227 - DNA, 25 bp, Homo sapiens
cactgatgtctgctacagatccagt

SEQ ID NO: 2228 - DNA, 25 bp, Homo sapiens
actgatgtctgctacagatccagtg

SEQ ID NO: 2229 - DNA, 25 bp, Homo sapiens
ctgatgtctgctacagatccagtga

SEQ ID NO: 2230 - DNA, 25 bp, Homo sapiens
tgatgtctgctacagatccagtgac

SEQ ID NO: 2231 - DNA, 25 bp, Homo sapiens
gatgtctgctacagatccagtgaca

SEQ ID NO: 2232 - DNA, 25 bp, Homo sapiens
atgtctgctacagatccagtgacag

SEQ ID NO: 2233 - DNA, 25 bp, Homo sapiens
tgtctgctacagatccagtgacagt

SEQ ID NO: 2234 - DNA, 25 bp, Homo sapiens
gtctgctacagatccagtgacagtg

SEQ ID NO: 2235 - DNA, 25 bp, Homo sapiens
tctgctacagatccagtgacagtgc

SEQ ID NO: 2236 - DNA, 25 bp, Homo sapiens
ctgctacagatccagtgacagtgct

SEQ ID NO: 2237 - DNA, 25 bp, Homo sapiens
tgctacagatccagtgacagtgctg

SEQ ID NO: 2238 - DNA, 25 bp, Homo sapiens
gctacagatccagtgacagtgctgg

SEQ ID NO: 2239 - DNA, 25 bp, Homo sapiens
ctacagatccagtgacagtgctggc

SEQ ID NO: 2240 - DNA, 25 bp, Homo sapiens
tacagatccagtgacagtgctggcc

SEQ ID NO: 2241 - DNA, 25 bp, Homo sapiens
acagatccagtgacagtgctggcca

SEQ ID NO: 2242 - DNA, 25 bp, Homo sapiens
cagatccagtgacagtgctggccat

SEQ ID NO: 2243 - DNA, 25 bp, Homo sapiens
agatccagtgacagtgctggccatt

SEQ ID NO: 2244 - DNA, 25 bp, Homo sapiens
gatccagtgacagtgctggccattt

SEQ ID NO: 2245 - DNA, 25 bp, Homo sapiens
atccagtgacagtgctggccatttt

SEQ ID NO: 2246 - DNA, 25 bp, Homo sapiens
tccagtgacagtgctggccattttc

SEQ ID NO: 2247 - DNA, 25 bp, Homo sapiens
ccagtgacagtgctggccattttcc

SEQ ID NO: 2248 - DNA, 25 bp, Homo sapiens
cagtgacagtgctggccattttcca

SEQ ID NO: 2249 - DNA, 25 bp, Homo sapiens
agtgacagtgctggccattttccat

SEQ ID NO: 2250 - DNA, 25 bp, Homo sapiens
gtgacagtgctggccattttccatg

SEQ ID NO: 2251 - DNA, 25 bp, Homo sapiens
tgacagtgctggccattttccatga

SEQ ID NO: 2252 - DNA, 25 bp, Homo sapiens
gacagtgctggccattttccatgaa

SEQ ID NO: 2253 - DNA, 25 bp, Homo sapiens
acagtgctggccattttccatgaac

SEQ ID NO: 2254 - DNA, 25 bp, Homo sapiens
cagtgctggccattttccatgaact

SEQ ID NO: 2255 - DNA, 25 bp, Homo sapiens
agtgctggccattttccatgaactg

SEQ ID NO: 2256 - DNA, 25 bp, Homo sapiens
gtgctggccattttccatgaactgc

SEQ ID NO: 2257 - DNA, 25 bp, Homo sapiens
tgctggccattttccatgaactgca

SEQ ID NO: 2258 - DNA, 25 bp, Homo sapiens
gctggccattttccatgaactgcac

SEQ ID NO: 2259 - DNA, 25 bp, Homo sapiens
ctggccattttccatgaactgcacg

SEQ ID NO: 2260 - DNA, 25 bp, Homo sapiens
tggccattttccatgaactgcacgt

SEQ ID NO: 2261 - DNA, 25 bp, Homo sapiens
ggccattttccatgaactgcacgtc

SEQ ID NO: 2262 - DNA, 25 bp, Homo sapiens
gccattttccatgaactgcacgtcg

SEQ ID NO: 2263 - DNA, 25 bp, Homo sapiens
ccattttccatgaactgcacgtcga

SEQ ID NO: 2264 - DNA, 25 bp, Homo sapiens
cattttccatgaactgcacgtcgac

SEQ ID NO: 2265 - DNA, 25 bp, Homo sapiens
attttccatgaactgcacgtcgacc

SEQ ID NO: 2266 - DNA, 25 bp, Homo sapiens
ttttccatgaactgcacgtcgaccc

SEQ ID NO: 2267 - DNA, 25 bp, Homo sapiens
tttccatgaactgcacgtcgaccct

SEQ ID NO: 2268 - DNA, 25 bp, Homo sapiens
ttccatgaactgcacgtcgaccctg

SEQ ID NO: 2269 - DNA, 25 bp, Homo sapiens
tccatgaactgcacgtcgaccctga

SEQ ID NO: 2270 - DNA, 25 bp, Homo sapiens
ccatgaactgcacgtcgaccctgac

SEQ ID NO: 2271 - DNA, 25 bp, Homo sapiens
catgaactgcacgtcgaccctgacc

SEQ ID NO: 2272 - DNA, 25 bp, Homo sapiens
atgaactgcacgtcgaccctgacct

SEQ ID NO: 2273 - DNA, 25 bp, Homo sapiens
tgaactgcacgtcgaccctgacctg

SEQ ID NO: 2274 - DNA, 25 bp, Homo sapiens
gaactgcacgtcgaccctgacctgt

SEQ ID NO: 2275 - DNA, 25 bp, Homo sapiens
aactgcacgtcgaccctgacctgta

SEQ ID NO: 2276 - DNA, 25 bp, Homo sapiens
actgcacgtcgaccctgacctgtac

SEQ ID NO: 2277 - DNA, 25 bp, Homo sapiens
ctgcacgtcgaccctgacctgtaca

SEQ ID NO: 2278 - DNA, 25 bp, Homo sapiens
tgcacgtcgaccctgacctgtacac

SEQ ID NO: 2279 - DNA, 25 bp, Homo sapiens
gcacgtcgaccctgacctgtacaca

SEQ ID NO: 2280 - DNA, 25 bp, Homo sapiens
cacgtcgaccctgacctgtacacac

SEQ ID NO: 2281 - DNA, 25 bp, Homo sapiens
acgtcgaccctgacctgtacacact

SEQ ID NO: 2282 - DNA, 25 bp, Homo sapiens
cgtcgaccctgacctgtacacactc

SEQ ID NO: 2283 - DNA, 25 bp, Homo sapiens
gtcgaccctgacctgtacacactct

SEQ ID NO: 2284 - DNA, 25 bp, Homo sapiens
tcgaccctgacctgtacacactctt

SEQ ID NO: 2285 - DNA, 25 bp, Homo sapiens
cgaccctgacctgtacacactcttg

SEQ ID NO: 2286 - DNA, 25 bp, Homo sapiens
gaccctgacctgtacacactcttgt

SEQ ID NO: 2287 - DNA, 25 bp, Homo sapiens
accctgacctgtacacactcttgtt

SEQ ID NO: 2288 - DNA, 25 bp, Homo sapiens
ccctgacctgtacacactcttgttt

SEQ ID NO: 2289 - DNA, 25 bp, Homo sapiens
cctgacctgtacacactcttgtttg

SEQ ID NO: 2290 - DNA, 25 bp, Homo sapiens
ctgacctgtacacactcttgtttgg

SEQ ID NO: 2291 - DNA, 25 bp, Homo sapiens
tgacctgtacacactcttgtttgga

SEQ ID NO: 2292 - DNA, 25 bp, Homo sapiens
gacctgtacacactcttgtttggag

SEQ ID NO: 2293 - DNA, 25 bp, Homo sapiens
acctgtacacactcttgtttggaga

SEQ ID NO: 2294 - DNA, 25 bp, Homo sapiens
cctgtacacactcttgtttggagag

SEQ ID NO: 2295 - DNA, 25 bp, Homo sapiens
ctgtacacactcttgtttggagaga

SEQ ID NO: 2296 - DNA, 25 bp, Homo sapiens
tgtacacactcttgtttggagagag

SEQ ID NO: 2297 - DNA, 25 bp, Homo sapiens
gtacacactcttgtttggagagagt

SEQ ID NO: 2298 - DNA, 25 bp, Homo sapiens
tacacactcttgtttggagagagtg

SEQ ID NO: 2299 - DNA, 25 bp, Homo sapiens
acacactcttgtttggagagagtgt

SEQ ID NO: 2300 - DNA, 25 bp, Homo sapiens
cacactcttgtttggagagagtgtg

SEQ ID NO: 2301 - DNA, 25 bp, Homo sapiens
acactcttgtttggagagagtgtgt

SEQ ID NO: 2302 - DNA, 25 bp, Homo sapiens
cactcttgtttggagagagtgtgtt

SEQ ID NO: 2303 - DNA, 25 bp, Homo sapiens
actcttgtttggagagagtgtgttg

SEQ ID NO: 2304 - DNA, 25 bp, Homo sapiens
ctcttgtttggagagagtgtgttga

SEQ ID NO: 2305 - DNA, 25 bp, Homo sapiens
tcttgtttggagagagtgtgttgaa

SEQ ID NO: 2306 - DNA, 25 bp, Homo sapiens
cttgtttggagagagtgtgttgaat

SEQ ID NO: 2307 - DNA, 25 bp, Homo sapiens
ttgtttggagagagtgtgttgaatg

SEQ ID NO: 2308 - DNA, 25 bp, Homo sapiens
tgtttggagagagtgtgttgaatga

SEQ ID NO: 2309 - DNA, 25 bp, Homo sapiens
gtttggagagagtgtgttgaatgat

SEQ ID NO: 2310 - DNA, 25 bp, Homo sapiens
tttggagagagtgtgttgaatgatg

SEQ ID NO: 2311 - DNA, 25 bp, Homo sapiens
ttggagagagtgtgttgaatgatgc

SEQ ID NO: 2312 - DNA, 25 bp, Homo sapiens
tggagagagtgtgttgaatgatgca

SEQ ID NO: 2313 - DNA, 25 bp, Homo sapiens
ggagagagtgtgttgaatgatgcag

SEQ ID NO: 2314 - DNA, 25 bp, Homo sapiens
gagagagtgtgttgaatgatgcagt

SEQ ID NO: 2315 - DNA, 25 bp, Homo sapiens
agagagtgtgttgaatgatgcagtg

SEQ ID NO: 2316 - DNA, 25 bp, Homo sapiens
gagagtgtgttgaatgatgcagtgg

SEQ ID NO: 2317 - DNA, 25 bp, Homo sapiens
agagtgtgttgaatgatgcagtggc

SEQ ID NO: 2318 - DNA, 25 bp, Homo sapiens
gagtgtgttgaatgatgcagtggcc

SEQ ID NO: 2319 - DNA, 25 bp, Homo sapiens
agtgtgttgaatgatgcagtggcca

SEQ ID NO: 2320 - DNA, 25 bp, Homo sapiens
gtgtgttgaatgatgcagtggccat

SEQ ID NO: 2321 - DNA, 25 bp, Homo sapiens
tgtgttgaatgatgcagtggccata

SEQ ID NO: 2322 - DNA, 25 bp, Homo sapiens
gtgttgaatgatgcagtggccatag

SEQ ID NO: 2323 - DNA, 25 bp, Homo sapiens
tgttgaatgatgcagtggccatagt

SEQ ID NO: 2324 - DNA, 25 bp, Homo sapiens
gttgaatgatgcagtggccatagtc

SEQ ID NO: 2325 - DNA, 25 bp, Homo sapiens
ttgaatgatgcagtggccatagtcc

SEQ ID NO: 2326 - DNA, 25 bp, Homo sapiens
tgaatgatgcagtggccatagtcct

SEQ ID NO: 2327 - DNA, 25 bp, Homo sapiens
gaatgatgcagtggccatagtcctt

SEQ ID NO: 2328 - DNA, 25 bp, Homo sapiens
aatgatgcagtggccatagtcctta

SEQ ID NO: 2329 - DNA, 25 bp, Homo sapiens
atgatgcagtggccatagtccttac

SEQ ID NO: 2330 - DNA, 25 bp, Homo sapiens
tgatgcagtggccatagtccttaca

SEQ ID NO: 2331 - DNA, 25 bp, Homo sapiens
gatgcagtggccatagtccttacat

SEQ ID NO: 2332 - DNA, 25 bp, Homo sapiens
atgcagtggccatagtccttacata

SEQ ID NO: 2333 - DNA, 25 bp, Homo sapiens
tgcagtggccatagtccttacatat

SEQ ID NO: 2334 - DNA, 25 bp, Homo sapiens
gcagtggccatagtccttacatatt

SEQ ID NO: 2335 - DNA, 25 bp, Homo sapiens
cagtggccatagtccttacatattc

SEQ ID NO: 2336 - DNA, 25 bp, Homo sapiens
agtggccatagtccttacatattct

SEQ ID NO: 2337 - DNA, 25 bp, Homo sapiens
gtggccatagtccttacatattcta

SEQ ID NO: 2338 - DNA, 25 bp, Homo sapiens
tggccatagtccttacatattctat

SEQ ID NO: 2339 - DNA, 25 bp, Homo sapiens
ggccatagtccttacatattctata

SEQ ID NO: 2340 - DNA, 25 bp, Homo sapiens
gccatagtccttacatattctatat

SEQ ID NO: 2341 - DNA, 25 bp, Homo sapiens
ccatagtccttacatattctatatc

SEQ ID NO: 2342 - DNA, 25 bp, Homo sapiens
catagtccttacatattctatatcc

SEQ ID NO: 2343 - DNA, 25 bp, Homo sapiens
atagtccttacatattctatatcca

SEQ ID NO: 2344 - DNA, 25 bp, Homo sapiens
tagtccttacatattctatatccat

SEQ ID NO: 2345 - DNA, 25 bp, Homo sapiens
agtccttacatattctatatccatt

SEQ ID NO: 2346 - DNA, 25 bp, Homo sapiens
gtccttacatattctatatccattt

SEQ ID NO: 2347 - DNA, 25 bp, Homo sapiens
tccttacatattctatatccattta

SEQ ID NO: 2348 - DNA, 25 bp, Homo sapiens
ccttacatattctatatccatttac

SEQ ID NO: 2349 - DNA, 25 bp, Homo sapiens
cttacatattctatatccatttaca

SEQ ID NO: 2350 - DNA, 25 bp, Homo sapiens
ttacatattctatatccatttacag

SEQ ID NO: 2351 - DNA, 25 bp, Homo sapiens
tacatattctatatccatttacagt

SEQ ID NO: 2352 - DNA, 25 bp, Homo sapiens
acatattctatatccatttacagtc

SEQ ID NO: 2353 - DNA, 25 bp, Homo sapiens
catattctatatccatttacagtcc

SEQ ID NO: 2354 - DNA, 25 bp, Homo sapiens
atattctatatccatttacagtccc

SEQ ID NO: 2355 - DNA, 25 bp, Homo sapiens
tattctatatccatttacagtccca

SEQ ID NO: 2356 - DNA, 25 bp, Homo sapiens
attctatatccatttacagtcccaa

SEQ ID NO: 2357 - DNA, 25 bp, Homo sapiens
ttctatatccatttacagtcccaag

SEQ ID NO: 2358 - DNA, 25 bp, Homo sapiens
tctatatccatttacagtcccaagg

SEQ ID NO: 2359 - DNA, 25 bp, Homo sapiens
ctatatccatttacagtcccaagga

SEQ ID NO: 2360 - DNA, 25 bp, Homo sapiens
tatatccatttacagtcccaaggag

SEQ ID NO: 2361 - DNA, 25 bp, Homo sapiens
atatccatttacagtcccaaggaga

SEQ ID NO: 2362 - DNA, 25 bp, Homo sapiens
tatccatttacagtcccaaggagaa

SEQ ID NO: 2363 - DNA, 25 bp, Homo sapiens
atccatttacagtcccaaggagaat

SEQ ID NO: 2364 - DNA, 25 bp, Homo sapiens
tccatttacagtcccaaggagaatc

SEQ ID NO: 2365 - DNA, 25 bp, Homo sapiens
ccatttacagtcccaaggagaatcc

SEQ ID NO: 2366 - DNA, 25 bp, Homo sapiens
catttacagtcccaaggagaatcca

SEQ ID NO: 2367 - DNA, 25 bp, Homo sapiens
atttacagtcccaaggagaatccaa

SEQ ID NO: 2368 - DNA, 25 bp, Homo sapiens
tttacagtcccaaggagaatccaaa

SEQ ID NO: 2369 - DNA, 25 bp, Homo sapiens
ttacagtcccaaggagaatccaaat

SEQ ID NO: 2370 - DNA, 25 bp, Homo sapiens
tacagtcccaaggagaatccaaatg

SEQ ID NO: 2371 - DNA, 25 bp, Homo sapiens
acagtcccaaggagaatccaaatgc

SEQ ID NO: 2372 - DNA, 25 bp, Homo sapiens
cagtcccaaggagaatccaaatgca

SEQ ID NO: 2373 - DNA, 25 bp, Homo sapiens
agtcccaaggagaatccaaatgcat

SEQ ID NO: 2374 - DNA, 25 bp, Homo sapiens
gtcccaaggagaatccaaatgcatt

SEQ ID NO: 2375 - DNA, 25 bp, Homo sapiens
tcccaaggagaatccaaatgcattt

SEQ ID NO: 2376 - DNA, 25 bp, Homo sapiens
cccaaggagaatccaaatgcatttg

SEQ ID NO: 2377 - DNA, 25 bp, Homo sapiens
ccaaggagaatccaaatgcatttga

SEQ ID NO: 2378 - DNA, 25 bp, Homo sapiens
caaggagaatccaaatgcatttgat

SEQ ID NO: 2379 - DNA, 25 bp, Homo sapiens
aaggagaatccaaatgcatttgatg

SEQ ID NO: 2380 - DNA, 25 bp, Homo sapiens
aggagaatccaaatgcatttgatgc

SEQ ID NO: 2381 - DNA, 25 bp, Homo sapiens
ggagaatccaaatgcatttgatgcc

SEQ ID NO: 2382 - DNA, 25 bp, Homo sapiens
gagaatccaaatgcatttgatgccg

SEQ ID NO: 2383 - DNA, 25 bp, Homo sapiens
agaatccaaatgcatttgatgccgc

SEQ ID NO: 2384 - DNA, 25 bp, Homo sapiens
gaatccaaatgcatttgatgccgca

SEQ ID NO: 2385 - DNA, 25 bp, Homo sapiens
aatccaaatgcatttgatgccgcag

SEQ ID NO: 2386 - DNA, 25 bp, Homo sapiens
atccaaatgcatttgatgccgcagc

SEQ ID NO: 2387 - DNA, 25 bp, Homo sapiens
tccaaatgcatttgatgccgcagca

SEQ ID NO: 2388 - DNA, 25 bp, Homo sapiens
ccaaatgcatttgatgccgcagcat

SEQ ID NO: 2389 - DNA, 25 bp, Homo sapiens
caaatgcatttgatgccgcagcatt

SEQ ID NO: 2390 - DNA, 25 bp, Homo sapiens
aaatgcatttgatgccgcagcattc

SEQ ID NO: 2391 - DNA, 25 bp, Homo sapiens
aatgcatttgatgccgcagcattct

SEQ ID NO: 2392 - DNA, 25 bp, Homo sapiens
atgcatttgatgccgcagcattctt

SEQ ID NO: 2393 - DNA, 25 bp, Homo sapiens
tgcatttgatgccgcagcattcttc

SEQ ID NO: 2394 - DNA, 25 bp, Homo sapiens
gcatttgatgccgcagcattcttcc

SEQ ID NO: 2395 - DNA, 25 bp, Homo sapiens
catttgatgccgcagcattcttcca

SEQ ID NO: 2396 - DNA, 25 bp, Homo sapiens
atttgatgccgcagcattcttccag

SEQ ID NO: 2397 - DNA, 25 bp, Homo sapiens
tttgatgccgcagcattcttccagt

SEQ ID NO: 2398 - DNA, 25 bp, Homo sapiens
ttgatgccgcagcattcttccagtc

SEQ ID NO: 2399 - DNA, 25 bp, Homo sapiens
tgatgccgcagcattcttccagtct

SEQ ID NO: 2400 - DNA, 25 bp, Homo sapiens
gatgccgcagcattcttccagtctg

SEQ ID NO: 2401 - DNA, 25 bp, Homo sapiens
atgccgcagcattcttccagtctgt

SEQ ID NO: 2402 - DNA, 25 bp, Homo sapiens
tgccgcagcattcttccagtctgtg

SEQ ID NO: 2403 - DNA, 25 bp, Homo sapiens
gccgcagcattcttccagtctgtgg

SEQ ID NO: 2404 - DNA, 25 bp, Homo sapiens
ccgcagcattcttccagtctgtggg

SEQ ID NO: 2405 - DNA, 25 bp, Homo sapiens
cgcagcattcttccagtctgtgggg

SEQ ID NO: 2406 - DNA, 25 bp, Homo sapiens
gcagcattcttccagtctgtgggga

SEQ ID NO: 2407 - DNA, 25 bp, Homo sapiens
cagcattcttccagtctgtggggaa

SEQ ID NO: 2408 - DNA, 25 bp, Homo sapiens
agcattcttccagtctgtggggaat

SEQ ID NO: 2409 - DNA, 25 bp, Homo sapiens
gcattcttccagtctgtggggaatt

SEQ ID NO: 2410 - DNA, 25 bp, Homo sapiens
cattcttccagtctgtggggaattt

SEQ ID NO: 2411 - DNA, 25 bp, Homo sapiens
attcttccagtctgtggggaatttc

SEQ ID NO: 2412 - DNA, 25 bp, Homo sapiens
ttcttccagtctgtggggaatttcc

SEQ ID NO: 2413 - DNA, 25 bp, Homo sapiens
tcttccagtctgtggggaatttcct

SEQ ID NO: 2414 - DNA, 25 bp, Homo sapiens
cttccagtctgtggggaatttcctg

SEQ ID NO: 2415 - DNA, 25 bp, Homo sapiens
ttccagtctgtggggaatttcctgg

SEQ ID NO: 2416 - DNA, 25 bp, Homo sapiens
tccagtctgtggggaatttcctggg

SEQ ID NO: 2417 - DNA, 25 bp, Homo sapiens
ccagtctgtggggaatttcctggga

SEQ ID NO: 2418 - DNA, 25 bp, Homo sapiens
cagtctgtggggaatttcctgggaa

SEQ ID NO: 2419 - DNA, 25 bp, Homo sapiens
agtctgtggggaatttcctgggaat

SEQ ID NO: 2420 - DNA, 25 bp, Homo sapiens
gtctgtggggaatttcctgggaatc

SEQ ID NO: 2421 - DNA, 25 bp, Homo sapiens
tctgtggggaatttcctgggaatct

SEQ ID NO: 2422 - DNA, 25 bp, Homo sapiens
ctgtggggaatttcctgggaatctt

SEQ ID NO: 2423 - DNA, 25 bp, Homo sapiens
tgtggggaatttcctgggaatcttc

SEQ ID NO: 2424 - DNA, 25 bp, Homo sapiens
gtggggaatttcctgggaatcttcg

SEQ ID NO: 2425 - DNA, 25 bp, Homo sapiens
tggggaatttcctgggaatcttcgc

SEQ ID NO: 2426 - DNA, 25 bp, Homo sapiens
ggggaatttcctgggaatcttcgct

SEQ ID NO: 2427 - DNA, 25 bp, Homo sapiens
gggaatttcctgggaatcttcgctg

SEQ ID NO: 2428 - DNA, 25 bp, Homo sapiens
ggaatttcctgggaatcttcgctgg

SEQ ID NO: 2429 - DNA, 25 bp, Homo sapiens
gaatttcctgggaatcttcgctggc

SEQ ID NO: 2430 - DNA, 25 bp, Homo sapiens
aatttcctgggaatcttcgctggct

SEQ ID NO: 2431 - DNA, 25 bp, Homo sapiens
atttcctgggaatcttcgctggctc

SEQ ID NO: 2432 - DNA, 25 bp, Homo sapiens
tttcctgggaatcttcgctggctca

SEQ ID NO: 2433 - DNA, 25 bp, Homo sapiens
ttcctgggaatcttcgctggctcat

SEQ ID NO: 2434 - DNA, 25 bp, Homo sapiens
tcctgggaatcttcgctggctcatt

SEQ ID NO: 2435 - DNA, 25 bp, Homo sapiens
cctgggaatcttcgctggctcattt

SEQ ID NO: 2436 - DNA, 25 bp, Homo sapiens
ctgggaatcttcgctggctcatttg

SEQ ID NO: 2437 - DNA, 25 bp, Homo sapiens
tgggaatcttcgctggctcatttgc

SEQ ID NO: 2438 - DNA, 25 bp, Homo sapiens
gggaatcttcgctggctcatttgca

SEQ ID NO: 2439 - DNA, 25 bp, Homo sapiens
ggaatcttcgctggctcatttgcaa

SEQ ID NO: 2440 - DNA, 25 bp, Homo sapiens
gaatcttcgctggctcatttgcaat

SEQ ID NO: 2441 - DNA, 25 bp, Homo sapiens
aatcttcgctggctcatttgcaatg

SEQ ID NO: 2442 - DNA, 25 bp, Homo sapiens
atcttcgctggctcatttgcaatgg

SEQ ID NO: 2443 - DNA, 25 bp, Homo sapiens
tcttcgctggctcatttgcaatggg

SEQ ID NO: 2444 - DNA, 25 bp, Homo sapiens
cttcgctggctcatttgcaatgggg

SEQ ID NO: 2445 - DNA, 25 bp, Homo sapiens
ttcgctggctcatttgcaatggggt

SEQ ID NO: 2446 - DNA, 25 bp, Homo sapiens
tcgctggctcatttgcaatggggtc

SEQ ID NO: 2447 - DNA, 25 bp, Homo sapiens
cgctggctcatttgcaatggggtct

SEQ ID NO: 2448 - DNA, 25 bp, Homo sapiens
gctggctcatttgcaatggggtctg

SEQ ID NO: 2449 - DNA, 25 bp, Homo sapiens
ctggctcatttgcaatggggtctgc

SEQ ID NO: 2450 - DNA, 25 bp, Homo sapiens
tggctcatttgcaatggggtctgcg

SEQ ID NO: 2451 - DNA, 25 bp, Homo sapiens
ggctcatttgcaatggggtctgcgt

SEQ ID NO: 2452 - DNA, 25 bp, Homo sapiens
gctcatttgcaatggggtctgcgta

SEQ ID NO: 2453 - DNA, 25 bp, Homo sapiens
ctcatttgcaatggggtctgcgtat

SEQ ID NO: 2454 - DNA, 25 bp, Homo sapiens
tcatttgcaatggggtctgcgtatg

SEQ ID NO: 2455 - DNA, 25 bp, Homo sapiens
catttgcaatggggtctgcgtatgc

SEQ ID NO: 2456 - DNA, 25 bp, Homo sapiens
atttgcaatggggtctgcgtatgcc

SEQ ID NO: 2457 - DNA, 25 bp, Homo sapiens
tttgcaatggggtctgcgtatgcca

SEQ ID NO: 2458 - DNA, 25 bp, Homo sapiens
ttgcaatggggtctgcgtatgccat

SEQ ID NO: 2459 - DNA, 25 bp, Homo sapiens
tgcaatggggtctgcgtatgccatc

SEQ ID NO: 2460 - DNA, 25 bp, Homo sapiens
gcaatggggtctgcgtatgccatca

SEQ ID NO: 2461 - DNA, 25 bp, Homo sapiens
caatggggtctgcgtatgccatcat

SEQ ID NO: 2462 - DNA, 25 bp, Homo sapiens
aatggggtctgcgtatgccatcatc

SEQ ID NO: 2463 - DNA, 25 bp, Homo sapiens
atggggtctgcgtatgccatcatca

SEQ ID NO: 2464 - DNA, 25 bp, Homo sapiens
tggggtctgcgtatgccatcatcac

SEQ ID NO: 2465 - DNA, 25 bp, Homo sapiens
ggggtctgcgtatgccatcatcaca

SEQ ID NO: 2466 - DNA, 25 bp, Homo sapiens
gggtctgcgtatgccatcatcacag

SEQ ID NO: 2467 - DNA, 25 bp, Homo sapiens
ggtctgcgtatgccatcatcacagc

SEQ ID NO: 2468 - DNA, 25 bp, Homo sapiens
gtctgcgtatgccatcatcacagca

SEQ ID NO: 2469 - DNA, 25 bp, Homo sapiens
tctgcgtatgccatcatcacagcac

SEQ ID NO: 2470 - DNA, 25 bp, Homo sapiens
ctgcgtatgccatcatcacagcact

SEQ ID NO: 2471 - DNA, 25 bp, Homo sapiens
tgcgtatgccatcatcacagcactg

SEQ ID NO: 2472 - DNA, 25 bp, Homo sapiens
gcgtatgccatcatcacagcactgt

SEQ ID NO: 2473 - DNA, 25 bp, Homo sapiens
cgtatgccatcatcacagcactgtt

SEQ ID NO: 2474 - DNA, 25 bp, Homo sapiens
gtatgccatcatcacagcactgttg

SEQ ID NO: 2475 - DNA, 25 bp, Homo sapiens
tatgccatcatcacagcactgttga

SEQ ID NO: 2476 - DNA, 25 bp, Homo sapiens
atgccatcatcacagcactgttgac

SEQ ID NO: 2477 - DNA, 25 bp, Homo sapiens
tgccatcatcacagcactgttgacc

SEQ ID NO: 2478 - DNA, 25 bp, Homo sapiens
gccatcatcacagcactgttgacca

SEQ ID NO: 2479 - DNA, 25 bp, Homo sapiens
ccatcatcacagcactgttgaccaa

SEQ ID NO: 2480 - DNA, 25 bp, Homo sapiens
catcatcacagcactgttgaccaaa

SEQ ID NO: 2481 - DNA, 25 bp, Homo sapiens
atcatcacagcactgttgaccaaat

SEQ ID NO: 2482 - DNA, 25 bp, Homo sapiens
tcatcacagcactgttgaccaaatt

SEQ ID NO: 2483 - DNA, 25 bp, Homo sapiens
catcacagcactgttgaccaaattt

SEQ ID NO: 2484 - DNA, 25 bp, Homo sapiens
atcacagcactgttgaccaaattta

SEQ ID NO: 2485 - DNA, 25 bp, Homo sapiens
tcacagcactgttgaccaaatttac

SEQ ID NO: 2486 - DNA, 25 bp, Homo sapiens
cacagcactgttgaccaaatttacc

SEQ ID NO: 2487 - DNA, 25 bp, Homo sapiens
acagcactgttgaccaaatttacca

SEQ ID NO: 2488 - DNA, 25 bp, Homo sapiens
cagcactgttgaccaaatttaccaa

SEQ ID NO: 2489 - DNA, 25 bp, Homo sapiens
agcactgttgaccaaatttaccaag

SEQ ID NO: 2490 - DNA, 25 bp, Homo sapiens
gcactgttgaccaaatttaccaagc

SEQ ID NO: 2491 - DNA, 25 bp, Homo sapiens
cactgttgaccaaatttaccaagct

SEQ ID NO: 2492 - DNA, 25 bp, Homo sapiens
actgttgaccaaatttaccaagctg

SEQ ID NO: 2493 - DNA, 25 bp, Homo sapiens
ctgttgaccaaatttaccaagctgt

SEQ ID NO: 2494 - DNA, 25 bp, Homo sapiens
tgttgaccaaatttaccaagctgtg

SEQ ID NO: 2495 - DNA, 25 bp, Homo sapiens
gttgaccaaatttaccaagctgtgt

SEQ ID NO: 2496 - DNA, 25 bp, Homo sapiens
ttgaccaaatttaccaagctgtgtg

SEQ ID NO: 2497 - DNA, 25 bp, Homo sapiens
tgaccaaatttaccaagctgtgtga

SEQ ID NO: 2498 - DNA, 25 bp, Homo sapiens
gaccaaatttaccaagctgtgtgag

SEQ ID NO: 2499 - DNA, 25 bp, Homo sapiens
accaaatttaccaagctgtgtgagt

SEQ ID NO: 2500 - DNA, 25 bp, Homo sapiens
ccaaatttaccaagctgtgtgagtt

SEQ ID NO: 2501 - DNA, 25 bp, Homo sapiens
caaatttaccaagctgtgtgagttc

SEQ ID NO: 2502 - DNA, 25 bp, Homo sapiens
aaatttaccaagctgtgtgagttcc

SEQ ID NO: 2503 - DNA, 25 bp, Homo sapiens
aatttaccaagctgtgtgagttccc

SEQ ID NO: 2504 - DNA, 25 bp, Homo sapiens
atttaccaagctgtgtgagttcccg

SEQ ID NO: 2505 - DNA, 25 bp, Homo sapiens
tttaccaagctgtgtgagttcccga

SEQ ID NO: 2506 - DNA, 25 bp, Homo sapiens
ttaccaagctgtgtgagttcccgat

SEQ ID NO: 2507 - DNA, 25 bp, Homo sapiens
taccaagctgtgtgagttcccgatg

SEQ ID NO: 2508 - DNA, 25 bp, Homo sapiens
accaagctgtgtgagttcccgatgc

SEQ ID NO: 2509 - DNA, 25 bp, Homo sapiens
ccaagctgtgtgagttcccgatgct

SEQ ID NO: 2510 - DNA, 25 bp, Homo sapiens
caagctgtgtgagttcccgatgctg

SEQ ID NO: 2511 - DNA, 25 bp, Homo sapiens
aagctgtgtgagttcccgatgctgg

SEQ ID NO: 2512 - DNA, 25 bp, Homo sapiens
agctgtgtgagttcccgatgctgga

SEQ ID NO: 2513 - DNA, 25 bp, Homo sapiens
gctgtgtgagttcccgatgctggaa

SEQ ID NO: 2514 - DNA, 25 bp, Homo sapiens
ctgtgtgagttcccgatgctggaaa

SEQ ID NO: 2515 - DNA, 25 bp, Homo sapiens
tgtgtgagttcccgatgctggaaac

SEQ ID NO: 2516 - DNA, 25 bp, Homo sapiens
gtgtgagttcccgatgctggaaacc

SEQ ID NO: 2517 - DNA, 25 bp, Homo sapiens
tgtgagttcccgatgctggaaaccg

SEQ ID NO: 2518 - DNA, 25 bp, Homo sapiens
gtgagttcccgatgctggaaaccgg

SEQ ID NO: 2519 - DNA, 25 bp, Homo sapiens
tgagttcccgatgctggaaaccggc

SEQ ID NO: 2520 - DNA, 25 bp, Homo sapiens
gagttcccgatgctggaaaccggcc

SEQ ID NO: 2521 - DNA, 25 bp, Homo sapiens
agttcccgatgctggaaaccggcct

SEQ ID NO: 2522 - DNA, 25 bp, Homo sapiens
gttcccgatgctggaaaccggcctg

SEQ ID NO: 2523 - DNA, 25 bp, Homo sapiens
ttcccgatgctggaaaccggcctgt

SEQ ID NO: 2524 - DNA, 25 bp, Homo sapiens
tcccgatgctggaaaccggcctgtt

SEQ ID NO: 2525 - DNA, 25 bp, Homo sapiens
cccgatgctggaaaccggcctgttt

SEQ ID NO: 2526 - DNA, 25 bp, Homo sapiens
ccgatgctggaaaccggcctgtttt

SEQ ID NO: 2527 - DNA, 25 bp, Homo sapiens
cgatgctggaaaccggcctgttttt

SEQ ID NO: 2528 - DNA, 25 bp, Homo sapiens
gatgctggaaaccggcctgtttttc

SEQ ID NO: 2529 - DNA, 25 bp, Homo sapiens
atgctggaaaccggcctgtttttcc

SEQ ID NO: 2530 - DNA, 25 bp, Homo sapiens
tgctggaaaccggcctgtttttcct

SEQ ID NO: 2531 - DNA, 25 bp, Homo sapiens
gctggaaaccggcctgtttttcctg

SEQ ID NO: 2532 - DNA, 25 bp, Homo sapiens
ctggaaaccggcctgtttttcctgc

SEQ ID NO: 2533 - DNA, 25 bp, Homo sapiens
tggaaaccggcctgtttttcctgct

SEQ ID NO: 2534 - DNA, 25 bp, Homo sapiens
ggaaaccggcctgtttttcctgctt

SEQ ID NO: 2535 - DNA, 25 bp, Homo sapiens
gaaaccggcctgtttttcctgcttt

SEQ ID NO: 2536 - DNA, 25 bp, Homo sapiens
aaaccggcctgtttttcctgctttc

SEQ ID NO: 2537 - DNA, 25 bp, Homo sapiens
aaccggcctgtttttcctgctttct

SEQ ID NO: 2538 - DNA, 25 bp, Homo sapiens
accggcctgtttttcctgctttctt

SEQ ID NO: 2539 - DNA, 25 bp, Homo sapiens
ccggcctgtttttcctgctttcttg

SEQ ID NO: 2540 - DNA, 25 bp, Homo sapiens
cggcctgtttttcctgctttcttgg

SEQ ID NO: 2541 - DNA, 25 bp, Homo sapiens
ggcctgtttttcctgctttcttgga

SEQ ID NO: 2542 - DNA, 25 bp, Homo sapiens
gcctgtttttcctgctttcttggag

SEQ ID NO: 2543 - DNA, 25 bp, Homo sapiens
cctgtttttcctgctttcttggagt

SEQ ID NO: 2544 - DNA, 25 bp, Homo sapiens
ctgtttttcctgctttcttggagtg

SEQ ID NO: 2545 - DNA, 25 bp, Homo sapiens
tgtttttcctgctttcttggagtgc

SEQ ID NO: 2546 - DNA, 25 bp, Homo sapiens
gtttttcctgctttcttggagtgcc

SEQ ID NO: 2547 - DNA, 25 bp, Homo sapiens
tttttcctgctttcttggagtgcct

SEQ ID NO: 2548 - DNA, 25 bp, Homo sapiens
ttttcctgctttcttggagtgcctt

SEQ ID NO: 2549 - DNA, 25 bp, Homo sapiens
tttcctgctttcttggagtgccttc

SEQ ID NO: 2550 - DNA, 25 bp, Homo sapiens
ttcctgctttcttggagtgccttcc

SEQ ID NO: 2551 - DNA, 25 bp, Homo sapiens
tcctgctttcttggagtgccttcct

SEQ ID NO: 2552 - DNA, 25 bp, Homo sapiens
cctgctttcttggagtgccttcctg

SEQ ID NO: 2553 - DNA, 25 bp, Homo sapiens
ctgctttcttggagtgccttcctgt

SEQ ID NO: 2554 - DNA, 25 bp, Homo sapiens
tgctttcttggagtgccttcctgtc

SEQ ID NO: 2555 - DNA, 25 bp, Homo sapiens
gctttcttggagtgccttcctgtct

SEQ ID NO: 2556 - DNA, 25 bp, Homo sapiens
ctttcttggagtgccttcctgtctg

SEQ ID NO: 2557 - DNA, 25 bp, Homo sapiens
tttcttggagtgccttcctgtctgc

SEQ ID NO: 2558 - DNA, 25 bp, Homo sapiens
ttcttggagtgccttcctgtctgcc

SEQ ID NO: 2559 - DNA, 25 bp, Homo sapiens
tcttggagtgccttcctgtctgccg

SEQ ID NO: 2560 - DNA, 25 bp, Homo sapiens
cttggagtgccttcctgtctgccga

SEQ ID NO: 2561 - DNA, 25 bp, Homo sapiens
ttggagtgccttcctgtctgccgag

SEQ ID NO: 2562 - DNA, 25 bp, Homo sapiens
tggagtgccttcctgtctgccgagg

SEQ ID NO: 2563 - DNA, 25 bp, Homo sapiens
ggagtgccttcctgtctgccgaggc

SEQ ID NO: 2564 - DNA, 25 bp, Homo sapiens
gagtgccttcctgtctgccgaggct

SEQ ID NO: 2565 - DNA, 25 bp, Homo sapiens
agtgccttcctgtctgccgaggctg

SEQ ID NO: 2566 - DNA, 25 bp, Homo sapiens
gtgccttcctgtctgccgaggctgc

SEQ ID NO: 2567 - DNA, 25 bp, Homo sapiens
tgccttcctgtctgccgaggctgcc

SEQ ID NO: 2568 - DNA, 25 bp, Homo sapiens
gccttcctgtctgccgaggctgccg

SEQ ID NO: 2569 - DNA, 25 bp, Homo sapiens
ccttcctgtctgccgaggctgccgg

SEQ ID NO: 2570 - DNA, 25 bp, Homo sapiens
cttcctgtctgccgaggctgccggc

SEQ ID NO: 2571 - DNA, 25 bp, Homo sapiens
ttcctgtctgccgaggctgccggcc

SEQ ID NO: 2572 - DNA, 25 bp, Homo sapiens
tcctgtctgccgaggctgccggcct

SEQ ID NO: 2573 - DNA, 25 bp, Homo sapiens
cctgtctgccgaggctgccggccta

SEQ ID NO: 2574 - DNA, 25 bp, Homo sapiens
ctgtctgccgaggctgccggcctaa

SEQ ID NO: 2575 - DNA, 25 bp, Homo sapiens
tgtctgccgaggctgccggcctaac

SEQ ID NO: 2576 - DNA, 25 bp, Homo sapiens
gtctgccgaggctgccggcctaaca

SEQ ID NO: 2577 - DNA, 25 bp, Homo sapiens
tctgccgaggctgccggcctaacag

SEQ ID NO: 2578 - DNA, 25 bp, Homo sapiens
ctgccgaggctgccggcctaacagg

SEQ ID NO: 2579 - DNA, 25 bp, Homo sapiens
tgccgaggctgccggcctaacaggg

SEQ ID NO: 2580 - DNA, 25 bp, Homo sapiens
gccgaggctgccggcctaacaggga

SEQ ID NO: 2581 - DNA, 25 bp, Homo sapiens
ccgaggctgccggcctaacagggat

SEQ ID NO: 2582 - DNA, 25 bp, Homo sapiens
cgaggctgccggcctaacagggata

SEQ ID NO: 2583 - DNA, 25 bp, Homo sapiens
gaggctgccggcctaacagggatag

SEQ ID NO: 2584 - DNA, 25 bp, Homo sapiens
aggctgccggcctaacagggatagt

SEQ ID NO: 2585 - DNA, 25 bp, Homo sapiens
ggctgccggcctaacagggatagtt

SEQ ID NO: 2586 - DNA, 25 bp, Homo sapiens
gctgccggcctaacagggatagttg

SEQ ID NO: 2587 - DNA, 25 bp, Homo sapiens
ctgccggcctaacagggatagttgc

SEQ ID NO: 2588 - DNA, 25 bp, Homo sapiens
tgccggcctaacagggatagttgct

SEQ ID NO: 2589 - DNA, 25 bp, Homo sapiens
gccggcctaacagggatagttgctg

SEQ ID NO: 2590 - DNA, 25 bp, Homo sapiens
ccggcctaacagggatagttgctgt

SEQ ID NO: 2591 - DNA, 25 bp, Homo sapiens
cggcctaacagggatagttgctgtt

SEQ ID NO: 2592 - DNA, 25 bp, Homo sapiens
ggcctaacagggatagttgctgttc

SEQ ID NO: 2593 - DNA, 25 bp, Homo sapiens
gcctaacagggatagttgctgttct

SEQ ID NO: 2594 - DNA, 25 bp, Homo sapiens
cctaacagggatagttgctgttctc

SEQ ID NO: 2595 - DNA, 25 bp, Homo sapiens
ctaacagggatagttgctgttctct

SEQ ID NO: 2596 - DNA, 25 bp, Homo sapiens
taacagggatagttgctgttctctt

SEQ ID NO: 2597 - DNA, 25 bp, Homo sapiens
aacagggatagttgctgttctcttc

SEQ ID NO: 2598 - DNA, 25 bp, Homo sapiens
acagggatagttgctgttctcttct

SEQ ID NO: 2599 - DNA, 25 bp, Homo sapiens
cagggatagttgctgttctcttctg

SEQ ID NO: 2600 - DNA, 25 bp, Homo sapiens
agggatagttgctgttctcttctgt

SEQ ID NO: 2601 - DNA, 25 bp, Homo sapiens
gggatagttgctgttctcttctgtg

SEQ ID NO: 2602 - DNA, 25 bp, Homo sapiens
ggatagttgctgttctcttctgtgg

SEQ ID NO: 2603 - DNA, 25 bp, Homo sapiens
gatagttgctgttctcttctgtgga

SEQ ID NO: 2604 - DNA, 25 bp, Homo sapiens
atagttgctgttctcttctgtggag

SEQ ID NO: 2605 - DNA, 25 bp, Homo sapiens
tagttgctgttctcttctgtggagt

SEQ ID NO: 2606 - DNA, 25 bp, Homo sapiens
agttgctgttctcttctgtggagtc

SEQ ID NO: 2607 - DNA, 25 bp, Homo sapiens
gttgctgttctcttctgtggagtca

SEQ ID NO: 2608 - DNA, 25 bp, Homo sapiens
ttgctgttctcttctgtggagtcac

SEQ ID NO: 2609 - DNA, 25 bp, Homo sapiens
tgctgttctcttctgtggagtcaca

SEQ ID NO: 2610 - DNA, 25 bp, Homo sapiens
gctgttctcttctgtggagtcacac

SEQ ID NO: 2611 - DNA, 25 bp, Homo sapiens
ctgttctcttctgtggagtcacaca

SEQ ID NO: 2612 - DNA, 25 bp, Homo sapiens
tgttctcttctgtggagtcacacaa

SEQ ID NO: 2613 - DNA, 25 bp, Homo sapiens
gttctcttctgtggagtcacacaag

SEQ ID NO: 2614 - DNA, 25 bp, Homo sapiens
ttctcttctgtggagtcacacaagc

SEQ ID NO: 2615 - DNA, 25 bp, Homo sapiens
tctcttctgtggagtcacacaagca

SEQ ID NO: 2616 - DNA, 25 bp, Homo sapiens
ctcttctgtggagtcacacaagcac

SEQ ID NO: 2617 - DNA, 25 bp, Homo sapiens
tcttctgtggagtcacacaagcaca

SEQ ID NO: 2618 - DNA, 25 bp, Homo sapiens
cttctgtggagtcacacaagcacat

SEQ ID NO: 2619 - DNA, 25 bp, Homo sapiens
ttctgtggagtcacacaagcacatt

SEQ ID NO: 2620 - DNA, 25 bp, Homo sapiens
tctgtggagtcacacaagcacatta

SEQ ID NO: 2621 - DNA, 25 bp, Homo sapiens
ctgtggagtcacacaagcacattat

SEQ ID NO: 2622 - DNA, 25 bp, Homo sapiens
tgtggagtcacacaagcacattata

SEQ ID NO: 2623 - DNA, 25 bp, Homo sapiens
gtggagtcacacaagcacattatac

SEQ ID NO: 2624 - DNA, 25 bp, Homo sapiens
tggagtcacacaagcacattatacc

SEQ ID NO: 2625 - DNA, 25 bp, Homo sapiens
ggagtcacacaagcacattatacct

SEQ ID NO: 2626 - DNA, 25 bp, Homo sapiens
gagtcacacaagcacattataccta

SEQ ID NO: 2627 - DNA, 25 bp, Homo sapiens
agtcacacaagcacattatacctac

SEQ ID NO: 2628 - DNA, 25 bp, Homo sapiens
gtcacacaagcacattatacctaca

SEQ ID NO: 2629 - DNA, 25 bp, Homo sapiens
tcacacaagcacattatacctacaa

SEQ ID NO: 2630 - DNA, 25 bp, Homo sapiens
cacacaagcacattatacctacaac

SEQ ID NO: 2631 - DNA, 25 bp, Homo sapiens
acacaagcacattatacctacaaca

SEQ ID NO: 2632 - DNA, 25 bp, Homo sapiens
cacaagcacattatacctacaacaa

SEQ ID NO: 2633 - DNA, 25 bp, Homo sapiens
acaagcacattatacctacaacaat

SEQ ID NO: 2634 - DNA, 25 bp, Homo sapiens
caagcacattatacctacaacaatc

SEQ ID NO: 2635 - DNA, 25 bp, Homo sapiens
aagcacattatacctacaacaatct

SEQ ID NO: 2636 - DNA, 25 bp, Homo sapiens
agcacattatacctacaacaatctg

SEQ ID NO: 2637 - DNA, 25 bp, Homo sapiens
gcacattatacctacaacaatctgt

SEQ ID NO: 2638 - DNA, 25 bp, Homo sapiens
cacattatacctacaacaatctgtc

SEQ ID NO: 2639 - DNA, 25 bp, Homo sapiens
acattatacctacaacaatctgtct

SEQ ID NO: 2640 - DNA, 25 bp, Homo sapiens
cattatacctacaacaatctgtctt

SEQ ID NO: 2641 - DNA, 25 bp, Homo sapiens
attatacctacaacaatctgtcttc

SEQ ID NO: 2642 - DNA, 25 bp, Homo sapiens
ttatacctacaacaatctgtcttcg

SEQ ID NO: 2643 - DNA, 25 bp, Homo sapiens
tatacctacaacaatctgtcttcgg

SEQ ID NO: 2644 - DNA, 25 bp, Homo sapiens
atacctacaacaatctgtcttcgga

SEQ ID NO: 2645 - DNA, 25 bp, Homo sapiens
tacctacaacaatctgtcttcggat

SEQ ID NO: 2646 - DNA, 25 bp, Homo sapiens
acctacaacaatctgtcttcggatt

SEQ ID NO: 2647 - DNA, 25 bp, Homo sapiens
cctacaacaatctgtcttcggattc

SEQ ID NO: 2648 - DNA, 25 bp, Homo sapiens
ctacaacaatctgtcttcggattcc

SEQ ID NO: 2649 - DNA, 25 bp, Homo sapiens
tacaacaatctgtcttcggattcca

SEQ ID NO: 2650 - DNA, 25 bp, Homo sapiens
acaacaatctgtcttcggattccaa

SEQ ID NO: 2651 - DNA, 25 bp, Homo sapiens
caacaatctgtcttcggattccaaa

SEQ ID NO: 2652 - DNA, 25 bp, Homo sapiens
aacaatctgtcttcggattccaaaa

SEQ ID NO: 2653 - DNA, 25 bp, Homo sapiens
acaatctgtcttcggattccaaaat

SEQ ID NO: 2654 - DNA, 25 bp, Homo sapiens
caatctgtcttcggattccaaaata

SEQ ID NO: 2655 - DNA, 25 bp, Homo sapiens
aatctgtcttcggattccaaaataa

SEQ ID NO: 2656 - DNA, 25 bp, Homo sapiens
atctgtcttcggattccaaaataag

SEQ ID NO: 2657 - DNA, 25 bp, Homo sapiens
tctgtcttcggattccaaaataaga

SEQ ID NO: 2658 - DNA, 25 bp, Homo sapiens
ctgtcttcggattccaaaataagaa

SEQ ID NO: 2659 - DNA, 25 bp, Homo sapiens
tgtcttcggattccaaaataagaac

SEQ ID NO: 2660 - DNA, 25 bp, Homo sapiens
gtcttcggattccaaaataagaact

SEQ ID NO: 2661 - DNA, 25 bp, Homo sapiens
tcttcggattccaaaataagaacta

SEQ ID NO: 2662 - DNA, 25 bp, Homo sapiens
cttcggattccaaaataagaactaa

SEQ ID NO: 2663 - DNA, 25 bp, Homo sapiens
ttcggattccaaaataagaactaaa

SEQ ID NO: 2664 - DNA, 25 bp, Homo sapiens
tcggattccaaaataagaactaaac

SEQ ID NO: 2665 - DNA, 25 bp, Homo sapiens
cggattccaaaataagaactaaaca

SEQ ID NO: 2666 - DNA, 25 bp, Homo sapiens
ggattccaaaataagaactaaacag

SEQ ID NO: 2667 - DNA, 25 bp, Homo sapiens
gattccaaaataagaactaaacagt

SEQ ID NO: 2668 - DNA, 25 bp, Homo sapiens
attccaaaataagaactaaacagtt

SEQ ID NO: 2669 - DNA, 25 bp, Homo sapiens
ttccaaaataagaactaaacagttg

SEQ ID NO: 2670 - DNA, 25 bp, Homo sapiens
tccaaaataagaactaaacagttgt

SEQ ID NO: 2671 - DNA, 25 bp, Homo sapiens
ccaaaataagaactaaacagttgtt

SEQ ID NO: 2672 - DNA, 25 bp, Homo sapiens
caaaataagaactaaacagttgttt

SEQ ID NO: 2673 - DNA, 25 bp, Homo sapiens
aaaataagaactaaacagttgtttg

SEQ ID NO: 2674 - DNA, 25 bp, Homo sapiens
aaataagaactaaacagttgtttga

SEQ ID NO: 2675 - DNA, 25 bp, Homo sapiens
aataagaactaaacagttgtttgaa

SEQ ID NO: 2676 - DNA, 25 bp, Homo sapiens
ataagaactaaacagttgtttgaat

SEQ ID NO: 2677 - DNA, 25 bp, Homo sapiens
taagaactaaacagttgtttgaatt

SEQ ID NO: 2678 - DNA, 25 bp, Homo sapiens
aagaactaaacagttgtttgaattt

SEQ ID NO: 2679 - DNA, 25 bp, Homo sapiens
agaactaaacagttgtttgaattta

SEQ ID NO: 2680 - DNA, 25 bp, Homo sapiens
gaactaaacagttgtttgaatttat

SEQ ID NO: 2681 - DNA, 25 bp, Homo sapiens
aactaaacagttgtttgaatttatg

SEQ ID NO: 2682 - DNA, 25 bp, Homo sapiens
actaaacagttgtttgaatttatga

SEQ ID NO: 2683 - DNA, 25 bp, Homo sapiens
ctaaacagttgtttgaatttatgaa

SEQ ID NO: 2684 - DNA, 25 bp, Homo sapiens
taaacagttgtttgaatttatgaac

SEQ ID NO: 2685 - DNA, 25 bp, Homo sapiens
aaacagttgtttgaatttatgaact

SEQ ID NO: 2686 - DNA, 25 bp, Homo sapiens
aacagttgtttgaatttatgaactt

SEQ ID NO: 2687 - DNA, 25 bp, Homo sapiens
acagttgtttgaatttatgaacttt

SEQ ID NO: 2688 - DNA, 25 bp, Homo sapiens
cagttgtttgaatttatgaactttt

SEQ ID NO: 2689 - DNA, 25 bp, Homo sapiens
agttgtttgaatttatgaacttttt

SEQ ID NO: 2690 - DNA, 25 bp, Homo sapiens
gttgtttgaatttatgaactttttg

SEQ ID NO: 2691 - DNA, 25 bp, Homo sapiens
ttgtttgaatttatgaactttttgg

SEQ ID NO: 2692 - DNA, 25 bp, Homo sapiens
tgtttgaatttatgaactttttggc

SEQ ID NO: 2693 - DNA, 25 bp, Homo sapiens
gtttgaatttatgaactttttggcg

SEQ ID NO: 2694 - DNA, 25 bp, Homo sapiens
tttgaatttatgaactttttggcgg

SEQ ID NO: 2695 - DNA, 25 bp, Homo sapiens
ttgaatttatgaactttttggcgga

SEQ ID NO: 2696 - DNA, 25 bp, Homo sapiens
tgaatttatgaactttttggcggag

SEQ ID NO: 2697 - DNA, 25 bp, Homo sapiens
gaatttatgaactttttggcggaga

SEQ ID NO: 2698 - DNA, 25 bp, Homo sapiens
aatttatgaactttttggcggagaa

SEQ ID NO: 2699 - DNA, 25 bp, Homo sapiens
atttatgaactttttggcggagaac

SEQ ID NO: 2700 - DNA, 25 bp, Homo sapiens
tttatgaactttttggcggagaacg

SEQ ID NO: 2701 - DNA, 25 bp, Homo sapiens
ttatgaactttttggcggagaacgt

SEQ ID NO: 2702 - DNA, 25 bp, Homo sapiens
tatgaactttttggcggagaacgtc

SEQ ID NO: 2703 - DNA, 25 bp, Homo sapiens
atgaactttttggcggagaacgtca

SEQ ID NO: 2704 - DNA, 25 bp, Homo sapiens
tgaactttttggcggagaacgtcat

SEQ ID NO: 2705 - DNA, 25 bp, Homo sapiens
gaactttttggcggagaacgtcatc

SEQ ID NO: 2706 - DNA, 25 bp, Homo sapiens
aactttttggcggagaacgtcatct

SEQ ID NO: 2707 - DNA, 25 bp, Homo sapiens
actttttggcggagaacgtcatctt

SEQ ID NO: 2708 - DNA, 25 bp, Homo sapiens
ctttttggcggagaacgtcatcttc

SEQ ID NO: 2709 - DNA, 25 bp, Homo sapiens
tttttggcggagaacgtcatcttct

SEQ ID NO: 2710 - DNA, 25 bp, Homo sapiens
ttttggcggagaacgtcatcttctg

SEQ ID NO: 2711 - DNA, 25 bp, Homo sapiens
tttggcggagaacgtcatcttctgt

SEQ ID NO: 2712 - DNA, 25 bp, Homo sapiens
ttggcggagaacgtcatcttctgtt

SEQ ID NO: 2713 - DNA, 25 bp, Homo sapiens
tggcggagaacgtcatcttctgtta

SEQ ID NO: 2714 - DNA, 25 bp, Homo sapiens
ggcggagaacgtcatcttctgttac

SEQ ID NO: 2715 - DNA, 25 bp, Homo sapiens
gcggagaacgtcatcttctgttaca

SEQ ID NO: 2716 - DNA, 25 bp, Homo sapiens
cggagaacgtcatcttctgttacat

SEQ ID NO: 2717 - DNA, 25 bp, Homo sapiens
ggagaacgtcatcttctgttacatg

SEQ ID NO: 2718 - DNA, 25 bp, Homo sapiens
gagaacgtcatcttctgttacatgg

SEQ ID NO: 2719 - DNA, 25 bp, Homo sapiens
agaacgtcatcttctgttacatggg

SEQ ID NO: 2720 - DNA, 25 bp, Homo sapiens
gaacgtcatcttctgttacatgggc

SEQ ID NO: 2721 - DNA, 25 bp, Homo sapiens
aacgtcatcttctgttacatgggcc

SEQ ID NO: 2722 - DNA, 25 bp, Homo sapiens
acgtcatcttctgttacatgggcct

SEQ ID NO: 2723 - DNA, 25 bp, Homo sapiens
cgtcatcttctgttacatgggcctg

SEQ ID NO: 2724 - DNA, 25 bp, Homo sapiens
gtcatcttctgttacatgggcctgg

SEQ ID NO: 2725 - DNA, 25 bp, Homo sapiens
tcatcttctgttacatgggcctggc

SEQ ID NO: 2726 - DNA, 25 bp, Homo sapiens
catcttctgttacatgggcctggca

SEQ ID NO: 2727 - DNA, 25 bp, Homo sapiens
atcttctgttacatgggcctggcac

SEQ ID NO: 2728 - DNA, 25 bp, Homo sapiens
tcttctgttacatgggcctggcact

SEQ ID NO: 2729 - DNA, 25 bp, Homo sapiens
cttctgttacatgggcctggcactg

SEQ ID NO: 2730 - DNA, 25 bp, Homo sapiens
ttctgttacatgggcctggcactgt

SEQ ID NO: 2731 - DNA, 25 bp, Homo sapiens
tctgttacatgggcctggcactgtt

SEQ ID NO: 2732 - DNA, 25 bp, Homo sapiens
ctgttacatgggcctggcactgttc

SEQ ID NO: 2733 - DNA, 25 bp, Homo sapiens
tgttacatgggcctggcactgttca

SEQ ID NO: 2734 - DNA, 25 bp, Homo sapiens
gttacatgggcctggcactgttcac

SEQ ID NO: 2735 - DNA, 25 bp, Homo sapiens
ttacatgggcctggcactgttcacg

SEQ ID NO: 2736 - DNA, 25 bp, Homo sapiens
tacatgggcctggcactgttcacgt

SEQ ID NO: 2737 - DNA, 25 bp, Homo sapiens
acatgggcctggcactgttcacgtt

SEQ ID NO: 2738 - DNA, 25 bp, Homo sapiens
catgggcctggcactgttcacgttc

SEQ ID NO: 2739 - DNA, 25 bp, Homo sapiens
atgggcctggcactgttcacgttcc

SEQ ID NO: 2740 - DNA, 25 bp, Homo sapiens
tgggcctggcactgttcacgttcca

SEQ ID NO: 2741 - DNA, 25 bp, Homo sapiens
gggcctggcactgttcacgttccag

SEQ ID NO: 2742 - DNA, 25 bp, Homo sapiens
ggcctggcactgttcacgttccaga

SEQ ID NO: 2743 - DNA, 25 bp, Homo sapiens
gcctggcactgttcacgttccagaa

SEQ ID NO: 2744 - DNA, 25 bp, Homo sapiens
cctggcactgttcacgttccagaat

SEQ ID NO: 2745 - DNA, 25 bp, Homo sapiens
ctggcactgttcacgttccagaatc

SEQ ID NO: 2746 - DNA, 25 bp, Homo sapiens
tggcactgttcacgttccagaatca

SEQ ID NO: 2747 - DNA, 25 bp, Homo sapiens
ggcactgttcacgttccagaatcat

SEQ ID NO: 2748 - DNA, 25 bp, Homo sapiens
gcactgttcacgttccagaatcata

SEQ ID NO: 2749 - DNA, 25 bp, Homo sapiens
cactgttcacgttccagaatcatat

SEQ ID NO: 2750 - DNA, 25 bp, Homo sapiens
actgttcacgttccagaatcatatc

SEQ ID NO: 2751 - DNA, 25 bp, Homo sapiens
ctgttcacgttccagaatcatatct

SEQ ID NO: 2752 - DNA, 25 bp, Homo sapiens
tgttcacgttccagaatcatatctt

SEQ ID NO: 2753 - DNA, 25 bp, Homo sapiens
gttcacgttccagaatcatatcttt

SEQ ID NO: 2754 - DNA, 25 bp, Homo sapiens
ttcacgttccagaatcatatcttta

SEQ ID NO: 2755 - DNA, 25 bp, Homo sapiens
tcacgttccagaatcatatctttaa

SEQ ID NO: 2756 - DNA, 25 bp, Homo sapiens
cacgttccagaatcatatctttaat

SEQ ID NO: 2757 - DNA, 25 bp, Homo sapiens
acgttccagaatcatatctttaatg

SEQ ID NO: 2758 - DNA, 25 bp, Homo sapiens
cgttccagaatcatatctttaatgc

SEQ ID NO: 2759 - DNA, 25 bp, Homo sapiens
gttccagaatcatatctttaatgct

SEQ ID NO: 2760 - DNA, 25 bp, Homo sapiens
ttccagaatcatatctttaatgctc

SEQ ID NO: 2761 - DNA, 25 bp, Homo sapiens
tccagaatcatatctttaatgctct

SEQ ID NO: 2762 - DNA, 25 bp, Homo sapiens
ccagaatcatatctttaatgctctt

SEQ ID NO: 2763 - DNA, 25 bp, Homo sapiens
cagaatcatatctttaatgctcttt

SEQ ID NO: 2764 - DNA, 25 bp, Homo sapiens
agaatcatatctttaatgctctttt

SEQ ID NO: 2765 - DNA, 25 bp, Homo sapiens
gaatcatatctttaatgctcttttt

SEQ ID NO: 2766 - DNA, 25 bp, Homo sapiens
aatcatatctttaatgctcttttta

SEQ ID NO: 2767 - DNA, 25 bp, Homo sapiens
atcatatctttaatgctctttttat

SEQ ID NO: 2768 - DNA, 25 bp, Homo sapiens
tcatatctttaatgctctttttata

SEQ ID NO: 2769 - DNA, 25 bp, Homo sapiens
catatctttaatgctctttttatac

SEQ ID NO: 2770 - DNA, 25 bp, Homo sapiens
atatctttaatgctctttttatact

SEQ ID NO: 2771 - DNA, 25 bp, Homo sapiens
tatctttaatgctctttttatactt

SEQ ID NO: 2772 - DNA, 25 bp, Homo sapiens
atctttaatgctctttttatacttg

SEQ ID NO: 2773 - DNA, 25 bp, Homo sapiens
tctttaatgctctttttatacttgg

SEQ ID NO: 2774 - DNA, 25 bp, Homo sapiens
ctttaatgctctttttatacttgga

SEQ ID NO: 2775 - DNA, 25 bp, Homo sapiens
tttaatgctctttttatacttggag

SEQ ID NO: 2776 - DNA, 25 bp, Homo sapiens
ttaatgctctttttatacttggagc

SEQ ID NO: 2777 - DNA, 25 bp, Homo sapiens
taatgctctttttatacttggagcc

SEQ ID NO: 2778 - DNA, 25 bp, Homo sapiens
aatgctctttttatacttggagcct

SEQ ID NO: 2779 - DNA, 25 bp, Homo sapiens
atgctctttttatacttggagcctt

SEQ ID NO: 2780 - DNA, 25 bp, Homo sapiens
tgctctttttatacttggagccttt

SEQ ID NO: 2781 - DNA, 25 bp, Homo sapiens
gctctttttatacttggagcctttc

SEQ ID NO: 2782 - DNA, 25 bp, Homo sapiens
ctctttttatacttggagcctttct

SEQ ID NO: 2783 - DNA, 25 bp, Homo sapiens
tctttttatacttggagcctttcta

SEQ ID NO: 2784 - DNA, 25 bp, Homo sapiens
ctttttatacttggagcctttctag

SEQ ID NO: 2785 - DNA, 25 bp, Homo sapiens
tttttatacttggagcctttctagc

SEQ ID NO: 2786 - DNA, 25 bp, Homo sapiens
ttttatacttggagcctttctagca

SEQ ID NO: 2787 - DNA, 25 bp, Homo sapiens
tttatacttggagcctttctagcaa

SEQ ID NO: 2788 - DNA, 25 bp, Homo sapiens
ttatacttggagcctttctagcaat

SEQ ID NO: 2789 - DNA, 25 bp, Homo sapiens
tatacttggagcctttctagcaatt

SEQ ID NO: 2790 - DNA, 25 bp, Homo sapiens
atacttggagcctttctagcaattt

SEQ ID NO: 2791 - DNA, 25 bp, Homo sapiens
tacttggagcctttctagcaatttt

SEQ ID NO: 2792 - DNA, 25 bp, Homo sapiens
acttggagcctttctagcaattttt

SEQ ID NO: 2793 - DNA, 25 bp, Homo sapiens
cttggagcctttctagcaatttttg

SEQ ID NO: 2794 - DNA, 25 bp, Homo sapiens
ttggagcctttctagcaatttttgt

SEQ ID NO: 2795 - DNA, 25 bp, Homo sapiens
tggagcctttctagcaatttttgtt

SEQ ID NO: 2796 - DNA, 25 bp, Homo sapiens
ggagcctttctagcaatttttgttg

SEQ ID NO: 2797 - DNA, 25 bp, Homo sapiens
gagcctttctagcaatttttgttgc

SEQ ID NO: 2798 - DNA, 25 bp, Homo sapiens
agcctttctagcaatttttgttgcc

SEQ ID NO: 2799 - DNA, 25 bp, Homo sapiens
gcctttctagcaatttttgttgcca

SEQ ID NO: 2800 - DNA, 25 bp, Homo sapiens
cctttctagcaatttttgttgccag

SEQ ID NO: 2801 - DNA, 25 bp, Homo sapiens
ctttctagcaatttttgttgccaga

SEQ ID NO: 2802 - DNA, 25 bp, Homo sapiens
tttctagcaatttttgttgccagag

SEQ ID NO: 2803 - DNA, 25 bp, Homo sapiens
ttctagcaatttttgttgccagagc

SEQ ID NO: 2804 - DNA, 25 bp, Homo sapiens
tctagcaatttttgttgccagagcc

SEQ ID NO: 2805 - DNA, 25 bp, Homo sapiens
ctagcaatttttgttgccagagcct

SEQ ID NO: 2806 - DNA, 25 bp, Homo sapiens
tagcaatttttgttgccagagcctg

SEQ ID NO: 2807 - DNA, 25 bp, Homo sapiens
agcaatttttgttgccagagcctgc

SEQ ID NO: 2808 - DNA, 25 bp, Homo sapiens
gcaatttttgttgccagagcctgca

SEQ ID NO: 2809 - DNA, 25 bp, Homo sapiens
caatttttgttgccagagcctgcaa

SEQ ID NO: 2810 - DNA, 25 bp, Homo sapiens
aatttttgttgccagagcctgcaac

SEQ ID NO: 2811 - DNA, 25 bp, Homo sapiens
atttttgttgccagagcctgcaaca

SEQ ID NO: 2812 - DNA, 25 bp, Homo sapiens
tttttgttgccagagcctgcaacat

SEQ ID NO: 2813 - DNA, 25 bp, Homo sapiens
ttttgttgccagagcctgcaacata

SEQ ID NO: 2814 - DNA, 25 bp, Homo sapiens
tttgttgccagagcctgcaacatat

SEQ ID NO: 2815 - DNA, 25 bp, Homo sapiens
ttgttgccagagcctgcaacatata

SEQ ID NO: 2816 - DNA, 25 bp, Homo sapiens
tgttgccagagcctgcaacatatat

SEQ ID NO: 2817 - DNA, 25 bp, Homo sapiens
gttgccagagcctgcaacatatatc

SEQ ID NO: 2818 - DNA, 25 bp, Homo sapiens
ttgccagagcctgcaacatatatcc

SEQ ID NO: 2819 - DNA, 25 bp, Homo sapiens
tgccagagcctgcaacatatatccc

SEQ ID NO: 2820 - DNA, 25 bp, Homo sapiens
gccagagcctgcaacatatatcccc

SEQ ID NO: 2821 - DNA, 25 bp, Homo sapiens
ccagagcctgcaacatatatcccct

SEQ ID NO: 2822 - DNA, 25 bp, Homo sapiens
cagagcctgcaacatatatcccctc

SEQ ID NO: 2823 - DNA, 25 bp, Homo sapiens
agagcctgcaacatatatcccctct

SEQ ID NO: 2824 - DNA, 25 bp, Homo sapiens
gagcctgcaacatatatcccctctc

SEQ ID NO: 2825 - DNA, 25 bp, Homo sapiens
agcctgcaacatatatcccctctcc

SEQ ID NO: 2826 - DNA, 25 bp, Homo sapiens
gcctgcaacatatatcccctctcct

SEQ ID NO: 2827 - DNA, 25 bp, Homo sapiens
cctgcaacatatatcccctctcctt

SEQ ID NO: 2828 - DNA, 25 bp, Homo sapiens
ctgcaacatatatcccctctccttc

SEQ ID NO: 2829 - DNA, 25 bp, Homo sapiens
tgcaacatatatcccctctccttcc

SEQ ID NO: 2830 - DNA, 25 bp, Homo sapiens
gcaacatatatcccctctccttcct

SEQ ID NO: 2831 - DNA, 25 bp, Homo sapiens
caacatatatcccctctccttcctc

SEQ ID NO: 2832 - DNA, 25 bp, Homo sapiens
aacatatatcccctctccttcctcc

SEQ ID NO: 2833 - DNA, 25 bp, Homo sapiens
acatatatcccctctccttcctcct

SEQ ID NO: 2834 - DNA, 25 bp, Homo sapiens
catatatcccctctccttcctcctg

SEQ ID NO: 2835 - DNA, 25 bp, Homo sapiens
atatatcccctctccttcctcctga

SEQ ID NO: 2836 - DNA, 25 bp, Homo sapiens
tatatcccctctccttcctcctgaa

SEQ ID NO: 2837 - DNA, 25 bp, Homo sapiens
atatcccctctccttcctcctgaat

SEQ ID NO: 2838 - DNA, 25 bp, Homo sapiens
tatcccctctccttcctcctgaatc

SEQ ID NO: 2839 - DNA, 25 bp, Homo sapiens
atcccctctccttcctcctgaatct

SEQ ID NO: 2840 - DNA, 25 bp, Homo sapiens
tcccctctccttcctcctgaatcta

SEQ ID NO: 2841 - DNA, 25 bp, Homo sapiens
cccctctccttcctcctgaatctag

SEQ ID NO: 2842 - DNA, 25 bp, Homo sapiens
ccctctccttcctcctgaatctagg

SEQ ID NO: 2843 - DNA, 25 bp, Homo sapiens
cctctccttcctcctgaatctaggc

SEQ ID NO: 2844 - DNA, 25 bp, Homo sapiens
ctctccttcctcctgaatctaggcc

SEQ ID NO: 2845 - DNA, 25 bp, Homo sapiens
tctccttcctcctgaatctaggccg

SEQ ID NO: 2846 - DNA, 25 bp, Homo sapiens
ctccttcctcctgaatctaggccga

SEQ ID NO: 2847 - DNA, 25 bp, Homo sapiens
tccttcctcctgaatctaggccgaa

SEQ ID NO: 2848 - DNA, 25 bp, Homo sapiens
ccttcctcctgaatctaggccgaaa

SEQ ID NO: 2849 - DNA, 25 bp, Homo sapiens
cttcctcctgaatctaggccgaaaa

SEQ ID NO: 2850 - DNA, 25 bp, Homo sapiens
ttcctcctgaatctaggccgaaaac

SEQ ID NO: 2851 - DNA, 25 bp, Homo sapiens
tcctcctgaatctaggccgaaaaca

SEQ ID NO: 2852 - DNA, 25 bp, Homo sapiens
cctcctgaatctaggccgaaaacag

SEQ ID NO: 2853 - DNA, 25 bp, Homo sapiens
ctcctgaatctaggccgaaaacaga

SEQ ID NO: 2854 - DNA, 25 bp, Homo sapiens
tcctgaatctaggccgaaaacagaa

SEQ ID NO: 2855 - DNA, 25 bp, Homo sapiens
cctgaatctaggccgaaaacagaag

SEQ ID NO: 2856 - DNA, 25 bp, Homo sapiens
ctgaatctaggccgaaaacagaaga

SEQ ID NO: 2857 - DNA, 25 bp, Homo sapiens
tgaatctaggccgaaaacagaagat

SEQ ID NO: 2858 - DNA, 25 bp, Homo sapiens
gaatctaggccgaaaacagaagatc

SEQ ID NO: 2859 - DNA, 25 bp, Homo sapiens
aatctaggccgaaaacagaagatcc

SEQ ID NO: 2860 - DNA, 25 bp, Homo sapiens
atctaggccgaaaacagaagatccc

SEQ ID NO: 2861 - DNA, 25 bp, Homo sapiens
tctaggccgaaaacagaagatcccc

SEQ ID NO: 2862 - DNA, 25 bp, Homo sapiens
ctaggccgaaaacagaagatcccct

SEQ ID NO: 2863 - DNA, 25 bp, Homo sapiens
taggccgaaaacagaagatcccctg

SEQ ID NO: 2864 - DNA, 25 bp, Homo sapiens
aggccgaaaacagaagatcccctgg

SEQ ID NO: 2865 - DNA, 25 bp, Homo sapiens
ggccgaaaacagaagatcccctgga

SEQ ID NO: 2866 - DNA, 25 bp, Homo sapiens
gccgaaaacagaagatcccctggaa

SEQ ID NO: 2867 - DNA, 25 bp, Homo sapiens
ccgaaaacagaagatcccctggaac

SEQ ID NO: 2868 - DNA, 25 bp, Homo sapiens
cgaaaacagaagatcccctggaact

SEQ ID NO: 2869 - DNA, 25 bp, Homo sapiens
gaaaacagaagatcccctggaactt

SEQ ID NO: 2870 - DNA, 25 bp, Homo sapiens
aaaacagaagatcccctggaacttt

SEQ ID NO: 2871 - DNA, 25 bp, Homo sapiens
aaacagaagatcccctggaactttc

SEQ ID NO: 2872 - DNA, 25 bp, Homo sapiens
aacagaagatcccctggaactttca

SEQ ID NO: 2873 - DNA, 25 bp, Homo sapiens
acagaagatcccctggaactttcag

SEQ ID NO: 2874 - DNA, 25 bp, Homo sapiens
cagaagatcccctggaactttcagc

SEQ ID NO: 2875 - DNA, 25 bp, Homo sapiens
agaagatcccctggaactttcagca

SEQ ID NO: 2876 - DNA, 25 bp, Homo sapiens
gaagatcccctggaactttcagcac

SEQ ID NO: 2877 - DNA, 25 bp, Homo sapiens
aagatcccctggaactttcagcaca

SEQ ID NO: 2878 - DNA, 25 bp, Homo sapiens
agatcccctggaactttcagcacat

SEQ ID NO: 2879 - DNA, 25 bp, Homo sapiens
gatcccctggaactttcagcacatg

SEQ ID NO: 2880 - DNA, 25 bp, Homo sapiens
atcccctggaactttcagcacatga

SEQ ID NO: 2881 - DNA, 25 bp, Homo sapiens
tcccctggaactttcagcacatgat

SEQ ID NO: 2882 - DNA, 25 bp, Homo sapiens
cccctggaactttcagcacatgatg

SEQ ID NO: 2883 - DNA, 25 bp, Homo sapiens
ccctggaactttcagcacatgatga

SEQ ID NO: 2884 - DNA, 25 bp, Homo sapiens
cctggaactttcagcacatgatgat

SEQ ID NO: 2885 - DNA, 25 bp, Homo sapiens
ctggaactttcagcacatgatgatg

SEQ ID NO: 2886 - DNA, 25 bp, Homo sapiens
tggaactttcagcacatgatgatgt

SEQ ID NO: 2887 - DNA, 25 bp, Homo sapiens
ggaactttcagcacatgatgatgtt

SEQ ID NO: 2888 - DNA, 25 bp, Homo sapiens
gaactttcagcacatgatgatgttt

SEQ ID NO: 2889 - DNA, 25 bp, Homo sapiens
aactttcagcacatgatgatgtttt

SEQ ID NO: 2890 - DNA, 25 bp, Homo sapiens
actttcagcacatgatgatgttttc

SEQ ID NO: 2891 - DNA, 25 bp, Homo sapiens
ctttcagcacatgatgatgttttca

SEQ ID NO: 2892 - DNA, 25 bp, Homo sapiens
tttcagcacatgatgatgttttcag

SEQ ID NO: 2893 - DNA, 25 bp, Homo sapiens
ttcagcacatgatgatgttttcagg

SEQ ID NO: 2894 - DNA, 25 bp, Homo sapiens
tcagcacatgatgatgttttcaggt

SEQ ID NO: 2895 - DNA, 25 bp, Homo sapiens
cagcacatgatgatgttttcaggtt

SEQ ID NO: 2896 - DNA, 25 bp, Homo sapiens
agcacatgatgatgttttcaggttt

SEQ ID NO: 2897 - DNA, 25 bp, Homo sapiens
gcacatgatgatgttttcaggtttg

SEQ ID NO: 2898 - DNA, 25 bp, Homo sapiens
cacatgatgatgttttcaggtttgc

SEQ ID NO: 2899 - DNA, 25 bp, Homo sapiens
acatgatgatgttttcaggtttgcg

SEQ ID NO: 2900 - DNA, 25 bp, Homo sapiens
catgatgatgttttcaggtttgcga

SEQ ID NO: 2901 - DNA, 25 bp, Homo sapiens
atgatgatgttttcaggtttgcgag

SEQ ID NO: 2902 - DNA, 25 bp, Homo sapiens
tgatgatgttttcaggtttgcgagg

SEQ ID NO: 2903 - DNA, 25 bp, Homo sapiens
gatgatgttttcaggtttgcgagga

SEQ ID NO: 2904 - DNA, 25 bp, Homo sapiens
atgatgttttcaggtttgcgaggag

SEQ ID NO: 2905 - DNA, 25 bp, Homo sapiens
tgatgttttcaggtttgcgaggagc

SEQ ID NO: 2906 - DNA, 25 bp, Homo sapiens
gatgttttcaggtttgcgaggagcg

SEQ ID NO: 2907 - DNA, 25 bp, Homo sapiens
atgttttcaggtttgcgaggagcga

SEQ ID NO: 2908 - DNA, 25 bp, Homo sapiens
tgttttcaggtttgcgaggagcgat

SEQ ID NO: 2909 - DNA, 25 bp, Homo sapiens
gttttcaggtttgcgaggagcgatc

SEQ ID NO: 2910 - DNA, 25 bp, Homo sapiens
ttttcaggtttgcgaggagcgatcg

SEQ ID NO: 2911 - DNA, 25 bp, Homo sapiens
tttcaggtttgcgaggagcgatcgc

SEQ ID NO: 2912 - DNA, 25 bp, Homo sapiens
ttcaggtttgcgaggagcgatcgca

SEQ ID NO: 2913 - DNA, 25 bp, Homo sapiens
tcaggtttgcgaggagcgatcgcat

SEQ ID NO: 2914 - DNA, 25 bp, Homo sapiens
caggtttgcgaggagcgatcgcatt

SEQ ID NO: 2915 - DNA, 25 bp, Homo sapiens
aggtttgcgaggagcgatcgcattt

SEQ ID NO: 2916 - DNA, 25 bp, Homo sapiens
ggtttgcgaggagcgatcgcatttg

SEQ ID NO: 2917 - DNA, 25 bp, Homo sapiens
gtttgcgaggagcgatcgcatttgc

SEQ ID NO: 2918 - DNA, 25 bp, Homo sapiens
tttgcgaggagcgatcgcatttgcc

SEQ ID NO: 2919 - DNA, 25 bp, Homo sapiens
ttgcgaggagcgatcgcatttgcct

SEQ ID NO: 2920 - DNA, 25 bp, Homo sapiens
tgcgaggagcgatcgcatttgcctt

SEQ ID NO: 2921 - DNA, 25 bp, Homo sapiens
gcgaggagcgatcgcatttgcctta

SEQ ID NO: 2922 - DNA, 25 bp, Homo sapiens
cgaggagcgatcgcatttgccttag

SEQ ID NO: 2923 - DNA, 25 bp, Homo sapiens
gaggagcgatcgcatttgccttagc

SEQ ID NO: 2924 - DNA, 25 bp, Homo sapiens
aggagcgatcgcatttgccttagct

SEQ ID NO: 2925 - DNA, 25 bp, Homo sapiens
ggagcgatcgcatttgccttagcta

SEQ ID NO: 2926 - DNA, 25 bp, Homo sapiens
gagcgatcgcatttgccttagctat

SEQ ID NO: 2927 - DNA, 25 bp, Homo sapiens
agcgatcgcatttgccttagctatt

SEQ ID NO: 2928 - DNA, 25 bp, Homo sapiens
gcgatcgcatttgccttagctattc

SEQ ID NO: 2929 - DNA, 25 bp, Homo sapiens
cgatcgcatttgccttagctattcg

SEQ ID NO: 2930 - DNA, 25 bp, Homo sapiens
gatcgcatttgccttagctattcgg

SEQ ID NO: 2931 - DNA, 25 bp, Homo sapiens
atcgcatttgccttagctattcgga

SEQ ID NO: 2932 - DNA, 25 bp, Homo sapiens
tcgcatttgccttagctattcggaa

SEQ ID NO: 2933 - DNA, 25 bp, Homo sapiens
cgcatttgccttagctattcggaac

SEQ ID NO: 2934 - DNA, 25 bp, Homo sapiens
gcatttgccttagctattcggaaca

SEQ ID NO: 2935 - DNA, 25 bp, Homo sapiens
catttgccttagctattcggaacac

SEQ ID NO: 2936 - DNA, 25 bp, Homo sapiens
atttgccttagctattcggaacaca

SEQ ID NO: 2937 - DNA, 25 bp, Homo sapiens
tttgccttagctattcggaacacag

SEQ ID NO: 2938 - DNA, 25 bp, Homo sapiens
ttgccttagctattcggaacacaga

SEQ ID NO: 2939 - DNA, 25 bp, Homo sapiens
tgccttagctattcggaacacagaa

SEQ ID NO: 2940 - DNA, 25 bp, Homo sapiens
gccttagctattcggaacacagaat

SEQ ID NO: 2941 - DNA, 25 bp, Homo sapiens
ccttagctattcggaacacagaatc

SEQ ID NO: 2942 - DNA, 25 bp, Homo sapiens
cttagctattcggaacacagaatct

SEQ ID NO: 2943 - DNA, 25 bp, Homo sapiens
ttagctattcggaacacagaatctc

SEQ ID NO: 2944 - DNA, 25 bp, Homo sapiens
tagctattcggaacacagaatctca

SEQ ID NO: 2945 - DNA, 25 bp, Homo sapiens
agctattcggaacacagaatctcag

SEQ ID NO: 2946 - DNA, 25 bp, Homo sapiens
gctattcggaacacagaatctcagc

SEQ ID NO: 2947 - DNA, 25 bp, Homo sapiens
ctattcggaacacagaatctcagcc

SEQ ID NO: 2948 - DNA, 25 bp, Homo sapiens
tattcggaacacagaatctcagccc

SEQ ID NO: 2949 - DNA, 25 bp, Homo sapiens
attcggaacacagaatctcagccca

SEQ ID NO: 2950 - DNA, 25 bp, Homo sapiens
ttcggaacacagaatctcagcccaa

SEQ ID NO: 2951 - DNA, 25 bp, Homo sapiens
tcggaacacagaatctcagcccaaa

SEQ ID NO: 2952 - DNA, 25 bp, Homo sapiens
cggaacacagaatctcagcccaaac

SEQ ID NO: 2953 - DNA, 25 bp, Homo sapiens
ggaacacagaatctcagcccaaaca

SEQ ID NO: 2954 - DNA, 25 bp, Homo sapiens
gaacacagaatctcagcccaaacaa

SEQ ID NO: 2955 - DNA, 25 bp, Homo sapiens
aacacagaatctcagcccaaacaaa

SEQ ID NO: 2956 - DNA, 25 bp, Homo sapiens
acacagaatctcagcccaaacaaat

SEQ ID NO: 2957 - DNA, 25 bp, Homo sapiens
cacagaatctcagcccaaacaaatg

SEQ ID NO: 2958 - DNA, 25 bp, Homo sapiens
acagaatctcagcccaaacaaatga

SEQ ID NO: 2959 - DNA, 25 bp, Homo sapiens
cagaatctcagcccaaacaaatgat

SEQ ID NO: 2960 - DNA, 25 bp, Homo sapiens
agaatctcagcccaaacaaatgatg

SEQ ID NO: 2961 - DNA, 25 bp, Homo sapiens
gaatctcagcccaaacaaatgatgt

SEQ ID NO: 2962 - DNA, 25 bp, Homo sapiens
aatctcagcccaaacaaatgatgtt

SEQ ID NO: 2963 - DNA, 25 bp, Homo sapiens
atctcagcccaaacaaatgatgttt

SEQ ID NO: 2964 - DNA, 25 bp, Homo sapiens
tctcagcccaaacaaatgatgttta

SEQ ID NO: 2965 - DNA, 25 bp, Homo sapiens
ctcagcccaaacaaatgatgtttac

SEQ ID NO: 2966 - DNA, 25 bp, Homo sapiens
tcagcccaaacaaatgatgtttacc

SEQ ID NO: 2967 - DNA, 25 bp, Homo sapiens
cagcccaaacaaatgatgtttacca

SEQ ID NO: 2968 - DNA, 25 bp, Homo sapiens
agcccaaacaaatgatgtttaccac

SEQ ID NO: 2969 - DNA, 25 bp, Homo sapiens
gcccaaacaaatgatgtttaccact

SEQ ID NO: 2970 - DNA, 25 bp, Homo sapiens
cccaaacaaatgatgtttaccacta

SEQ ID NO: 2971 - DNA, 25 bp, Homo sapiens
ccaaacaaatgatgtttaccactac

SEQ ID NO: 2972 - DNA, 25 bp, Homo sapiens
caaacaaatgatgtttaccactacg

SEQ ID NO: 2973 - DNA, 25 bp, Homo sapiens
aaacaaatgatgtttaccactacgc

SEQ ID NO: 2974 - DNA, 25 bp, Homo sapiens
aacaaatgatgtttaccactacgct

SEQ ID NO: 2975 - DNA, 25 bp, Homo sapiens
acaaatgatgtttaccactacgctg

SEQ ID NO: 2976 - DNA, 25 bp, Homo sapiens
caaatgatgtttaccactacgctgc

SEQ ID NO: 2977 - DNA, 25 bp, Homo sapiens
aaatgatgtttaccactacgctgct

SEQ ID NO: 2978 - DNA, 25 bp, Homo sapiens
aatgatgtttaccactacgctgctc

SEQ ID NO: 2979 - DNA, 25 bp, Homo sapiens
atgatgtttaccactacgctgctcc

SEQ ID NO: 2980 - DNA, 25 bp, Homo sapiens
tgatgtttaccactacgctgctcct

SEQ ID NO: 2981 - DNA, 25 bp, Homo sapiens
gatgtttaccactacgctgctcctc

SEQ ID NO: 2982 - DNA, 25 bp, Homo sapiens
atgtttaccactacgctgctcctcg

SEQ ID NO: 2983 - DNA, 25 bp, Homo sapiens
tgtttaccactacgctgctcctcgt

SEQ ID NO: 2984 - DNA, 25 bp, Homo sapiens
gtttaccactacgctgctcctcgtg

SEQ ID NO: 2985 - DNA, 25 bp, Homo sapiens
tttaccactacgctgctcctcgtgt

SEQ ID NO: 2986 - DNA, 25 bp, Homo sapiens
ttaccactacgctgctcctcgtgtt

SEQ ID NO: 2987 - DNA, 25 bp, Homo sapiens
taccactacgctgctcctcgtgttc

SEQ ID NO: 2988 - DNA, 25 bp, Homo sapiens
accactacgctgctcctcgtgttct

SEQ ID NO: 2989 - DNA, 25 bp, Homo sapiens
ccactacgctgctcctcgtgttctt

SEQ ID NO: 2990 - DNA, 25 bp, Homo sapiens
cactacgctgctcctcgtgttcttc

SEQ ID NO: 2991 - DNA, 25 bp, Homo sapiens
actacgctgctcctcgtgttcttca

SEQ ID NO: 2992 - DNA, 25 bp, Homo sapiens
ctacgctgctcctcgtgttcttcac

SEQ ID NO: 2993 - DNA, 25 bp, Homo sapiens
tacgctgctcctcgtgttcttcact

SEQ ID NO: 2994 - DNA, 25 bp, Homo sapiens
acgctgctcctcgtgttcttcactg

SEQ ID NO: 2995 - DNA, 25 bp, Homo sapiens
cgctgctcctcgtgttcttcactgt

SEQ ID NO: 2996 - DNA, 25 bp, Homo sapiens
gctgctcctcgtgttcttcactgtc

SEQ ID NO: 2997 - DNA, 25 bp, Homo sapiens
ctgctcctcgtgttcttcactgtct

SEQ ID NO: 2998 - DNA, 25 bp, Homo sapiens
tgctcctcgtgttcttcactgtctg

SEQ ID NO: 2999 - DNA, 25 bp, Homo sapiens
gctcctcgtgttcttcactgtctgg

SEQ ID NO: 3000 - DNA, 25 bp, Homo sapiens
ctcctcgtgttcttcactgtctggg

SEQ ID NO: 3001 - DNA, 25 bp, Homo sapiens
tcctcgtgttcttcactgtctgggt

SEQ ID NO: 3002 - DNA, 25 bp, Homo sapiens
cctcgtgttcttcactgtctgggta

SEQ ID NO: 3003 - DNA, 25 bp, Homo sapiens
ctcgtgttcttcactgtctgggtat

SEQ ID NO: 3004 - DNA, 25 bp, Homo sapiens
tcgtgttcttcactgtctgggtatt

SEQ ID NO: 3005 - DNA, 25 bp, Homo sapiens
cgtgttcttcactgtctgggtattt

SEQ ID NO: 3006 - DNA, 25 bp, Homo sapiens
gtgttcttcactgtctgggtatttg

SEQ ID NO: 3007 - DNA, 25 bp, Homo sapiens
tgttcttcactgtctgggtatttgg

SEQ ID NO: 3008 - DNA, 25 bp, Homo sapiens
gttcttcactgtctgggtatttgga

SEQ ID NO: 3009 - DNA, 25 bp, Homo sapiens
ttcttcactgtctgggtatttggag

SEQ ID NO: 3010 - DNA, 25 bp, Homo sapiens
tcttcactgtctgggtatttggagg

SEQ ID NO: 3011 - DNA, 25 bp, Homo sapiens
cttcactgtctgggtatttggagga

SEQ ID NO: 3012 - DNA, 25 bp, Homo sapiens
ttcactgtctgggtatttggaggag

SEQ ID NO: 3013 - DNA, 25 bp, Homo sapiens
tcactgtctgggtatttggaggagg

SEQ ID NO: 3014 - DNA, 25 bp, Homo sapiens
cactgtctgggtatttggaggagga

SEQ ID NO: 3015 - DNA, 25 bp, Homo sapiens
actgtctgggtatttggaggaggaa

SEQ ID NO: 3016 - DNA, 25 bp, Homo sapiens
ctgtctgggtatttggaggaggaac

SEQ ID NO: 3017 - DNA, 25 bp, Homo sapiens
tgtctgggtatttggaggaggaaca

SEQ ID NO: 3018 - DNA, 25 bp, Homo sapiens
gtctgggtatttggaggaggaacaa

SEQ ID NO: 3019 - DNA, 25 bp, Homo sapiens
tctgggtatttggaggaggaacaac

SEQ ID NO: 3020 - DNA, 25 bp, Homo sapiens
ctgggtatttggaggaggaacaacc

SEQ ID NO: 3021 - DNA, 25 bp, Homo sapiens
tgggtatttggaggaggaacaaccc

SEQ ID NO: 3022 - DNA, 25 bp, Homo sapiens
gggtatttggaggaggaacaacccc

SEQ ID NO: 3023 - DNA, 25 bp, Homo sapiens
ggtatttggaggaggaacaaccccc

SEQ ID NO: 3024 - DNA, 25 bp, Homo sapiens
gtatttggaggaggaacaaccccca

SEQ ID NO: 3025 - DNA, 25 bp, Homo sapiens
tatttggaggaggaacaacccccat

SEQ ID NO: 3026 - DNA, 25 bp, Homo sapiens
atttggaggaggaacaacccccatg

SEQ ID NO: 3027 - DNA, 25 bp, Homo sapiens
tttggaggaggaacaacccccatgt

SEQ ID NO: 3028 - DNA, 25 bp, Homo sapiens
ttggaggaggaacaacccccatgtt

SEQ ID NO: 3029 - DNA, 25 bp, Homo sapiens
tggaggaggaacaacccccatgttg

SEQ ID NO: 3030 - DNA, 25 bp, Homo sapiens
ggaggaggaacaacccccatgttga

SEQ ID NO: 3031 - DNA, 25 bp, Homo sapiens
gaggaggaacaacccccatgttgac

SEQ ID NO: 3032 - DNA, 25 bp, Homo sapiens
aggaggaacaacccccatgttgact

SEQ ID NO: 3033 - DNA, 25 bp, Homo sapiens
ggaggaacaacccccatgttgactt

SEQ ID NO: 3034 - DNA, 25 bp, Homo sapiens
gaggaacaacccccatgttgacttg

SEQ ID NO: 3035 - DNA, 25 bp, Homo sapiens
aggaacaacccccatgttgacttgg

SEQ ID NO: 3036 - DNA, 25 bp, Homo sapiens
ggaacaacccccatgttgacttggc

SEQ ID NO: 3037 - DNA, 25 bp, Homo sapiens
gaacaacccccatgttgacttggct

SEQ ID NO: 3038 - DNA, 25 bp, Homo sapiens
aacaacccccatgttgacttggctt

SEQ ID NO: 3039 - DNA, 25 bp, Homo sapiens
acaacccccatgttgacttggcttc

SEQ ID NO: 3040 - DNA, 25 bp, Homo sapiens
caacccccatgttgacttggcttca

SEQ ID NO: 3041 - DNA, 25 bp, Homo sapiens
aacccccatgttgacttggcttcag

SEQ ID NO: 3042 - DNA, 25 bp, Homo sapiens
acccccatgttgacttggcttcaga

SEQ ID NO: 3043 - DNA, 25 bp, Homo sapiens
cccccatgttgacttggcttcagat

SEQ ID NO: 3044 - DNA, 25 bp, Homo sapiens
ccccatgttgacttggcttcagatc

SEQ ID NO: 3045 - DNA, 25 bp, Homo sapiens
cccatgttgacttggcttcagatca

SEQ ID NO: 3046 - DNA, 25 bp, Homo sapiens
ccatgttgacttggcttcagatcag

SEQ ID NO: 3047 - DNA, 25 bp, Homo sapiens
catgttgacttggcttcagatcaga

SEQ ID NO: 3048 - DNA, 25 bp, Homo sapiens
atgttgacttggcttcagatcagag

SEQ ID NO: 3049 - DNA, 25 bp, Homo sapiens
tgttgacttggcttcagatcagagt

SEQ ID NO: 3050 - DNA, 25 bp, Homo sapiens
gttgacttggcttcagatcagagtt

SEQ ID NO: 3051 - DNA, 25 bp, Homo sapiens
ttgacttggcttcagatcagagttg

SEQ ID NO: 3052 - DNA, 25 bp, Homo sapiens
tgacttggcttcagatcagagttgg

SEQ ID NO: 3053 - DNA, 25 bp, Homo sapiens
gacttggcttcagatcagagttggc

SEQ ID NO: 3054 - DNA, 25 bp, Homo sapiens
acttggcttcagatcagagttggcg

SEQ ID NO: 3055 - DNA, 25 bp, Homo sapiens
cttggcttcagatcagagttggcgt

SEQ ID NO: 3056 - DNA, 25 bp, Homo sapiens
ttggcttcagatcagagttggcgtg